(19) United States
(12) Patent Application Publication
(10) Pub. No.: US 2013/0024958 A1
(43) Pub. Date: Jan. 24, 2013

(54) LENTIVIRAL VECTORS THAT PROVIDE IMPROVED EXPRESSION AND REDUCED VARIEGATION AFTER TRANSGENESIS

(71) Applicants: Patrick Stern, Cambridge, MA (US); Stephen Kissler, Prevessin-Moens (FR)

(72) Inventors: Patrick Stern, Cambridge, MA (US); Stephen Kissler, Prevessin-Moens (FR)

(73) Assignee: MASSACHUSETTS INSTITUTE OF TECHNOLOGY, Cambridge, MA (US)

(21) Appl. No.: 13/463,214

(22) Filed: May 3, 2012

Related U.S. Application Data
(63) Continuation of application No. 12/282,466, filed on Apr. 9, 2009, now abandoned.
(60) Provisional application No. 60/783,449, filed on Mar. 17, 2006.

(51) Int. Cl. C12N 15/867; A01K 67/027; C12N 5/10

(52) U.S. Cl. 800/13; 435/320.1; 435/325; 435/349; 435/366; 435/354; 435/456; 800/23

(57) ABSTRACT

The present invention provides new lentiviral vectors that include an anti-repressor element (ARE) and, optionally, a scaffold attachment region (SAR). The lentiviral vectors provide expression of a heterologous nucleic acid in at least 50% of the cells of multiple cell types when used for lentiviral transgenesis. In certain embodiments of the invention the heterologous nucleic acid encodes an RNAi agent such as an shRNA. The invention further provides transgenic nonhuman animals generated using a lentiviral vector that includes an ARE and optional SAR. In addition, the invention provides a variety of methods for using the vectors including for achieving gene silencing in eukaryotic cells and transgenic animals, and methods of treating disease. The invention also provides animal models of human disease in which one or more genes is functionally silenced using a lentiviral vector of the invention.

### RELATED APPLICATIONS

The present application is related to and claims priority under 35 U.S.C. §119(e) to U.S. Ser. No. 60/783,449, filed Mar. 17, 2006 (the '449 application). The entire contents of the '449 application are incorporated herein by reference.

### BACKGROUND OF THE INVENTION

Viral vectors are efficient gene delivery tools in eukaryotic cells. Retroviruses have proven to be versatile and effective gene transfer vectors for a variety of applications since they are easy to manipulate, typically do not induce a strong anti-viral immune response, and are able to integrate into the genome of a host cell, leading to stable gene expression. If provided with an appropriate envelope, retroviruses can infect almost any type of cell. Due to these advantages, a large number of retroviral vectors have been developed for in vitro gene transfer. In addition, use of retroviruses for purposes such as the creation of transgenic or knockout animals or for gene therapy has been explored.

Considerable attention has focused on lentiviruses, a group of complex retroviruses that includes the human immunodeficiency virus (HIV). In addition to the major retroviral genes gag, pol, and env, lentiviruses typically include genes that play regulatory or structural roles. Unlike simple retroviruses, lentiviruses are able to integrate into the genome of non-dividing cells and are thus particularly appealing for applications in which it is desired to transduce a wide variety of cell types. Accordingly, a variety of lentiviral vectors have been developed, and their use for a variety of purposes including creating transgenic animals has been described. However, it has been noted that expression of heterologous sequences by such transgenic animals can be variable both among different cell types or lineages and among cells of a single type or lineage (Lois, 2002; Lu, 2004).

RNA interference (RNAi) has emerged as a rapid and efficient means to silence gene function in eukaryotic (e.g. mammalian and avian) cells. Short interfering RNAs (siRNAs) can silence gene expression in a sequence-specific manner when delivered to mammalian cells. Intracellular expression of short hairpin RNAs (shRNAs) also results in efficient silencing of target genes. However, the use of RNAi, particularly RNAi resulting from expression of transgenes encoding shRNAs in transgenic organisms, has not yet achieved its full promise. Accordingly, there is a need in the art for improved reagents and methods that would facilitate the use of RNAi in transgenic organisms.

### SUMMARY OF THE INVENTION

The present invention provides novel lentiviral vectors and methods of use thereof. In one aspect, the invention provides lentiviral vectors comprising nucleic acid comprising (i) a eukaryotic anti-repressor element (ARE); and (ii) sequences sufficient for reverse transcription and packaging, wherein said sequences are, at least in part, derived from a lentivirus. In certain embodiments of the invention the nucleic acid further comprises a eukaryotic scaffold attachment region (SAR). In certain embodiments of the invention the ARE is derived from either human or mouse genome. The lentiviral vector may be a lentiviral transfer plasmid or an infectious lentiviral particle.

In some aspects, the invention provides cells, e.g., mammalian or avian cells that comprise inventive lentiviral vectors or at least some lentiviral sequences derived therefrom, e.g., a provirus derived therefrom. The invention further provides transgenic non-human animals whose genome comprises a lentivirally transferred transgene and at least some lentiviral sequences. The invention further provides methods for making transgenic non-human animals, the cells of which comprise a lentivirally transferred transgene and at least some lentiviral sequences.

In some aspects, the invention provides methods of expressing a heterologous nucleic acid in a target cell comprising (i) introducing a lentiviral vector of the invention into a target cell, wherein the lentiviral vector comprises a nucleic acid comprising regulatory sequences for transcription operably linked to a heterologous nucleic acid; and (ii) expressing the heterologous nucleic acid in the cell. In certain embodiments of the invention, the heterologous nucleic acid encodes an RNAi agent, e.g., an shRNA.

In some aspects, the invention provides methods of silencing a gene in a target cell comprising (i) introducing a lentiviral vector of the invention into a target cell, wherein the lentiviral vector comprises a nucleic acid comprising regulatory sequences for transcription operably linked to a nucleic acid that encodes an RNAi agent targeted to the gene; and (ii) expressing the nucleic acid in the cell, thereby producing an RNAi agent that inhibits expression of the target gene. The RNAi agent may be an shRNA. The target gene may be a disease-associated gene.

The invention further provides a transgenic nonhuman animal that expresses a lentivirally transferred transgene, wherein at least 50% of the cells of 2, 3, 4, or more different cell types in an animal express the transgene. In certain embodiments of the invention, the transgene is expressed in at least 50% of peripheral white blood cells, e.g., between 50% and 90% of peripheral white blood cells express the transgene. In certain embodiments of the invention, between 50% and 90% of the cells of 2, 3, 4, or more different cell types in an animal express the transgene.

The invention provides methods of creating infectious lentiviral particles and of creating producer cell lines that produce infectious lentiviral particles. Lentiviral particles may, but need not be, derived from lentiviral transfer plasmids, described herein.

The invention further provides methods for expressing a heterologous nucleic acid in a target cell comprising introducing a lentiviral vector of the invention into a target cell and expressing a heterologous nucleic acid therein. In various embodiments of the invention, the heterologous nucleic acid is operably linked to a constitutive, a regulatable, or a cell type specific, lineage specific, or tissue specific promoter, allowing conditional expression of the nucleic acid.

In one aspect, the invention provides methods for achieving controlled expression of a heterologous nucleic acid in a cell comprising steps of: (i) introducing a lentiviral vector of the invention that comprises a heterologous nucleic acid located between sites for a recombinase to a cell and; (ii) subsequently inducing expression of the recombinase within the cell, thereby preventing expression of the heterologous nucleic acid within the cell.

In another aspect, the invention provides a lentiviral vector comprising a nucleic acid that comprises an ARE and, optionally, a SAR, wherein the lentiviral vector comprises regulatory sequences for transcription operably linked to a nucleic acid segment that encodes an RNAi agent or strand thereof. Following introduction of the vector into a cell, transcription of one or more ribonucleic acids (RNAs) that self-hybridize or hybridize to each other results in formation of an RNAi agent such as a short hairpin RNA (shRNA) or short interfering RNA (siRNA) that inhibits expression of at least one target transcript in the cell. In certain embodiments of the invention, the lentiviral vector comprises a nucleic acid segment operably linked to a promoter, so that transcription directed by the promoter results in synthesis of an RNA comprising complementary regions that hybridize to form an shRNA targeted to a target transcript. According to certain embodiments of the invention, an shRNA comprises a base-paired region between about 17-29 nucleotides in length, e.g., approximately 19 nucleotides long. In certain embodiments of the invention, a lentiviral vector comprises a nucleic acid segment flanked by two promoters in opposite orientation, wherein the promoters are operably linked to the nucleic acid segment, so that transcription from the promoters results in synthesis of two complementary RNAs that hybridize with each other to form an siRNA targeted to the target transcript. According to certain embodiments of the invention, an siRNA comprises a base-paired region between about 17-29 nucleotides in length, e.g., approximately 19 nucleotides long. In certain embodiments of the invention, a lentiviral vector comprises at least two promoters and at least two nucleic acid segments, wherein each promoter is operably linked to a nucleic acid segment, so that transcription from the promoters results in synthesis of two complementary RNAs that hybridize with each other to form an siRNA targeted to the target transcript.

Lentiviral vectors of the invention may be lentiviral transfer plasmids or infectious lentiviral particles. Where reference is made herein to elements such as cloning sites, promoters, regulatory elements, heterologous nucleic acids, etc., it is to be understood that the sequences of these elements are present in RNA form in the lentiviral particles of the invention and are present in DNA form in the DNA plasmids of the invention. Furthermore, where a sequence such as a sequence that encodes an RNAi agent is provided to a cell by a lentiviral particle, it is understood that the lentiviral RNA must undergo reverse transcription and second strand synthesis to produce DNA.

The invention further provides pharmaceutical compositions comprising any of the inventive lentiviral vectors and one or more pharmaceutically acceptable carriers.

The invention includes a variety of therapeutic applications for inventive lentiviral vectors. In particular, lentiviral vectors are useful for gene therapy. The invention provides methods of treating and/or preventing infection by an infectious agent, the method comprising administering to a subject prior to, simultaneously with, or after exposure of the subject to the infectious agent a composition comprising an effective amount of a lentiviral vector, wherein the lentiviral vector directs transcription of at least one RNA that hybridizes to form an shRNA or siRNA that is targeted to a transcript produced during infection by the infectious agent, which transcript is characterized in that reduction in levels of the transcript delays, prevents, and/or inhibits one or more aspects of infection by and/or replication of the infectious agent.

The invention provides methods of treating a disease or clinical condition, the method comprising: (i) removing a population of cells from a subject at risk of or suffering from the disease or clinical condition; (ii) engineering or manipulating the cells to comprise an effective amount of an RNAi agent targeted to a transcript by infecting or transfecting the cells with a lentiviral vector, wherein the transcript is characterized in that its degradation delays, prevents, and/or inhibits one or more aspects of the disease or clinical condition; (iii) and returning at least a portion of the cells to the subject. Suitable lentiviral vectors are described herein. Without limitation, therapeutic approaches may find particular use in diseases such as cancer, in which a mutation in a cellular gene is responsible for or contributes to the pathogenesis of the disease, and in which specific inhibition of the target transcript bearing the mutation may be achieved by expressing an RNAi agent targeted to the target transcript within the cells, without interfering with expression of the normal (i.e. non-mutated) allele. According to certain embodiments of the invention, rather than removing cells from the body of a subject, infecting or transfecting them in tissue culture, and then returning them to the subject, inventive lentiviral vectors or lentiviruses are delivered directly to the subject.

In certain embodiments of the invention, lentiviral vectors are an improvement relative to lentiviral vectors known in the art in at least one of the following respects: (i) they comprise an ARE and, in certain embodiments a SAR; (ii) they provide enhanced expression after lentiviral transgenesis; (iii) the provide reduced variegation after lentiviral transgenesis. In certain embodiments of the invention the transgenie animals are an improvement relative to transgenic animals generated using lentiviral vectors known in the art in at least one of the following respects: (i) they comprise higher percentages of cells (e.g., cells of at least 1, 2, 3, 4, or more cell types) that express a transgene of interest than do transgenic animals generated using lentiviral vectors known in the art; (ii) they comprise higher percentages of cells (e.g., cells of at least 1, 2, 3, 4, or more cell types) in which expression of a gene of interest is inhibited by a lentivirally transferred RNAi agent than do transgenic animals generated using lentiviral vectors known in the art; (iii) they display reduced variegation relative to transgenic animals generated using lentiviral vectors known in the art.

This application refers to various patents, journal articles, and other publications, all of which are incorporated herein by reference. In addition, the following publications are incorporated herein by reference: Current Protocols in Molecular Biology, Current Protocols in Immunology, Current Protocols in Protein Science, and Current Protocols in Cell Biology, John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Sohail, M. (ed.) Gene silencing by RNA interference: technology and application, Boca Raton, Fla.: CRC Press, 2005; Engelke, D R (ed.) RNA interference (RNAi): nuts & bolts of RNAi technology; Eagleville, Pa.: DNA Press, 2003. In the event of a conflict or inconsistency between any of the incorporated references and the instant specification or the understanding of one of ordinary skill in the art, the specification shall control. The determination of whether a conflict or inconsistency exists is within the discretion of the inventors and can be made at any time.

### BRIEF DESCRIPTION OF THE DRAWING

FIG. 1: Map of the lentivirus vector pLL3.7.

FIG. 2: Schematic diagrams of the HIV provirus (upper panel) and relevant portions of representative packaging and Env-coding plasmids (middle and lower panels, respectively) for a three plasmid system.

FIG. 3: Structure of an exemplary siRNA.

FIG. 4: Schematic diagrams of structures of a variety of exemplary shRNAs.

FIG. 5: Schematic diagram of a nucleic acid that can serve as a template for transcription of an RNA that hybridizes to form an shRNA and shows the RNA before and after hybridization.

FIG. 6a: Schematic representation of a portion of the lentivirus vector pLL3.7. Key: SIN-LTR: self-inactivating long terminal repeat; Ψ: HIV packaging signal; cPPT: central polypurine tract; U6: U6 (RNA polymerase III) promoter; MCS: multiple cloning site; CMV: cytomegalovirus (RNA polymerase II) promoter; EGFP: enhanced green fluorescent protein; WRE: woodchuck hepatitis virus response element.

FIG. 6b: Sequence of the CD8 stem loop used to generate pLL3.7 CD8. A sequence known to silence CD8 as an siRNA (McManus, 2002) was adapted with a loop sequence (from Paddison, 2002) to create the final sequence. The presumed transcription initiation site is indicated by a+1. Nucleotides which form the loop structure are indicated (loop). A pol III terminator (a sequence of Us in the RNA) is indicated (terminator).

FIG. 6c: Predicted structure of the CD8 stem-loop RNA produced from pLL3.7 CD8.

FIG. 6d: Nramp1 stem loop sequence used to generate pLB-Nramp1-915. The presumed transcription initiation site is indicated by a+1. Nucleotides which form the loop structure are indicated (loop). The poi III terminator (a sequence of Us in the RNA) is indicated (terminator). The lower portion of the figure shows the Nramp1 shRNA predicted to form following transcription.

FIGS. 7a-7d: Protective effect of the B10-derived Idd5.2 allele. (a) Schematic representation of the Idd5.1 (2.1 Mb) and Idd5.2 (1.52 Mb) B10-derived regions (filled area) on chromosome 1 in NOD congenic mice. The Idd5.2 region contains 42 genes, including Nramp1. F1 mice are B10 homozygous at Idd5.1 and heterozygous at Idd5.2. (b) Percent survival over time in Idd5.1 Idd5.1/Idd5.2 (n=55), and F1 (n=71) mice. (c) Schematic representation of the chromosome 1 region in Idd5.2 congenic mice. Filled regions are B10-derived. (d) Percent survival over time in NOD (n=67), Idd5.2 (n=67), and Idd5.2 heterozygous (n=53) female mice. Differences were analyzed using the Gehan-Wilcoxon test: NOD vs. Idd5.1 P<0.0001; NOD vs. NODx Idd5.2 P=0.0021; Idd5.2 vs. NOD xldd5.2 P=0.0521.

FIGS. 8a-8d: Design of a lentiviral vector for Nramp1 knock-down and demonstration of its effectiveness in reducing Nramp1 levels in hematopoietic cells in) transgenic mice created using the vector. (a) Peripheral blood from a pLL3.7-CD8 shRNA lentiviral transgenic NOD mouse (right panels) and a non-transgenic littermate (left panels) was analyzed by flow cytometry. Top panels: CD3 expression in the lymphocyte population. Middle panels: CD4 and GFP expression (gated on CD4⁺ cells). Bottom panels: CD8 and GFP expression (gated on CD8⁺ cells). (b) Schematic representation of pLL3.7 and of the new pLB vector that comprises the anti-repressor element #40 and scaffold-attached region (SAR). U6 and CMV promoters drive shRNA and GFP expression, respectively. (c) Peripheral blood from a pLB lentiviral transgenic NOD mouse (right panels) and a non-transgenic littermate (left panels) was stained for TCR (T cell marker), B220 (B cell marker) and CD11b (macrophage marker) for analysis by flow cytometry. The top, middle and bottom panels are gated on TCR⁺, B220⁺, and B220⁻ CD11b⁺ cells, respectively. Lineage marker and GFP expression are shown for each population. (d) 293FT cells were co-transfected with a Renilla/firefly dual-luciferase reporter, in which Nramp1 cDNA was present or absent, together with pLB vectors comprising different shRNA sequences against Nramp1. Relative luminescence units (RLU) generated by Renilla luciferase activity for each lysate (normalized for firefly luciferase activity) are shown +/−SEM.

FIG. 9a: Variegated expression in pLL3.7 transgenic mice as demonstrated by analysis of GFP expression in the peripheral blood of a pLL3.7 transgenic male founder and of its progeny by flow cytometry. Percentage GFP-positive cells is indicated for each sample.

FIG. 9b: Lentiviral construct expression in pLB-915 transgenic founder. Flow cytometry of peripheral blood cells from the pLB-915 transgenic founder Idd5.1 congenic mouse (right panels) and non-transgenic littermate (left panels). Panels from top to bottom were gated on B cells (B220⁺), T cells (CD4⁺ and CD8⁺), and macrophages (B220⁻ CD11b⁺), respectively. Lineage marker and GFP expression are shown for each population.

FIGS. 10a-10c: Silencing of Nramp expression in cells of various lineages isolated from Nramp1 knock-down (KD) Idd5.1 congenic NOD mice. (a) Expression of the OFF marker in peripheral blood cells from the pLB-915 founder (F0) and positive mice in subsequent generations. F1: n=17, F2: n=100, F3: n=10, F4 n=6. Horizontal bar denotes mean percentage of GFP-positive cells. (b) Flow cytometry analysis of lymph node cells from a pLB-915 F2 mouse (NRAMP1 KD, right panels) and non-transgenic littermate (control, left panels). Panels from top to bottom were gated on 13 cells (B220⁺,) T cells (CD4⁺ and CD8⁺), and macrophages (B220⁺ CD11b⁺), respectively. Lineage marker and GFP expression are shown for each population. (c) Western blot analysis of cell lysates from activated peritoneal macrophages (control: non-transgenic littermate; NRAMP1 KD: pLB-915 F2 transgenic).

FIGS. 11a-11b: Effect of Nramp1 silencing on Salmonella enterica infection and diabetes frequency. (a) pLB-915 transgenic Idd5.1 males (Idd5.1 KD, n=8), their non-transgenic male littermates (Idd5.1, n=8), and Idd5.1/Idd5.2 male mice (n=7) were injected intravenously with approximately 10⁷ colony forming units (CFU) of Salmonella enterica. Mice were monitored daily for survival: Combined survival curves from two similar experiments are shown. Logrank-test: P=0.0477 between Idd5.1 and Idd5.1 KD groups. (b) The frequency of diabetes was determined in cohorts of female pLB-915 transgenic Idd5.1 mice (Idd5.1 KD, n=37) and their female non-transgenic littermates (Idd5.1, n=56). Survival curves are shown. Logrank-test: P=0.0027.

FIGS. 12a-12b: Reduced expression possibly caused by interference between lentiviral constructs. (a) Expression of GFP in peripheral blood cells from F0 founder pLB-915 Idd5.1 congenic mouse, F1, F2, F3 and F4 mice (out-bred to non-transgenic Idd5.1 congenic mice), and progeny of F1×F1 and F3×F3 crosses. The percent GFP positive cells in hematopoietic cells is shown. (b) Southern blot of EcoRI-digested genomic DNA from GFP positive and negative pLB-915 progeny. The locus found in all positives, but not in negative mice is indicated (star). For F1×F1 progeny, a low expressor (46%) and high expressor (71%) are shown. The intensity of the bands that correlate with expression suggests a homozygous genotype in the low expressing mouse and a heterozygous genotype of the high expressor. c F3×F3 male mice with expression in either a high (73%) or low (40%) percentage of cells were bred with non-transgenic females. Off-spring from the high-expressing (High) and low expressing (Low) breeders were tested for expression, and all GFP-positive animals were found to express high levels (73% and 74% average, respectively), suggesting that segregation of the homozygous lentiviral integrants re-established full expression.

FIG. 13: Mean EAE score of Idd5.1 Nramp1 knock-down mice (Idd5.1 KD, n=13) and non-transgenic Idd5.1 littermates (n=18), demonstrating that Nramp1 gene silencing protects against experimental autoimmune encephalomyelitis (EAE).

FIG. 14: Sequence of a mouse anti-repressor element (SEQ ID NO: 122), which is a fragment of mouse ARE 40.

FIG. 15: Sequences of additional AREs of use in the invention.

FIG. 16. Lentiviral construct expression in pLB-915 transgenic heterozygotes and homozygotes. Flow cytometry of peripheral blood cells from progeny from a cross between a non-transgenic male and a heterozygous pLB-915 transgenic founder Idd5.1 congenic mouse (top panels). Flow cytometry of peripheral blood cells from progeny from a cross between two heterozygous pLB-915 transgenic founder Idd5.1 congenic mice (bottom panels). GFP expression is shown for each population. The number in the lower right corner represents the percent of peripheral blood cells expressing GFP.

### DEFINITIONS

“Approximately” or “about” in reference to a number generally includes numbers that fall within a range of 5% of the number in either direction (greater than or less than the number) unless otherwise stated or otherwise evident from the context (except where such number would exceed 100% of a possible value).

“Cell type” is used herein consistently with its meaning in the art to refer to a form of cell having a distinct set of morphological, biochemical, and/or functional characteristics that define the cell type. One of skill in the art will recognize that a cell type can be defined with varying levels of specificity. For example, B cells and T cells are distinct cell types, which can be distinguished from one another but share certain features that are characteristic of the broader “lymphocyte” cell type of which both are members. Typically, cells of different types may be distinguished from one another based on their differential expression of a variety of genes which are referred to in the art as “markers” of a particular cell type or types (e.g., cell types of a particular lineage). A cell type specific marker is a gene product or modified version thereof that is expressed at a significantly greater level by one or more cell types than by all or most other cell types and whose expression is characteristic of that cell type. Many cell type specific markers are recognized as such in the art. Similarly, a lineage specific marker is one that is expressed at a significantly greater level by cells of one or more lineages than by cells of all or most other lineages. A tissue specific marker is one that is expressed at a significantly greater level by cells of a type that is characteristic of a particular tissue than by cells that are characteristic of most or all other tissues.

“Complementary” is used herein in accordance with its art-accepted meaning to refer to the capacity for precise pairing between particular bases, nucleosides, nucleotides or nucleic acids via formation of hydrogen bonds. For example, adenine (A) and uridine (U), adenine (A) and thymidine (T), or guanine (G) and cytosine (C), are complementary to one another. If a nucleotide at a certain position of a first nucleic acid is complementary to a nucleotide located opposite in a second nucleic acid, the nucleotides form a complementary base pair, and the nucleic acids are complementary at that position. One of ordinary skill in the art will appreciate that the nucleic acids are aligned in antiparallel orientation (i.e., one nucleic acid is in 5′ to 3′ orientation while the other is in 3′ to 5′ orientation).

The term “defective” as used herein with respect to a nucleic acid refers to a nucleic acid that is modified with respect to a wild type sequence such that the nucleic acid does not encode a functional gene product that would be encoded by the wild type sequence or does not perform the function of the wild type sequence. For example, a defective env gene sequence does not encode a functional Env protein; a defective packaging signal will not facilitate the packaging of a nucleic acid molecule that includes the defective packaging signal; a defective polyadenylation sequence will not promote polyadenylation of a nucleic acid comprising the sequence. A nucleic acid may be defective for some but not all of its functions. For example, a defective LTR may fail to promote transcription of downstream sequences while still retaining the ability to direct integration. Nucleic acid sequences may be made defective by any means known in the art, including by mutagenesis, by the deletion of some or all of the sequence, by inserting a heterologous sequence into the nucleic acid sequence, by placing the sequence out-of-frame, or by otherwise blocking the sequence. Defective sequences may also occur naturally, i.e., without human intervention, such as by mutation, and may be isolated from viruses in which they arise. Proteins that are encoded by a defective nucleic acid and are therefore not functional may be referred to as defective proteins. A virus or viral particle is “defective” with respect to particular function if it is unable to perform the function. For example, a virus or viral particle is replication defective if it cannot produce infectious viral particles following its introduction into a cell. It is to be understood that the term “defective” is relative. In other words, the function need not be completely eliminated but is typically substantially reduced relative to the comparable wild type function. Generally, a defective entity exhibits less than approximately 10%, less than approximately 5%, less than approximately 2%, less than approximately 1%, less than approximately 0.5%, or approximately 0%, i.e., below the limits of detection, of the function of the comparable wild type entity.

A “disease-associated” gene is a gene whose expression or lack thereof contributes to or is essential to an unwanted cellular or organismal phenotype, e.g., aberrant expression of the gene is at least in part responsible for causing an undesirable disease state or condition or a manifestation thereof. The gene may be one that is or becomes expressed at an abnormally high level or one that is or becomes expressed at an abnormally low level, where the altered expression correlates with and is generally at least in part responsible for the occurrence and/or progression of the disease or wherein expression of a particular allele or mutant form of the gene correlates with and is generally at least in part responsible for the occurrence and/or progression of the disease. Also encompassed are genes wherein expression of an allele of the gene has a protective effect, e.g., individuals who express the allele have reduced susceptibility to an undesirable disease state or condition or a manifestation thereof, relative to the susceptibility of individuals who do not express the allele or express an alternate allele. A disease-associated gene also refers to genes possessing mutation(s) or genetic variation that is in linkage disequilibrium with a gene whose aberrant expression is at least in part responsible for the occurrence, progression, or any manifestation of a disease. The expression product(s) of such disease-associated genes may be known or unknown, and may be at normal or abnormal level.

The term “encode” is used herein to refer to the capacity of a nucleic acid to serve as a template for transcription of RNA or the capacity of a nucleic acid to be translated to yield a polypeptide. Thus a DNA sequence that is transcribed to yield an RNA is said to “encode” the RNA. If a nucleic acid sequence is transcribed to yield an RNA that is translated to yield a polypeptide, both the nucleic acid and the RNA are said to encode the polypeptide. “Transcription” as used herein includes reverse transcription, where appropriate.

The phrase “essential lentiviral protein” as used herein refer to those viral protein(s), other than envelope protein, that are required for the lentiviral life cycle. Essential lentiviral proteins include those required for reverse transcription and integration and for the encapsidation (e.g., packaging) of a retroviral genome.

“Expression” typically refers to the production of one or more particular RNA product(s), polypeptides(s) and/or protein(s), in a cell. In the case of RNA products, it refers to the process of transcription. In the case of polypeptide products, it refers to the processes of transcription, translation and, optionally, post-translational modifications (e.g., glycosylation, phosphorylation, etc.), and/or assembly into a multimeric protein in the case of polypeptides that are components of multimeric proteins. With respect to a gene, “expression” refers to transcription of at least a portion of the gene and, where appropriate, translation of the resulting mRNA transcript to produce a polypeptide. A transferred gene, or transgene, is “expressed” in a cell (or in a descendant of the cell into which the physical nucleic acid material was introduced) if the cell produces an expression product of the gene (e.g., an RNA transcript and/or a polypeptide). At least a portion of the gene is used as a template for transcription of an RNA, which may then translated in the case of mRNA. In the case of DNA, a transferred gene may be integrated into the cell's genomic DNA prior to transcription. In the case of transfer of a lentiviral genome or portion thereof by a lentiviral vector, the transferred RNA is reverse transcribed prior to integration. An “expression cassette” is a nucleic acid sequence capable of providing expression of an RNA and, optionally, a polypeptide encoded by the RNA in the case of a nucleic acid sequence that comprises an open reading frame. An expression cassette typically comprises a functional promoter, a portion that encodes an RNA of interest, and a functional terminator, all in operable association. A functional promoter is a promoter that is capable of initiating transcription in a particular cell under appropriate conditions, which may include the presence of an inducing agent in the case of a regulatable promoter. In certain embodiments of the present invention, a gene that is transferred to a cell (or to an ancestor of the cell) is considered to be “expressed” by the cell if an RNA and/or protein expression product of the gene can be directly or indirectly detected in the cell (or, as appropriate, on the cell surface or secreted by the cell) by any suitable means of detection at a level at least 5-fold as great as the background level that would be detected in otherwise similar or identical cells that do not comprise an endogenous or heterologous copy of the gene or at a level at least 20% greater than the level that would be detected in otherwise similar or identical cells that comprise an endogenous copy of the gene. As will be evident, expression of a gene that encodes an RNAi agent may be detected by detecting a decrease in the level of a target transcript or its encoded protein or by detecting a phenotypic consequence of such decreased level. In certain embodiments of the present invention, a cell is considered to express a transferred gene that encodes an RNAi agent if the level of a target transcript or its encoded protein in the cell is decreased by at least approximately 20% to approximately 100% relative to the level of the target transcript or its encoded protein that would be detected in otherwise similar or identical cells that do not comprise a copy of the gene encoding the RNAi agent and/or if the level of a target transcript or its encoded protein is decreased by at least approximately 50% of the decrease that would be observed if otherwise similar or identical cells were exposed in culture, under conditions accepted in the art as being suitable for efficient siRNA uptake, to an siRNA having an antisense strand that comprises a sequence identical to at least the portion of the RNAi agent that hybridizes with the target transcript. It will be appreciated that in the case of cell surface or secreted proteins “in the cell” includes, as appropriate, protein on the cell surface or secreted by the cell.

The term “gene” refers to a nucleic acid comprising a nucleotide sequence that encodes a polypeptide or a biologically active ribonucleic acid (RNA) such as a tRNA, shRNA, miRNA, etc. The nucleic acid can include regulatory elements (e.g., expression control sequences such as promoters, enhancers, etc.) and/or introns.

A “gene product” or “expression product” of a gene is an RNA transcribed from the gene (e.g., pre- or post-processing) or a polypeptide encoded by an RNA transcribed from the gene (e.g., pre- or post-modification).

“Hematopoietic cells” are cell types found in the blood and/or lymph. These cell types include the myeloid cells (erythrocytes, thrombocytes, granulocytes (neutrophils, eosinophils, basophils) monocytes and macrophages, mast cells) and the lymphoid cells (B cells, various types of T cells, NK cells). These cells typically arise from hematopoietic stem cells in the bone marrow. It will be appreciated that certain hematopoietic cells, e.g., macrophages, may be present in tissues outside of the vascular or lymphatic systems. White blood cells (e.g., granulocytes (neutrophils, eosinophils, basophils, monocytes, macrophages, mast cells, and lymphoid cells) are a subset of hematopoietic cells.

The term “heterologous” as used herein in reference to a nucleic acid, refers to a first nucleic acid that is inserted into a second nucleic acid such as a plasmid or other vector. For example, the term refers to a nucleic acid that is not naturally present in a wild type vector from which a recombinant vector is derived. The term also refers to a nucleic acid that is introduced into a cell, tissue, organism, etc., by artificial means including, but not limited to, transfection or infection with a viral vector. Generally the heterologous nucleic acid is either not naturally found in the cell, tissue, or organism or, if naturally found therein, its expression is altered by introduction of the additional copy of the nucleic acid or it is present at a different location in the genome. The term “heterologous polypeptide” is used to refer to a polypeptide encoded by a heterologous nucleic acid. If a heterologous sequence is introduced into a cell or organism, the sequence is also considered heterologous to progeny of the cell or organism that inherit it.

“Infectious,” as used herein in reference to a recombinant lentivirus or lentiviral particle, indicates that the lentivirus or lentiviral particle is able to enter cells and to perform at least one of the functions associated with infection by a wild type lentivirus, e.g., release of the viral genome in the host cell cytoplasm, entry of the viral genome into the nucleus, reverse transcription, and/or integration of the viral genome into the host cell's DNA. It is not intended to indicate that the virus or viral particle is capable of undergoing replication or of completing the viral life cycle.

“Inhibition of gene expression” refers to the absence of an mRNA and/or polypeptide expression product of a target gene or to an observable decrease in the level of the expression product. Typically the level will be reduced by at least approximately 50%, at least approximately 60%, at least approximately 70%, at least approximately 80%, at least approximately 90%, or more relative to the level in the absence of an inhibitory agent such as an RNAi agent. “Specificity” refers to the ability to inhibit expression of a target gene without significant or equivalent effects on most or all other genes of the cell. Methods for determining the extent of inhibition include examining one or more relevant phenotypes, e.g., by detecting visible consequences of inhibition or through the use of techniques such as RNA solution hybridization, nuclease protection, Northern hybridization, reverse transcription, gene expression monitoring with a microarray, antibody binding, enzyme linked immunosorbent assay (ELISA), Western blotting, immunoassay (RIA), fluorescence activated cell analysis (FACS), etc.

“Lineage” refers to a set of cell types that are committed to or capable of differentiating into a particular fully differentiated cell type.

A “microRNA” (miRNA) is a naturally occurring single-stranded RNA molecule that is generated by intracellular processing of an endogenous precursor RNA containing a stem-loop (hairpin) structure. An miRNA hybridizes with a target site in a target transcript and reduces expression of the target transcript by translational repression, i.e., it blocks or prevents translation. Both the stem of the precursor RNA and the duplex formed by the miRNA and the target transcript are imperfect and typically comprise up to several areas of mismatched or unpaired nucleotides that form bulges. Bulges may, for example, comprise at least two consecutive noncomplementary base pairs exist or include one or more “extra” unpaired nucleotide(s) located between two regions of perfect base pair complementarity. Nucleic acid molecules or precursors thereof that mimic the sequence of naturally occurring miRNA precursors or are designed to form a similar structure when self-hybridized or hybridized to a target transcript can be introduced into or expressed within cells and can cause translational repression (See, e.g., Doench, J., et al., Genes and Dev., 17:438-442, 2003). A nucleic acid that mediates RNAi by repressing translation of a target transcript, and that comprises a portion that binds to a target transcript to form a duplex structure comprising one or more bulges, resembling that formed by an miRNA and its target transcript, is said herein to act via an miRNA translational repression pathway, and the portion that binds to the target may be referred to as an miRNA-like molecule. A description and examples of miRNAs and the mechanism by which they mediate silencing are found in Lagos-Quintana, M., et al., RNA, 9(2):175-9, 2003; and Bartel, D., Cell, 116:281-297, 2004.

The term “non-dividing cell” refers to a cell that does not go through mitosis. Non-dividing cells may be blocked at any point or within any stage in the cell cycle as long as the cell is not actively progressing through the cell cycle. The cell may be naturally non-dividing or its division may be blocked by any of a variety of treatments known in the art.

The term “nucleic acid” refers to polynucleotides such as DNA or RNA. Nucleic acids can be single-stranded, partly or completely, double-stranded, and in some cases partly or completely triple-stranded. Nucleic acids include genomic DNA, cDNA, mRNA, etc. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. The term “nucleic acid sequence” as used herein can refer to the nucleic acid material itself and is not restricted to the sequence information (i.e. the succession of letters chosen among the five base letters A, G, C, T, or U) that biochemically characterizes a specific nucleic acid, e.g., a DNA or RNA molecule. A nucleic acid sequence is presented in the 5′ to 3′ direction unless otherwise indicated. The term “nucleic acid segment” is used herein to refer to a nucleic acid sequence that is a portion of a longer nucleic acid sequence.

“Operably linked” or “operably associated” refers to a functional relationship between two nucleic acids, wherein the expression, activity, localization, etc., of one of the sequences is controlled by, directed by, regulated by, modulated by, etc., the other nucleic acid. The two nucleic acids are said to be operably linked or operably associated or in operable association. “Operably linked” or “operably associated” can also refers to a relationship between two polypeptides wherein the expression of one of the polypeptides is controlled by, directed by, regulated by, modulated by, etc., the other polypeptide. For example, transcription of a nucleic acid is directed by an operably linked promoter; post-transcriptional processing of a nucleic acid is directed by an operably linked processing sequence; translation of a nucleic acid is directed by an operably linked translational regulatory sequence such as a translation initiation sequence; transport, stability, or localization of a nucleic acid or polypeptide is directed by an operably linked transport or localization sequence such as a secretion signal sequence; and post-translational processing of a polypeptide is directed by an operably linked processing sequence. Typically a first nucleic acid sequence that is operably linked to a second nucleic acid sequence, or a first polypeptide that is operatively linked to a second polypeptide, is covalently linked, either directly or indirectly, to such a sequence, although any effective three-dimensional association is acceptable. One of ordinary skill in the art will appreciate that multiple nucleic acids, or multiple polypeptides, may be operably linked or associated with one another.

As used herein, a “packaging signal,” “packaging sequence,” or “psi sequence” is any nucleic acid sequence sufficient to direct packaging of a nucleic acid whose sequence comprises the packaging signal into a retroviral particle. The term includes naturally occurring packaging sequences and also engineered variants thereof. Packaging signals of a number of different retroviruses, including lentiviruses, are known in the art.

“Recombinant” is used consistently with its usage in the art to refer to a nucleic acid sequence that comprises portions that do not naturally occur together as part of a single sequence or that have been rearranged relative to a naturally occurring sequence. A recombinant nucleic acid is created by a process that involves the hand of man and/or is generated from a nucleic acid that was created by hand of man (e.g., by one or more cycles of replication, amplification, transcription, etc.). A recombinant virus is one that comprises a recombinant nucleic acid. A recombinant cell is one that comprises a recombinant nucleic acid.

The term “regulatory sequence” or “regulatory element” is used herein to describe a nucleic acid sequence that regulates one or more steps in the expression (particularly transcription, but in some cases other events such as splicing or other processing) of nucleic acid sequence(s) with which it is operatively linked. The term includes promoters, enhancers and other transcriptional control elements that direct or enhance transcription of an operatively linked nucleic acid. Regulatory sequences may direct constitutive expression (e.g., expression in most or all cell types under typical physiological conditions in culture or in an organism), cell type specific, lineage specific, or tissue specific expression, and/or regulatable (inducible or repressible) expression. For example, expression may be induced or repressed by the presence or addition of an inducing agent such as a hormone or other small molecule, by an increase in temperature, etc. Non-limiting examples of cell type, lineage, or tissue specific promoters appropriate for use in mammalian cells include lymphoid-specific promoters (see, for example, Calame et al., Adv. Immunol. 43:235, 1988) such as promoters of T cell receptors (see, e.g., Winoto et al., EMBO J. 8:729, 1989) and immunoglobulins (see, for example, Banerji et al., Cell 33:729, 1983; Queen et al., Cell 33:741, 1983), and neuron-specific promoters (e.g., the neurofilament promoter; Byrne et al., Proc. Natl. Acad. Sci. USA 86:5473, 1989). Developmentally-regulated promoters include hox promoters (see, e.g., Kessel et al., Science 249:374, 1990) and the α-fetoprotein promoter (Campes et al., Genes Dev. 3:537, 1989). Some regulatory elements may inhibit or decrease expression of an operatively linked nucleic acid. Such regulatory elements may be referred to as “negative regulatory elements.” A regulatory element whose activity can be induced or repressed by exposure to an inducing or repressing agent and/or by altering environmental conditions is referred to herein as a “regulatable” element.

“RNAi agent” refers to an at least partly double-stranded RNA having a structure characteristic of molecules that are known in the art to mediate inhibition of gene expression through an RNAi mechanism or an RNA strand comprising at least partially complementary portions that hybridize to one another to form such a structure. When an RNA comprises complementary regions that hybridize with each other, the RNA will be said to self-hybridize. An RNAi agent includes a portion that is substantially complementary to a target gene. An RNAi agent optionally includes one or more nucleotide analogs or modifications. One of ordinary skill in the art will recognize that RNAi agents that are synthesized in vitro can include ribonucleotides, deoxyribonucleotides, nucleotide analogs, modified nucleotides or backbones, etc., whereas RNAi agents synthesized intracellularly, e.g., encoded by DNA templates, typically consist of RNA, which may be modified following transcription. Of particular interest herein are short RNAi agents, i.e., RNAi agents consisting of one or more strands that hybridize or self-hybridize to form a structure that comprises a duplex portion between about 15-29 nucleotides in length, optionally having one or more mismatched or unpaired nucleotides within the duplex. RNAi agents include short interfering RNAs (siRNAs), short hairpin RNAs (shRNAs), and other RNA species that can be processed intracellularly to produce shRNAs including, but not limited to, RNA species identical to a naturally occurring miRNA precursor or a designed precursor of an miRNA-like RNA.

The term “short, interfering RNA” (siRNA) refers to a nucleic acid that includes a double-stranded portion between about 15-29 nucleotides in length and optionally further comprises a single-stranded overhang (e.g., 1-6 nucleotides in length) on either or both strands. The double-stranded portion is typically between 17-21 nucleotides in length, e.g., 19 nucleotides in length. The overhangs are typically present on the 3′ end of each strand, are usually 2 nucleotides long, and are composed of DNA or nucleotide analogs. An siRNA may be formed from two RNA strands that hybridize together, or may alternatively be generated from a longer double-stranded RNA or from a single RNA strand that includes a self-hybridizing portion, such as a short hairpin RNA. One of ordinary skill in the art will appreciate that one or more, mismatches or unpaired nucleotides may be present in the duplex formed by the two siRNA strands. One strand of an siRNA (the “antisense” or “guide” strand) includes a portion that hybridizes with a target nucleic acid, e.g., an mRNA transcript. Typically the antisense strand is perfectly complementary to the target over about 15-29 nucleotides, typically between 17-21 nucleotides, e.g., 19 nucleotides, meaning that the siRNA hybridizes to the target transcript without a single mismatch over this length. However, one of ordinary skill in the art will appreciate that one or more mismatches or unpaired nucleotides may be present in a duplex formed between the siRNA strand and the target transcript.

The term “short hairpin RNA” refers to a nucleic acid molecule comprising at least two complementary portions hybridized or capable of hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. The structure may further comprise an overhang. The duplex formed by hybridization of self-complementary portions of the shRNA has similar properties to those of siRNAs and, as described below, shRNAs are processed into siRNAs by the conserved cellular RNAi machinery. Thus shRNAs are precursors of siRNAs and are similarly capable of inhibiting expression of a target transcript. As is the case for siRNA, an shRNA includes a portion that hybridizes with a target nucleic acid, e.g., an mRNA transcript and is usually the perfectly complementary to the target over about 15-29 nucleotides, typically between 17-21 nucleotides, e.g., 19 nucleotides. However, one of ordinary skill in the art will appreciate that one or more mismatches or unpaired nucleotides may be present in a duplex formed between the shRNA strand and the target transcript.

The term “subject” as used herein, refers to any organism to which a lentiviral vector of the invention is administered or delivered for any purpose. In some embodiments, subjects include mammals, particularly rodents (e.g., mice and rats), avians, domesticated or agriculturally significant mammals (e.g., dogs, cats, cows, goats, etc.), primates, or humans. It is noted that although certain aspects of the present invention relate to gene therapy, the claims of this invention should be construed to explicitly exclude any embodiment that would entail patenting a human being to the extent that human beings constitute non-statutory subject matter.

An RNAi agent is considered to be “targeted” to a transcript and to the gene that encodes the transcript if (1) the RNAi agent comprises a portion, e.g., a strand, that is at least approximately 80%, approximately 85%, approximately 90%, approximately 91%, approximately 92%, approximately 93%, approximately 94%, approximately 95%, approximately 96%, approximately 97%, approximately 98%, approximately 99%, or approximately 100% complementary to the transcript over a region about 15-29 nucleotides in length, e.g., a region at least approximately 15, approximately 17, approximately 18, or approximately 19 nucleotides in length; and/or (2) the Tm of a duplex formed by a stretch of 15 nucleotides of one strand of the RNAi agent and a 15 nucleotide portion of the transcript, under conditions (excluding temperature) typically found within the cytoplasm or nucleus of mammalian cells and/or in a Drosophila lysate as described, e.g., in US Pubs. 20020086356 and 20040229266, is no more than approximately 15° C. lower or no more than approximately 10° C. lower, than the Tm of a duplex that would be formed by the same 15 nucleotides of the RNAi agent and its exact complement; and/or (3) the stability of the transcript is reduced in the presence of the RNAi agent as compared with its absence. An RNAi agent targeted to a transcript is also considered targeted to the gene that encodes and directs synthesis of the transcript. A “target region” is a region of a target transcript that hybridizes with an antisense strand of an RNAi agent. A “target transcript” is any RNA that is a target for inhibition by RNA interference. The terms “target RNA” and “target transcript” are used interchangeably herein.

“Variegation” as used herein refers to non-uniformity or variation in the expression of a transgene between cells of different cell types or cell lineages in a transgenic animal. For example, if different percentages of cells of different cell types or cell lineages express the transgene above a certain threshold level, then variegation is present. If expression can fall within multiple different ranges and different cell types or cell lineages in a transgenic animal differ with respect to the percentages of cells falling within the various ranges, then variegation is present.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (typically DNA plasmids, but RNA plasmids are also of use), cosmids, and viral vectors. As will be evident to one of skill in the art, the term “viral vector” is widely used refer either to a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). In particular, the terms “lentiviral vector,” “lentiviral expression vector,” etc. may be used to refer to lentiviral transfer plasmids and/or lentiviral particles of the invention as described herein.

The terms “viral particle” and “virus” are used interchangeably herein. For example, the phrase “production of virus” typically refers to production of viral particles.

### DETAILED DESCRIPTION OF CERTAIN PREFERRED EMBODIMENTS OF THE INVENTION

### Lentiviral Vectors Comprising an ARE and Optional SAR

The present invention provides novel lentiviral vectors and methods of use thereof, e.g., for transfer of nucleic acid sequences to mammalian and avian cells and expression of nucleic acid sequences therein. The invention further provides improved tools and methods for gene silencing that involve using lentiviral vectors to express RNAi agents such as short hairpin RNAs (shRNAs) in mammalian cells. The invention further provides transgenic non-human mammals generated using lentiviral vectors. Genomes of transgenic mammals in accordance with the invention comprise integrated transgenes transferred by inventive lentiviral vectors. In certain embodiments of the invention transgenic mammals display more uniform expression of a transgene among multiple cell lineages than has been achieved using lentiviral vectors previously known in the art. In certain embodiments of the invention a transgene encodes an RNAi agent such as an shRNA. The invention further provides animal models for human disease. Animal models are generated by using a lentiviral vector of the invention to create a transgenic non-human mammal that expresses an RNAi agent that specifically inhibits expression of a disease-associated gene.

Lentiviruses belong to the retrovirus family. Retroviruses comprise a diploid RNA genome that is reverse transcribed following infection of a cell to yield a double-stranded DNA intermediate that becomes stably integrated into the chromosomal DNA of the cell. The integrated DNA intermediate is referred to as a provirus and is inherited by the cell's progeny. Wild type retroviral genomes and proviral DNA include gag, pol, and env genes, flanked by two long terminal repeat sequences (LTRs). 5′ and 3′ LTRs comprise sequence elements that promote transcription (promoter-enhancer elements) and polyadenylation of viral RNA. LTRs also include additional cis-acting sequences required for viral replication. Retroviral genomes include sequences needed for reverse transcription and a packaging signal referred to as psi (Ψ) that is necessary for encapsidation (packaging) of a retroviral genome.

The retroviral infective cycle begins when a virus attaches to the surface of a susceptible cell through interaction with cell surface receptor(s) and fuses with the cell membrane. The viral core is delivered to the cytoplasm, where viral matrix and capsid become dismantled, releasing the viral genome. Viral reverse transcriptase (RT) copies the RNA genome into DNA, which integrates into host cell DNA, a process that is catalyzed by the viral integrase (IN) enzyme. Transcription of proviral DNA produces new viral genomes and mRNA from which viral Gag and Gag-Pol polyproteins are synthesized. These polyproteins are processed into matrix (MA), capsid (CA), and nucleocapsid (NC) proteins (in the case of Gag), or the matrix, capsid, protease (PR), reverse transcriptase (RT), and integrase (INT) proteins (in the case of Gag-Pol). Transcripts for other viral proteins, including envelope glycoproteins, are produced via splicing events. Viral structural and replication-related proteins associate with one another, with viral genomes, and with envelope proteins at the cell membrane, eventually resulting in extrusion of a viral particle having a lipid-rich coat punctuated with envelope glycoproteins and comprising a viral genome packaged therein.

Retroviruses are widely used for in vitro and in vivo transfer and expression of heterologous nucleic acids, a process often referred to as gene transfer. For retroviral gene transfer, a nucleic acid sequence (e.g., all or part of a gene of interest), optionally including regulatory sequences such as a promoter, is inserted into a viral genome in place of some of the wild type viral sequences to produce a recombinant viral genome. The recombinant viral genome is delivered to a cell, where it is reverse transcribed and integrated into the cellular genome. Transcription from an integrated sequence may occur from the viral LTR promoter-enhancer and/or from an inserted promoter. If an inserted sequence includes a coding region and appropriate translational control elements, translation results in expression of the encoded polypeptide by the cell. For purposes of the present invention, sequences that are present in the genome of a cell as a result of a process involving reverse transcription and integration of a nucleic acid delivered to the cell (or to an ancestor of the cell) by a retroviral vector are considered a “provirus.” It will be recognized that while such sequences comprise retrovirus derived nucleic acids (e.g., at least a portion of one or more LTRs, sequences required for integration, packaging sequences, eta), they will typically lack genes for various essential viral proteins and may have mutations or deletions in those viral sequences that they do contain, relative to the corresponding wild type sequences.

Lentiviruses such as HIV differ from the simple retroviruses described above in that their genome encodes a variety of additional proteins such as Vif, Vpr, Vpu, Tat, Rev, and Nef and may also include regulatory elements not found in the simple retroviruses. The genes encoding these proteins overlap with the gag, pol, and env genes. Certain of these proteins are encoded in more than one exon, and their mRNAs are derived by alternative splicing of longer mRNAs. In contrast to simple retroviruses, lentiviruses are able to transduce and productively infect nondividing cells such as resting T cells, dendritic cells, and macrophages. Nondividing cell types of interest include, but are not limited to, cells found in the liver (e.g., hepatocytes), skeletal or cardiac muscle (e.g., myocytes), nervous system (e.g., neurons), retina, and various cells of the system. Lentiviral vectors can transfer genes to hematopoietic stem cells with superior gene transfer efficiency and without affecting the repopulating capacity of these cells (see, e.g., Mautino et al., 2002, AIDS Patient Care STDS 16:11; Somia et al., 2000, J. Virol., 74:4420; Miyoshi et al., 1999, Science, 283:682; and U.S. Pat. No. 6,013,516). Further discussion of retroviruses and lentiviruses is found in Coffin, J., et al. (eds.), Retroviruses, Cold Spring Harbor Laboratory Press, Cold Spring Harbor, 1997, and Fields, B., et al., Fields' Virology, 4^{th}. ed., Philadelphia: Lippincott Williams & Wilkins, 2001. See also the Web site with URL www.ncbi.nlm.nih.gov/ICTVdb/ICTVdB, accessed Feb. 14, 2006. As used herein, a retroviral vector is considered a “lentiviral vector” if at least approximately 50% of the retrovirus derived LTR and packaging sequences in the vector are derived from a lentivirus and/or if the LTR and packaging sequences are sufficient to allow an appropriately sized nucleic acid comprising the sequences to be reverse transcribed and packaged in a mammalian or avian cell that expresses the appropriate lentiviral proteins. Typically at least approximately 60%, approximately 70%, approximately 80%, approximately 90%, or more of retrovirus derived LTR and packaging sequences in a vector are derived from a lentivirus. For example, LTR and packaging sequences may be at least approximately 50%, approximately 60%, approximately 70%, approximately 80%, approximately 90%, or identical to lentiviral LTR and packaging sequences. In certain embodiments of the invention between approximately 90 and approximately 100% of the LTR and packaging sequences are derived from a lentivirus. For example, the LTR and packaging sequences may be between approximately 90% and approximately 100% identical to lentiviral LTR and packaging sequences.

Lentiviral vectors of the present invention comprise a nucleic acid that comprises (i) a eukaryotic anti-repressor element (ARE); and (ii) lentivirus derived sequences sufficient for reverse transcription and packaging. In certain embodiments of the invention a nucleic acid further comprises a scaffold attachment region (SAR). AREs and SARs are described below. A nucleic acid may comprise one or more regulatory sequences sufficient to promote transcription of an operably associated sequence of interest, which may be inserted downstream of regulatory sequences. The invention further provides lentiviral transfer plasmids and multi-plasmid systems, wherein at least one of the plasmids comprises a nucleic acid that comprises (i) a eukaryotic anti-repressor element (ARE); and (ii) lentivirus derived sequences sufficient for reverse transcription and packaging. The invention further provides lentiviral particles having a genome that comprises a nucleic acid that comprises (i) a eukaryotic anti-repressor element (ARE); and (ii) sequences sufficient for reverse transcription and packaging, wherein said sequences are at least in part derived from a lentivirus. For example, sequences may include a lentiviral U3 region, a lentiviral U5 region, a lentiviral psi sequence, or any combination of the foregoing. It will be appreciated that “nucleic acid sequences sufficient for reverse transcription and packaging” means that sequences are sufficient when present in a nucleic acid in the RNA form but that the sequences may be in the RNA or DNA form in the lentiviral vector, e.g., the nucleic acid component of the vector need not be RNA if the vector is a transfer plasmid.

The invention further provides retroviral vectors comprising a nucleic acid that comprises (i) a eukaryotic anti-repressor element (ARE); and (ii) sequences sufficient for reverse transcription and packaging, wherein said sequences are at least in part derived from a retrovirus. In certain embodiments of the invention a nucleic acid further comprises a scaffold attachment region (SAR). In certain embodiments of the invention at least approximately 50% of retrovirus derived sequences (e.g., LTR and packaging sequences) are derived from a retrovirus that is not a lentivirus. Retroviral vectors may be used for any of a variety of purposes described herein for lentiviral vectors of the invention and may be similarly produced.

A nucleic acid that comprises (i) a eukaryotic anti-repressor element (ARE); and (ii) retrovirus derived sequences sufficient for reverse transcription and packaging is contemplated by the present invention. In certain embodiments of the invention a nucleic acid comprises a scaffold attachment region (SAR). Retrovirus derived sequences may be at least in part or entirely derived from a lentivirus. Retrovirus derived sequences may include one or more portions of an LTR, e.g., a U3 region and a U5 region. An ARE may be located between LTRs or portions thereof.

Anti-repressor elements (AREs) are nucleic acids derived from a eukaryotic genome that, when present in cis in a DNA sequence that comprises a gene, enhance expression of the gene when the DNA sequence is present in cultured eukaryotic cells, e.g., mammalian cell lines. Without wishing to be bound by any theory, an ARE may, for example, counteract the gene suppressive effects of certain eukaryotic chromatin associated repressor proteins for which binding sites are present in the DNA sequence. A chromatin associated repressor protein can be, e.g., a Polycomb group complex protein, binding sites for which are known in the art. A gene comprises regulatory sequences sufficient for transcription of an operably linked nucleic acid. Regulatory sequences may comprise a promoter, an internal ribosome entry site (IRES), etc.

A nucleic acid can be tested to in a variety of ways to determine whether it functions as an ARE. For example, a candidate ARE can be inserted into a vector that comprises (i) a binding site for a eukaryotic chromatin associated repressor protein and (ii) a reporter gene that encodes a detectable or selectable marker. A selectable or detectable marker is a nucleic acid or protein whose presence can be detected (either directly or indirectly) in a cell. A candidate ARE may, for example, range from about 50 to about 50,000 base pairs in length. For example, a candidate ARE may be between about 100 and 5000, or between 100 and 1000 base pairs in length. A vector that expresses the chromatin associated repressor protein is introduced into eukaryotic cells. Any suitable method known in the art may be used to introduce a vector into cells. If a candidate ARE does not function as an ARE, expression of a reporter gene is low so that cells are not detected or selected, while if a candidate ARE does function as an ARE, expression is increased so that cells are detected or selected. Average expression may, for example, be at least approximately 2-fold, at least approximately 5-fold, at least approximately 10-fold, etc., as great in the presence of an ARE as in its absence. Alternately or additionally, the percentage of cells that express a reporter gene at a selected level in the presence of an ARE is greater than in its absence. Expression levels can be qualitatively and/or quantitatively determined in any of a variety of ways. For example, if a reporter gene encodes a selectable marker, the number of cell colonies formed under particular selective conditions in the presence of the nucleic acid can be compared with the number formed in the absence of the nucleic'acid. A nucleic acid may be identified as an ARE if the number of colonies formed in the presence of the nucleic acid is greater than in its absence by a factor of at least approximately 2, at least approximately 5, at least approximately 10, etc. If a reporter gene encodes a fluorescent marker, expression can be assessed using fluorescence activated cell sorting (FACS), etc.

A wide variety of detectable or selectable markers known to those of skill in the art can be used in the above methods to determine whether any particular nucleic acid functions as an ARE. A detectable marker can be, for example, a fluorescent or chemiluminescent molecule (e.g., green fluorescent protein or a variant thereof, luciferase, etc.) or an enzyme, such as β-galactosidase, capable of metabolizing a substrate to produce a detectable substance. A detectable marker may also be referred to as a “reporter.” Reporters are discussed in more detail below. A selectable marker can be nucleic acid or protein that inactivates a lethal or growth-inhibitory compound and thereby protects a cell from compound's effects. Drug resistance markers are a non-limiting example of a class of selectable marker that can be used to select cells that express the marker. In the presence of an appropriate concentration of drug (selective conditions), such a marker confers a growth advantage on a cell that expresses the marker. Thus cells that express the drug resistance marker are able to survive and/or proliferate in the presence of drug while cells that do not express the drug resistance marker are not able to survive and/or are unable to proliferate in the presence of drug. For example, a selectable marker of this type that is commonly used in mammalian cells is the neomycin resistance gene (an aminoglycoside 3′-phosphotransferase, 3′ APH II). Expression of this selectable marker renders cells resistant to various drugs such as G418, Additional selectable markers of this type include enzymes conferring resistance to Zeocin™, hygromycin, puromycin, etc. These enzymes and the genes encoding them are well-known in the art. A second non-limiting class of selectable markers is nutritional markers. Such markers are generally enzymes that function in a biosynthetic pathway to produce a compound that is needed for cell growth or survival. In general, under nonselective conditions the required compound is present in the environment or is produced by an alternative pathway in the cell. Under selective conditions, functioning of the biosynthetic pathway in which the marker is involved is needed to produce the compound. Two examples of nutritional markers that are suitable for use in the invention are hypoxanthine phosphoribosyl transferase (HPRT) and thymidine kinase (TK).

To systemically identify naturally occurring AREs, fragments of DNA from a eukaryotic genome can be inserted into a vector such as that described above to create a library. Fragments can, for example, be generated using restriction enzymes or by shearing genomic DNA. A library is introduced into eukaryotic cells. Cells that express a reporter gene are selected or detected. Vector is then isolated from the cells. A fragment is isolated from the vector and can then be manipulated and/or modified using standard molecular biology techniques known in the art. If desired, a fragment can be sequenced and/or its chromosomal location determined. If desired, the portion(s) of a fragment that possess anti-repressor activity can be narrowed down to a minimal effective region by producing derivatives of the original fragment, in which certain portions are deleted, mutated, or altered, and then testing them in the assay described above. For example, it will often be possible to reduce the size of a fragment by making deletions at either the 5′ or 3′ end. Furthermore, since AREs are often highly conserved among different species, portions of an ARE that extend beyond the boundaries of an identified fragment may be identified by comparing the sequence of the ARE with homologous sequences in a different organism. Once an ARE is identified in a first organism, homologous AREs in other organisms may be identified by searching sequence databases using part or all of the nucleotide sequence of the ARE as a query sequence, by low stringency hybridization (e.g., of genomic DNA libraries) using all or part of the ARE as a probe, etc. Furthermore, a number of changes can be made in a naturally occurring ARE, e.g., using standard molecular biology techniques, without significantly diminishing its activity and possible even resulting in increased activity. It will thus be appreciated that the term “eukaryotic ARE” encompasses both naturally occurring AREs and modified versions thereof that possess anti-repressing activity.

Scaffold attachment regions (SARs), also referred to as matrix attachment regions (MARs), are eukaryotic DNA sequences that bind to an isolated nuclear scaffold or matrix (proteinaceous network of the nucleus) with high affinity (Cockerill, P. N., and W. T. Garrard. Cell 44:273-282, 1986). In cells, these sequences serve to attach chromatin fiber to the nuclear matrix and thereby subdivide the eukaryotic genome into structural and functional domains. They are found at the base of the chromatin loops into which the eukaryotic genome appears to be organized. SARs have an average size of about 500 base pairs and are located about every 30 kB in the genome. A large number of SAR sequences have been isolated and their functional properties demonstrated. Many SAR sequences share a number of characteristics. For example, many are AT rich (70%) and enriched in binding sites for a variety of nuclear proteins such as DNA topoisomerase II. However, no consensus sequence has yet been identified. Methods for identifying and functionally characterizing SARs are well known in the art and are described (e.g., Boulikas, “Chromatin Domains and Prediction of SAR Sequences” in Berezney et al., The Nuclear Matrix, San Diego: Academic Press, 1995). For example, DNA fragments may be incubated with isolated nuclear matrix of scaffold proteins and bound DNA fragments may be separated from unbound DNA by centrifugation. Micrococcal nuclease digestion of chromatin loops in intact nuclei can be used to trim the loops down to the attachment points to the nuclear matrix. Several computer programs are available to predict which sequences within a nucleic acid sequence such as a genomic region are likely to function as MARs. Examples include Mar-finder (www.futuresoft.org/MAR-Wiz), marscan (bioweb.pasteur.fr/seqanal/interfaces/marscan.html), and ChrClass (Glazko, et al., 2001, Biochim Biophys Acta, 1517:351).

Once a genomic fragment comprising an SAR is isolated, its sequence can be manipulated and/or modified using standard molecular biology techniques known in the art. If desired, a SAR can be sequenced and/or its chromosomal location determined. If desired, the portion(s) of a genomic fragment comprising a SAR can be narrowed down to a minimal effective region by producing derivatives of the original fragment, in which certain portions are deleted, mutated, or altered, and then testing them to determine whether they bind to nuclear matrix. It will often be possible to reduce the size of the fragment by making deletions at either the 5′ or 3′ end. Furthermore, a number of changes can be made in a naturally occurring SAR, e.g., using standard molecular biology techniques, without significantly diminishing its activity and possible even resulting in increased activity. It will thus be appreciated that the term “eukaryotic SAR” encompasses both naturally occurring SARs and modified versions thereof that possess anti-repressing activity.

The present encompasses the recognition that lentiviral vectors that comprise a eukaryotic ARE and, optionally, a eukaryotic SAR, possess significant advantages, e.g., for purposes of creating transgenic nonhuman animals using lentiviral vectors and for expressing RNAi agents in isolated eukaryotic cells and/or in transgenic animals using lentiviral vectors. Surprisingly, as described in further detail below and in the Examples, transgenic animals created using a lentiviral vector comprising a nucleic acid that comprises a eukaryotic ARE, a eukaryotic SAR, and an expression cassette comprising a transgene display an increase in the overall percentage of cells that express the transgene in multiple cell types, including cell types arising from different lineages. Such animals displayed reduced variegation relative to that observed in transgenic animals created using an otherwise identical lentiviral vector lacking an ARE and SAR. For example, transgenic mice created using a lentiviral vector of the present invention comprising an ARE, a SAR, and an expression cassette comprising a transgene encoding a detectable marker expressed the transgene in more than 50% of non-erythroid hematopoietic cells; e.g., expression of the detectable marker was observed in approximately 70% of peripheral white blood cells (71% of T cells, 70% of B cells, and 71% of macrophages). Thus the percentage of cells that expressed the transgene was almost identical among multiple hematopoietic cell types. In contrast, the overall percentage of hematopoietic cells expressing the transgene in transgenic mice created using an otherwise essentially identical lentiviral vector was much lower and varied significantly between different cell types; e.g., expression was observed in 34% of CD4⁺ T cells and only 11% of B cells and 17.5% of granulocytes. Presence of the ARE and SAR increased the percentage of cells that expressed transgene by between about 2 and 6 fold, depending on the cell type.

Similar increases in the percentages of expressing cells among multiple hematopoietic cell types and reduced variegation were observed in transgenic animals generated using a lentiviral vector of the invention comprising an ARE, a SAR, a first expression cassette comprising a first transgene encoding a detectable marker and a second expression cassette comprising a second transgene encoding an shRNA. The detectable marker was expressed in 70% of CD4⁺ T cells, 71% of CD8⁺ T cells, 65% of B cells, and 65% of macrophages. Thus, the percentage of cells that expressed the transgene varied by less than 10% between multiple hematopoietic cell types. Increased percentages and reduced variegation persisted over multiple generations. When transgenic founder mice generated using a lentiviral vector of the invention were bred to congenic, nontransgenic mice, the resulting F1 mice, and subsequent generations, also displayed higher overall percentages of hematopoietic cells that expressed the transgene. Some variegation was observed in the F1 generation; e.g., expression was detected in 45-75% of hematopoietic cells. The increased percentage of cells expressing the transgene and the reduced variegation remained stable and consistent over the F2, F3, and F4 generations.

A lentiviral vector of the present invention can comprise any ARE known in the art or discovered hereafter. An ARE may originate from a genome of any eukaryotic organism, e.g., mammalian, avian, plant, etc. In certain embodiments of the invention an ARE is a mammalian ARE, such as a primate (e.g. human) or rodent (e.g., mouse, rat, hamster) ARE. In certain embodiments of the invention, an ARE is derived from an avian or plant genome, e.g., from Arabidopsis thaliana. An ARE may be highly conserved between different organisms over part or all of its length. For example, useful AREs may be at least approximately 40%, approximately 50%, approximately 60%, approximately 70%, approximately 80%, or approximately 90% identical between mouse and human over at least approximately 200, approximately 300, approximately 400, approximately 500, approximately 600, approximately 700, approximately 800, approximately 900, approximately 1000, or more base pairs (allowing the introduction of gaps). Certain AREs may comprise more than one highly conserved region. A naturally occurring ARE typically consists entirely of noncoding sequences. However, AREs that comprise or consist of coding sequences may also be used.

In certain embodiments of the invention, a lentiviral vector comprises an ARE that is approximately or precisely 100% identical to a genomic region of a eukaryotic organism, e.g., mouse or human, over at least approximately 200, approximately 300, approximately 400, approximately 500, approximately 600, approximately 700, approximately 800, approximately 900, approximately 1000, or more base pairs. In certain embodiments of the invention a lentiviral vector comprises an ARE that is at least approximately 40%, approximately 50%, approximately 60%, approximately 70%, approximately 80%, or approximately 90% identical between mouse and human over at least approximately 50, approximately 100, approximately 150, approximately 200, approximately 300, approximately 400, approximately 500, approximately 600, approximately 700, approximately 800, approximately 900, approximately 1000, or more base pairs (allowing the introduction of gaps).

An ARE that is precisely identical to a genomic region of a eukaryotic organism or is generated by making one or more alterations to an ARE that is precisely identical to a genomic region of a eukaryotic organism, where such alterations result in a sequence that is at least approximately 90% identical to the original sequence over at least approximately 200 base pairs is said to originate from that organism. In certain embodiments of the invention an ARE is between approximately 50 to approximately 100, approximately 100 to approximately 200, approximately 200 to approximately 500, approximately 200 to approximately 1000, approximately 200 to approximately 1500, or approximately 200 to approximately 2000 base pairs in length, or any shorter fragment within any of the foregoing ranges, e.g., between approximately 300 to approximately 500, approximately 300 to approximately 600, approximately 400 to approximately 500 base pairs, etc.

In certain embodiments of the invention an ARE is a composite ARE, by which is meant that it includes portions from two or more different AREs, in which case the ARE may “originate from” more than two or more different organisms. In certain embodiments of the invention a lentiviral vector comprises two, three, or more AREs adjacent to one another. Two AREs are considered adjacent if the 3′ end of a first ARE is separated from the 5′ end of a second ARE by no more than approximately 200 nucleotides.

An ARE of use in the invention may display anti-repressor activity in cells of the organism from which it originates and/or in cells of one or more other eukaryotic organisms. For example, certain AREs of rodent (e.g., mouse) origin function in both rodent and primate cells, e.g., in both mouse and human cells. Certain AREs of primate (e.g., human) origin function in both rodent and primate cells, e.g., in both mouse and human cells. In certain embodiments of the invention an ARE is functional in many different cell types, e.g., most or essentially all cell types. In some embodiments of the invention an ARE is functional in a subset of cell types, e.g., one to several different cell types. In certain embodiments of the invention an ARE is functional in a single lineage or in multiple lineages. For example, an ARE may be functional in one or more hematopoietic lineages.

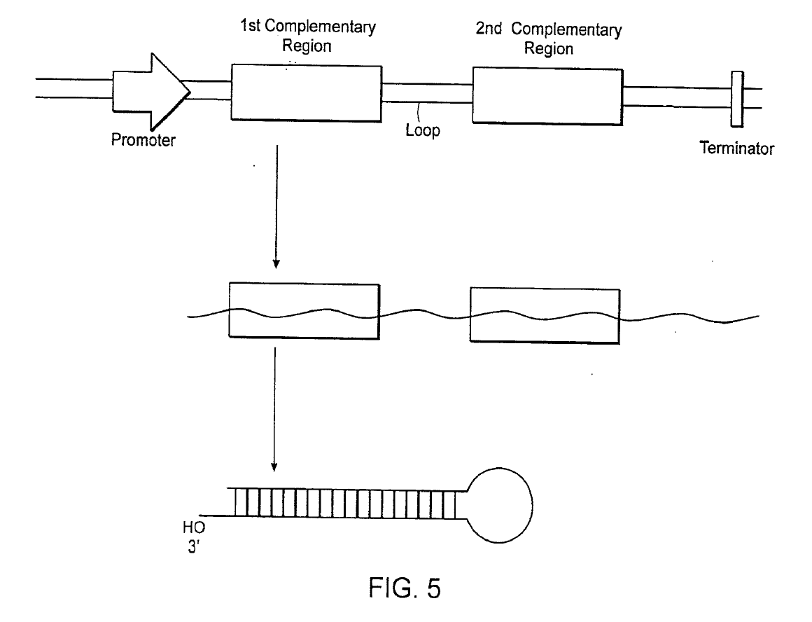

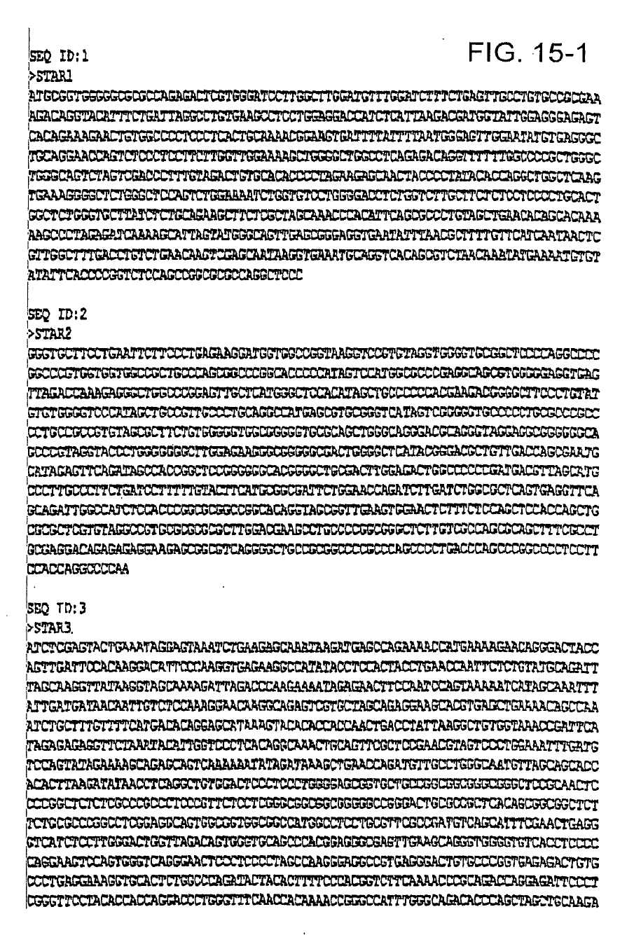

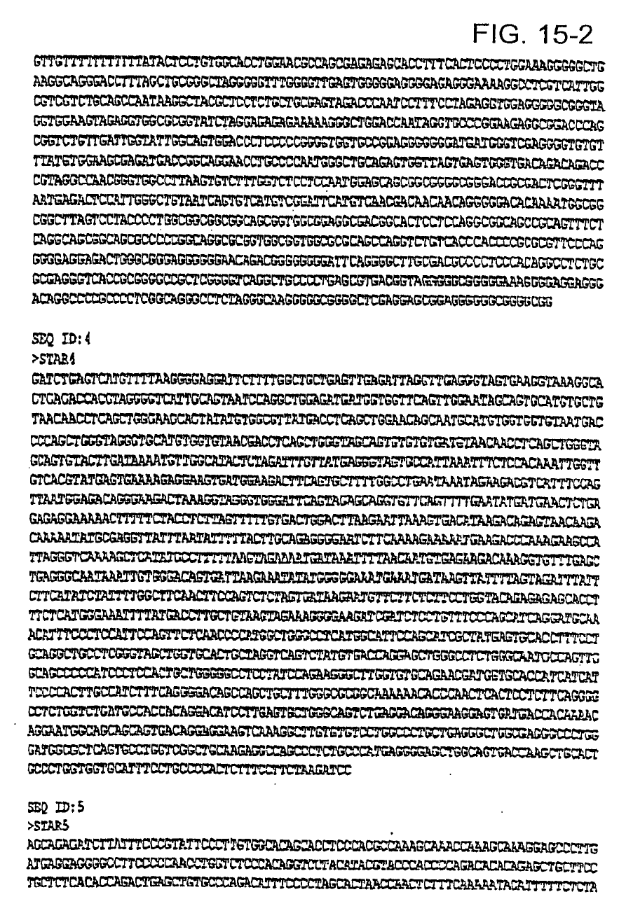

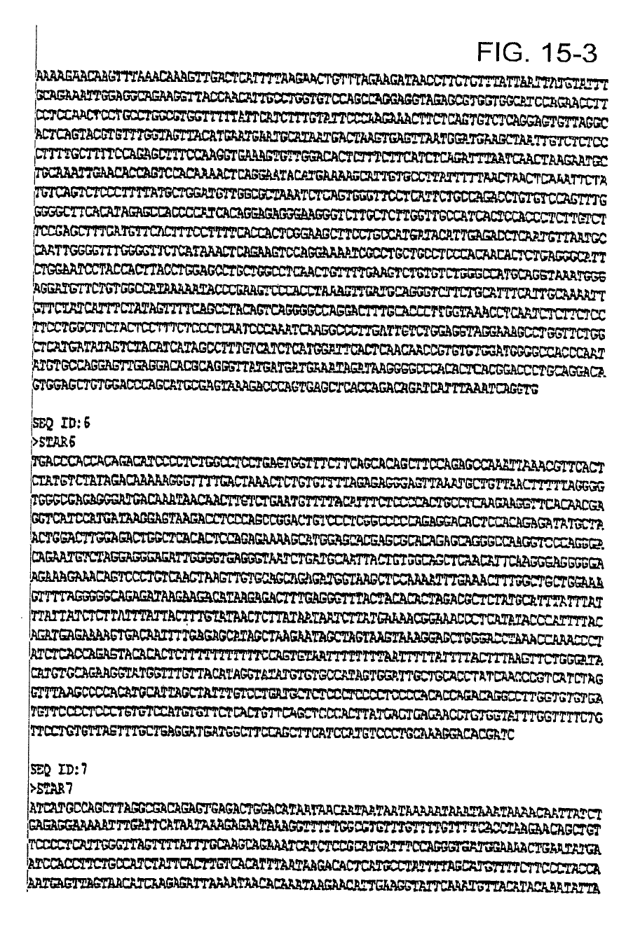

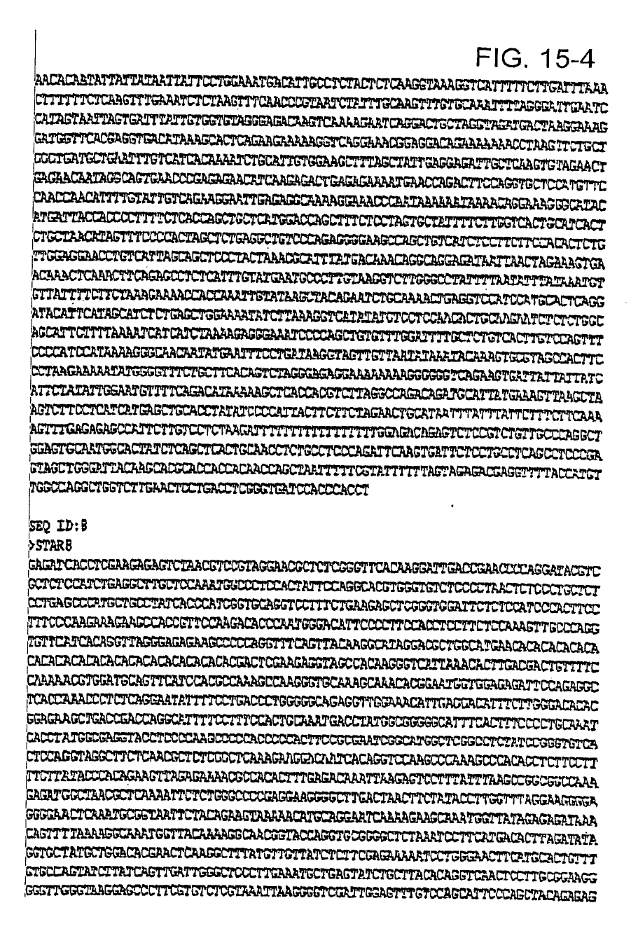

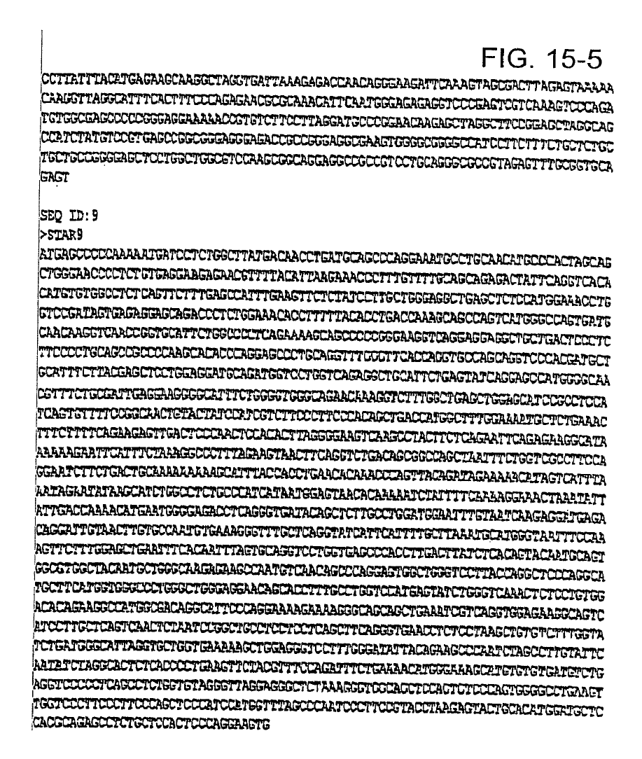

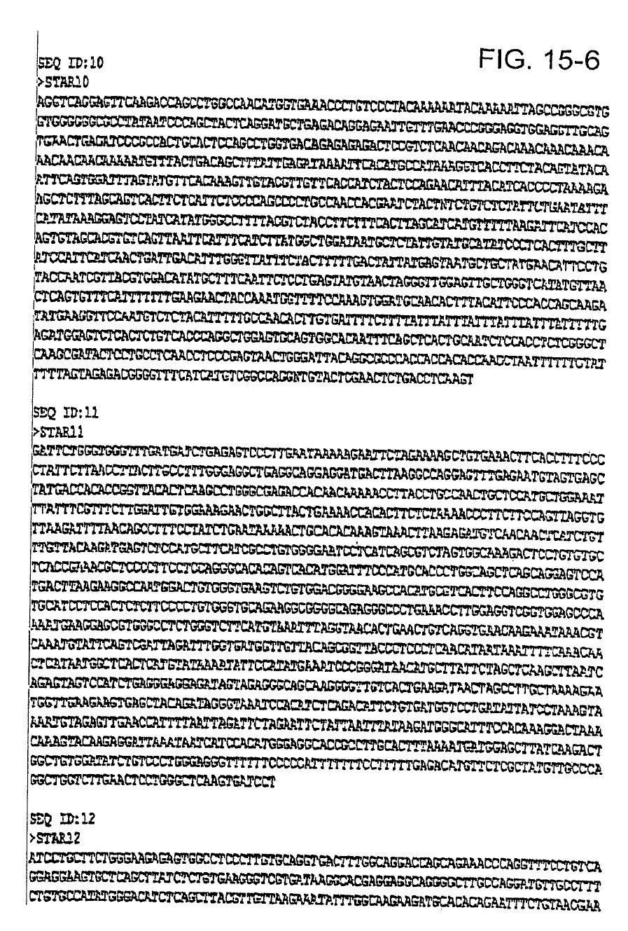

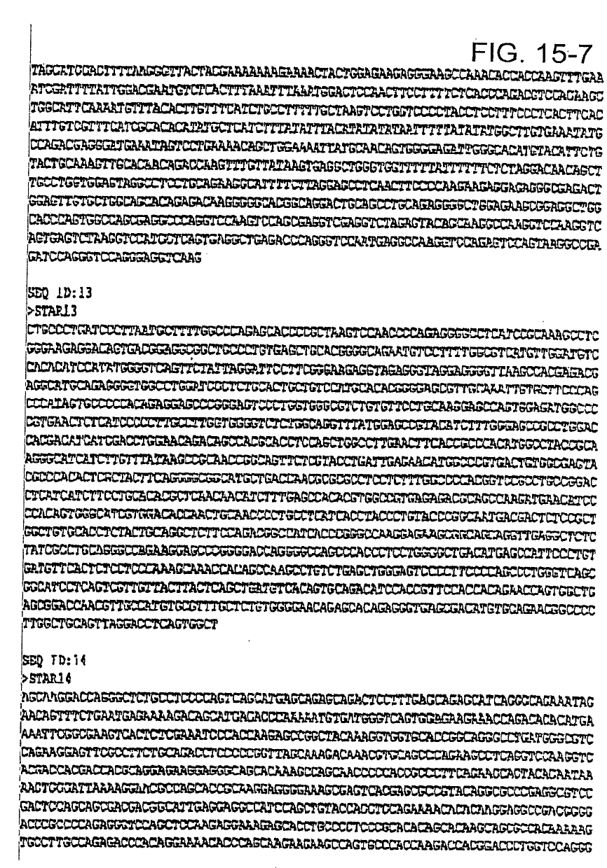

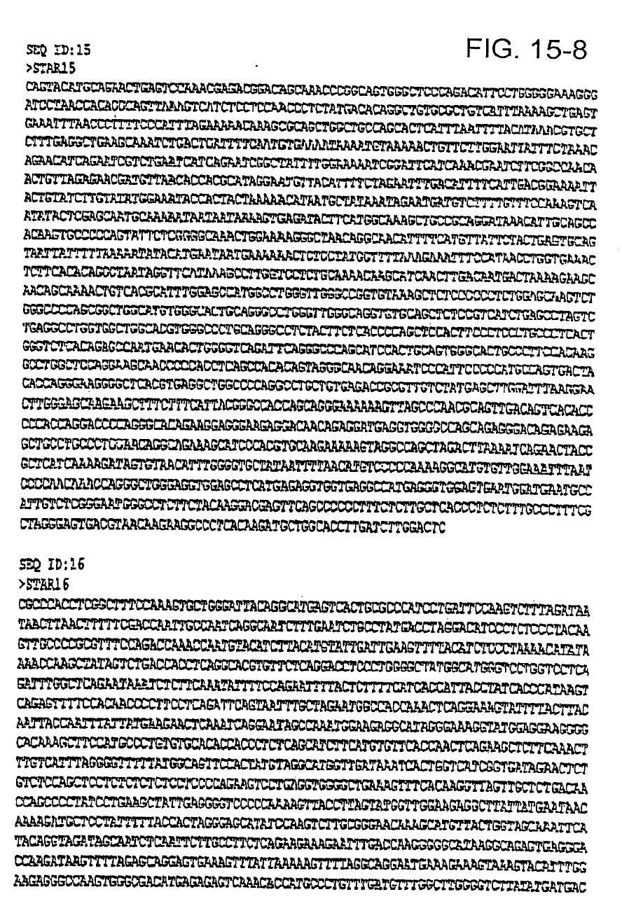

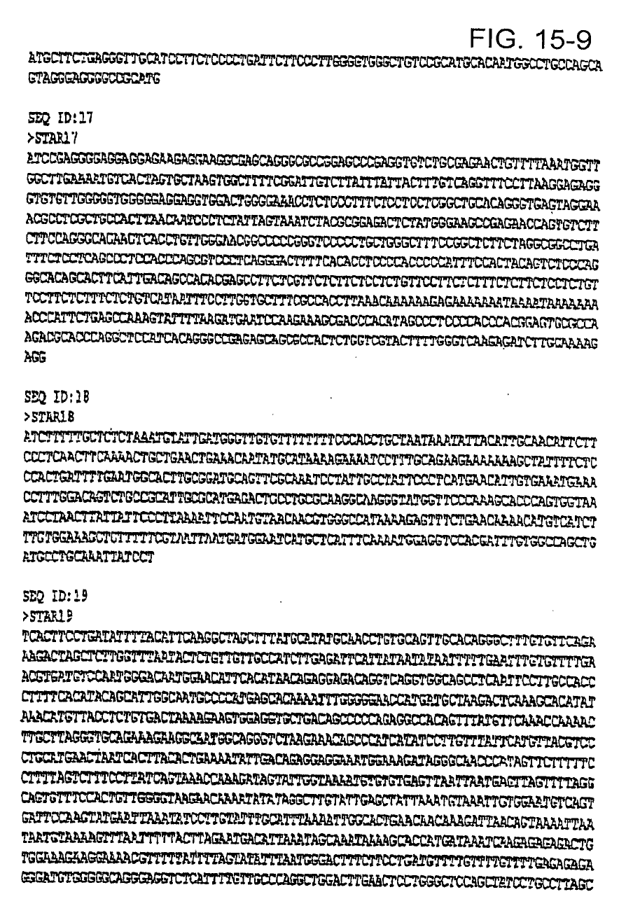

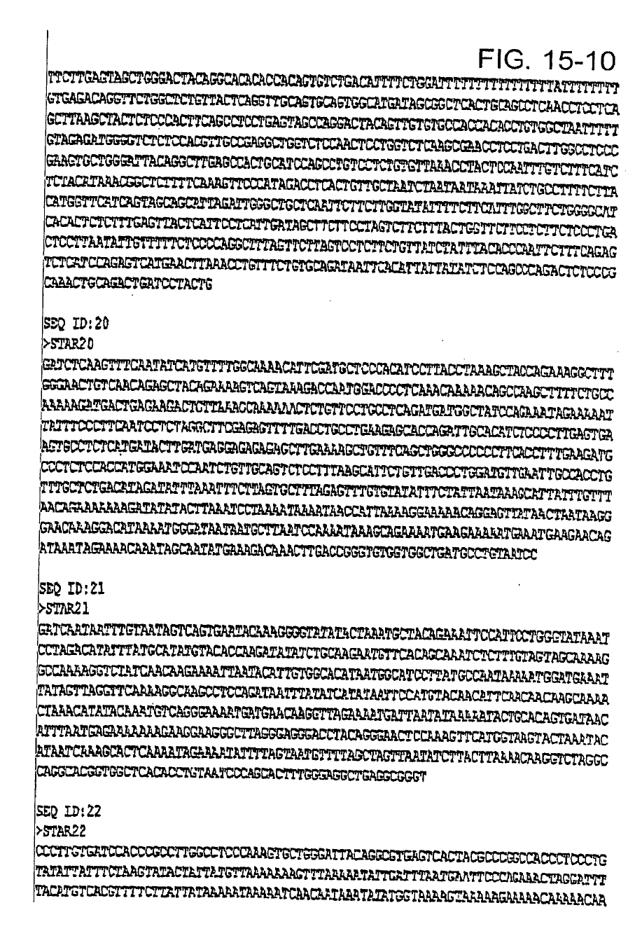

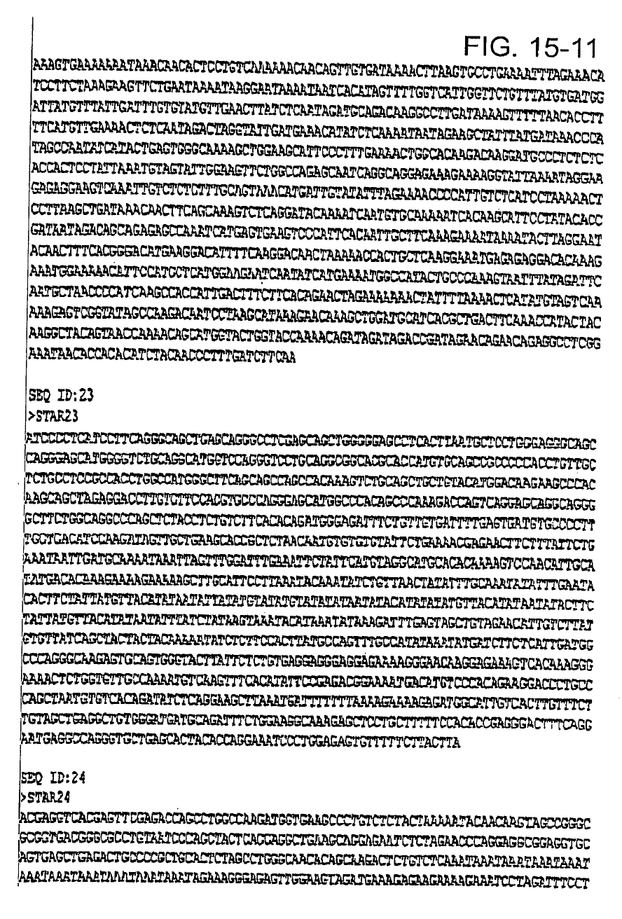

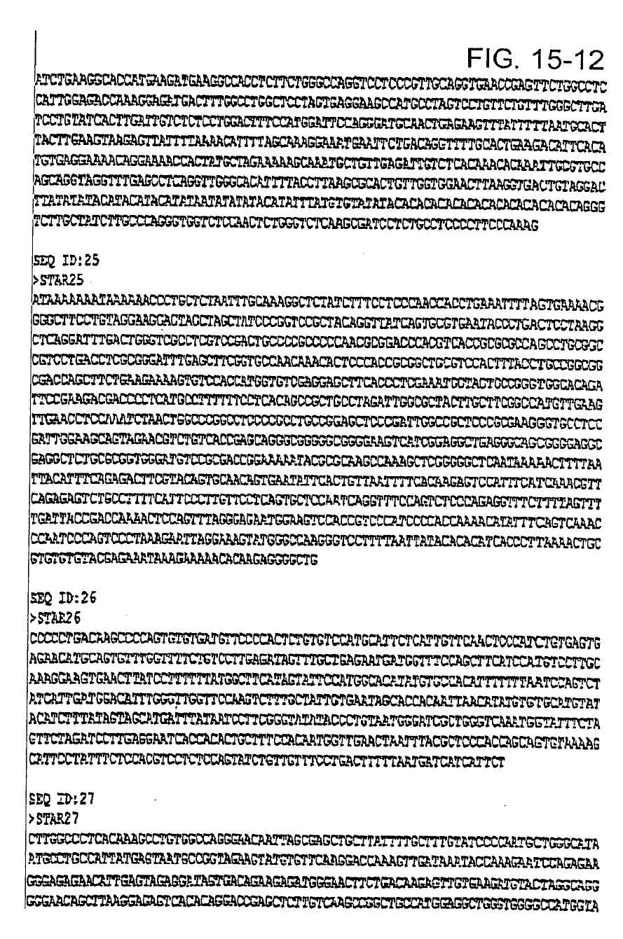

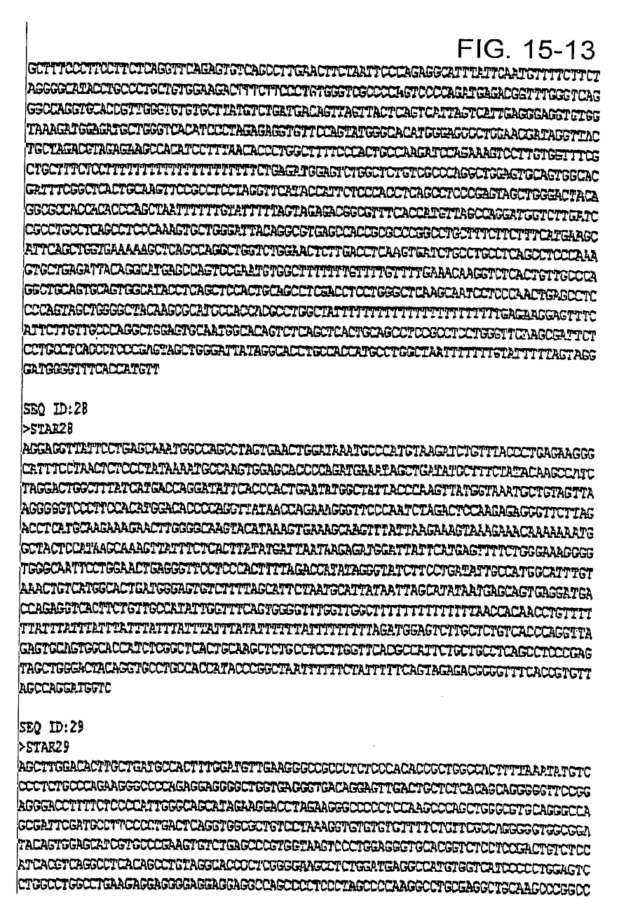

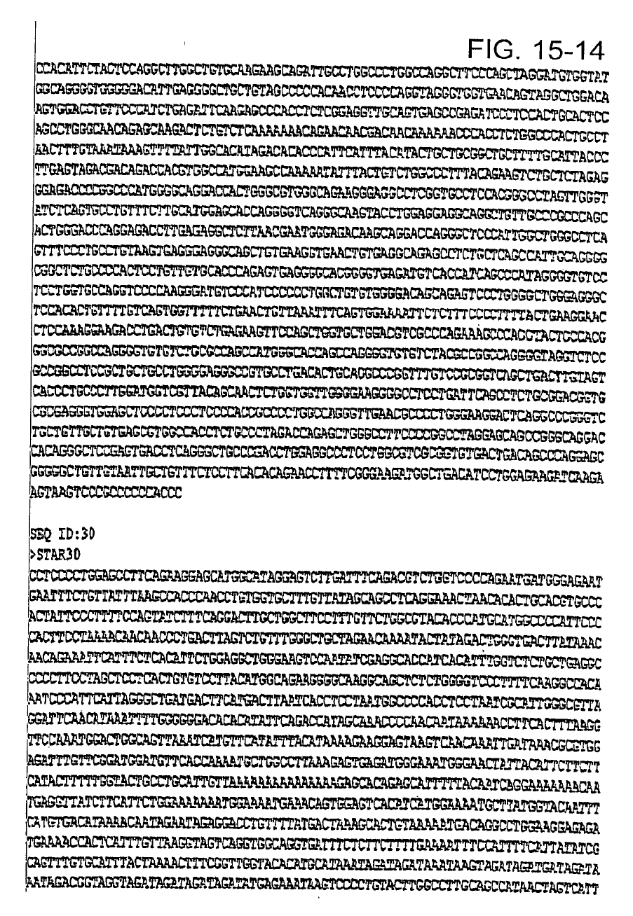

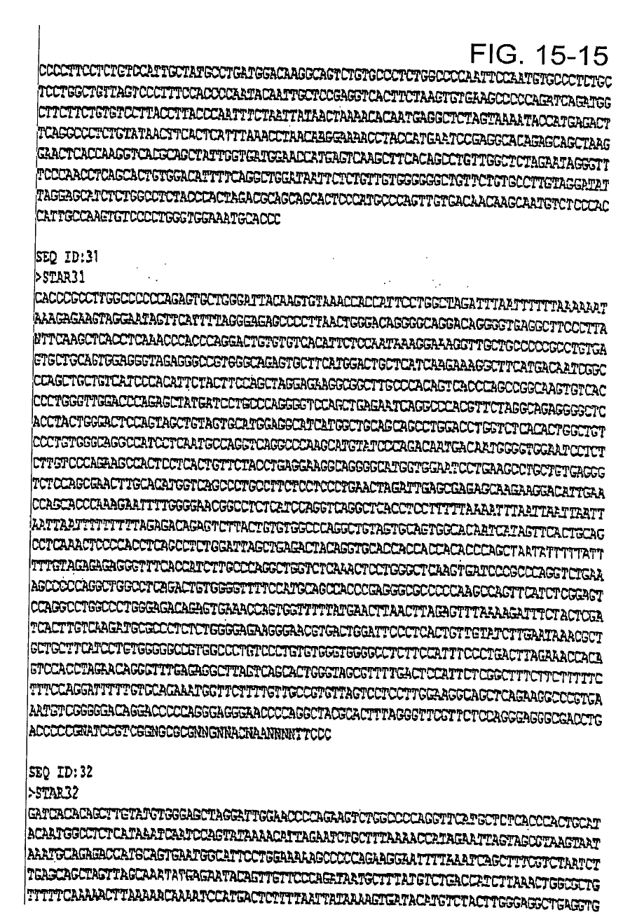

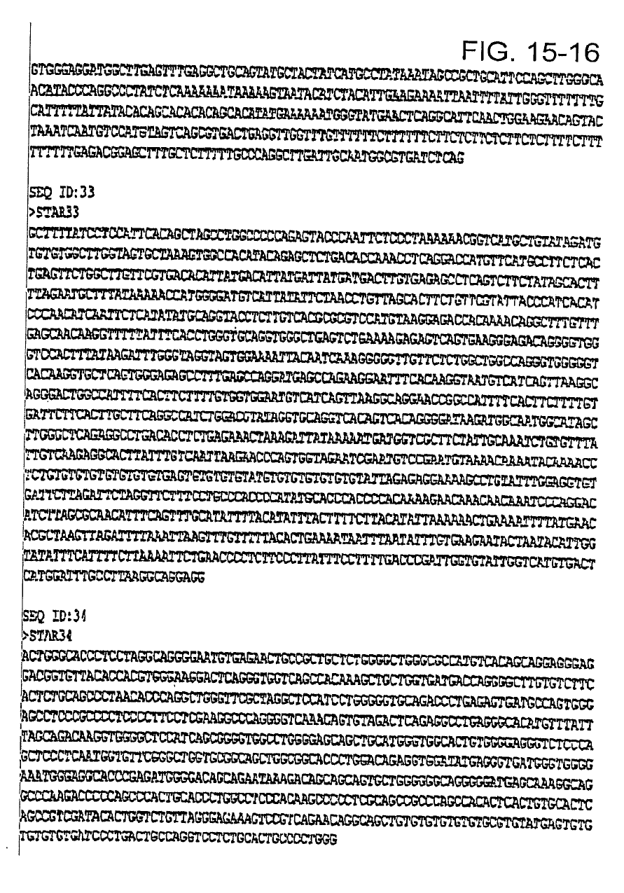

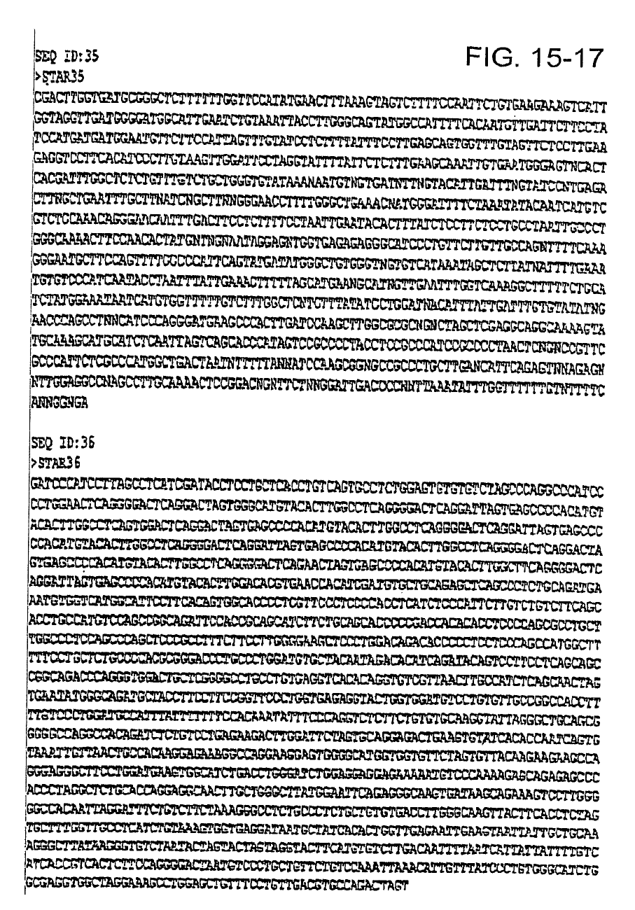

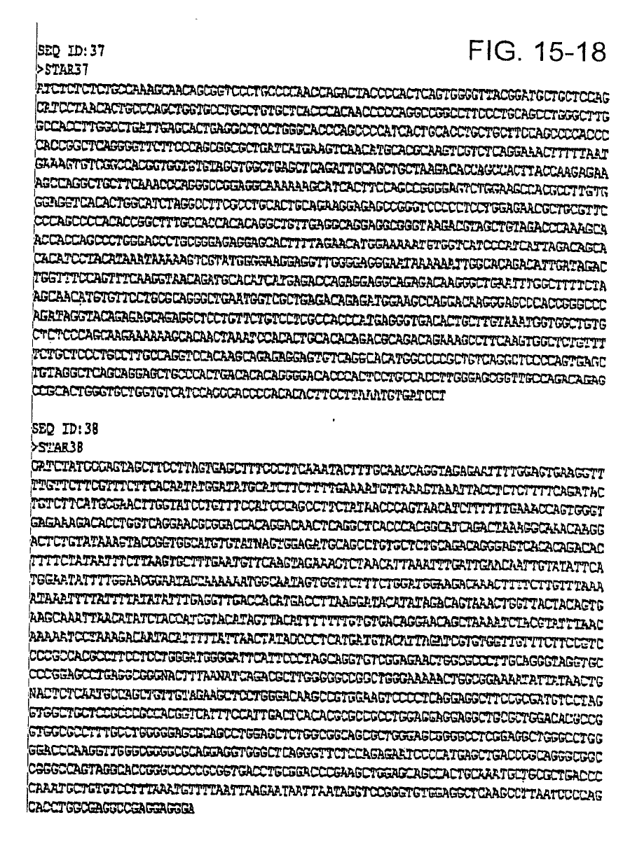

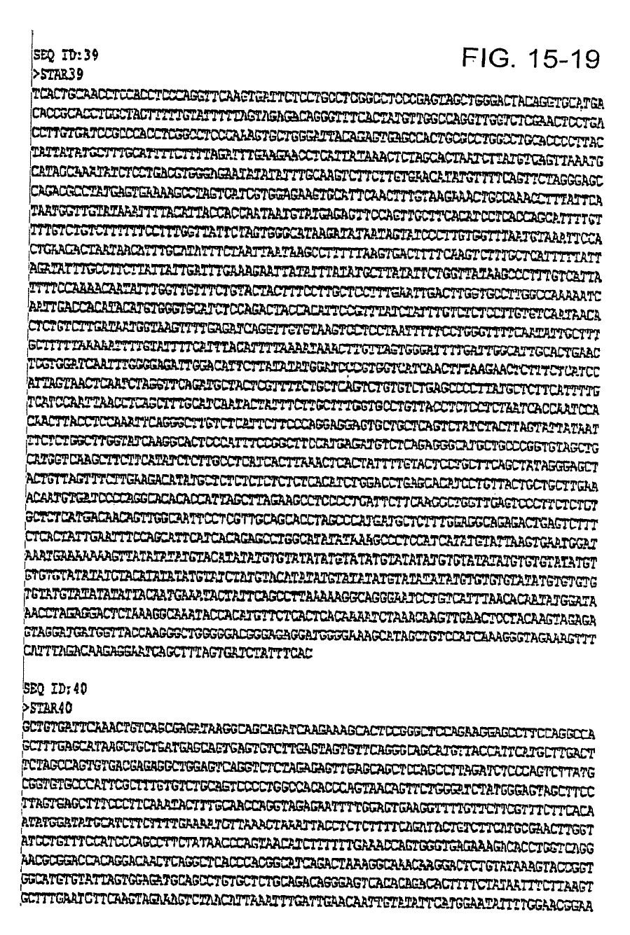

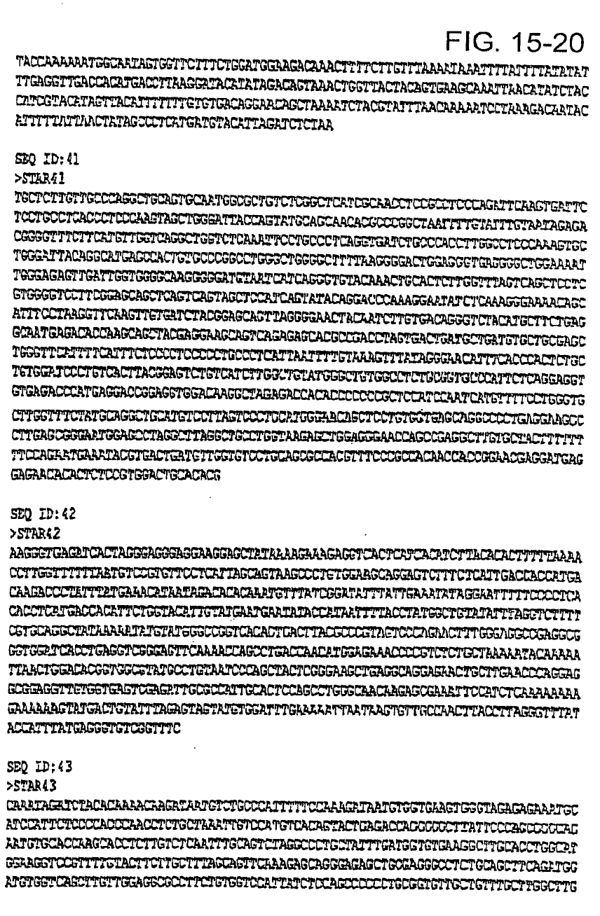

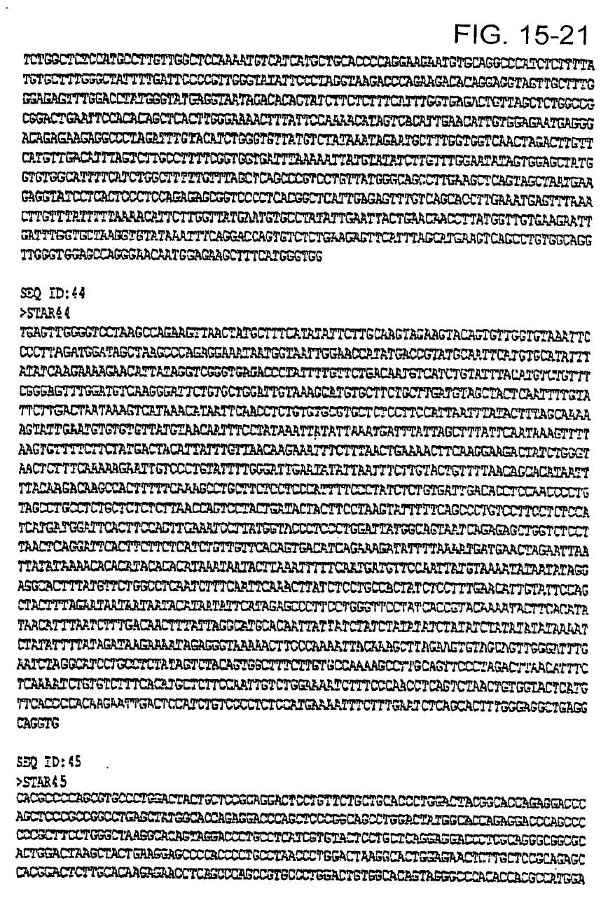

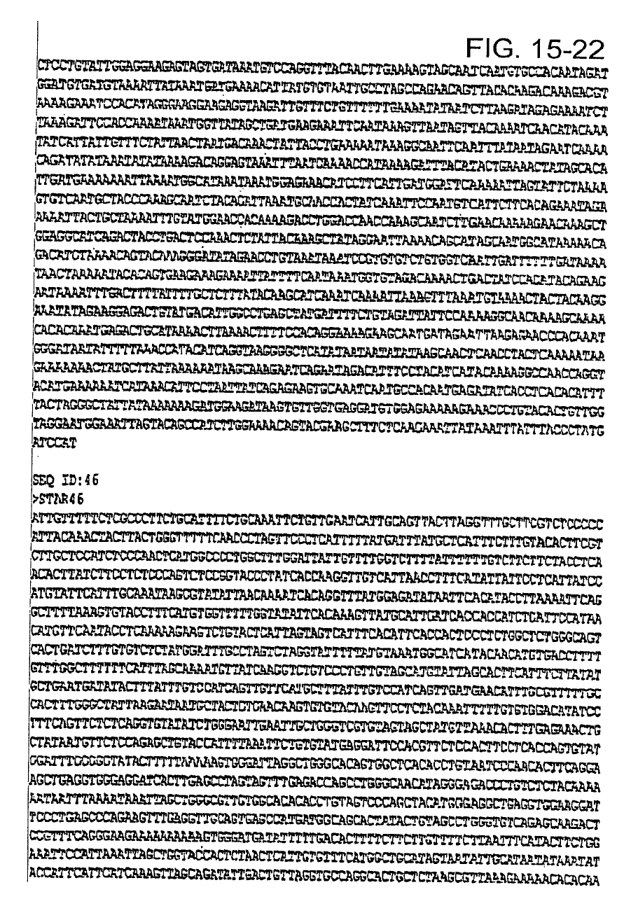

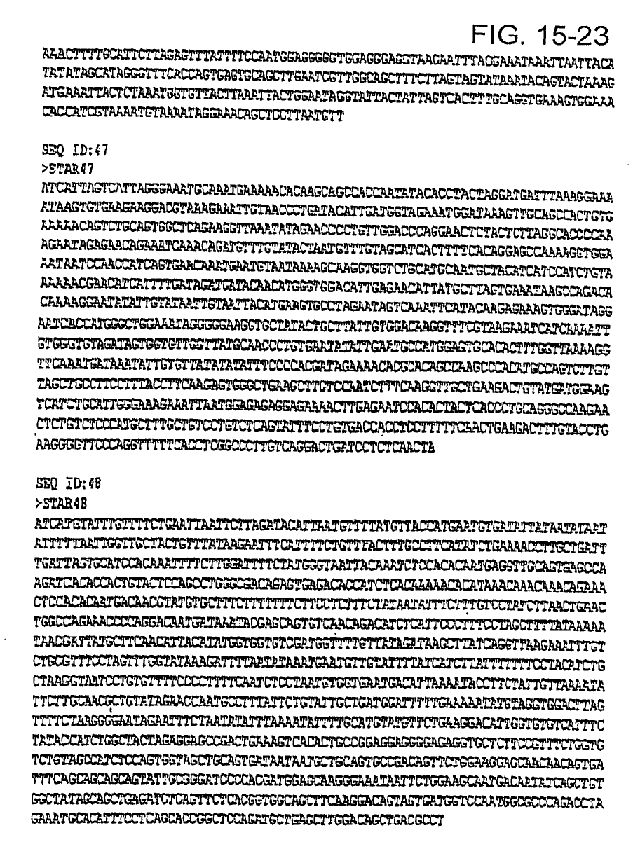

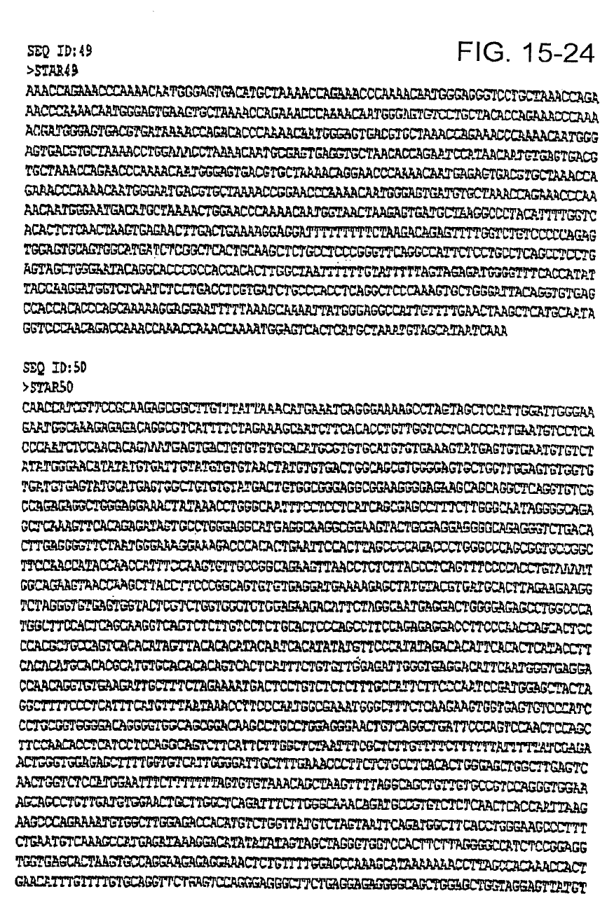

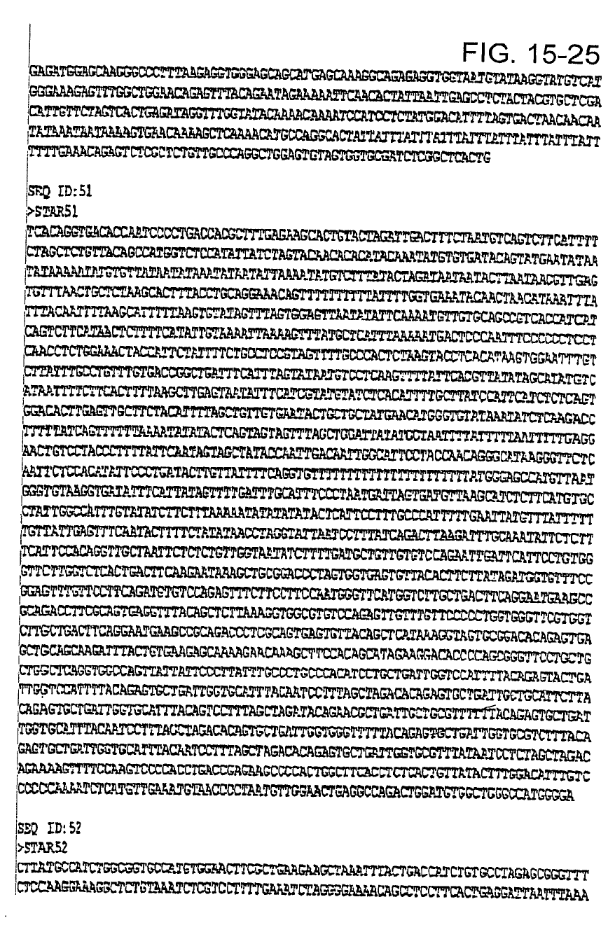

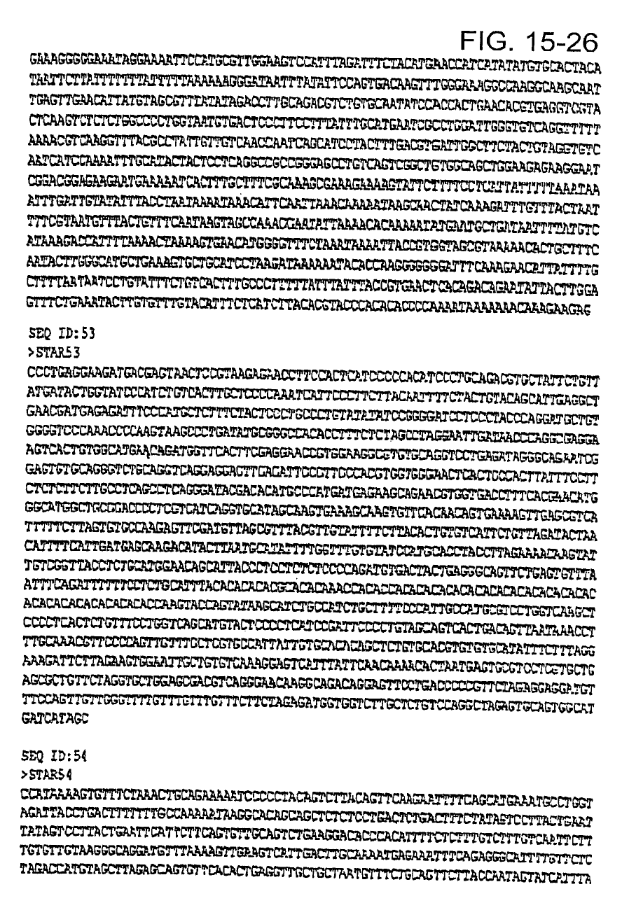

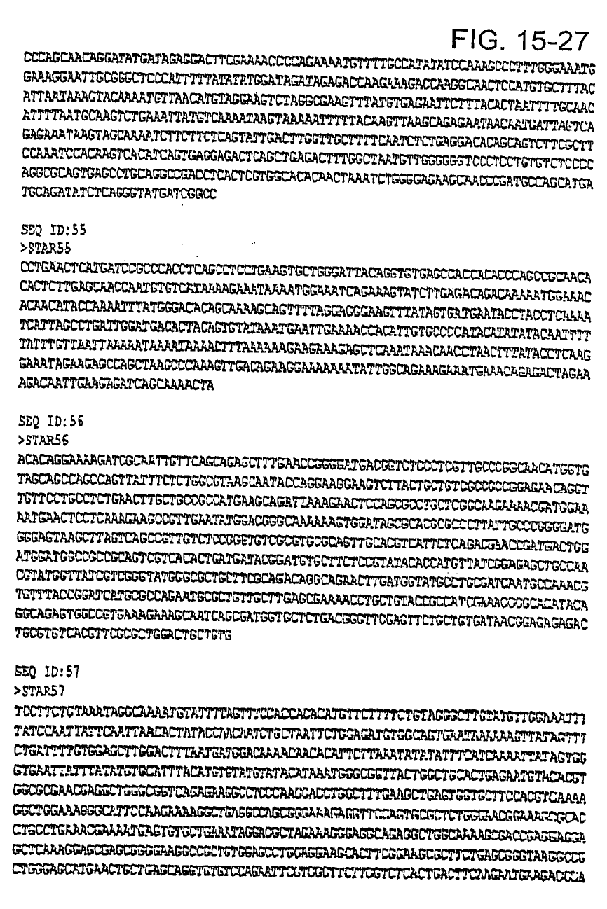

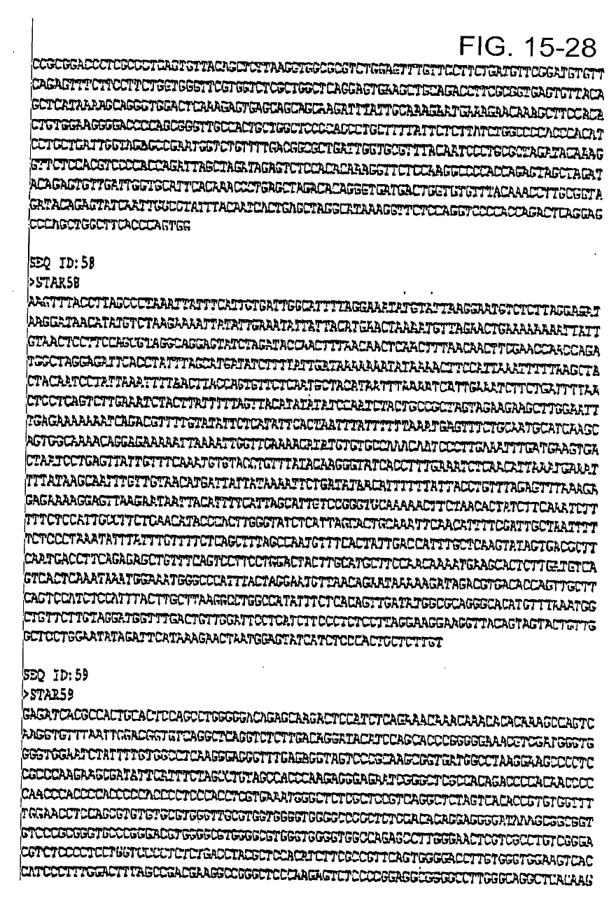

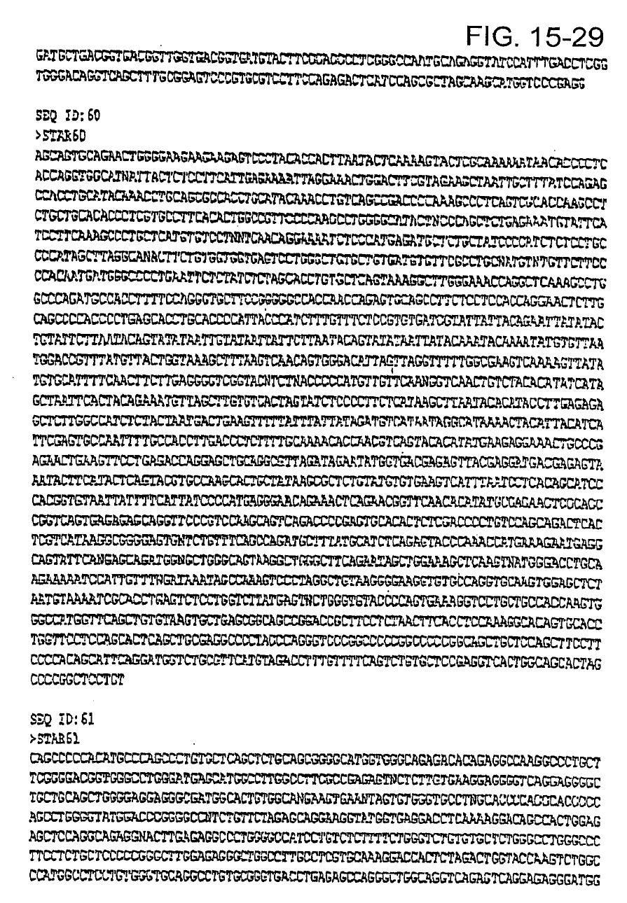

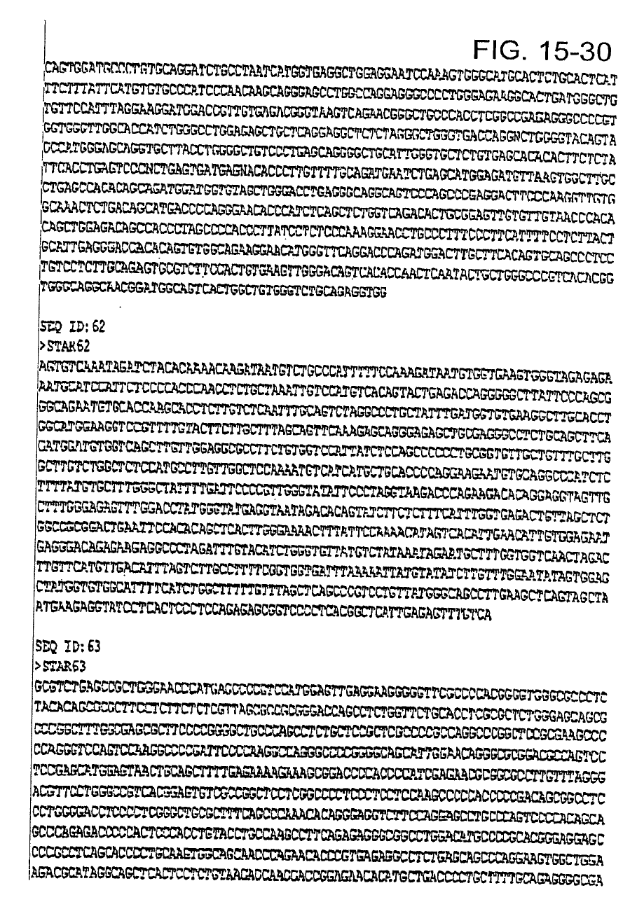

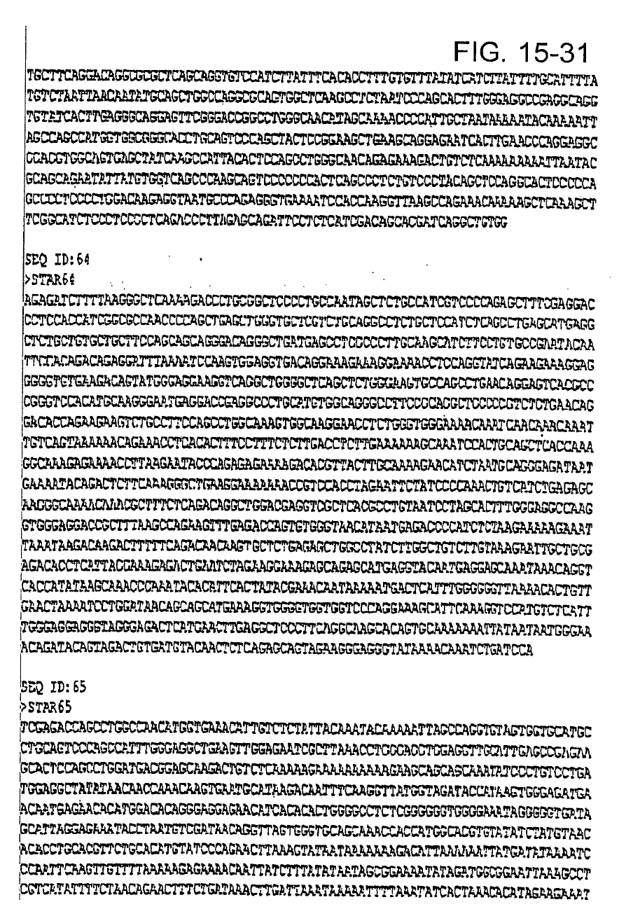

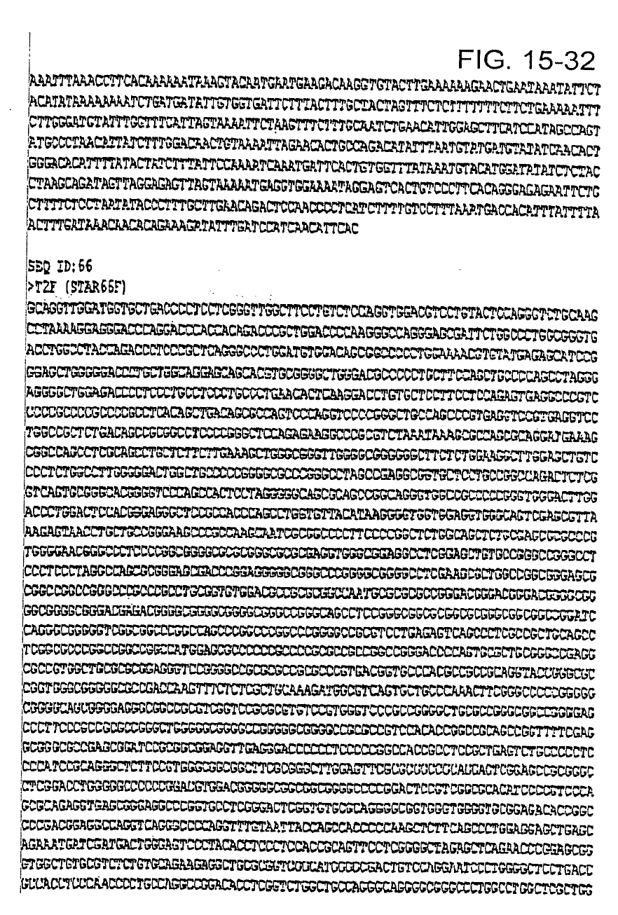

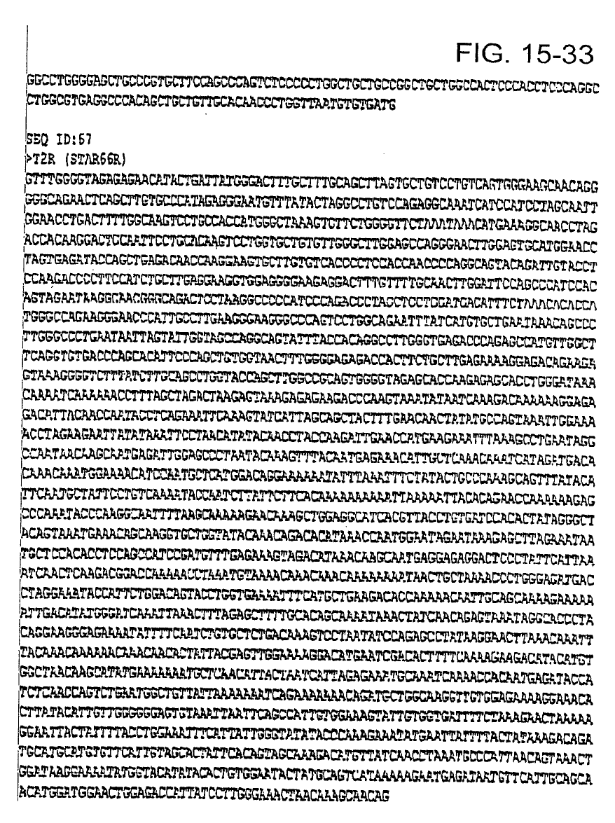

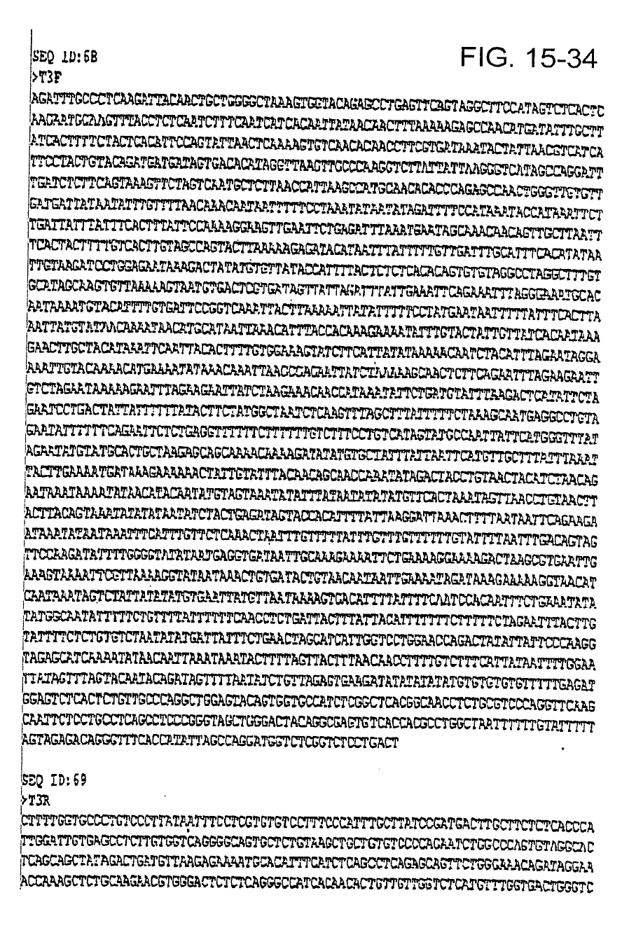

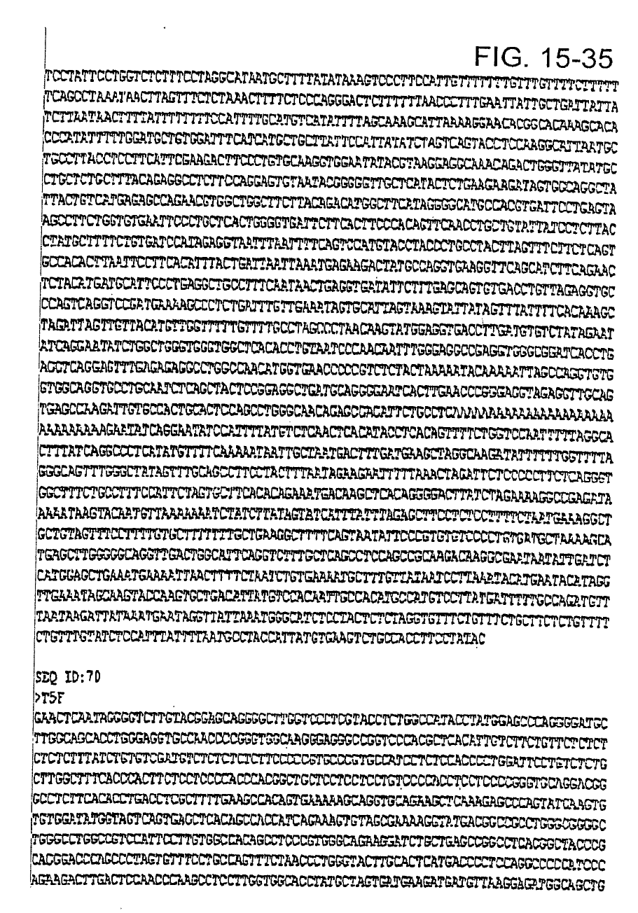

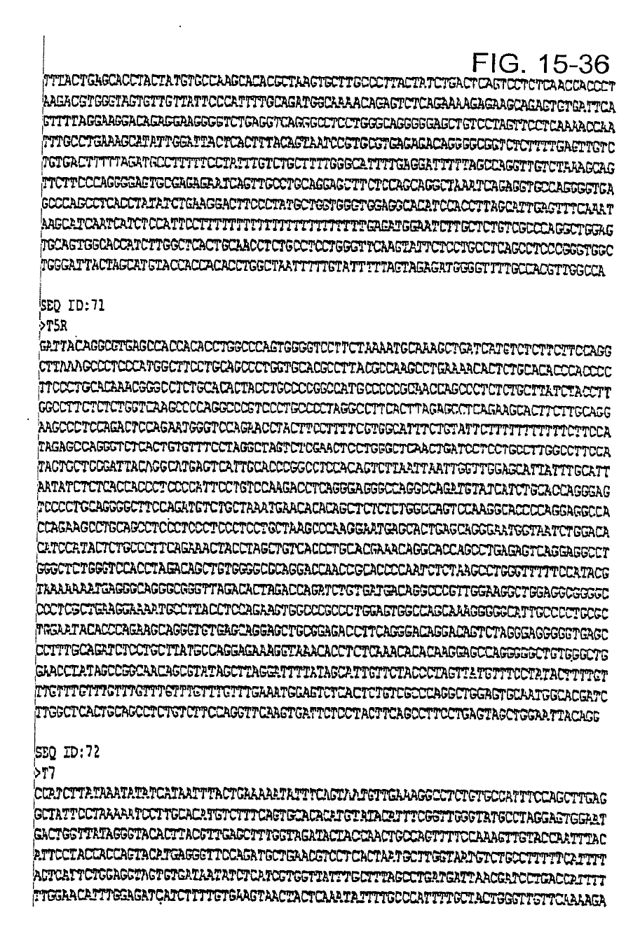

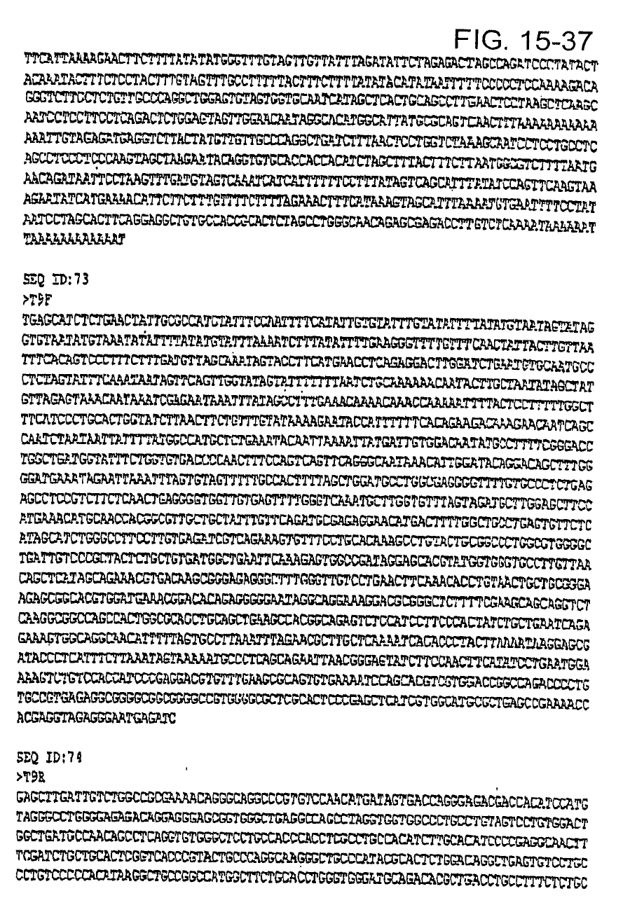

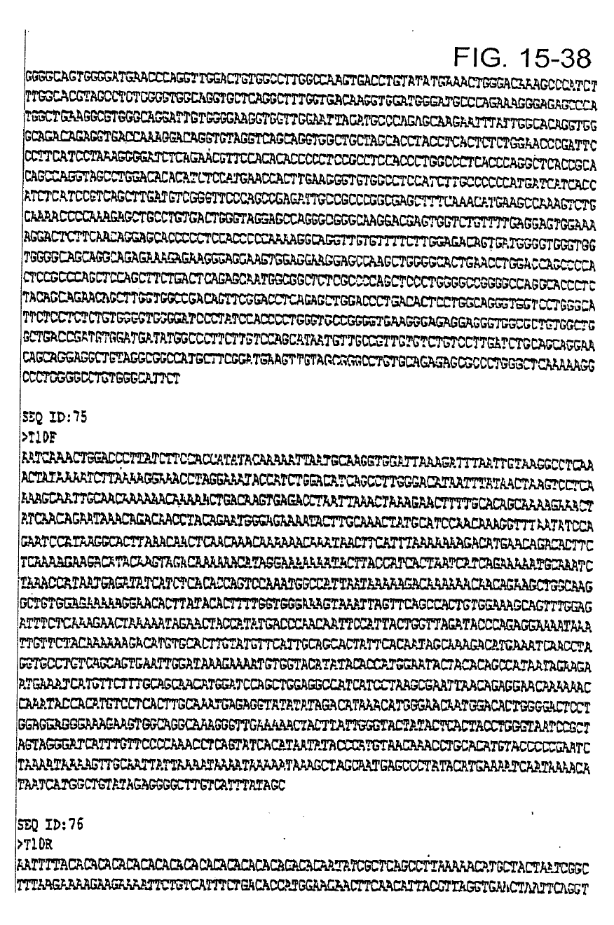

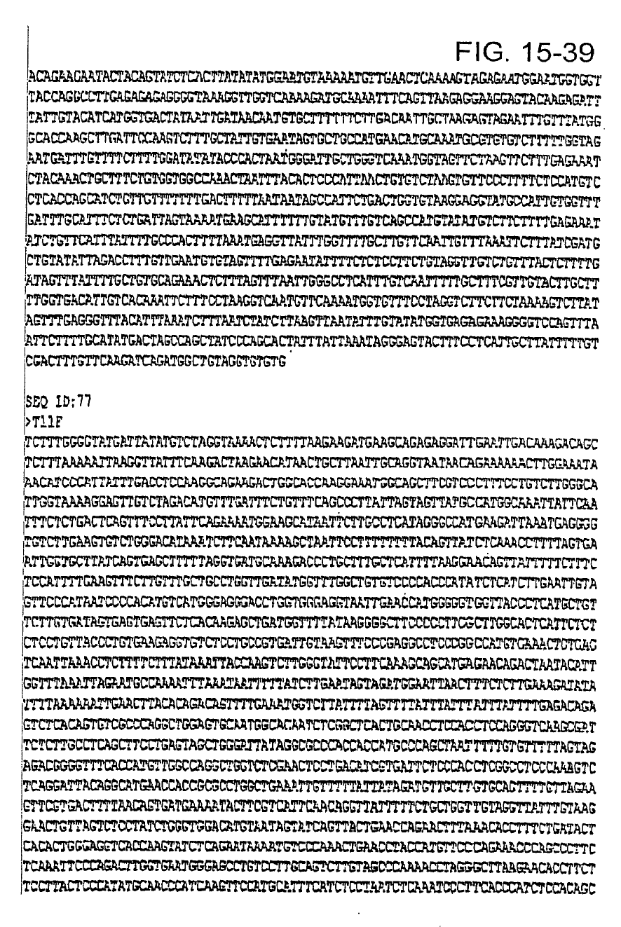

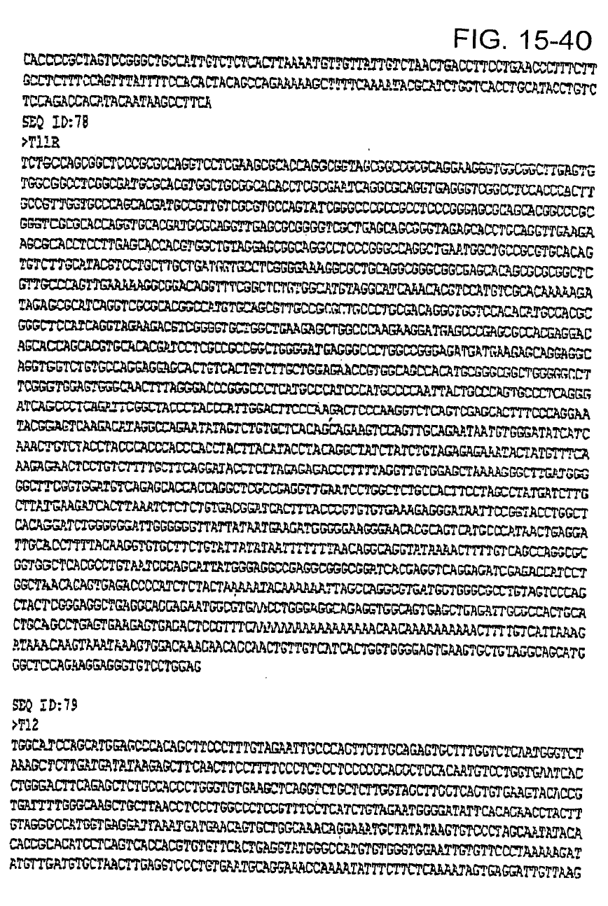

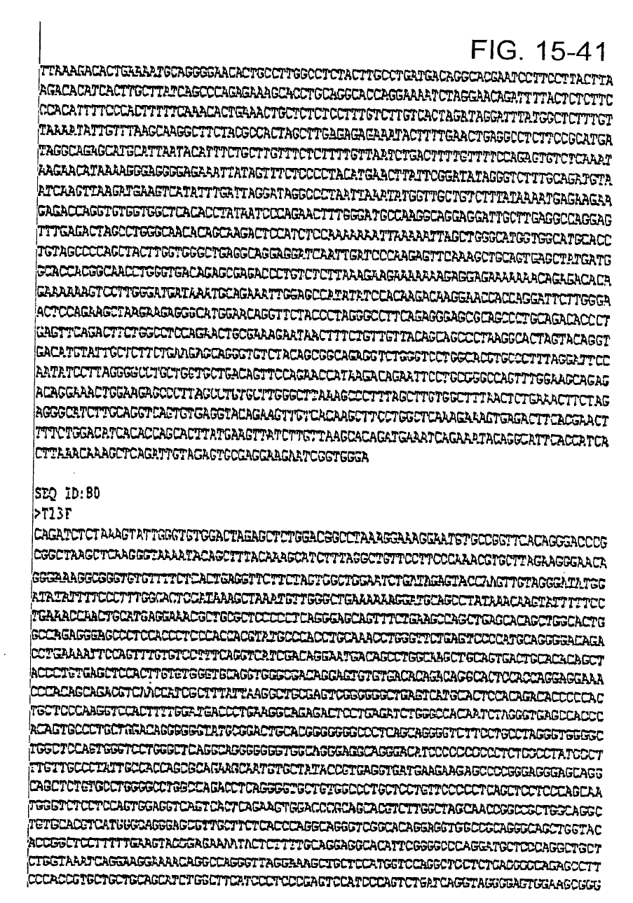

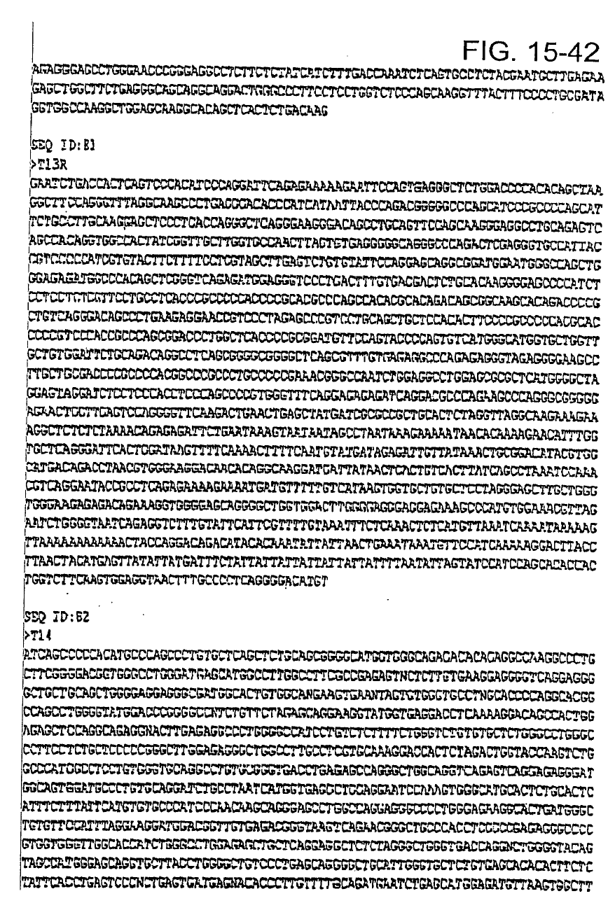

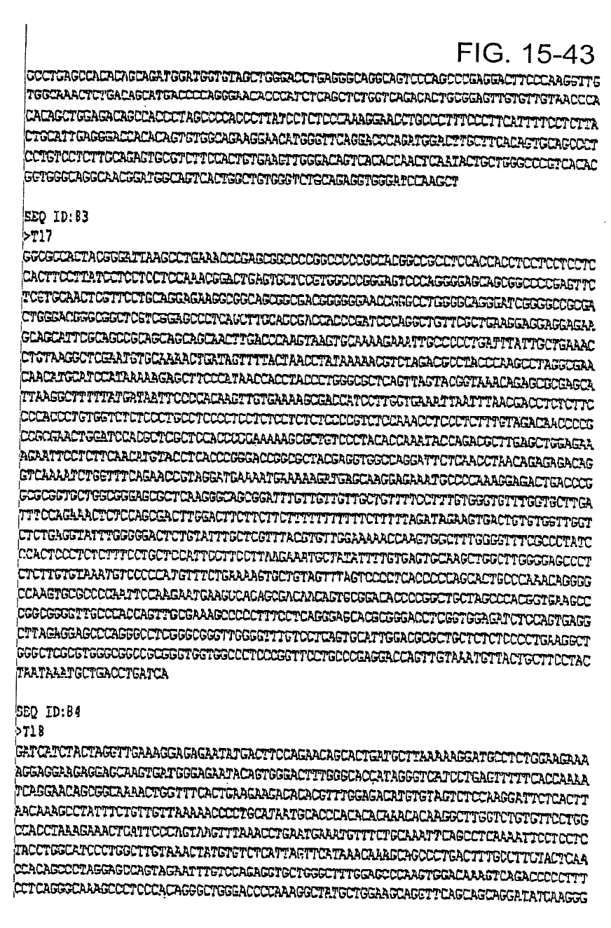

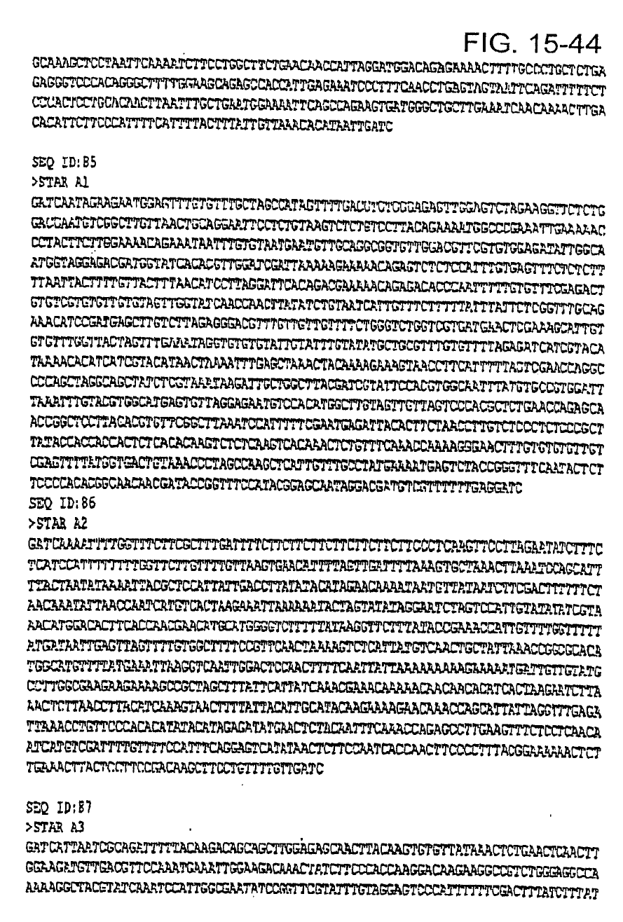

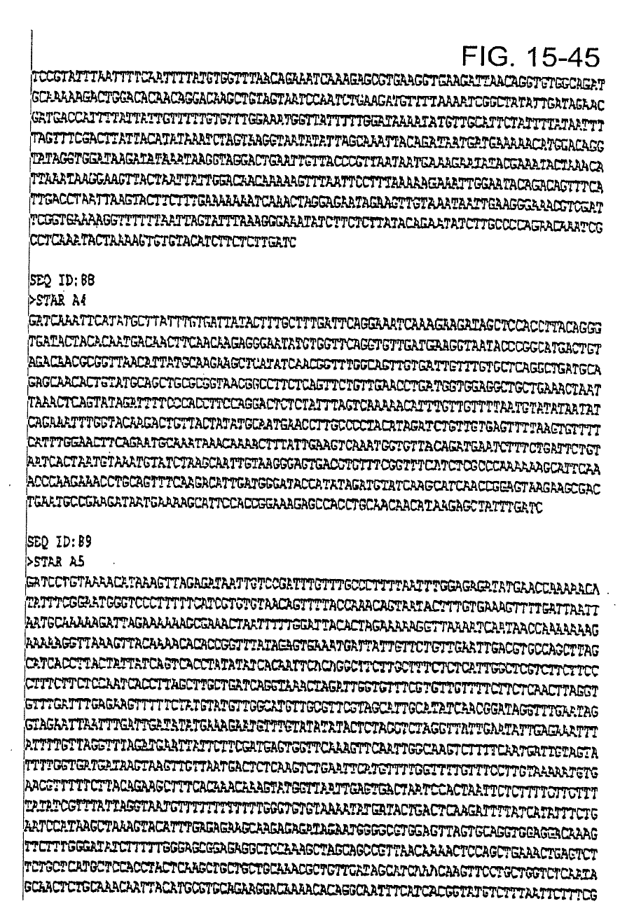

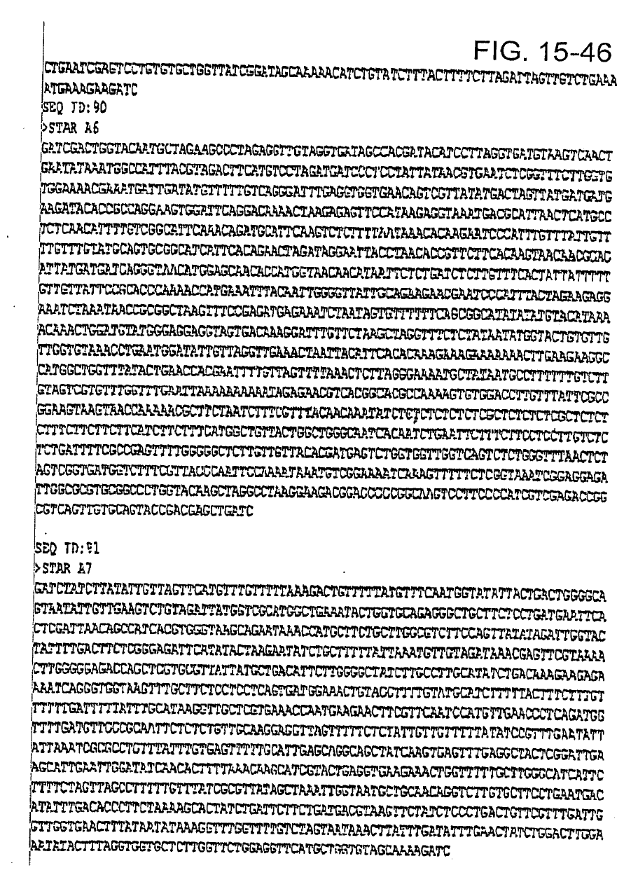

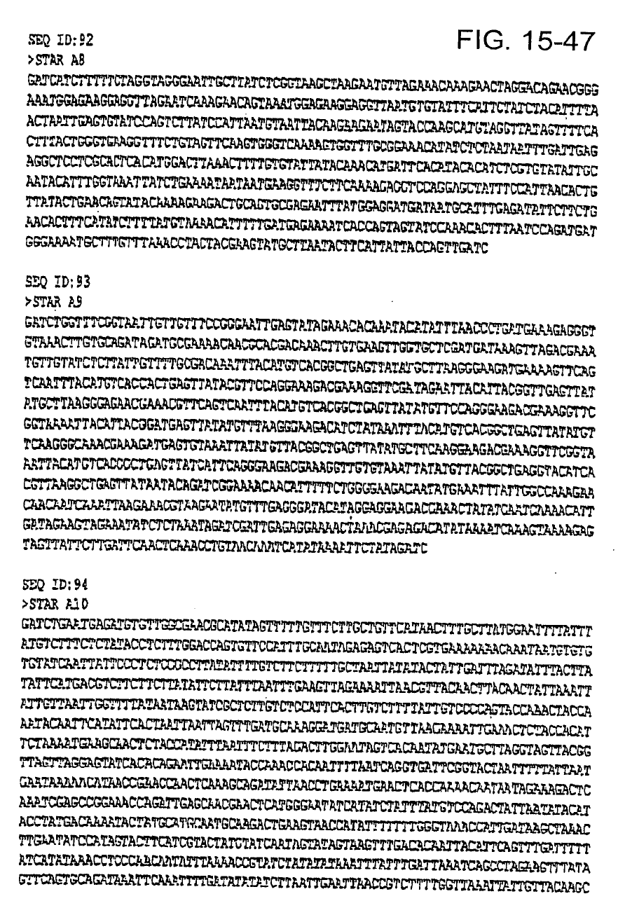

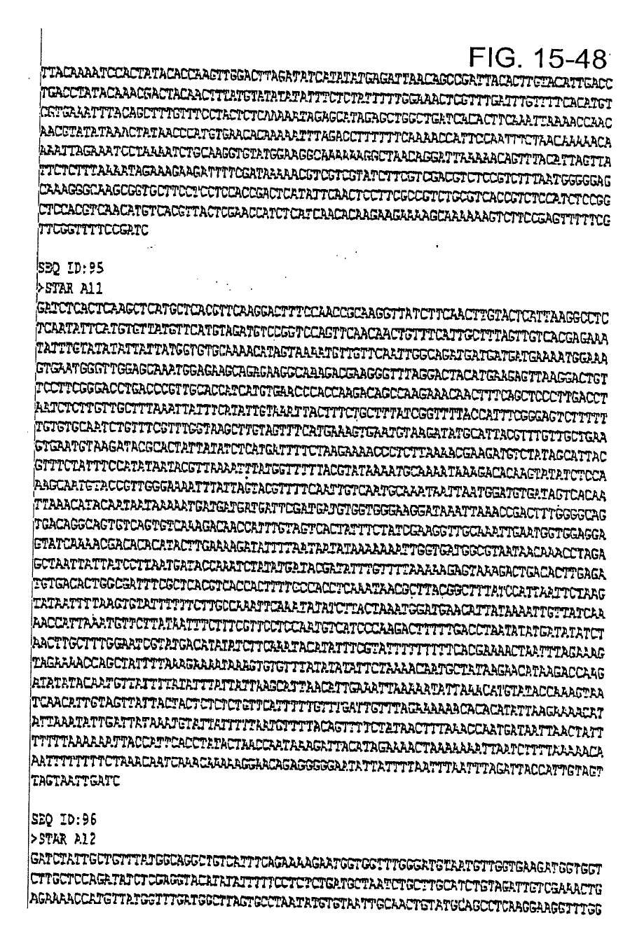

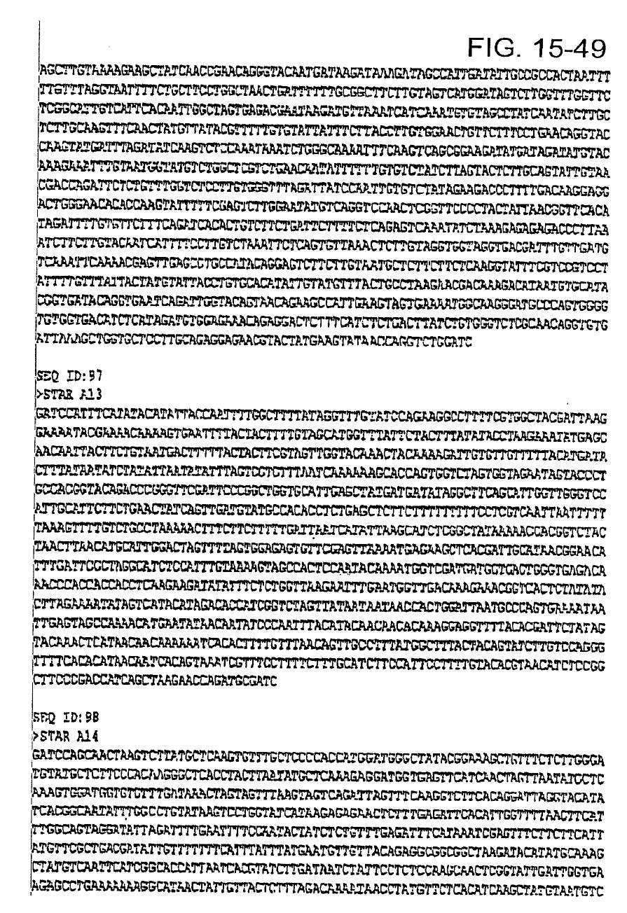

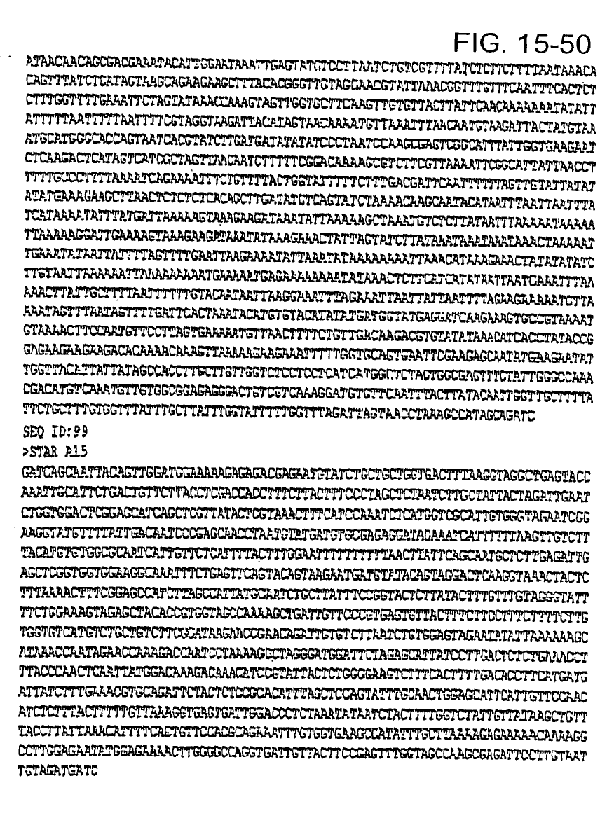

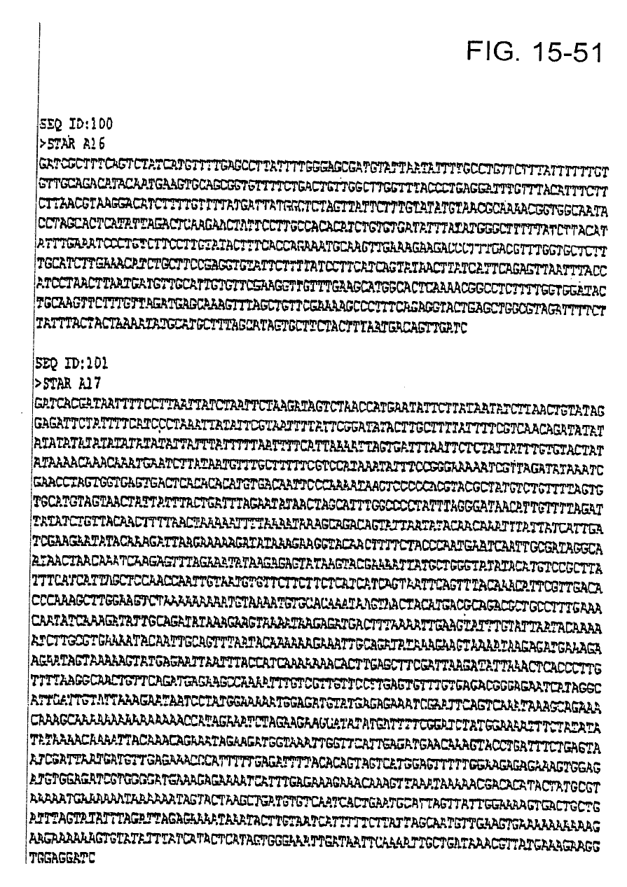

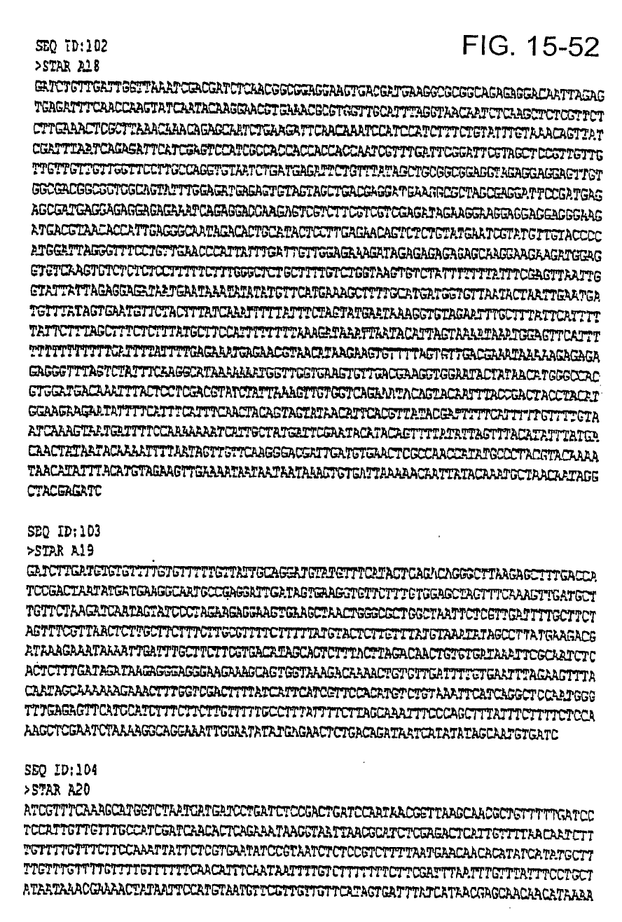

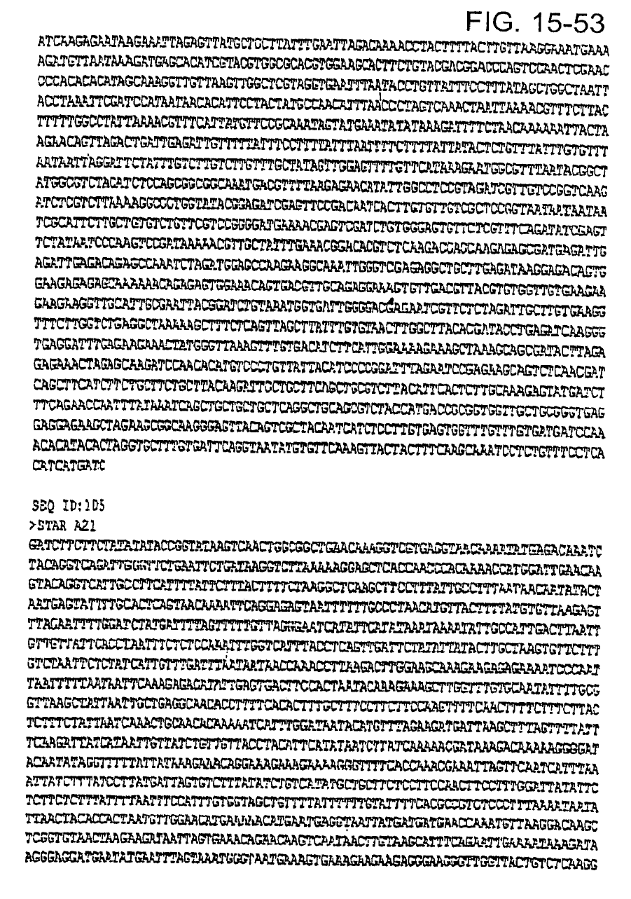

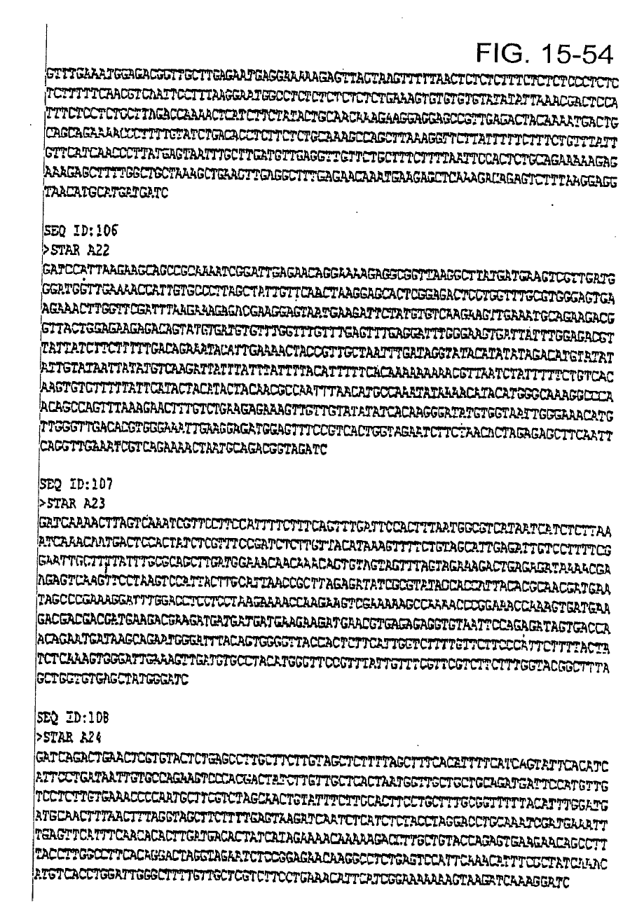

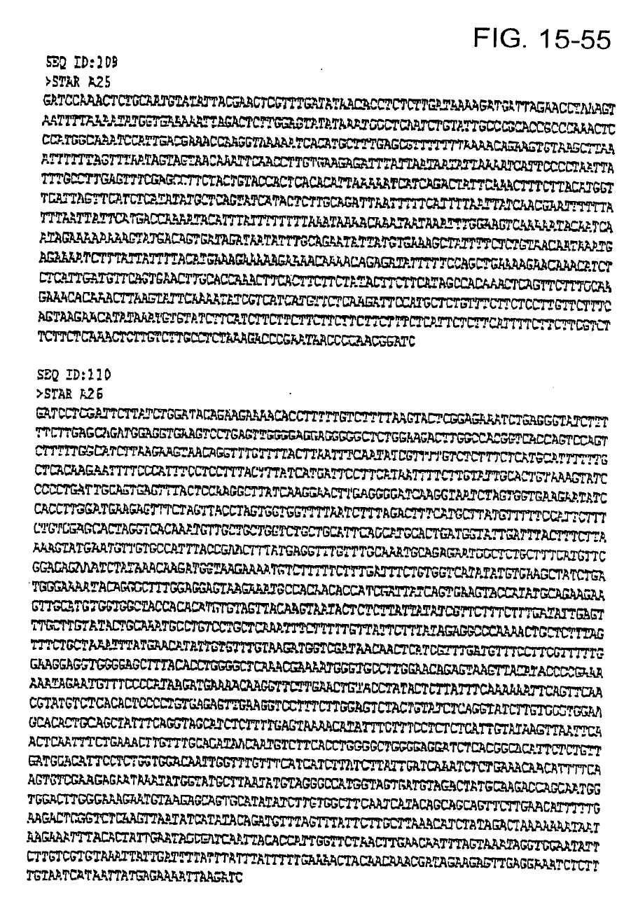

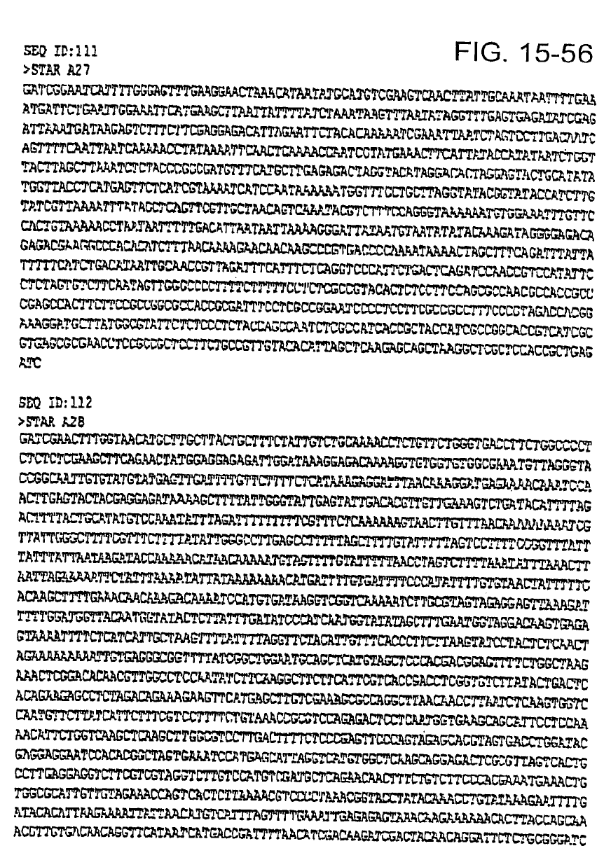

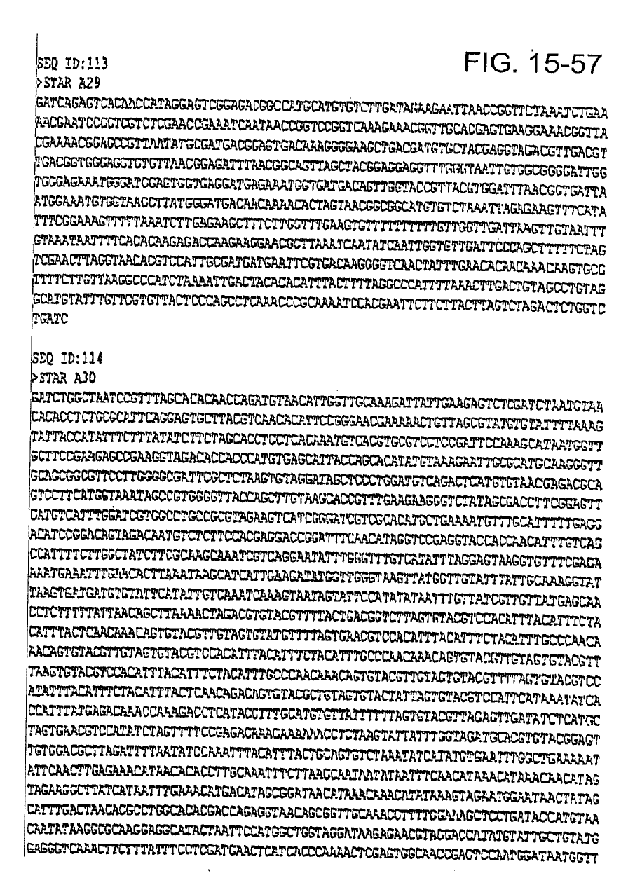

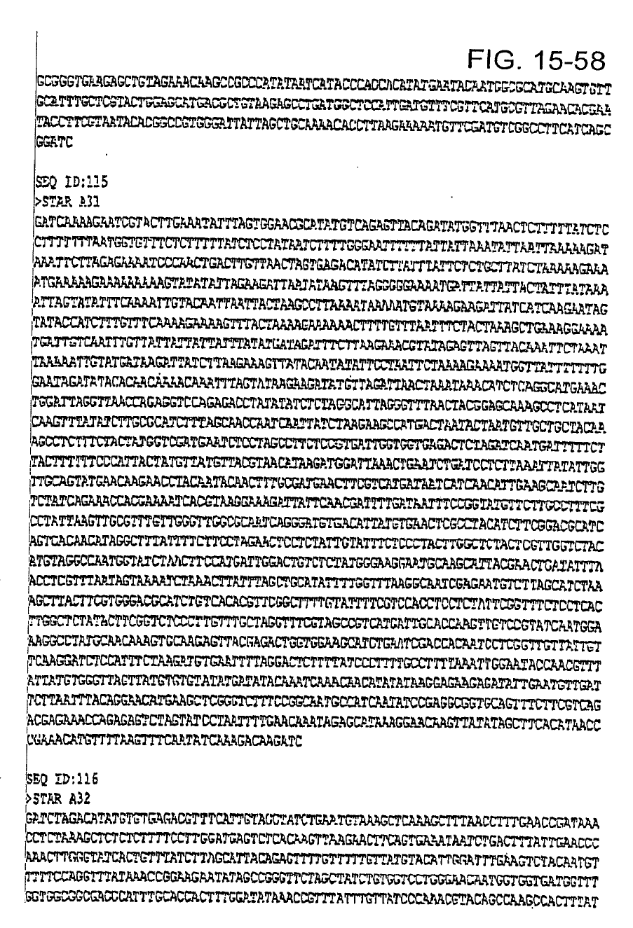

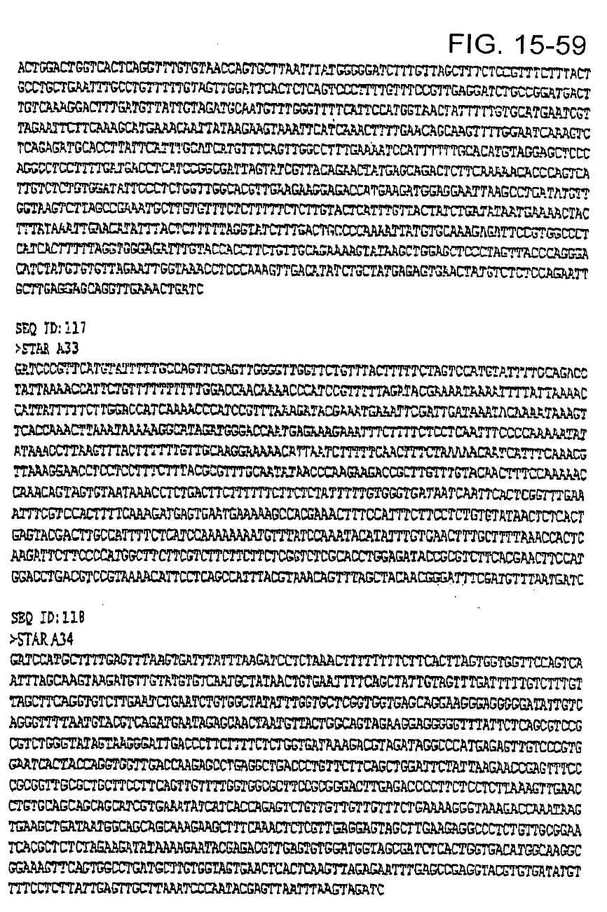

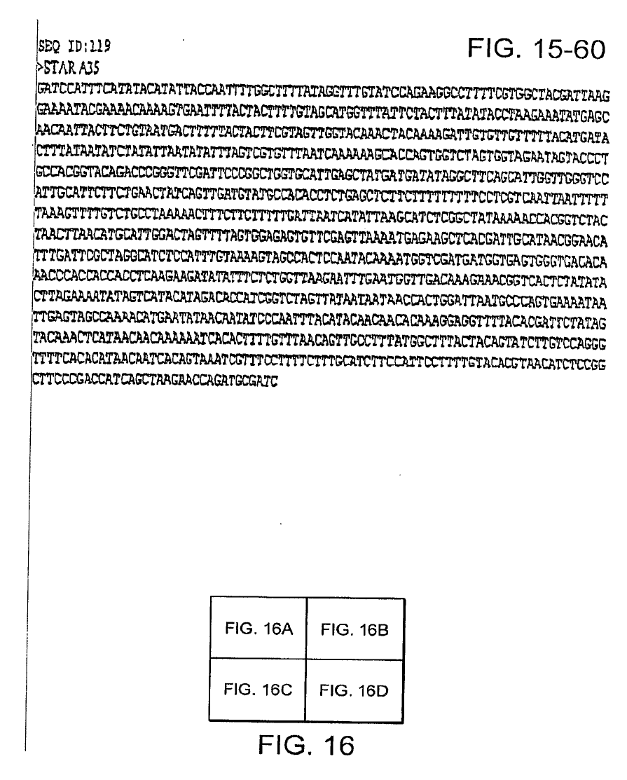

Suitable AREs for use in the present invention are described (e.g. in Kwaks et al. Nature Biotechnology, 21:553; and U.S. Patent Publication 2003/0199468, wherein AREs are referred to as “STAR” sequences). Sequences of exemplary AREs are provided by SEQ ID NOs: 1-119 of U.S. Patent Publication 2003/0199468, which are included herein as SEQ ID NOs: 1-119 (FIG. 15a), and in FIG. 5B of Kwaks et al., incorporated herein by reference as SEQ ID NOs: 121 and 122. For example, SEQ ID NOs: 1-66 provide certain human ARE sequences. Chromosomal locations of mouse homologs are also provided, and the corresponding nucleotide sequence can be readily identified from the publicly available sequence of the mouse genome. Genomic locations of additional human AREs are provided in Table 6 of U.S. Patent Publication 2003/0199468. The complete sequence of an ARE or a functional portion thereof, wherein the functional portion is at least approximately 50, at least approximately 100, at least approximately 150, or at least approximately 200 nucleotides in length, can be used. In certain embodiments an ARE comprises or consists of at least approximately 50, at least approximately 100, at least approximately 150, or at least approximately 200 nucleotides of the 3′ terminal portion of mouse homolog of anti-repressor 40, provided in SEQ ID NO: 122 or has a sequence at least approximately 80% to approximately 90% identical to any of SEQ ID NOs: 1-122 over at least approximately 50, approximately 100, approximately 150, or approximately 200 nucleotides, allowing the introduction of gaps.

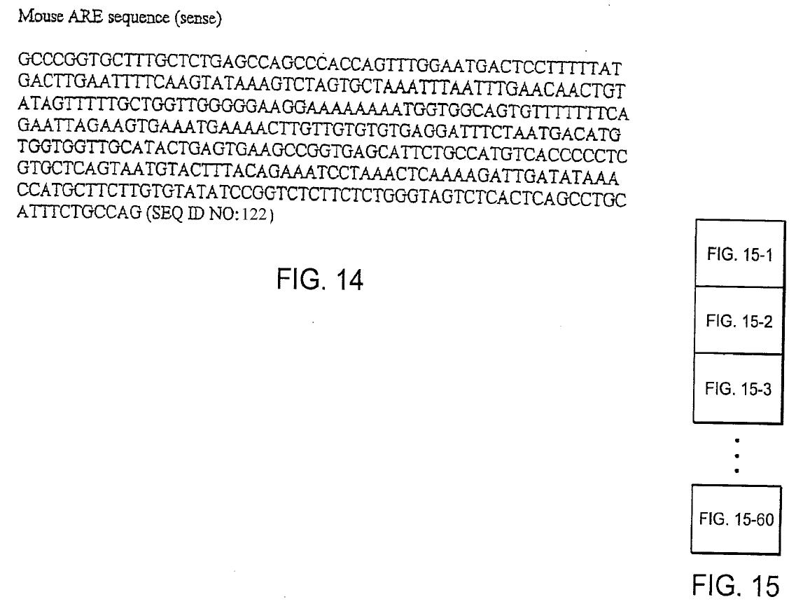

In one embodiment, an ARE comprises at least approximately 200 nucleotides of SEQ ID NO: 120 (FIG. 14) and/or comprises at least approximately 200 nucleotides of either of the sequences depicted in FIG. 5 of Kwaks et al., referred to as anti-repressor 40 (SEQ ID NOs: 121 and 122). For example, in one embodiment, an ARE comprises a portion of human or mouse anti-repressor 40 between approximately 200 to approximately 1000 base pairs in length. The portion may, for example, consist of between approximately 200 to approximately 1000 nucleotides of the 3′ terminal portion of anti-repressor 40, e.g., between approximately 200 to approximately 600 nucleotides, or between approximately 300 to approximately 500 nucleotides of the 3′ terminal portion of anti-repressor 40. In certain embodiments an ARE comprises or consists of at least approximately 50, at least approximately 100, at least approximately 150, or at least approximately 200 nucleotides of the 3′ terminal portion of mouse homolog of anti-repressor 40, provided in SEQ ID NO: 120 or has a sequence at least approximately 80% identical to SEQ ID NO: 120, 121, or 122 over at least approximately 50, approximately 100, approximately 150, or approximately 200 nucleotides, allowing the introduction of gaps. For example, an ARE may comprise or consist of any subsequence of SEQ ID NO: 120, 121, or 122 that is between approximately 50 and 381 nucleotides in length, e.g., between approximately 100 and 381, between approximately 150 and 381, between approximately 200 and 381 nucleotides in length; or may have a sequence at least approximately 80% identical to any subsequence of SEQ ID NO: 120, 121, or 122 that is between approximately 50 and 381, approximately 100 and 381, approximately 150 and 381, or approximately 200 and 381 nucleotides in length over at least approximately 50, approximately 100, approximately 150, or approximately 200 nucleotides respectively, allowing the introduction of gaps. For purposes of brevity, these individual sequences are not set forth herein.

A lentiviral vector of the present invention can comprise any SAR known in the art or discovered hereafter. In certain embodiments of the invention a SAR is a mammalian SAR, e.g., a human or rodent (e.g., mouse, rat, hamster) SAR. In certain embodiments of the invention a SAR is an avian (e.g., chicken) or plant (e.g., Arabadopsis) derived SAR. Many SARs are named based on their location relatively close to a particular gene, e.g., within approximately 1 to approximately 30 kB away from the gene. Exemplary SARs of use in the invention include, but are not limited to, the interferon-β (IFN-β) SAR (Klehr et al., 1991, Biochemistry, 30:1264), the Chinese hamster dihydrofolate reductase (DHFR) gene SARs (Kas et al. 1987, Mol. Biol., 198:677), the hypoxanthine-guanine phosphoribosyltransferase (HPRT) gene MAR (Sykes et al., 1988, Mol. Gen. Genet., 212:301); immunoglobulin heavy chain enhancer MAR (Cockerill et al., 1987, J. Biol. Chem., 262:5394; and Lutzko et al., 2003, J. Virol., 77:7341); immunoglobulin-kappa (Igkappa) SAR (Park et al., 2001, Mol. Ther., 4:164). In certain embodiments of the invention a SAR is one that is naturally located relatively close to a gene that is expressed in most or all cell types (e.g., a “housekeeping gene”). In certain embodiments of the invention a SAR is one that is naturally located relatively close to a tissue specific, lineage specific, or cell type specific gene, e.g., within about 30 kB of the gene. Such SARs may provide tissue specific, lineage specific, or cell type specific enhancement of expression. The immunoglobulin heavy chain SAR, which enhances expression in B cells, is but one example.

A typical ARE for use in the present invention increases the percentage of cells of multiple different types that express a transgene following lentiviral transgenesis. In other words, a transgenic animal generated using a lentiviral vector comprising an ARE expresses a lentivirally transferred transgene in a greater percentage of cells of multiple different types than a transgenic animal generated using an otherwise identical lentiviral vector not comprising an ARE. In certain embodiments of the invention the effect of an ARE is increased by and/or requires presence of a SAR in the lentiviral vector in addition to the ARE. Multiple cell types may, for example, be at least 2, 3, 4, or more different cell types. Cell types may be hematopoietic cell types such as T cells, B cells, granulocytes (e.g., neutrophils), macrophages, etc.

The ability of any ARE or any ARE and SAR to increase the percentage of cells that express a transgene may be determined by comparing the percentage of cells that express a transgene in transgenic animals generated using a lentiviral vector comprising an ARE or an ARE and SAR with the percentage of cells that express a transgene in transgenic animals generated using an otherwise identical lentiviral vector that does not comprise an ARE. A typical ARE is one whose presence in a lentiviral vector results in expression of a lentivirally transferred transgene in at least approximately 50% of the cells of 2, 3, 4, or more different cell types, e.g., any 2, 3, 4, or more hematopoietic cell types such as 13 cell, T cell, macrophages, granulocytes (e.g., neutrophils), etc., in a transgenic animal generated using the vector and/or in descendants of the transgenic animal. In certain embodiments of the invention the percentage of cells of multiple different types that express the transgene averages between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%.

In certain embodiments of the invention the effect of an ARE is increased by and/or requires presence of a SAR in the lentiviral vector in addition to an ARE. SARs can be similarly tested to determine whether they enhance the effect of any particular ARE on expression in multiple cell types following lentiviral transgenesis when present in a lentiviral vector that comprises an ARE. In certain embodiments of the invention an ARE or an ARE and SAR provide a stable increase in the percentage of cells that express a transgene in at least 2, 3, or 4 generations of descendants of the transgenic animal. For example, the percentage of cells of multiple different types that express a transgene averages between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%, in the F2, F3, and F4 generation. The cells may be, e.g., any 2, 3, 4, or more hematopoietic cell types such as B cell, T cell, macrophages, granulocytes, etc.

An ARE and optional SAR are preferably positioned in operable association with a regulatory sequence in a lentiviral vector of the invention. An ARE is considered to be in operable association with a regulatory sequence if it provides improved expression of a nucleic acid sequence that is positioned in operable association with the regulatory sequence in multiple cell types following lentiviral transgenesis, as described above, as compared with the expression that would be obtained without the ARE. A SAR is considered to be in operable association with an ARE and a regulatory sequence if it provides improved expression of a nucleic acid sequence that is positioned in operable association with the regulatory sequence in multiple cell types following lentiviral transgenesis as compared with the expression that would be obtained without the SAR. It will be appreciated that the position of an ARE and optional SAR with respect to regulatory sequence(s) can be varied and, if desired, can be optimized to provide desirable, e.g., maximum, percentages of transgene-expressing cells of any one or more cell types.

Lentiviral particles of the present invention include viral Gag, Pol, and Env proteins and a viral genome that comprises a nucleic acid comprising an ARE, sequences sufficient for reverse transcription and packaging, and optionally a SAR. In certain embodiments of the invention the viral genome further comprises regulatory sequences sufficient to promote transcription of an operably linked sequence of interest. In certain specific embodiments of the invention, recombinant lentiviral particles are replication-defective, i.e., the viral genome does not encode functional forms of all the proteins necessary for the infective cycle. For example, sequences encoding a structural protein or a protein required for replication may be mutated or disrupted or may be partly or completely deleted and/or replaced by a different nucleic acid sequence, e.g., a nucleic acid sequence of interest that is to be introduced into a target cell. However, sequences required for reverse transcription, integration, and packaging are typically functional.

Lentiviral particles of the invention may be produced using methods known in the art. To produce infectious viral particles that can be used to deliver a recombinant lentiviral genome to cells and mediate reverse transcription and integration, required viral proteins are provided in trans. Proteins may be provided by a packaging cell that has been engineered to produce them, e.g., by integrating coding regions of gag, pal, and env genes into the cellular genome, operably linked to suitable regulatory sequences for transcription of the coding region, which may or may not be derived from a virus. Packaging cell lines that express retrovirus proteins are well known in the art and include Ψ2, PA137, and PA12, etc. (see, e.g., U.S. Pat. Nos. 4,650,764, 5,955,331, and 6,013,516; and Sheridan et al., 2000, Molecular Therapy, 2:262). To produce a recombinant virus, a packaging cell is stably or transiently transfected with a vector, e.g., a plasmid, that provides a replication defective viral genome comprising functional sequences for reverse transcription, integration; and packaging. Viral genomes transcribed from the vector are packaged with viral enzymes, yielding infectious viral particles. Alternatively or additionally, a helper virus can be used.

Instead of using packaging cell lines that stably express required viral proteins, cells can be transfected with vectors, e.g., plasmids, that comprise nucleic acid sequences encoding the proteins, operably linked to regulatory sequences for transcription of the coding region, that may or may not be derived from a virus (see, e.g., U.S. Pat. No. 6,013,516; Naldini et al., 1996, Proc. Natl. Acad. Sci., USA, 93:11382; and Naldini et al., 1996, Science, 272:263). For example, three vectors can be used to produce recombinant lentiviral particles. A first vector comprises sequences encoding structural proteins and enzymes of a lentivirus. A second vector comprises sequences encoding an envelope protein. These vectors can, and preferably do, lack functional cis-acting viral sequences needed for reverse transcription, integration, and packaging. Thus they typically lack LTRs and instead use a non-LTR promoter to drive transcription.

A third vector includes cis-acting viral sequences necessary for reverse transcription, integration, and packaging, which typically include at least a portion of one or both LTRs. The third vector includes a site (e.g., a restriction site) into which a nucleic acid sequence of interest is or can be inserted. In some embodiments, insertion may destroy the restriction site. Such a vector is referred to in the art and herein as a “transfer vector,” “transfer construct,” or “transfer plasmid.” A lentiviral transfer vector comprising an ARE and optionally a SAR is an aspect of the present invention. Optionally a transfer vector may include an internal promoter or other regulatory sequence(s) that can drive expression of an operably linked nucleic acid sequence of interest. Following insertion of the nucleic acid sequence of interest into a transfer vector, the three vectors are co-transfected into suitable cells for production of viral particles. Many different types of cell may be used to generate infectious viral particles, provided that the cells are permissive for transcription from the promoters employed. Suitable host cells include, for example, 293 cells and derivatives thereof such as 293.T, 293FT (Invitrogen), 293F, NIH3T3 cells and derivatives thereof, etc.

The various proteins need not originate from the same virus. For example, gag and poi genes may be derived from any of a wide variety of retroviruses or lentiviruses. According to certain embodiments of the invention gag and pol genes are derived from a lentivirus. According to certain embodiments of the invention gag and poi genes are derived from HIV, e.g., HIV-1 or HIV-2. Envelope protein can be derived from the same virus from which the other viral proteins are derived, from a different retrovirus or lentivirus, or can include portions of envelope proteins that originate from two or more retroviruses or lentiviruses. Alternatively or additionally, a non-retroviral envelope protein such as the VSV G glycoprotein is used. Use of a non-retroviral envelope protein can significantly reduce or eliminate the possibility of generating replication competent virus during vector manufacturing or after introduction of the vectors into cells and can expand the range of cell types and/or species that virus can enter. Thus the envelope protein may be one that allows virus to enter cells of only a single species (e.g., cells of a species that is a natural host for virus from which the envelope protein is derived) or may allow virus to enter cells of multiple different species. For example, envelope protein may limit the range of species whose cells can be entered to mice and/or other rodents, or may limit the range to humans and/or other primates or may allow entry of rodent and primate cells.

A lentiviral vector comprising a nucleic acid that comprises an ARE and, optionally, a SAR, can be constructed using any suitable method known in the art. Lentiviral transfer plasmids may be constructed using standard methods of molecular biology. An ARE or SAR can be amplified from genomic DNA, e.g., using PCR, and appropriate amplification primers. An ARE or SAR can be provided as a restriction fragment that can be linked to other nucleic acids to construct a plasmid or recombinant lentiviral genome. Alternatively or additionally, an ARE or SAR can be inserted into an existing plasmid or lentiviral genome. An ARE and, optionally, a SAR, can be inserted into any lentiviral transfer plasmid known in the art or any newly designed lentiviral transfer plasmid or recombinant lentiviral genome. Examples of useful transfer plasmids into which an ARE and optional SAR can be inserted include the pLL series of vectors (U.S. Patent Publication 2005/0251872; Rubinson, et al., 2003), pFUGW or pBFGW (Lois et al. 2002, Science, 295:868), pCCL (Zufferey et al., 1998, J. Virol., 72:9873), and variants of any of the foregoing, e.g., transfer plasmids that comprise different or additional promoters or other regulatory sequences. The resulting lentiviral transfer plasmid may be used to produce lentiviral particles whose genome comprises an ARE and optional SAR or for any of a variety of other purposes described herein. Alternatively or additionally, an ARE and optional SAR can be inserted directly into any nucleic acid comprising a naturally occurring or recombinant lentiviral genome known in the art.

Either an ARE, the SAR, or both, can be present in a lentiviral vector in either orientation relative to its naturally occurring orientation in a eukaryotic genome. Certain SARs such as the IFN-β SAR are desirably present in reverse orientation in the lentiviral vector relative to their naturally occurring orientation.

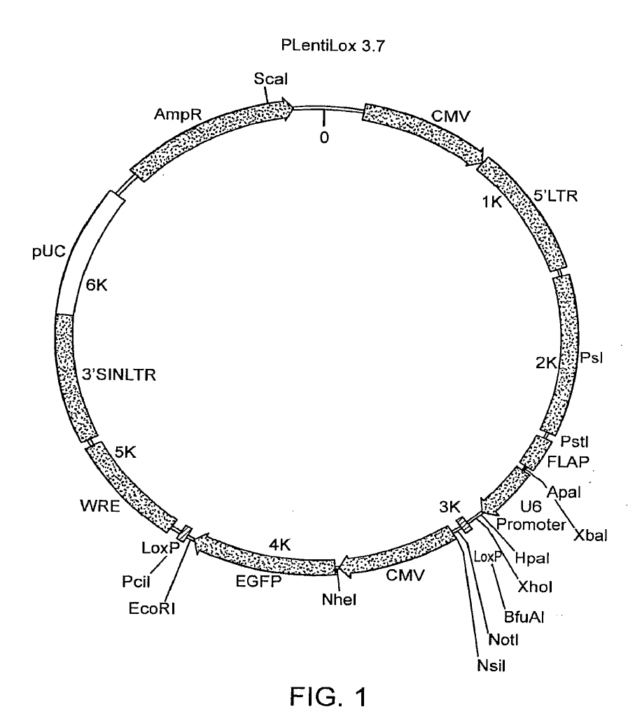

An exemplary lentiviral transfer plasmid, pLL3.7 is shown in FIG. 1, prior to introduction of an ARE and optional SAR (see U.S. Patent Publication 2005/0251872 for the nucleotide sequence of this plasmid). For purposes of description, nucleotides are numbered in a clockwise direction with reference to nucleotide 0 (indicated on the Figure), and elements having lower nucleotide numbers are considered 5′ to elements having higher nucleotide numbers. Thus, for example, the cauliflower mosaic virus (CMV) element is 5′ to all other elements shown. Various sequence elements depicted in the map are not shown to scale. Presence of a particular element on a map is not intended to indicate that the entire sequence element is necessarily present. For example, according to certain embodiments of the invention a portion of the 5′ LTR is deleted.

An ARE and, optionally, a SAR can be inserted in a variety of different locations in a lentiviral vector such as pLL3.7. Typically an ARE and optional SAR are inserted between portions of the vector that comprise sequences for reverse transcription and packaging. In certain embodiments of the invention the vector comprises 5′ and 3′ LTRs and the ARE and optional SAR are located between the 5′ and 3′ LTR. The ARE and SAR may be located in the 3′ direction from a packaging sequence. The ARE may be located 5′ to the SAR or 3′ to the SAR. The ARE and SAR may flank regulatory sequences sufficient to promote transcription of an operably linked nucleic acid sequence, e.g., a sequence that encodes an RNA of interest, e.g., an RNAi agent or a coding sequence for a polypeptide of interest. Regulatory sequences may comprise an RNA polymerase I or III (Pol I or Pol III) promoter functional in eukaryotic cells, e.g., mammalian or avian cells. Regulatory sequences may be located upstream of a site for insertion of a heterologous nucleic acid. An ARE and SAR may flank two or more distinct regulatory sequences, e.g., two different promoters, each capable of promoting transcription of an operably linked nucleic acid. An ARE and SAR may flank an expression cassette that encodes an RNA of interest, e.g., an RNAi agent or a coding sequence for a polypeptide of interest. Typically an ARE and optional SAR are positioned appropriately with respect to the regulatory sequence(s) so that the ARE and optional SAR provide improved expression of a nucleic acid sequence in operable association with the regulatory sequences in multiple cell types following lentiviral transgenesis, as described above. An ARE may be separated from a regulatory sequence by between, e.g., approximately 10 nucleotides and approximately 1000 nucleotides or any intervening number of nucleotides in various embodiments of the invention. A SAR may be separated from the 3′ end of a heterologous nucleic acid in operable association with a regulatory sequence by, e.g., between approximately 10 nucleotides and approximately 1000 nucleotides or any intervening number of nucleotides.

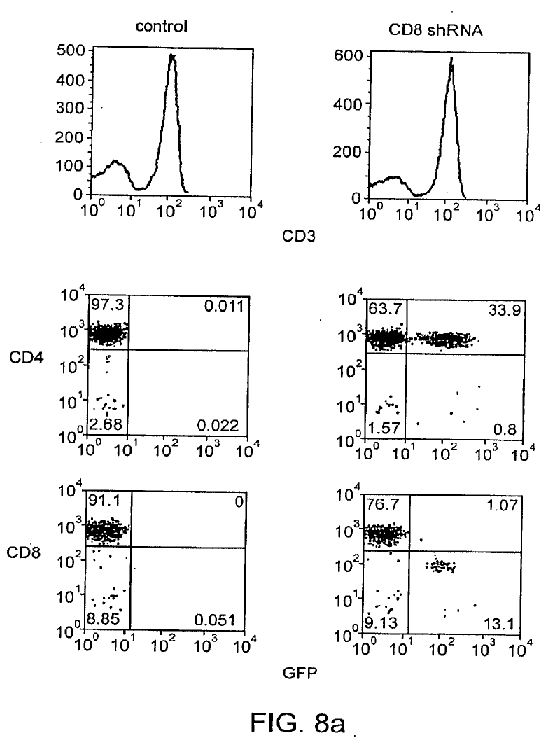

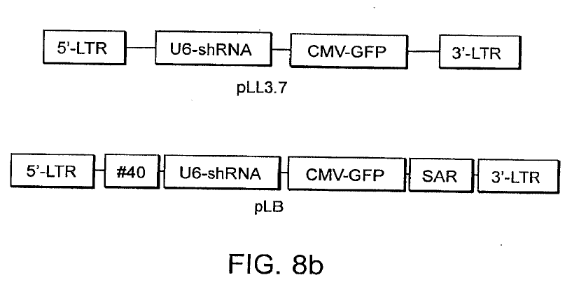

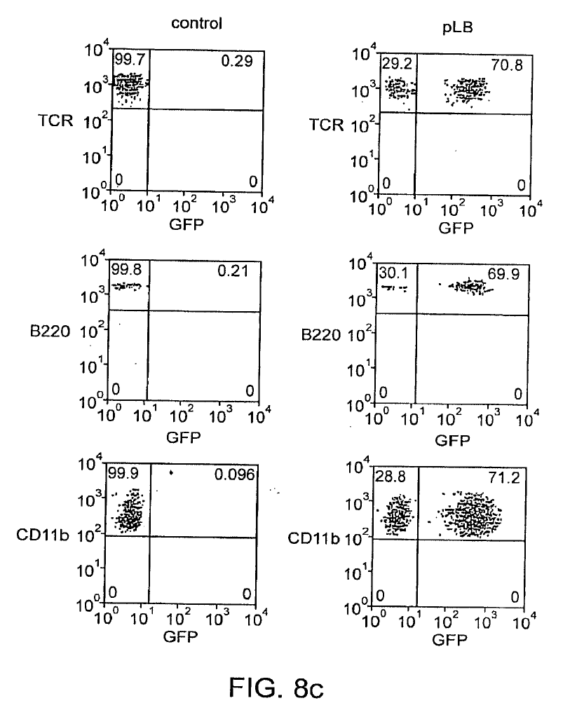

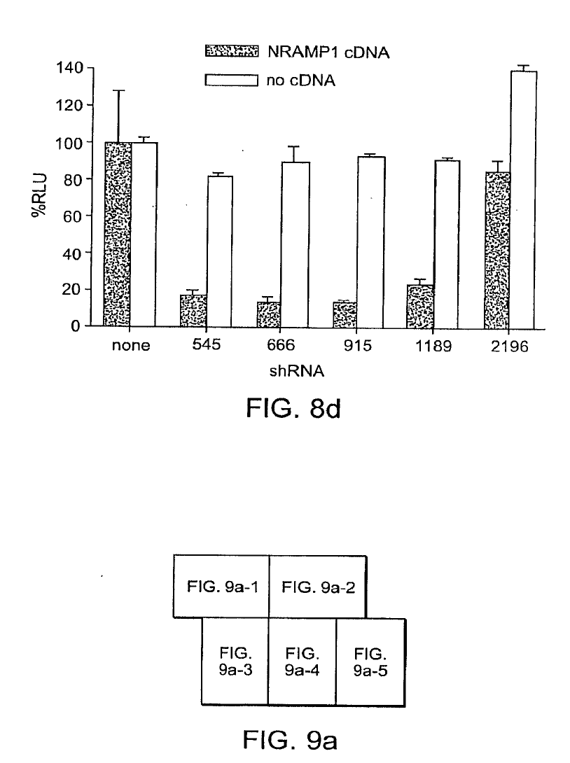

The upper portion of FIG. 8b depicts a portion of pLL3.7 prior to insertion of an ARE and SAR. Certain sequence elements that may be present, some of which are described below, are omitted. For example, the vector may comprise an HIV FLAP element, a posttranscriptional regulatory element, etc. The portion of the vector as shown in FIG. 8b encodes an shRNA in operable association with the U6 promoter, but it is to be understood that a vector of the invention includes versions either with or without a heterologous sequence in operable association with the regulatory sequences included in the vector. The lower portion of FIG. 8b shows a portion of an exemplary vector of the present invention, pLB, which was created by inserting an ARE (a portion of anti-repressor 40) and an SAR into pLL3.7. As shown in FIG. 8b, the ARE is located in the 3′ direction from the 5′ LTR and the SAR is located in the 5′ direction from the 3′ LTR. The ARE and SAR flank two expression cassettes, one of which comprises a template for transcription of an RNA that self-hybridizes to form an shRNA and the other of which comprises a coding sequence for a reporter. It is to be understood that a vector of the invention includes versions either with or without a heterologous sequence in operable association with the regulatory sequences included in the vector. It will be appreciated that a variety of additional elements may be included in the cassette whose borders are defined by the LTRs and that the elements may be provided in a variety of orders.

Representative exemplary arrangements of the various sequence elements in a lentiviral vector of the invention are: 5′LTR-ARE-regulatory sequence-SAR-3′ LTR or 5′LTR-SAR-regulatory sequence-ARE-3′LTR or 5′LTR-ARE-regulatory sequence 1-regulatory sequence 2-SAR-3′ LTR or 5′LTR-ARE-regulatory sequence 1 SAR-regulatory sequence 2-3′ LTR or 5′LTR-SAR-regulatory sequence 1 regulatory sequence 2-ARE-3′ LTR or 5′LTR-ARE-regulatory sequence 1-SAR-regulatory sequence 2-3′. If the cassette includes additional elements such as a FLAP element and/or PRE, the order may be 5′LTR-FLAP-ARE-regulatory sequence 1-SAR-PRE-3′ LTR 5′LTR-FLAP-SAR-regulatory sequence 1-ARE-PRE-3′ LTR or 5′LTR-FLAP-ARE-regulatory sequence 1-regulatory sequence 2-SAR-PRE-3′ LTR- or 5′LTR-FLAP-SAR-regulatory sequence 1-regulatory sequence 2-ARE-PRE-3′ LTR or 5′LTR-FLAP-ARE-regulatory sequence 1-SAR-regulatory sequence 2-PRE-3′ LTR. In certain embodiments of the present invention a first regulatory sequence comprises a pol l or pol III promoter and a second regulatory sequence comprises a Pol II promoter. The invention provides vectors that comprise heterologous nucleic acids operably linked to regulatory sequences and vectors that do not comprise heterologous nucleic acids but into which heterologous nucleic acids may be inserted. In some embodiments vectors include at least one cloning site, e.g., a restriction site. Either or both regulatory sequences may have a cloning site situated in proximity to it, e.g., in the 3′ direction, such that a heterologous nucleic acid sequence inserted into the cloning site would be in operable association with the regulatory sequence. The cloning site may be a multiple cloning site (MCS) comprising at least two restriction sites, e.g., 2, 3, 4, 5, or more restriction sites.

According to certain embodiments of the invention, lentiviral vectors are HIV-based. As used herein, a lentiviral vector is said to be “based on” a particular lentivirus species (e.g., HIV-1) or group (e.g., primate lentivirus group) if (i) at least approximately 50% of the lentiviral sequences found in the vector are derived from a lentivirus of that particular species or group or (ii) the lentiviral sequences are at least approximately 50% identical to either a particular lentivirus species or group member, or (iii) the lentiviral sequences display greater identity or homology to a lentivirus of that particular species or group than to other known lentiviruses. In certain embodiments of the invention at least approximately 50%, approximately 60%, approximately 70%, approximately 80%, approximately 90% or more, e.g., all, of the lentiviral sequences are derived from (i.e., originate from), HIV-1 or HIV-2. Whether a sequence is derived from a particular Lentivirus can be determined by sequence comparison using, e.g., a program such as BLAST, BLASTNR, or CLUSTALW (or variations thereof), which are well known in the art. BLAST is described (Altschul et al., 1990, J. Mol. Biol., 215:403; Altschul and Gish, Methods in Enzymology). Searches and sequence comparisons can be performed using default parameters and matrices (e.g., BLOSUM substitution matrix), typically allowing gaps so as to maximize identity.

As noted above, a lentiviral vector typically comprises a nucleic acid that includes cis-acting sequence elements required to support reverse transcription of a lentiviral genome and also cis-acting sequence elements necessary for packaging and integration. These sequences typically include the Psi (Ψ) packaging sequence, reverse transcription signals, integration signals, promoter or promoter/enhancer, polyadenylation sequence, tRNA binding site, and origin for second strand DNA synthesis. According to certain embodiments of the invention the vector comprises a Rev Response Element (RRE) such as that located at positions 7622-8459 in the HIV NL4-3 genome (Genbank accession number AF003887). RREs from other strains of HIV could also be used. Such sequences are readily available from Genbank or from the database with URL hiv-web.lanl.gov/content/index. In certain embodiments of the invention a vector comprises a 5′ HIV R-U5-del gag element such as that located at positions 454-1126 in the HIV NL4-3 genome. In certain specific embodiments of the invention the transfer plasmid comprises a sequence encoding a selectable marker and an origin of replication that allows the plasmid to replicate within bacterial cells. Any of a variety of genes encoding a selectable marker known in the art could be used, e.g., the ampicillin resistance gene (AmpR), kanamycin resistance gene (KanR), etc. Any of a variety of origins of replication known in the art could be used, e.g., the pUC origin. Further details of various features and elements mentioned above (and others) are more fully described in the following sections.

Lentiviral Sequences

Lentiviral transfer vectors and lentiviral particles of the invention may include lentiviral sequences derived from any of a wide variety of lentiviruses including, but not limited to, primate lentivirus group viruses such as human immunodeficiency viruses HIV-1 and HIV-2 or simian immunodeficiency virus (SIV); feline lentivirus group viruses such as feline immunodeficiency virus (FIV); ovine/caprine immunodeficiency group viruses such as caprine arthritis encephalitis virus (CAEV); bovine immunodeficiency-like virus (BIV); equine lentivirus group viruses such as equine infectious anemia virus (EIAV); and visna/maedi (VMV) virus. It will be appreciated that each of these viruses exists in multiple variants or strains.

According to certain specific embodiments of the invention, most or all of the lentiviral sequences are derived from HIV-1. However, it is to be understood that many different sources of lentiviral sequences can be used, and numerous substitutions and alterations in certain of the lentiviral sequences may be accommodated without impairing the ability of a transfer plasmid to perform the functions described herein. Such variations are within the scope of the invention. The ability of any particular lentiviral transfer plasmid to transfer nucleic acids and/or to be used to produce a lentiviral particle capable of infecting and transducing cells may readily be tested by methods known in the art, some of which are described herein and/or in the references.

Long Terminal Repeats (LTRs)

A lentiviral transfer plasmid or the genome of a lentiviral particle of the invention typically comprises at least one LTR or portion thereof. In certain embodiments of the invention the lentiviral transfer plasmid or genome comprises two LTRs or portions thereof, wherein the two LTRs or portions thereof flank regulatory sequences that are sufficient to promote transcription of an operably linked nucleic acid. According to certain embodiments of the invention the transfer vector includes a self-inactivating (SIN) LTR. As is known in the art, during the retroviral life cycle, the U3 region of the 3′ LTR is duplicated to form the corresponding region of the 5′ LTR in the course of reverse transcription and viral DNA synthesis. In one embodiment, creation of a SIN LTR is achieved by inactivating the U3 region of the 3′ LTR (e.g., by deletion of a portion thereof as described in Miyoshi, et al., 2003). The alteration is transferred to the 5′ LTR after reverse transcription, thus eliminating the transcriptional unit of the LTRs in the provirus, which should prevent mobilization by replication competent virus. An additional safety enhancement is provided by replacing the U3 region of the LTR with a heterologous promoter to drive transcription of the viral genome during production of viral particles. Appropriate promoters include, e.g., the CMV promoter or promoter-enhances (Schmidt, 1990). Typical promoters are able to drive high levels of transcription in a Tat-independent manner. This replacement reduces the possibility of recombination to generate replication-competent virus because there is no complete U3 sequence in the virus production system. Thus, in certain embodiments of the invention, a transfer plasmid includes a self-inactivating (SIN) 3′ LTR. In certain embodiments of the invention, a transfer plasmid includes a 5′ LTR in which the U3 region is replaced with a heterologous promoter. The heterologous promoter drives transcription during transient transfection, but after reverse transcription, it gets replaced by a copy of U3 from the 3′ LTR, which in the case of a SIN LTR comprises a deletion that makes it unable to drive transcription. Thus all transcription is driven by the internal promoter after integration.

FLAP Element

According to certain embodiments of the invention a transfer plasmid includes a FLAP element. As used herein, the term “FLAP element” refers to a nucleic acid whose sequence includes the central polypurine tract and central termination sequences (cPPT and CTS) of a retrovirus. Typically the retrovirus is a lentivirus, e.g., HIV-1 or HIV-2. Suitable FLAP elements are described in U.S. Pat. No. 6,682,907. As described therein and in Zennou, et al., (2000, Cell, 101:173), during HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus.

Expression-Stimulating Posttranscriptional Regulatory Element

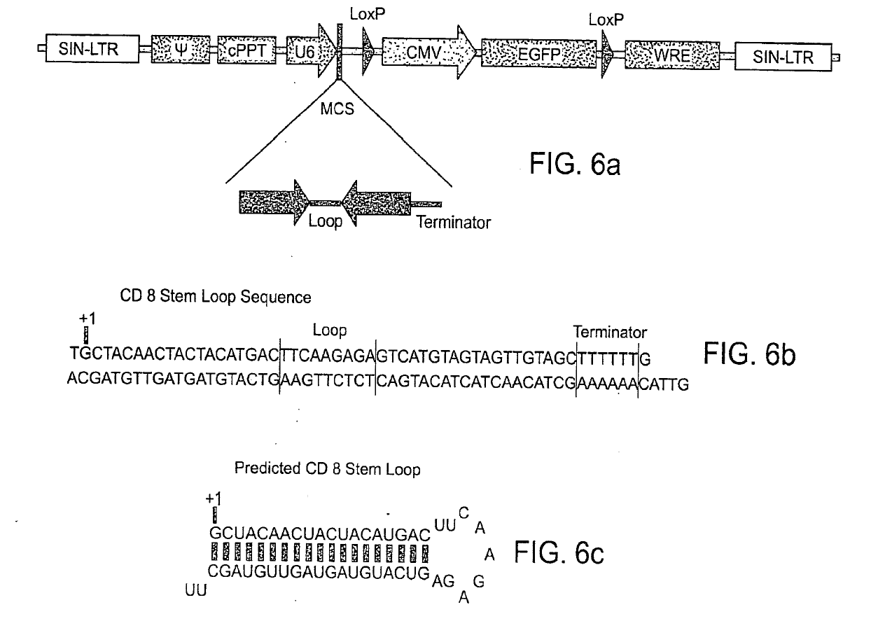

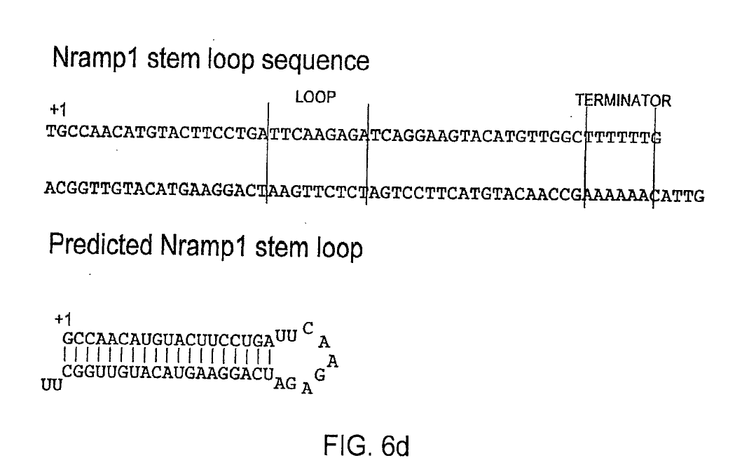

In certain embodiments of the invention, lentiviral vectors comprise any of a variety of posttranscriptional regulatory elements whose presence within a transcript increases expression of the heterologous nucleic acid at the protein level. One example is the posttranscriptional regulatory element (PRE) is the woodchuck hepatitis virus regulatory element (WRE) as described (Zufferey et al., 1999, J. Virol., 73:2886): Other posttranscriptional regulatory elements that may be used include the posttranscriptional processing element present within the genome of various viruses such as that present within the thymidine kinase gene of herpes simplex virus (Liu et al., 1995, Genes Dev., 9:1766), and the posttranscriptional regulatory element (PRE) present in hepatitis B virus (HBV) (Huang et al., Mol. Cell. Biol., 5:3864). The posttranscriptional regulatory element is positioned so that a heterologous nucleic acid inserted into the transfer plasmid in the 5′ direction from the element will result in production of a transcript that includes the posttranscriptional regulatory element at the 3′ end. FIG. 1 shows an example of a transfer plasmid incorporating a WRE downstream of sites for insertion of one or more heterologous nucleic acid sequences. FIG. 6 shows an example of a transfer plasmid in which a heterologous nucleic acid encoding EGFP has been inserted in the 5′ direction from a WRE and the ubiquitin C (UbC) promoter has been inserted upstream of the sequence encoding EGFP. This configuration results in synthesis of a transcript whose 5′ portion comprises EGFP coding sequences and whose 3′ portion comprises the WRE sequence.

Insulators

According to certain embodiments of the invention, a lentiviral vector further comprises an insulator. Insulators are elements that can help to preserve the independent function of genes or transcription units embedded in a genome or genetic context in which their expression may otherwise be influenced by regulatory signals within the genome or genetic context (see, e.g., Burgess-Beusse et al., 2002, Proc. Natl. Acad. Sci., USA, 99:16433; and Zhan et al., 2001, Hum. Genet., 109:471). In the context of the present invention, insulators may contribute to protecting lentivirus-expressed sequences from integration site effects, which may be mediated by cis-acting elements present in genomic DNA and lead to deregulated expression of transferred sequences. The invention provides transfer vectors in which an insulator sequence is inserted into one or both LTRs or elsewhere in the region of the vector that integrates into the cellular genome.

Promoters and Other Transcription Promoting Regulatory Elements

Any of a wide variety of regulatory sequences sufficient to promote transcription of an operably linked nucleic acid may be included in lentiviral vectors of the present invention. A vector may include one, two, or more heterologous promoters or promoter/enhancer regions, where “heterologous” here means that the regulatory sequence is not derived from the same lentivirus as the sequences sufficient for reverse transcription and/or packaging. They may be derived from a eukaryotic organism, from a virus other than a lentivirus, or from a different lentivirus. The regulatory sequences may be in the same or in opposite orientation with respect to each other.

One of ordinary skill in the art will readily be able to select appropriate regulatory sequences depending upon the particular application. For example, sometimes it will be desirable to achieve constitutive, non-tissue specific, high level expression of a heterologous nucleic acid sequence. For such purposes viral promoters or promoter/enhancers such as the SV40 promoter, CMV promoter or promoter/enhancer, etc., may be employed. Mammalian promoters such as the beta-actin promoter, ubiquitin C promoter, elongation factor 1α promoter, tubulin promoter, etc., may also be used. If the vectors are to be used in non-mammalian cells, e.g., avian cells, appropriate promoters for such cells should be selected. It may be desirable to achieve cell type specific, lineage specific, or tissue-specific expression of a heterologous nucleic acid sequence (e.g., to express a particular heterologous nucleic acid in only a subset of cell types or tissues or during specific stages of development), tissue-specific promoters may be used. For example, it may be desirable to achieve conditional expression in the case of transgenic animals or for therapeutic applications, including gene therapy. As used herein, the terms “cell type specific” or “tissue specific promoter” refers to a regulatory element (e.g., promoter, promoter/enhancer or portion thereof) that preferentially directs transcription in only a subset of cell or tissue types, or during discrete stages in the development of a cell, tissue, or organism. A tissue specific promoter may direct transcription in only a single cell type or in multiple cell types (e.g., two to several different cell types) that are characteristically found in a particular tissue and not in most or all other tissues. Numerous cell type or tissue-specific promoters are known, and one of ordinary skill in the art will readily be able to identify tissue specific promoters (or to determine whether any particular promoter is a tissue specific promoter) from the literature or by performing experiments such as Northern blots, immunoblots, etc. in which expression of either an endogenous gene or a reporter gene operably linked to the promoter is compared in different cell or tissue types). For example, the nestin, neural specific enolase, NeuN, and GFAP promoters direct transcription in various neural or glial lineage cells; the keratin 5 promoter directs transcription in keratinocytes; the MyoD promoter directs transcription in skeletal muscle cells; the insulin promoter directs transcription, in pancreatic beta cells; the CYP450 3A4 promoter directs transcription in hepatocytes. A lineage specific promoter directs transcription in cells of a particular lineage and not in fully differentiated cells of most or all other lineages. For example, the promoter may direct transcription in cells types of the B cell lineage, T cell lineage, macrophage lineage, etc.

The invention therefore provides lentiviral transfer vectors as described above comprising a cell type or tissue-specific promoter and methods of using the transfer plasmids and lentiviral particles derived therefrom to achieve cell type or tissue specific expression. In general, promoters are active in mammalian cells. According to certain embodiments of the invention a cell type specific promoter is specific for cell types found in the brain (e.g., neurons, glial cells), liver (e.g., hepatocytes), pancreas, skeletal muscle (e.g., myocytes), immune system (e.g., T cells, B cells, macrophages), heart (e.g., cardiac myocytes), retina, skin (e.g., keratinocytes), bone (e.g., osteoblasts or osteoclasts), etc.

Certain embodiments of the invention provide conditional expression of a heterologous nucleic acid sequence, e.g., expression is controlled by subjecting a cell; tissue, organism, etc., to a treatment or condition that causes the heterologous nucleic acid to be expressed or that causes an increase or decrease in expression of the heterologous nucleic acid. As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue-specific expression.

One approach to achieving conditional expression involves the use of inducible promoters. As used herein, the term “inducible promoter” refers to a regulatory element (e.g., a promoter, promoter/enhancer, or portion thereof) whose transcriptional activity may be regulated by exposing a cell or tissue comprising a nucleic acid sequence operably linked to the promoter to a treatment or condition that alters the transcriptional activity of the promoter, resulting in increased transcription of the nucleic acid sequence. For convenience, as used herein, the term “inducible promoter” also includes repressible promoters, i.e., promoters whose transcriptional activity may be regulated by exposing a cell or tissue comprising a nucleic acid sequence operably linked to the promoter to a treatment or condition that alters the transcriptional activity of the promoter, resulting in decreased transcription of the nucleic acid sequence. Typical inducible promoters are active in mammalian cells. Inducible promoters include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), etc. The invention therefore provides lentiviral transfer plasmids as described above comprising a tissue-specific promoter and methods of using transfer plasmids and lentiviral particles derived therefrom to achieve cell type or tissue specific expression.

Another approach to achieving conditional expression involves use of binary transgenic systems, in which gene expression is controlled by the interaction of two components: a “target” transgene and an “effector” transgene, whose product acts on the target transgene. See, e.g., Lewandoski, 2001, Nature Reviews Genetics, 2:743 and articles referenced therein, all of which are incorporated herein by reference, for reviews of methods for achieving conditional expression in mice.

In general, binary transgenic systems fall into two categories. In the first type of system, the effector transactivates transcription of the target transgene. For example, in tetracycline-dependent regulatory systems (Gossen, M. & Bujard, H, Proc. Natl Acad. Sci. USA 89, 5547-5551, 1992), the effector is a fusion of sequences that encode the VP16 transactivation domain and the Escherichia coli tetracycline repressor (TetR) protein, which specifically binds both tetracycline and the 19-bp operator sequences (tetO) of the tet operon in the target transgene, resulting in its transcription. In the original system, the tetracycline-controlled transactivator (tTA) cannot bind DNA when the inducer is present, while in a modified version, the “reverse tTA” (rtTA) binds DNA only when the inducer is present (“tet-on”; Gossen et al., Science 1995, 268:766). The current inducer of choice is doxycycline (Dox). The invention therefore provides lentiviral transfer plasmids as described above comprising a tetracycline-controlled transactivator or reverse tetracycline-controlled transactivator, lentiviral transfer plasmids comprising operator sequences of the tet operon to which the tetracycline-controlled transactivator or reverse tetracycline-controlled transactivator specifically bind, and methods of using the transfer plasmids and lentiviral particles derived therefrom to achieve conditional expression, including the generation of transgenic animals in which conditional expression is achieved. Another example is the “GeneSwitch” mifepristone-regulatable system (Sirin et al., 2003, Gene, 323:67).

In the second type of system, the effector is a site-specific DNA recombinase that rearranges the target gene, thereby activating or silencing it, as further described below. In order to achieve conditional expression in cells or tissues having a particular physiological, biological, or disease state, a promoter that is selectively active in cells or tissue having that particular physiological, biological, or disease state may be used.

In certain embodiments of the invention a promoter recognized by RNA polymerase III (pol III promoter), such as the U6 or H1 promoter, or a promoter recognized by RNA polymerase I (pol I promoter), such as a tRNA promoter, is used. According to certain embodiments of the invention the pol I or pol III promoter is inducible see, e.g., van de Wetering Met al., 2003, EMBO Rep., 4:609).

Recombination Sites for Site-Specific Recombinase

According to certain embodiments of the invention the transfer plasmid includes at least one (typically two) site(s) for recombination mediated by a site-specific recombinase. Site-specific recombinases catalyze introduction or excision of DNA fragments from a longer DNA molecule. These enzymes recognize a relatively short, unique nucleic acid sequence, which serves for both recognition and recombination. Typically a recombination site is composed of short inverted repeats (6, 7, or 8 base pairs in length) and the length of the DNA-binding element is typically approximately 11 to approximately 13 bp in length.

The vectors may comprise one or more recombination sites for any of a wide variety of site-specific recombinases. It is to be understood that the target site for a site-specific recombinase is in addition to any site(s) required for integration of the lentiviral genome. According to various embodiments of the invention, a lentiviral vector includes one or more sites for a recombinase enzyme selected from the group consisting of Cre, XerD, HP1 and Flp. These enzymes and their recombination sites are well known in the art (see, for example, Sauer et al., 1989, Nucleic Acids Res., 17:147; Gorman et al., 2000, Curr. Op. Biotechnol., 11:455; O'Gorman et al., 1991, Science, 251:1351; Kolb, 2002, Cloning Stem Cells, 4:65; Kuhn et al., 2002, Methods Mol. Biol., 180:175).

These recombinases catalyze a conservative DNA recombination event between two 34-bp recognition sites (loxP and FRT, respectively). Placing a heterologous nucleic acid sequence operably linked to a promoter element between two loxP sites (in which case the sequence is “floxed”) allows for controlled expression of the heterologous sequence following transfer into a cell. By inducing expression of Cre within the cell, the heterologous nucleic acid sequence is excised, thus preventing further transcription and effectively eliminating expression of the sequence. This system has a number of applications including Cre-mediated gene activation (in which either heterologous or endogenous genes may be activated, e.g., by removal of an inhibitory element or a polyadenylation site), creation of transgenic animals exhibiting temporal control of Cre expression, cell-lineage analysis in transgenic animals, and generation of tissue-specific knockouts or knockdowns in transgenic animals.

According to certain embodiments of the invention, a lentiviral vector includes two loxP sites. Furthermore, in certain specific embodiments of the invention, a vector includes a cloning site, e.g., a unique restriction site, between two loxP sites, which allows for convenient insertion of a heterologous nucleic acid sequence. According to certain embodiments of the invention, a vector includes a MCS between two loxP sites. According to certain embodiments of the invention, the two loxP sites are located between an HIV FLAP element and a WRE. According to certain embodiments of the invention, a vector comprises a unique restriction site between the 3′ loxP site and the WRE.

As described above, positioning a heterologous nucleic acid sequence between loxP sites allows for controlled expression of the heterologous sequence following transfer into a cell. By inducing Cre expression within the cell, the heterologous nucleic acid sequence is excised, thus preventing further transcription and effectively eliminating expression of the sequence. Cre expression may be induced in any of a variety of ways. For example, Cre may be present in the cells under control of an inducible promoter, and Cre expression may be induced by activating the promoter. Alternatively or additionally, Cre expression may be induced by introducing an expression vector that directs expression of Cre into the cell. Any suitable expression vector can be used, including, but not limited to, viral vectors such as adenoviral vectors. The phrase “inducing Cre expression” as used herein refers to any process that results in an increased level of Cre within a cell.

Lentiviral transfer plasmids comprising two loxP sites are useful in any applications for which standard vectors comprising two loxP sites can be used. For example, selectable markers may be placed between the loxP sites. This allows for sequential and repeated targeting of multiple genes to a single cell (or its progeny). After introduction of a transfer plasmid comprising a floxed selectable marker into a cell, stable transfectants may be selected. After isolation of a stable transfectant, the marker can be excised by induction of Cre. The marker may then be used to target a second gene to the cell or its progeny. Lentiviral particles comprising a lentiviral genome derived from the transfer plasmids may be used in the same manner.

As another example, standard gene-targeting techniques may be used to produce a mouse in which an essential region of a gene of interest is foxed, so that tissue-specific Cre expression results in the inactivation of this allele. The transfer plasmids may be introduced into cells (e.g., ES cells) using pronuclear injection. Alternately, the cells may be injected or infected with lentiviral particles comprising a lentiviral genome derived from the transfer plasmid. Tissue-specific Cre expression may be achieved by crossing a mouse line with a conditional allele (e.g., a foxed nucleic acid sequence) to an effector mouse line that expresses cre in a tissue-specific manner, so that progeny are produced in which the conditional allele is inactivated only in those tissues or cells that express Cre. Suitable transgenic lines are known in the art and may be found, for example, in the Cre Transgenic Database at the Web site having URL www.mshri.on.ca/nagy/Cre-pub.html. When lentiviral vectors are used for RNAi (see below), this approach may allow for silencing of genes whose expression is essential during only part of an animal's development at a time following the stage during which expression is required.

Transfer plasmids and lentiviral particles of the invention may be used to achieve constitutive, conditional, reversible, or tissue-specific expression in cells, tissues, or organisms, including transgenic animals (see below). The invention provides a method of reversibly expressing a transcript in a cell comprising: (i) delivering a lentiviral vector to the cell, wherein the lentiviral vector comprises a heterologous nucleic acid, and wherein the heterologous nucleic acid is located between sites for a site-specific recombinase; and (ii) inducing expression of the site-specific recombinase within the cell, thereby preventing synthesis of the transcript within those cells. According to certain embodiments of the invention, the cell is a mammalian cell. According to certain embodiments of the invention, the step of inducing the site-specific recombinase comprises introducing a vector encoding the site-specific recombinase into the cell. According to some embodiments of the invention, a nucleic acid encoding the site-specific recombinase is operably linked to an inducible promoter, and the inducing step comprises inducing the promoter as described above.

The invention provides a variety of methods for achieving conditional and/or tissue-specific expression. For example, the invention provides methods for expressing a transcript in a mammal in a cell type or tissue-specific manner comprising: (i) delivering a lentiviral transfer plasmid or lentiviral particle to cells of the mammal, wherein the lentiviral transfer plasmid or lentiviral particle comprises a heterologous nucleic acid, and wherein the heterologous nucleic acid is located between sites for a site-specific recombinase; and (ii) inducing expression of the site-specific recombinase in a subset of the cells of the mammal, thereby preventing synthesis of the transcript within those cells. According to certain embodiments, the recombinase is Cre. According to certain embodiments of the invention the step of inducing the site-specific recombinase comprises introducing a vector encoding the site-specific recombinase into the cell. According to some embodiments of the invention a nucleic acid encoding the site-specific recombinase is operably linked to an inducible promoter, and the inducing step comprises inducing the promoter as described above. In certain embodiments of the invention the nucleic acid encoding the site-specific recombinase is operably linked to a cell type or tissue-specific promoter, so that synthesis of the recombinase takes place only in cells or tissues in which that promoter is active.

Internal Ribosome Entry Site (IRES)

In some embodiments, a lentiviral vector may include an IRES. IRES elements function as initiators of the efficient translation of reading frames. An IRES allows ribosomes to start the translation process anew with whatever is immediately downstream and regardless of whatever was upstream. In particular, an IRES allows for the translation of two different genes on a single transcript. For example, an IRES allows the expression of a marker such as EGFP off the same transcript as a transgene, which has a number of advantages: (1) the transgene is native and does not have any fused open reading frames that might affect function; (2) since the EGFP is from the same transcript, its levels should be an accurate representation of the levels of the upstream transgene. IRES elements are known in the art and are further described (see, e.g., Kim et al., 1992, Mol. Cell. Biol., 12:3636; and McBratney et al., 1993, Curr. Opin. Cell Biol., 5:961). Any of a wide variety of sequences of viral, cellular, or synthetic origin which mediate internal binding of the ribosomes can be used as an IRES. Examples include those IRES elements from poliovirus Type I, the 5′UTR of encephalomyocarditis virus (EMV), of Thelier's murine encephalomyelitis virus (TMEV) of foot and mouth disease virus (FMDV) of bovine enterovirus (BEV), of coxsackie B virus (CBV), or of human rhinovirus (HRV), or the human immunoglobulin heavy chain binding protein (BIP) 5′UTR, the Drosophila antennapediae 5′UTR or the Drosophila ultrabithorax 5′UTR, or genetic hybrids or fragments from the above-listed sequences.

Episomal Elements

The presence of appropriate genetic elements from various papovaviruses allows plasmids to be maintained as episomes within mammalian cells. Such plasmids are faithfully distributed to daughter cells. In particular, viral elements of various polyomaviruses and papillomaviruses such as BK virus (BKV), bovine papilloma virus 1 (BPV-1) and Epstein-Barr virus (EBV), among others, are useful in this regard. The invention therefore provides lentiviral transfer plasmids comprising a viral element sufficient for stable maintenance of the transfer plasmid as an episome within mammalian cells. Appropriate genetic elements and their use are described, for example, in Van Craenenbroeck et al. (2000, Eur. Biochem., 267:5665 and references therein, all of which are incorporated herein by reference).

The invention further provides cell lines comprising transfer plasmids described above, i.e., cell lines in which transfer plasmids are stably maintained as episomes. In particular, the invention provides producer cell lines (cell lines that produce proteins needed for production of infectious lentiviral particles) in which transfer plasmids are stably maintained as episomes. According to certain embodiments of the invention, these cell lines constitutively produce lentiviral particles.

According to some embodiments of the invention, one or more necessary viral proteins is under the control of an inducible promoter. Thus the invention provides helper cell lines in which transfer plasmids are stably expressed as episomes, wherein at least one viral protein expressed by the cell line is under control of an inducible promoter. This allows cells to be expanded under conditions that are not permissive for viral production. Once cells have reached a desired density (e.g., confluence), a desired cell number, etc., the protein whose expression is under control of the inducible promoter can be induced, allowing production of viral particles to begin. This system offers a number of advantages. In particular, since every cell has the required components, titer is increased. In addition, it avoids the necessity of performing a transfection each time a particular virus is desired. Any of a variety of inducible promoters known in the art may be used. One of ordinary skill in the art will readily be able to select an appropriate inducible promoter and apply appropriate techniques to induce expression therefrom.

The invention thus provides methods of producing lentiviral particles comprising introducing a lentiviral transfer plasmid of the invention, which lentiviral transfer plasmid comprises a genetic element (e.g., a viral element) sufficient for stable maintenance of the transfer plasmid as an episome in mammalian cells, into a helper cell that produces proteins needed for production of infectious lentiviral particles; and culturing the cell for a period sufficient to allow production of lentiviral particles. The invention further provides a method of producing lentiviral particles comprising introducing a lentiviral transfer plasmid of the invention, which lentiviral transfer plasmid comprises a genetic element sufficient for stable maintenance of the transfer plasmid as an episome in mammalian cells, into a helper cell that expresses a protein required for production of lentiviral particles, wherein expression of the protein is under control of an inducible promoter; inducing expression of the protein required for production of lentiviral particles; and culturing the cell for a period sufficient to allow production of lentiviral particles.

Vectors Comprising Heterologous Nucleic Acids

The invention provides lentiviral vectors that comprise any of a variety of heterologous nucleic acids, preferably operably linked to regulatory sequences sufficient for transcription of the heterologous nucleic acid. The heterologous nucleic acid may be inserted at any available site within the vector including, but not limited to, at a restriction site within an MCS. A heterologous nucleic acid may be a naturally occurring sequence or variant thereof or an artificial sequence. Heterologous nucleic acids may already comprise one or more regulatory sequences such as promoters, initiation sequences, processing sequences, etc. Alternatively or additionally, such regulatory elements may be present within the vector prior to insertion of the heterologous nucleic acid.

According to certain embodiments of the invention, the inserted heterologous sequence is a reporter gene sequence. A reporter gene sequence, as used herein, is any gene sequence which, when expressed, results in the production of a protein whose presence or activity can be monitored. Suitable reporter gene sequences include, but are not limited to, sequences encoding chemiluminescent or fluorescent proteins such as green fluorescent protein (GFP) and variants thereof such as enhanced green fluorescent protein (EGFP); cyan fluorescent protein; yellow fluorescent protein; blue fluorescent protein; dsRed or dsRed2, luciferase, aequorin, etc. Many of these markers and their uses are reviewed in van Roessel et al. (2002, Nature Cell Biology, 4:E15 and references therein, all of which are incorporated herein by reference). Additional examples of suitable reporter genes include the gene for galactokinase, beta-galactosidase, chloramphenicol acetyltransferase, beta-lactamase, etc. Alternatively, the reporter gene sequence may be any gene sequence whose expression produces a gene product which affects cell physiology or phenotype. In general, a reporter gene sequence typically encodes a protein that is not normally present within a cell into which the transfer plasmid is to be introduced.

According to certain embodiments of the invention the inserted heterologous sequence is a selectable marker gene sequence, which term is used herein to refer to any gene sequence capable of expressing a protein whose presence permits the selective maintenance and/or propagation of a cell which contains it. Examples of selectable marker genes include gene sequences capable of conferring host resistance to antibiotics (e.g., puromycin, ampicillin, tetracycline, kanamycin, and the like), or of conferring host resistance to amino acid analogues, or of permitting the growth of cells on additional carbon sources or under otherwise impermissible culture conditions. A gene sequence may be both a reporter gene and a selectable marker gene sequence. In general, reporter or selectable marker gene sequences are sufficient to permit the recognition or selection of the plasmid in normal cells.

The heterologous sequence may also comprise the coding sequence of a desired product such as a biologically active protein or polypeptide (e.g., a therapeutically active protein or polypeptide) and/or an immunogenic or antigenic protein or polypeptide. Introduction of the transfer plasmid into a suitable cell thus results in expression of the protein or polypeptide by the cell. Alternatively, the heterologous gene sequence may comprise a template for transcription of an antisense RNA, a ribozyme, or, preferably, one or more strands of an RNAi agent such as a short interfering RNA (siRNA) or a short hairpin RNA (shRNA). As described further below, RNAi agents such as siRNAs and shRNAs targeted to cellular transcripts inhibit expression of such transcripts. Introduction of the vector into a suitable cell thus results in production of the RNAi agent, which inhibits expression of the target transcript.

### Three and Four Plasmid Systems

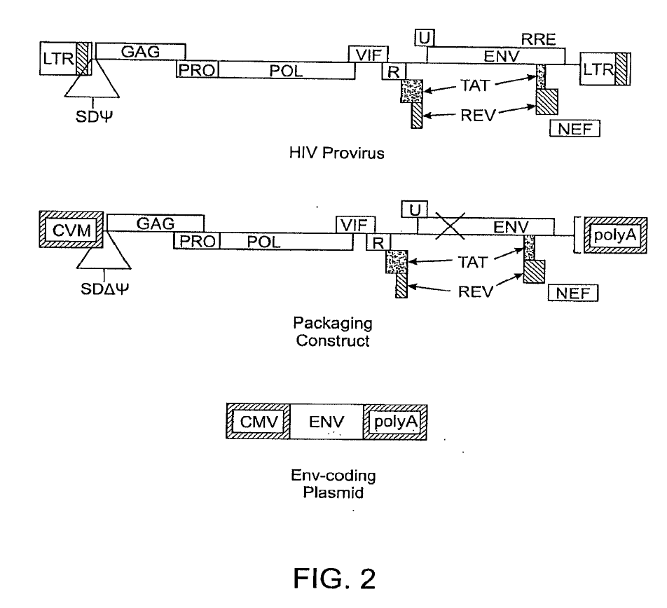

The invention further provides a recombinant lentiviral system comprising three plasmids. The first plasmid is constructed to comprise mutations that prevent lentivirus-mediated transfer of viral genes. Such a mutation may be a deletion of sequences in the viral env gene, thus preventing the generation of replication-competent lentivirus, or may be deletions of certain cis-acting sequence elements at the 3′ end of the genome required for viral reverse transcription and integration. Thus even if viral genes from such a construct are packaged into viral particles, they will not be replicated and replication-competent wild-type viruses will not be produced. The first plasmid (packaging plasmid) comprises a nucleic acid sequence of at least part of a lentiviral genome, wherein the vector (i) encodes at least one essential lentiviral protein and lacks a functional sequence encoding a viral envelope protein; and (ii) lacks a functional packaging signal. The second plasmid (Env-coding plasmid) comprises a nucleic acid sequence of a virus, wherein the vector (i) encodes a viral envelope protein, and (ii) lacks a functional packaging signal. The third plasmid is any of the inventive lentiviral transfer plasmids described above. The first and second plasmids are further described below, and schematic diagrams of relevant portions of representative first and second plasmids (packaging and Env-coding) are presented in FIG. 2 (see U.S. Pat. No. 6,013,516). It will be appreciated that a wide variety of regulatory sequences sufficient to direct transcription in eukaryotic cells could be used in place of the CMV transcriptional regulatory element in the packaging and/or Env-coding plasmid.

Packaging Plasmid

In certain embodiments of the invention the first vector is a gag/pol expression vector, i.e., a plasmid capable of directing expression of functional forms of a retroviral gag gene product and a retroviral pol gene product. These proteins are necessary for assembly and release of viral particles from cells. The first plasmid may also express sequences encoding various accessory lentiviral proteins including, but not limited to, Vif, Vpr, Vpu, Tat, Rev, and Nef. In particular, the first plasmid may express a sequence encoding Rev. In general, gag and pol sequences may be derived from any retrovirus, and accessory sequences may be derived from any lentivirus. According to certain embodiments of the invention, gag and poi sequences and any accessory sequences are derived from HIV-1. gag, pol, and accessory protein sequences need not be identical to wild type versions but instead may comprise mutations, deletions, etc., that do not significantly impair the ability of the proteins to perform their function(s).

The first plasmid is preferably constructed to comprise mutations that exclude retroviral-mediated transfer of viral genes. Such mutations may be a deletion or mutation of sequences in the viral env gene, thus excluding the possibility of generating replication-competent lentivirus. Alternatively or additional to deletion or mutation of env, according to certain embodiments of the invention, the plasmid sequence may comprise deletions of certain cis-acting sequence elements at the 3′ end of the genome required for viral reverse transcription and integration. Accordingly, even if viral genes from this construct are packaged into viral particles, they will not be replicated and replication-competent wild-type viruses will not be generated. Any of a wide variety of packaging plasmids may be used in the three plasmid lentiviral expression system of the invention including, but not limited to, those described in Naldini, 1996; Lois, 2002; Miyoshi, 1998; and Dull, 1998.

Env-Coding Plasmid

This plasmid directs expression of a viral envelope protein and, therefore, comprises a nucleic acid sequence encoding a viral envelope protein under the control of a suitable promoter. The promoter can be any promoter capable of directing transcription in cells into which the plasmid is to be introduced. One of ordinary skill in the art will readily be able to select an appropriate promoter among, for example, the promoters mentioned above. The Env-coding plasmid usually comprises any additional sequences needed for efficient transcription, processing, etc., of the env transcript including, but not limited to, a polyadenylation signal such as any of those mentioned above.

The host range of cells that viral vectors of the present invention can infect may be altered (e.g., broadened or narrowed) by utilizing an envelope gene from a different virus. Thus is possible to alter, increase, or decrease the host range of vectors of the present invention by taking advantage of the ability of the envelope proteins of certain viruses to participate in the encapsidation of other viruses. In certain specific embodiments, the G-protein of vesicular-stomatitis virus (VSV-G; see, e.g., Rose et al., 1981, J. Virol., 39:519; and Rose et al., 1982, Cell, 30:753), or a fragment or derivative thereof, is the envelope protein expressed by the second plasmid. VSV-G efficiently forms pseudotyped virions with genome and matrix components of other viruses. As used herein, the term “pseudotype” refers to a viral particle that comprises nucleic acid of one virus but the envelope protein of another virus. In general, VSV-G pseudotyped viruses have a broad host range, and may be pelleted to titers of high concentration by ultracentrifugation (e.g., according to the method of Burns, et al., 1993, Proc. Natl. Acad. Sci., USA, 90:8033), while still retaining high levels of infectivity.

Additional envelope proteins that may be used include ecotropic or amphotropic MLV envelopes, 10A1 envelope, truncated forms of the HIV env, GALV, BAEV, SIV, FeLV-B, RD114, SSAV, Ebola, Sendai, FPV (Fowl plague virus), and influenza virus envelopes. Similarly, genes encoding envelopes from RNA viruses (e.g. RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples include FIV, FeLV, RSV, VEE, HFVW, WDSV, SFV, Rabies, ALV, BIV, BLV, EBV, CAEV, HTLV, SNV, ChTLV, STLV, MPMV, SMRV, RAV, FuSV, MH2, AEV, AMV, CT10, EIAV.

In addition to the above, hybrid envelopes (e.g. envelope comprising regions of more than one of the above), may be employed. According to certain embodiments of the invention the envelope recognizes a unique cellular receptor (e.g., a receptor found only on a specific cell type or in a specific species). According to certain embodiments of the invention the envelope recognizes multiple different receptors. According to certain embodiments of the invention the second plasmid encodes a cell or tissue specific targeting envelope. Cell or tissue specific targeting may be achieved, for example, by incorporating particular sequences within the envelope sequence (e.g., sequences encoding ligands for cell or tissue-specific receptors, antibody sequences, etc.). Thus any of a wide variety of Env-coding plasmids may be used in the three plasmid lentiviral expression system of the invention including, but not limited to, those described in Naldini, 1996; Lois, 2002; Miyoshi, 1998; and Dull, 1998.

Variations on the Three Plasmid System

The invention further provides a four plasmid lentiviral expression system comprising a three plasmid lentiviral expression system as described herein and a fourth plasmid comprising a nucleic acid sequence encoding the Rev protein (in which case the rev gene is generally not included in the other plasmids. Rev increases the level of transcription during production of lentiviral particles. A variety of alternative three or four plasmid systems may be employed while maintaining the feature that no sequence of recombination event(s) between only two of the three or four plasmids is sufficient to generate replication-competent virus. For example, either Gag or Pol or any of the accessory proteins may be encoded by the plasmid referred to as the Env-coding plasmid. Alternately, Gag, Pol, or any of the accessory proteins may be encoded by the transfer plasmid. In addition, sequences encoding Rev may be provided on the same plasmid that encodes Gag, Pot, or Env. According to certain embodiment's of the invention sequences encoding a functional Tat protein are absent from the plasmids, and sequences encoding Rev are provided on a separate plasmid rather than on the same plasmid as sequences encoding other viral genes, as described (Dull, 1998). The fourth plasmid encoding Rev typically comprises an expression cassette comprising regulatory sequences sufficient to direct transcription in eukaryotic cells, operably linked to a nucleic acid segment that encodes Rev, and a polyadenylation signal (Dull, 1998).

Transfer plasmids and three-plasmid recombinant lentiviral expression systems of the invention may be used to produce infectious, replication-defective lentiviral particles according to methods known to those skilled in the art, some of which have been mentioned above. In the case of the recombinant lentiviral expression system of the invention the methods include (i) transfecting a lentivirus-permissive cell with the three-plasmid lentiviral expression system of the present invention; (ii) producing the lentivirus-derived particles in the transfected cell; and (iii) collecting the virus particles from the cell. The step of transfecting the lentivirus-permissive cell can be carried out according to any suitable means known to those skilled in the art. For example, the three-plasmid expression system described herein may be used to generate lentivirus-derived retroviral vector particles by transient transfection. The plasmids may be introduced into cells by any suitable means, including, but not limited to, calcium phosphate or calcium chloride co-precipitation, DEAE-dextran-mediated transfection, lipofection, injection, electroporation, etc.

Transfer plasmids of the invention may be used to produce infectious, replication-defective lentiviral particles in a similar manner using helper cells that express the necessary viral proteins as known in the art and mentioned above. In general, transfer plasmids may be used to produce infectious, replication-defective lentiviral particles in conjunction with any system using any combination of plasmids and/or helper cell lines that provides the appropriate combination of required genes: gag, pol, env, and, preferably, rev in cases where transcription occurs from a gag/pol expression cassette comprising a Rev-response element (or alternately a system that supplies the various proteins encoded by these genes).

Infectious virus particles may be collected using conventional techniques. For example, infectious particles may be collected by cell lysis or by collection of cell culture supernatant, as is known in the art. Optionally, collected virus particles may be purified. Suitable purification techniques are well known to those skilled in the art. Methods for titering virus particles are also well known in the art. Further details are provided in the Examples.

When a host cell permissive for production of lentiviral particles is transfected with the plasmids of the three-plasmid system, the cell becomes a producer cell, i.e., a cell that produces infectious lentiviral particles. Similarly, when a helper cell that produces the necessary viral proteins is transfected with a transfer plasmid of the invention, the cell becomes a producer cell. The invention therefore provides producer cells and corresponding producer cell lines and methods for the production of such cells and cell lines. In particular, the invention provides a method of creating a producer cell line comprising introducing a transfer plasmid of the invention into a host cell; and introducing a packaging plasmid and an envelope plasmid into the host cell. The invention provides another method of creating a producer cell line comprising introducing a transfer plasmid of the invention into a helper cell that produces viral proteins necessary for encapsidation of a lentiviral genome and subsequent infectivity of a lentiviral particle resulting from encapsidation.

### Applications and Additional Embodiments

Lentiviral vectors and systems of the invention have a variety of uses, some of which have been described above. Transfer plasmids may be used for any application in which a non-retroviral vector is typically employed, e.g., for expression of a nucleic acid sequence in isolated eukaryotic cells, for creating transgenic animals, etc. Plasmids may be introduced into cells via conventional techniques such as transfection, electroporation, etc. Cells are maintained under suitable culture conditions for a suitable period of time. Optionally, stable cell lines in which all or a portion of a plasmid is integrated into the cellular genome are generated. If a plasmid comprises an expression cassette comprising a sequence that encodes an RNA, e.g., an mRNA, the RNA is transcribed, and optionally translated in the cells and can be harvested therefrom using methods known in the art. The expression cassette will typically comprise regulatory sequences for transcription, transcriptional termination, etc.

Lentiviral particles may also be introduced into cells using methods well known in the art. Such methods typically involve incubating cells in an appropriate medium in the presence of lentiviral particles and a reagent such as polybrene that facilitates infection. Lentiviral particles may be introduced into cells via conventional techniques such as incubation in the presence of polybrene, etc. Cells are maintained under suitable culture conditions for a suitable period of time. Optionally, stable cell lines in which all or a portion of the lentiviral genome is integrated into the cellular genome are generated. If the lentiviral genome comprises an expression cassette comprising a sequence that encodes an RNA, e.g., an mRNA, the RNA is transcribed, and optionally translated in the cells and can be harvested therefrom using methods known in the art.

Gene Silencing in Isolated Eukaryotic Cells and Transgenic Animals

The invention provides lentiviral vectors that are of use for inhibiting gene expression by RNA interference (RNAi) in isolated eukaryotic cells and/or in transgenic animals. The invention provides lentiviral vectors that comprise a nucleic acid that comprises (i) a eukaryotic anti-repressor element (ARE); (ii) lentivirus derived sequences sufficient for reverse transcription and packaging; and (iii) an expression cassette that encodes one or more strands of an RNAi agent. For example, in certain embodiments of the invention the expression cassette comprises regulatory sequences for transcription operably associated with a nucleic acid sequence that encodes an shRNA. The expression cassette may comprise additional sequences such as a transcriptional termination signal, etc.

RNAi is an evolutionarily conserved process in which presence of an at least partly double-stranded RNA molecule in a eukaryotic cell leads to sequence-specific inhibition of gene expression. RNAi was first described as a phenomenon in which introduction of long dsRNA (typically hundreds of nucleotides) into a cell results in degradation of mRNA containing a region complementary to one strand of the dsRNA (U.S. Pat. No. 6,506,559). Studies in Drosophila showed that long dsRNAs are processed by an intracellular RNase III-like enzyme called Dicer into smaller dsRNAs primarily comprised of two approximately 21 nucleotide (nt) strands that form a 19 base pair duplex with 2 nt 3′ overhangs at each end and 5′-phosphate and 3′-hydroxyl groups (see, e.g., PCT Publication WO 01/75164; U.S. Patent Publications 2002/0086356 and 2003/0108923; Zamore et al., 2000; and Elbashir et al., 2001a and 2001b).

Short dsRNAs having this structure, referred to as siRNAs, silence expression of target genes that include a region that is substantially complementary to one of the two strands. This strand is referred to as the “antisense” or “guide” strand of the siRNA, while the other strand is often referred to as the “sense” strand. The siRNA is incorporated into a ribonucleoprotein complex termed the RNA-induced silencing complex (RISC) that contains member(s) of the Argonaute protein family. Following association of the siRNA with RISC, a helicase activity unwinds the duplex, allowing an alternative duplex to form the guide strand and a target mRNA containing a portion substantially complementary to the guide strand. An endonuclease activity associated with the Argonaute protein(s) present in RISC cleaves or “slices” the target mRNA, which is then further degraded by cellular machinery.

Exogenous introduction of siRNAs into eukaryotic cells, e.g., mammalian or avian cells can effectively reduce expression of target genes in a sequence-specific manner via this mechanism. A typical siRNA structure includes an approximately 17 to approximately 29 nucleotide (e.g., approximately 19 nucleotide) double-stranded portion comprising a guide strand and an antisense strand. Each strand has a 2 nt 3′ overhang. The guide strand of the siRNA is substantially complementary to its target gene and mRNA transcript over approximately 15 to approximately 29 nucleotides, e.g., at least approximately 17 to approximately 19 nucleotides, and the two strands of the siRNA are substantially complementary to each other over the duplex portion of the structure (e.g., over approximately 15 to approximately 29 nt, e.g., approximately 19 nucleotides); thus the sense strand is typically substantially identical to the target transcript over approximately 15 to approximately 29 nucleotides, e.g., approximately 19 nucleotides. Typically the guide strand of the shRNA is perfectly complementary to its target gene and mRNA transcript over approximately 15 to approximately 29 nucleotides, e.g., approximately 17 to approximately 19 nucleotides, and the two strands of the siRNA are perfectly complementary to each other over the duplex portion of the structure. However, as will be appreciated by one of ordinary skill in the art, perfect complementarity is not required. Instead, one or more mismatches in the duplex formed by the guide strand and the target mRNA is often tolerated, particularly at certain positions, without reducing the silencing activity below useful levels. For example, there may be 1, 2, 3, or even more mismatches between the target mRNA and the guide strand (disregarding the overhangs). Thus, as used herein, two nucleic acid portions such as a guide strand (disregarding overhangs) and a portion of a target mRNA are “substantially complementary” if they are perfectly complementary (i.e., they hybridize to one another to form a duplex in which each nucleotide is a member of a complementary base pair) or have a lesser degree of complementarity sufficient for hybridization to occur. Typically at least approximately 80%, at least approximately 90%, or more of the nucleotides in the guide strand of an effective siRNA are complementary to the target mRNA and to the sense strand over at least approximately 17 to approximately 19 contiguous nucleotides. Methods for predicting the effect of mismatches on silencing efficacy and the locations at which mismatches may most readily be tolerated have been developed (Reynolds, et al., 2004). Two nucleic acid portions such as a sense strand (disregarding overhangs) and a portion of a target mRNA are “substantially identical” if they are perfectly identical (i.e., they have the same sequence) or have a lesser degree of complementarity sufficient for hybridization to occur between one of the sequences and the complement of the other sequence. Typically, substantially identical nucleic acid portions such as a sense strand and a target mRNA are at least approximately 80% or at least approximately 90% identical over at least approximately 17 to approximately 19 contiguous nucleotides.

It will be appreciated that molecules having the appropriate structure and degree of complementarity to a target gene will exhibit a range of different silencing efficiencies. A variety of design criteria have been developed to assist in the selection of effective siRNA sequences. It may be preferable to use sequences that have a GC content between approximately 30% to approximately 50% and to avoid consecutive strings of 4 or more of the same residue, e.g., AAAA or TTTT. A number of software programs that can be used to choose siRNA sequences that are predicted to be particularly effective to silence a target gene of choice are available (Yuan et al., 2004; Santoyo et al., 2005). Furthermore, sequences of effective siRNAs are already known in the art for many genes. For example, siRNA designs are currently available from Ambion for >98% of the human, mouse, and rat genes that are listed in the National Center for Biotechnology Information's RefSeq database (Ambion, Austin, Tex.). It has been estimated that more than half of randomly designed siRNAs provide at least a 50% reduction in target mRNA levels and approximately 1 of 4 siRNAs provide a 75%-95% reduction (Ambion Technical Bulletin #506, Ambion). Candidate sequences complementary to different portions of the target can be tested in cell culture to identify those that result in a desired level of inhibition.

Structures referred to as short hairpin RNAs (shRNAs) are also capable of mediating RNA interference. An shRNA is a single RNA strand that comprises two substantially complementary regions that hybridize to one another to form a double-stranded “stem,” with the two substantially complementary regions being connected by a single-stranded loop that extends from the 3′ end of one complementary region to the 5′ end of the other complementary region. shRNAs are processed intracellularly by Dicer to form an siRNA structure comprising a guide strand and an antisense strand. In the present invention, intracellular synthesis of shRNA is achieved by introducing a lentiviral vector of the invention comprising an shRNA expression cassette into a cell, e.g., to create a stable cell line or transgenic organism. The shRNA expression cassette comprises regulatory sequences operably linked to a nucleic acid that encodes the shRNA. The nucleic acid provides a template for transcription of an RNA that self-hybridizes to form an shRNA.

The shRNA expression cassette is often constructed to comprise, in a 5′ to 3′ direction, the sense strand (substantially identical to the target transcript), followed by a short spacer that forms the loop, followed by the antisense strand (substantially complementary to the target), in that order. In certain embodiments of the invention the reverse order is used. The stem can range from approximately 17 to approximately 29 nucleotides in length, e.g., approximately 19 to approximately 21, approximately 21 to approximately 24, or approximately 25 to approximately 29 nucleotides in length. The loop can range in length from approximately 3 nucleotides to considerably longer, e.g., up to approximately 25 nucleotides. A variety of different sequences can serve as the loop sequence. Examples of specific loop sequences that have been demonstrated to function in shRNAs include UUCAAGAGA, CCACACC, AAGCUU, CTCGAG, CCACC, and UUCG. In certain embodiments of the invention the loop is derived from a miRNA. In certain embodiments of the invention the guide strand is perfectly complementary to the target gene over approximately 17 to approximately 29 nucleotides, and the guide strand and the sense strand are substantially but not perfectly complementary to each other over approximately 17 to approximately 29 nucleotides, e.g., the duplex formed by the guide and sense strands comprises 114 mismatches or bulges (Miyagishi, 2004). The sense, guide, and loop sequences will of course utilize T rather than U when in DNA form, e.g., when used to construct a lentiviral transfer plasmid of the invention.

In certain embodiments of the invention, a regulatory sequence that directs expression of the one or more RNAs that self-hybridize or hybridize with each other to form an shRNA or siRNA comprises a promoter for RNA polymerase III (Pol III). Pol III directs synthesis of small transcripts that terminate within a stretch of 4-5 T residues. Certain Pol III promoters such as the U6 or H1 promoters do not require cis-acting regulatory elements (other than the first transcribed nucleotide) within the transcribed region and readily permit the selection of desired RNA sequences. In the case of naturally occurring U6 promoters the first transcribed nucleotide is typically guanosine, while in the case of naturally occurring promoters the first transcribed nucleotide is adenine. In certain embodiments of the invention, e.g., where transcription is driven by a U6 promoter, the 5′ nucleotide of an RNA sequence that hybridizes or self-hybridizes to form an shRNAs or siRNA is G. In certain embodiments of the invention, e.g., where transcription is driven by an H1 promoter, the 5′ nucleotide may be A. Methods for designing nucleic acids that encode short hairpin RNAs for intracellular expression are described in Medina et al., 1999, Curr. Opin. Mol. Ther., 1:580; Yu et al., 2002, Proc. Natl. Acad. Sci., USA, 99:6047; Sui et al., 2002, Proc. Natl. Acad. Sci., USA, 99:5515; Paddison et al., 2002, Genes Dev., 16:948; Brummelkamp et al., 2002, Science, 296:550; Miyagashi et al., 2002, Nat. Biotech., 20:497; Paul et al., 2002, Nat. Biotech., 20:505; and Tuschl et al., 2002, Nat. Biotech., 20:446. Pol II promoters can also be used to achieve intracellular expression of an RNAi agent (Xia et al., 2002, Nat. Biotech., 20:1006).

As will be appreciated by one of ordinary skill in the art, RNAi may be effectively mediated by RNA molecules having a variety of structures that differ in one or more respects from those described above. For example, the length of the duplex can be varied (e.g., from approximately 17 to approximately 29 nucleotides); the overhangs need not be present and, if present, their length and the identity of the nucleotides in the overhangs can vary. Furthermore additional mechanisms of sequence-specific silencing mediated by short RNA species are also known. The invention provides lentiviral vectors that comprise expression cassettes that encode such RNA species. For example, post-transcriptional gene silencing mediated by small RNA molecules can occur by mechanisms involving translational repression. Certain endogenously expressed RNA molecules form hairpin structures comprising an imperfect duplex portion in which the duplex is interrupted by one or more mismatches and/or bulges. These hairpin structures are processed intracellularly to yield single-stranded RNA species referred to as known as microRNAs (miRNAs), which mediate translational repression of a target transcript to which they hybridize with less than perfect complementarity. siRNA-like molecules designed to mimic the structure of miRNA precursors have been shown to result in translational repression of target genes when administered to mammalian cells. The invention provides lentiviral vectors that comprise an expression cassette that encodes an RNA species that inhibits gene expression by a translational repression mechanism, e.g., an RNA species whose structure mimics or is identical to that of a microRNA precursor and/or that is processed intracellularly to yield a structure that resembles microRNAs in terms of the hybrid that it forms with a target transcript.

The mechanism by which an RNAi agent inhibits gene expression may thus depend at least in part on the structure of the duplex portion of the RNAi agent and/or the structure of the hybrid formed by one strand of the RNAi agent and a target transcript. RNAi mechanisms and the structure of various RNA molecules known to mediate RNAi, e.g., siRNA, shRNA, miRNA and their precursors, have been extensively reviewed (see, e.g., Novina et al., 2004; Dyxhoorn et al., 2003; and Bartel, supra). It is to be expected that future developments will reveal additional mechanisms by which RNAi may be achieved and will reveal additional effective short RNAi agents. The invention includes embodiments in which any currently known or hereafter discovered short RNAi agent that can be synthesized intracellularly, or a precursor thereof, is encoded by a lentiviral vector comprising an ARE and, optionally, a SAR.

In general, RNAi agents are capable of reducing target transcript level and/or level of a polypeptide encoded by the target transcript by at least about 2 fold, at least about 5 fold, at least about 10 fold, at least about 25 fold, at least about 50 fold, or to an even greater degree relative to the level that would be present in the absence of the inhibitory RNA. Certain specific RNAi agents are capable of reducing the target transcript level and/or level of a polypeptide encoded by the target transcript by at least approximately 20%, approximately 30%, approximately 40%, approximately 50%, approximately 60%, approximately 70%, approximately 80%, approximately 90%, or approximately 100%. For example, the average expression level of the gene of interest may be between approximately 0% (undetectable) and approximately 10%, approximately 20%, approximately 30%, approximately 40%, approximately 50%, approximately 60%, approximately 70%, or approximately 80% of the level that would exist in the absence of the RNAi agent. An RNAi agent is “capable of” inhibiting expression if it does so under conditions recognized in the art as suitable for RNAi (e.g., appropriate concentration of RNAi agent, appropriate conditions for uptake or intracellular expression of the RNAi agent, typical levels of expression of the target transcript, etc.). It may be desirable to test a guide strand sequence by administering siRNAs having that guide strand sequence in cell culture in order to determine whether an shRNA that incorporates a guide strand having the same sequence is likely to have a desired inhibitory effect when expressed in a cell. Many potential guide stand sequences can be tested in this manner in order to identify those having preferred inhibitory efficacies.

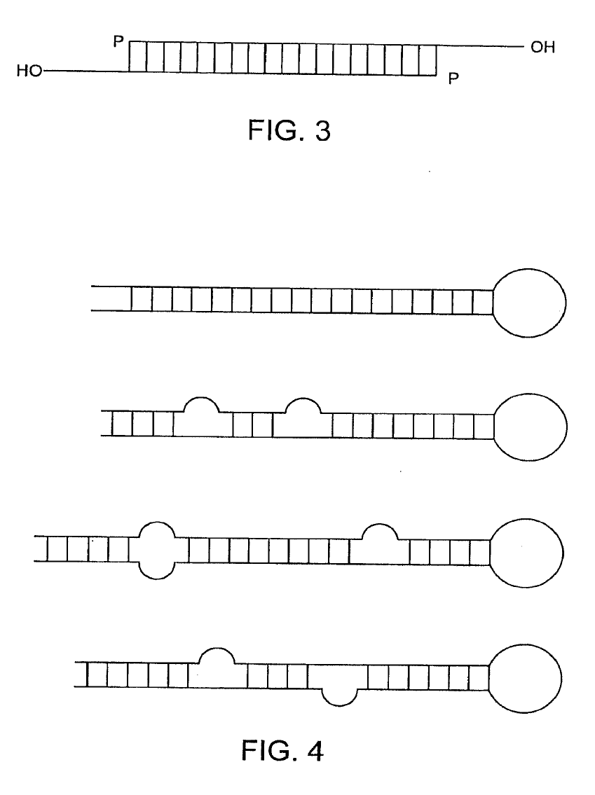

FIGS. 3-5 presents schematic diagrams of various RNAi agents that can be encoded by a lentiviral vector of the present invention and utilized to mediate RNAi in isolated eukaryotic cells, e.g., mammalian or avian cells, and/or in transgenic animals. FIG. 6b shows the sequence and structure of a nucleic acid comprising a segment which, when present in a lentiviral vector of the invention in operable association with a suitable regulatory sequence, can be transcribed to produced an RNA that comprises two complementary elements that hybridize to one another to form a stem and a loop structure (shRNA) targeted to the CD8 molecule. FIG. 6c depicts the shRNA that results following hybridization of the complementary portions of an RNA transcribed from the nucleic acid in FIG. 6b. FIG. 6d (upper portion) shows the sequence and structure of a nucleic acid comprising a segment which, when present in a lentiviral vector of the invention in operable association with a suitable regulatory sequence, can be transcribed to produced an RNA that comprises two complementary elements that hybridize to one another to form a stem and a loop structure (shRNA) targeted to the CD8 molecule. FIG. 6d (lower portion) depicts the shRNA that results following hybridization of the complementary portions of an RNA transcribed from the nucleic acid depicted in the upper portion of FIG. 6d.

A lentiviral vector for use in mediating RNAi may be created using standard methods of molecular biology by inserting a nucleic acid sequence that encodes one or more strands of an RNAi agent, e.g., a nucleic acid sequence that encodes an shRNA, into a transfer plasmid optimized for RNAi that already comprises an ARE and, optionally, a SAR, and comprises a suitable promoter, e.g., a plasmid such as pLB. Alternatively or additionally, an expression cassette comprising suitable regulatory sequences operably linked to the sequence that encodes one or more strands of an RNAi agent may be inserted into a transfer plasmid that lacks appropriate regulatory sequences. A nucleic acid to be inserted into a lentiviral vector to provide an RNAi expression cassette may include a terminator for RNA polymerase I, II, or III. Alternatively or additionally, the vector may comprise a terminator positioned so that a nucleic acid inserted upstream with respect to the terminator will direct transcription of an RNA that is appropriately terminated. An expression cassette to be inserted into a lentiviral vector of the invention may comprise appropriate 5′ or 3′ overhanging ends for directional cloning into restriction site(s) in the vector. Plasmids constructed according to either of these approaches, or others, may be used to generate lentiviral particles. Similar methods may of course be used to construct a transfer plasmid that comprises an expression cassette that encodes any RNA of interest and to generate lentiviral particles therefrom.

As discussed above, in addition to their use for synthesis of RNAs that self-hybridize to form shRNAs, lentiviral vectors of the invention may be used for synthesis of various other RNAs that mediate RNAi. In particular, two separate RNA strands may be generated, each of which comprises an approximately 15 to approximately 29 nucleotide region, e.g., an approximately 19 nucleotide region at least partly complementary to the other, and individual strands may hybridize together to generate an siRNA structure. Accordingly, the invention encompasses a lentiviral vector comprising two transcribable regions, each of which provides a template for synthesis of a transcript comprising a region complementary to the other. In addition, the invention provides a lentiviral vector that comprises oppositely directed promoters flanking a nucleic acid segment and positioned so that two different transcripts having complementary regions approximately 15 to approximately 29 nucleotides, e.g., approximately 19 nucleotides in length, are generated. It will be appreciated that appropriate terminators should be supplied. In cases in which an RNA structure undergoes one or more processing steps, those of ordinary skill in the art will appreciate that the nucleic acid segment will typically be designed to include sequences that may be necessary for processing of the RNA. A large number of variations are possible. For example, the lentiviral vector may comprise multiple expression cassettes or nucleic acid segments, each of which provides a template for synthesis of one or more RNAs that self-hybridize or hybridize with each other to form shRNAs or siRNAs, which shRNAs or siRNAs may target the same transcript or different transcripts. Alternatively or additionally, according to certain embodiments of the invention a single expression cassette or nucleic acid segment may provide a template for synthesis of a plurality of RNAs that self-hybridize or hybridize with each other to form a plurality of siRNAs or siRNA precursors. For example, a single promoter may direct synthesis of a single RNA transcript comprising multiple self-complementary regions, each of which may hybridize to generate a plurality of stem-loop structures. These structures may be cleaved in vivo, e.g., by Dicer, to generate multiple different siRNAs. It will be appreciated that such transcripts typically comprise a termination signal at the 3′ end of the transcript but not between the sequences encoding an siRNA or shRNA strand.

The invention provides methods of inhibiting or reducing expression of a target transcript in a eukaryotic cell comprising delivering a lentiviral vector to the cell, wherein the lentiviral vector comprises an ARE, optionally a SAR, and comprises one or more expression cassette(s) that encode an RNAi agent. The presence of the lentiviral vector within the cell results in synthesis of one or more RNAs that self-hybridize or hybridize with each other to form an shRNA or siRNA that inhibits expression of the target transcript. The RNA(s) may undergo further processing within the cell to form an inhibitory structure. The invention encompasses administration of a lentiviral vector of the invention to a cell, e.g., a mammalian or avian cell, to inhibit or reduce expression of any target transcript or gene, wherein the lentiviral vector comprises a nucleic acid segment that comprises a template for synthesis of one or more RNAs that self-hybridize or hybridize to form an RNAi agent such as an shRNA or siRNA that is targeted to the target transcript or gene. In general, the nucleic acid segment may provide a template for synthesis of any RNA structure capable of being processed in vivo to an RNAi agent such as an shRNA or siRNA, wherein the RNA preferably does not cause undesirable effects events such as induction of the interferon response. A lentiviral vector may be delivered to cells in culture or administered to an animal subject. As used herein, terms such as “introducing,” “delivering,” “administering,” and the like when used in reference to a lentiviral vector of the invention or a composition or cell comprising a lentiviral vector of the invention or comprising nucleic acid sequences derived therefrom refers to any method that provides effective contact between the material to be introduced, delivered, or administered, and the cells whose uptake of the material is desired so that uptake can be achieved. The cells may be in cell culture or in a subject.

The invention further provides methods for reversibly inhibiting or reducing expression of a target transcript in a cell comprising: (i) delivering to the cell a lentiviral vector that comprises a nucleic acid comprising an ARE and, optionally, a SAR, and, wherein the nucleic acid comprises a portion that encodes an RNAi agent or strand thereof located between sites for a site-specific recombinase; and (ii) inducing expression of the site-specific recombinase within the cell, thereby preventing synthesis of the RNAi agent or strand thereof. The nucleic acid may further comprise a SAR. The vector can be a lentiviral transfer plasmid or lentiviral particle.

The invention also provides methods for reversibly inhibiting or reducing expression of a transcript in an animal in a cell type specific, lineage specific, or tissue-specific manner comprising: (i) delivering to the animal a lentiviral vector that comprises a nucleic acid comprising an ARE, wherein the nucleic acid comprises a portion that encodes an RNAi agent or strand thereof located between sites for a site-specific recombinase; and (ii) inducing expression of the site-specific recombinase in a subset of the cells of the mammal, thereby preventing synthesis of the RNAi agent or strand thereof within the subset of cells. The nucleic acid may further comprise a SAR. The vector can be a lentiviral transfer plasmid or lentiviral particle.

In any of the above methods, the cell may be a mammalian or avian cell, the site-specific recombinase may be Cre, and the sites may be loxP sites.

The invention provides methods of reducing or inhibiting expression of target genes and/or transcripts (which need not necessarily encode proteins) by expressing one or more RNAi agents in eukaryotic cells either in culture or in transgenic animals using lentiviral vectors of the invention. The invention further provides methods of inhibiting or reducing expression of a target transcript in a cell comprising introducing a lentiviral vector of the invention (e.g., a lentiviral transfer plasmid or lentiviral particle) into the cell, wherein the lentiviral vector encodes an RNAi agent. In some embodiments the invention provides methods of inhibiting or reducing expression of a target transcript in a nonhuman animal comprising generating a nonhuman transgenic animal using a lentiviral vector of the invention (e.g., a lentiviral transfer plasmid or lentiviral particle), wherein the lentiviral vector encodes an RNAi agent. In some embodiments the RNAi agent is an shRNA. In some embodiments the RNAi agent is a precursor RNA that is processed within a cell to produce an shRNA. In some embodiments the vector comprises an expression cassette that encodes an RNA that self-hybridizes to form an shRNA that is targeted to the target transcript. In some embodiments the target transcript may be one that is transcribed from an endogenous or heterologous disease-associated gene.

Lentiviral vectors of the invention that comprise an expression cassette that encodes an RNAi agent may be used for a variety of purposes. In certain embodiments of the invention a lentiviral vector is used to silence a disease-associated gene in mammalian or avian cells and/or to render mammalian cells resistant to an infectious agent. For example, an RNAi agent may be targeted to a gene that encodes a receptor for the infectious agent. Cells in which the gene is silenced are resistant to infection by the infectious agent. The lentiviral vector may be delivered to cells in culture using any appropriate method, e.g., transfection, infection, etc. Cells that express the RNAi agent may be administered to a subject for therapeutic purposes. For example, such cells may provide a pool of cells that are resistant to infection or that provide an enhanced immune system response to infection. The lentiviral vector may be administered to a subject for therapeutic or other purposes.

The invention also provides lentiviral vectors that comprise expression cassettes that encode other RNA species that are capable of inhibiting expression of a target gene. For example, lentivira vectors that encode antisense RNA molecules, ribozymes, etc., and methods of use thereof are also an aspect of the invention.

Cells

The present invention encompasses any cell manipulated to comprise a lentiviral vector of the invention (e.g., a lentiviral transfer plasmid or lentiviral particle) or nucleic acid sequences (e.g., a lentiviral genome or provirus) derived therefrom and descendants of such cells. A lentiviral vector comprises an ARE and in certain embodiments of the invention also comprises a SAR. Some or all of the sequences may be integrated into the genome of the cell. In certain embodiments of the invention the vector comprises one or more regulatory sequences for transcription of an operably linked nucleic acid. In certain embodiments of the invention the vector comprises an expression cassette or cassettes that encodes an RNA of interest. The RNA of interest may be an RNAi agent such as an shRNA. The cell may contain an expression cassette that comprises regulatory sequences for transcription operably linked to a nucleic acid segment that encodes one or more than one RNAi agent or strand thereof. The cell may contain two or more expression cassettes, each of which comprises regulatory sequences for transcription operably linked to a nucleic acid segment that encodes one or more than one RNAi agent or a strand thereof. RNAi agents may be targeted to the same gene or to two or more different genes. For example, a first RNAi agent may be targeted to a first candidate disease gene and a second RNAi agent may be targeted to a second candidate disease gene. The invention encompasses lentiviral vectors that encode 1, 2, 3, 4, 5, or more RNAi agents or strands thereof.

Cells may be eukaryotic cells, e.g., mammalian or avian cells. According to certain embodiments of the invention a cell is a mouse or human cell. They may be dividing cells or non-dividing cells of any cell type. They may be cells that divide intermittently, e.g., that remain in the GO phase of the cell cycle for extended periods of time (e.g., weeks, months, years), or cells that divide only after being stimulated to do so. The cells may be primary cells, e.g., cells that are isolated from the body of a multicellular organism, which may have undergone one or more cycles of cell division following their isolation (e.g., 1-5 or 1-10 cycles of cell division). The cells may be immortalized cells, e.g., cells capable of continuous and prolonged growth in culture, e.g., they may be capable of undergoing hundreds or thousands of cell division cycles. The cells may be from cell lines, e.g., populations of cells derived from a single progenitor cell. The cells may be stem cells, e.g., embryonic or adult stem cells (Pfeifer, 2002). The cells may be isolated cells. In certain embodiments of the invention the cell is isolated from or present in a transgenic nonhuman animal. In certain embodiments of the invention the cell is one that has been administered to a subject.

Transgenic Animals and Uses Thereof

Lentiviral vectors of the invention may be used to generate transgenic animals. As used herein, a “transgenic animal” is a non-human animal, preferably a mammal or avian, in which one or more of the cells of the animal, typically essentially all cells of the animal, includes a transgene integrated into the genome. Examples of transgenic animals include non-human primates, rodents such as mice or rats, sheep, dogs, cows, goats, chickens, amphibians, and the like. Transgenic animals typically carry a gene that has been introduced into the germline of the animal, or an ancestor of the animal, at an early (usually one-cell) developmental stage. In general, a transgene is heterologous DNA, which is typically present in the genome of cells of a transgenic animal but is not present in the genome of non-transgenic animals of the same species or, if present, is located at a different position in the genome. Transgene sequences may include endogenous sequences but typically also include additional sequences that do not naturally occur in the animal. Integration of a transgene may lead to a deletion of endogenous chromosomal DNA, e.g., by homologous recombination, such that the function of a gene of interest is impaired or eliminated. In this case the resulting animal is referred to as a knockdown or knockout animal. A similar effect may be obtained if the transgene encodes an RNAi agent targeted to the gene of interest.

The present invention provides transgenic nonhuman animals generated using any of the lentiviral vectors of the present invention. The genome of the transgenic animal comprises sequences, e.g., a provirus, derived from a lentiviral vector of the present invention. A cell whose genome comprises a lentivirally transferred transgene may be distinguished from a cell whose genome comprises a transgene introduced into the genome without use of a lentiviral vector in that the genome also comprises sequences, e.g., lentiviral sequences, derived from a lentiviral vector. Lentiviral sequences are typically located within about 10 kB (e.g., between about 1 kB and about 10 kB) from the 5′ and/or 3′ end of the transgene. Sequences may include (i) one or more LTRs or portions thereof; (ii) packaging sequence; (iii) sequences required for integration; and/or (iv) FLAP element, etc. A transgene may be located between lentiviral sequences. Progeny and descendants of a transgenic animal generated using a lentiviral vector of the present invention are also considered to be generated using the lentiviral vector.

In certain embodiments of the invention the genome of the transgenic animal comprises (i) heterologous lentivirus derived sequences, e.g., at least a first LTR or portion thereof and at least a second LTR or portion thereof, wherein the lentivirus derived sequences are sufficient for reverse transcription and integration; and (ii) an ARE and, in some embodiments of the invention also comprises a SAR, wherein the ARE and SAR are located between lentivirus derived sequences. In certain embodiments of the invention the genome of transgenic animals further comprises one or more heterologous expression cassettes provided by a lentiviral vector of the invention, each of which comprises regulatory sequences operably linked to a sequence that encodes an RNA of interest. As discussed further below, in certain embodiments of the invention RNA(s) of interest hybridize or self-hybridize to form an RNAi agent such as an shRNA or siRNA or another RNA structure that undergoes further processing in the cell to generate an active RNAi agent. Alternately, one or more of the expression cassettes may comprise a transgene that encodes an mRNA that encodes a polypeptide of interest.

The invention provides a transgenic animal that expresses a lentivirally transferred transgene in at least approximately 50% of the cells of 2, 3, 4, or more different cell types, e.g., any 2, 3, 4, or more hematopoietic cell types such as B cell, T cell, macrophages, granulocytes (e.g., neutrophils), etc. In certain embodiments of the invention the percentage of cells of multiple different types that express the transgene averages between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%. In certain embodiments of the invention the percentage of cells that express the transgene remains stable in at least 2, 3, or 4 generations of descendants of the transgenic animal. For example, the percentage of cells of multiple different types that express the transgene averages between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%, in the F2, F3, and F4 generation. The cells may be, e.g., any 2, 3, 4, or more hematopoietic cell types such as B cell, T cell, macrophages, granulocytes, etc.

In certain specific embodiments, a lentiviral vector of the invention can be used to create a transgenic nonhuman animal as described above, wherein the transgenic animal expresses an RNAi agent, e.g., an shRNA, that is targeted to a target gene of interest. The invention provides transgenic animals in which expression of a gene of interest is inhibited in at least approximately 50% of the cells of 2, 3, 4, or more different cell types, e.g., any 2, 3, 4, or more hematopoietic cell types such as B cell, T cell, macrophages, granulocytes (e.g., neutrophils), etc., by a lentivirally transferred transgene that encodes an RNAi agent such as an shRNA targeted to the gene of interest. In certain embodiments of the invention the percentage of cells of multiple different types in which the gene of interest is inhibited is between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%. In certain embodiments of the invention the percentage of cells in which the gene of interest is inhibited remains stable in at least 2, 3, or 4 generations of descendants of the transgenic animal. For example, the percentage of cells of multiple different types in which the gene of interest is inhibited averages between approximately 50% and approximately 60%, approximately 70%, approximately 80%, approximately 90%, or approximately 100%; between approximately 60% and approximately 70%, approximately 80%, approximately 90%, or approximately 100%; between approximately 70% and approximately 80%, approximately 90%, or approximately 100%; between approximately 80% and approximately 90% or approximately 100%; or between approximately 90% and approximately 100%, in the F2, F3, and F4 generation. The cells may be, e.g., any 2, 3, 4, or more hematopoietic cell types such as B cell, T cell, macrophages, granulocytes, etc.

In any of these embodiments expression of the gene of interest may be inhibited by at least approximately 20%, approximately 30%, approximately 40%, approximately 50%, approximately 60%, approximately 70%, approximately 80%, approximately 90%, or approximately 100% in 2, 3, 4, or more cell types. For example, the average expression level of the gene of interest may be between approximately 0% (undetectable) and approximately 10%, approximately 20%, approximately 30%, approximately 40%, approximately 50%, approximately 60%, approximately 70%, or approximately 80% of the level that would exist in a congenic, nontransgenic animal in 2, 3, 4, or more cell types. In certain embodiments of the invention the average expression level of the gene of interest is between approximately 0% (undetectable) and approximately 10%, approximately 20%, approximately 30%, approximately 40%, or approximately 50% of the level that would exist in a congenic, nontransgenic animal in 2, 3, 4, or more cell types.

The gene of interest that is expressed or inhibited in a transgenic animal may be any gene. In certain embodiments of the invention the gene is a disease-associated gene. Genes of interest include genes whose inhibition results in a desirable trait such as increased growth, increased lifespan, or alteration in any phenotypic characteristic of interest.

The genome of the transgenic animal may contain an expression cassette that comprises regulatory sequences for transcription operably linked to a nucleic acid segment that encodes one or more than one RNAi agent or a strand thereof. The genome may comprise two or more expression cassettes, each of which comprises regulatory sequences for transcription operably linked to a nucleic acid segment that encodes one or more than one RNAi agent or a strand thereof. The RNAi agents may be targeted to the same gene or to two or more different genes. For example, a first RNAi agent may be targeted to a first candidate disease gene and a second RNAi agent may be targeted to a second candidate disease gene. The invention encompasses transgenic animals that express one or more 1, 2, 3, 4, 5, or more RNAi agents or strands thereof.

Transgenic animals that express an RNAi agent targeted to a disease-associated gene can serve as animal models of the disease. In general, the disease-associated gene is to be targeted by an RNAi agent is a gene characterized in that reduced or absent expression of the gene (or a particular allele of the gene) correlates with and is generally at least in part responsible for an increased incidence of development, progression, and/or severity of one or more manifestations of a disease. For example, transgenic animals in which expression of the Nramp1 gene is inhibited as a result of expression of an shRNA targeted to the Nramp1 transcript develop diabetes at a significantly decreased frequency relative to congenic animals that do not express the shRNA and also display increased susceptibility to bacterial infection. These transgenic animals and any transgenic animal obtained therefrom are aspects of this invention. Any gene that is or has been contemplated as a target for conventional “knockout” strategies can be targeted by RNAi using a lentiviral vector of the invention. Examples include, but are not limited to, tumor suppressor genes, kinases, phosphatases, receptors, channels, transporters, G proteins, cyclins, biosynthetic enzymes, cytokines, growth factors, genes that encode structural proteins, etc. In certain embodiments of the invention the gene is one whose expression is essential during one or more developmental stages or in one or more tissues of the organism. The use of RNAi agents whose expression is either regulatable or that allow for significant, though reduced, levels of expression of the target gene allow creation of transgenic animals under conditions in which conventional gene deletion strategies may be unsuccessful. In certain embodiments of the invention the animal is of a type or strain in which direct targeted gene-disruption using nonviral methods has not yet been achieved.

Transgenic animals that express a disease-associated gene characterized in that increased or inappropriate expression of the gene (or a particular allele or mutant form of the gene) correlates with and is generally at least in part responsible for an increased incidence of development, progression, and/or severity of one or more manifestations of a disease can also serve as animal models for disease. For example, transgenic animals that express any of a variety of activated oncogenes have a significantly greater incidence of cancer than congenic nontransgenic mice.

Transgenic animals may be used for a variety of purposes. For example, if inhibiting, expression of a gene results in a phenotypic effect that replicates one or more manifestations of a disease, this observation can confirm the role of the gene in the disease and validate it as a target for therapeutic intervention (e.g., by administering an agent that acts as an inhibitor or antagonist). Alternatively or additionally, if inhibiting expression of a gene results in a decreased incidence of development, progression, and/or severity of one or more manifestations of a disease, this observation can confirm the role of the gene in conferring a protective effect and validate it as a target for therapeutic intervention (e.g., by administering an agent that acts as an agonist, activator, or mimetic). Transgenic animals in which expression of a gene is inhibited, or in which a gene is overexpressed or aberrantly expressed can be used to study the role of the gene product in normal physiological processes and/or in pathologic processes. Creating a transgenic animal can help determine whether a candidate gene or an allele, or variant of the gene or a mutation in the gene plays a causative role in a disease or confers a protective effect. A “candidate gene” may be any gene that is suspected of being potentially relevant to a disease. For example, a candidate gene may be in linkage disequilibrium with the disease, e.g., one or more variants, alleles, or mutations of the gene may be present in a higher or lower percentage of individuals having the disease than individuals not having the disease. Alternatively or additionally, the known or putative function of the gene product may suggest a role in the disease. If a candidate gene plays a causative role in a disease then a transgenic animal that overexpresses the gene or in which expression of the gene is inhibited may exhibit features of the disease. The invention therefore provides a method of determining whether a candidate gene plays a causative role in disease comprising (i) creating a transgenic animal using a lentiviral vector of the invention that encodes the candidate gene or encodes an RNAi agent targeted to the gene; and (ii) determining that the candidate gene plays a role in the disease if the transgenic animal exhibits one or more features of the disease.

Potential therapeutic agents can be administered to the animal models of disease and the ability of the agent(s) to provide a beneficial effect, e.g., to reduce the risk that the animal will develop the disease, to inhibit disease progression, to reduce one or more symptoms or signs of the disease, to extend lifespan, etc., can be assessed. The disease can be a monogenic disease displaying a Mendelian single gene inheritance pattern or a multigenic disease, e.g., a disease in which alleles or mutations at multiple different genetic loci confer increased susceptibility or play a protective role. Exemplary diseases of interest for which animal models can be created include allergy, asthma, autoimmune diseases, atherosclerosis, cancer, diabetes, susceptibility to various infections, neurodegenerative diseases, neuropsychiatric diseases such as depression, epilepsy, schizophrenia; etc. Transgenic animals that express one or more RNAi agents targeted to different disease-associated genes can be bred to one another to create animal models of multigenic diseases.

Transgenic animals of the invention can also be used to test diagnostic or imaging reagents.

In some embodiments, an RNAi agent is targeted to a gene that encodes or plays a role in synthesis of a polypeptide or other molecule that would be antigenic in humans. The transgenic animal is deficient in the antigenic molecule. Such animals may be used as sources of organs for organ transplantation. In embodiments of the invention in which the nucleic acid segment that encodes an RNAi agent or a strand thereof is foxed, inhibition of the target transcript may be reversed by expressing Cre, thereby excising the nucleic acid from the genome of cells in which Cre is expressed. Thus the invention allows conditional and tissue-specific expression of target transcripts in cells or tissues of a transgenic animal.

Transgenic animals generated using the lentiviral vectors of the present invention may be used to produce an RNA or polypeptide of interest. For example, transgenic goats, cattle, pigs, etc., may express the polypeptide in their milk, from which the polypeptide can be harvested. Transgenic avians, e.g., chickens, can produce the polypeptide of interest in their eggs, e.g., in egg white. Appropriate regulatory sequences to achieve cell or tissue specific expression of a transgene in the mammary gland or in eggs (e.g., a promoter derived from a protein present in milk such as casein or whey acid protein, or in egg white such as ovalbumin or lysein, can be used; Houdebine, 2000; Lillico, 2005; and references therein). A polypeptide of interest may be, e.g., a polypeptide of pharmaceutical or diagnostic interest such as a monoclonal antibody, enzyme, clotting factor, recombinant receptor

Lentiviral vectors of the invention may be used to generate transgenic methods using any suitable method known in the art. Lentiviral particles of the invention may be used to create transgenic animals, wherein the transgene is a heterologous nucleic acid contained in the genome of the lentiviral particle. For example, lentiviral particles of the invention may be injected into the perivitelline space of single-cell embryos, which may then be implanted and carried to term. Alternately, the zona pellucida may be removed and the denuded embryo incubated with lentiviral suspension prior to implantation (Lois, 2002). This approach offers a convenient and efficient method of creating a variety of transgenic animals, e.g., birds, mice, rats, pigs, cattle, and other mammals. Lentiviral transgenesis is recognized as being an effective means of generating transgenic animals of a wide variety of types, and methods for doing so are readily available in the literature (Pfeifer, 2004; Hofmann, 2003; Fässler, 2004; and references in any of the foregoing.)

Alternatively or additionally, transgenic animals may be generated through standard (non-viral) means such as pronuclear injection of a transfer plasmid of the invention. Briefly, these methods include (i) introducing a transfer plasmid of the invention comprising a transgene into nuclei of fertilized eggs by microinjection, followed by transfer of the egg into the genital tract of a pseudopregnant female; or (ii) introducing a transfer plasmid of the invention comprising a transgene into a cultured somatic cell (e.g., using any convenient technique such as transfection, electroporation, etc.), selecting cells in which the transgene has integrated into genomic DNA, transferring the nucleus from a selected cell into an oocyte or zygote, optionally culturing the oocyte or zygote in vitro to the morula or blastula stage, and transferring the embryo into a recipient female. Cytoplasmic microinjection of an appropriate lentiviral transfer plasmid into an oocyte or embryonic cell can also be used. Heterozygous or chimeric animals obtained using these methods are identified and bred to produce homozygotes.

Methods for making transgenic avians are known in the art and include those described above and variations thereof. Methods suitable for production of transgenic avians and other transgenic animals are described, for example, in U.S. Pat. No. 6,730,822; U.S. Patent Publications 2002/0108132 and 2003/0126629; and references in these, and can be used to generate transgenic animals using the vectors of the present invention.

### Kits

The invention provides a variety of kits comprising one or more of the lentiviral vectors of the invention. For example, the invention provides a kit comprising a lentiviral vector comprising a nucleic acid comprising (i) a eukaryotic anti-repressor element (ARE); and (ii) lentivirus derived sequences sufficient for reverse transcription and packaging. A nucleic acid may further comprise an SAR. Any of the lentiviral vectors described herein may be included in the kit. In certain embodiments of the invention a lentiviral vector is a lentiviral transfer plasmid. A kit may comprise multiple different lentiviral vectors and may include one or more lentiviral vectors that do not comprise an ARE. A kit may comprise any of a number of additional components or reagents in any combination. The various combinations are not set forth explicitly but each combination is included in the scope of the invention. For example, one or more of the following items: (i) one or more vectors, e.g., plasmids, that collectively comprise nucleic acid sequences coding for retroviral or lentiviral Gag and Pol proteins and an envelope protein. The set of vectors may include two or more vectors. According to certain embodiments of the invention the kit includes (in addition to a lentiviral vector of the invention) at least two vectors (e.g., plasmids), one of which provides nucleic acid sequences coding for Gag and Pol and the other of which provides nucleic acid segments coding for an envelope protein; (ii) cells (e.g., a cell line) that are permissive for production of lentiviral particles (e.g., 293T cells); (iii) packaging cells, e.g., a cell line that is permissive for production of lentiviral particles and provides the proteins Gag, Pol, Env, and, optionally, Rev; (iv) cells suitable for use in titering lentiviral particles; (v) a transfection-enhancing agent such as Lipofectamine; (vii) an infection/transduction enhancing agent such as polybrene; (vii) a selection agent such as an antibiotic, preferably corresponding to an antibiotic resistance gene in the lentiviral transfer plasmid; (viii) a lentiviral vector comprising a heterologous nucleic acid segment such as a reporter gene that may serve as a positive control (referred to as a “positive control vector”); (ix) a lentiviral vector (“silencing control vector”) comprising a heterologous nucleic acid that encodes an RNAi agent targeted to a selected gene (“control gene”) for use as a control for gene silencing. Any gene may be selected as a control gene. The control gene may be, e.g., an abundantly and/or ubiquitously expressed gene such as the gene encoding cyclophilin. The RNAi agent is preferably one that is known to effectively silence the control gene. The kit may include (x) a vector for testing a sequence of an RNAi agent to determine whether it effectively silences a target gene of interest. For example, the vector can be a Renilla/firefly dual-luciferase reporter gene into which a target gene of interest, or a portion thereof, can be cloned. Alternatively or additionally the kit may include any of the following: (xi) one or more restriction enzymes; (xii) DNA oligonucleotide primers or linkers compatible with the lentiviral vector for use in cloning shRNA-encoding DNA into the vector (e.g., the primers or linkers may be at least in part complementary or identical to a portion of the vector that comprises a restriction site or portion thereof; (xiii) DNA ligation or amplification enzymes, e.g., DNA ligase, DNA polymerase (e.g., heat-stable DNA polymerase such as Taq polymerase); (xiv) one or more reaction buffers.

According to certain embodiments of the invention a kit comprises a set of lentiviral vector comprising a variety of different promoters and/or reporter genes. For example, a kit may comprise a first lentiviral vector that comprises a Pol I or Pol III promoter and a second lentiviral vector that comprises a heterologous Pol II promoter.

Kits typically include instructions for use of lentiviral vectors. Instructions may, for example, comprise protocols and/or describe conditions for transfection, transduction, infection, production of lentiviral particles, gene silencing, etc. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### Collections

The invention provides “sets” or “collections” comprising multiple lentiviral vectors of the invention, each of which encodes a polypeptide of interest or an RNAi agent of interest. A collection may include vectors that collectively comprise at least approximately 10% of the coding sequences of a eukaryotic organism of interest, e.g., a rodent (e.g., mouse, rat, hamster), primate (e.g., human), etc., or that collectively encode at least approximately 10% of the polypeptides expressed in a eukaryotic cell or organism of interest. A collection may include vectors that collectively comprise between approximately 10% and approximately 100% of the coding sequences of a eukaryotic organism of interest, or any intervening range. A collection may include vectors that collectively encode RNAi agents targeted to coding sequences of a eukaryotic organism of interest, e.g., a rodent (e.g., mouse, rat, hamster), primate (e.g., human), etc., or that collectively encode RNAi agents targeted to at least approximately 10% of the genes that encode polypeptides expressed in a eukaryotic cell or organism of interest. A collection may include vectors that collectively encode RNAi agents targeted to between approximately 10% and approximately 100% of the coding sequences of a eukaryotic organism of interest, or any intervening range.

The invention further provides collections of transgenic animals generated using collections of lentiviral vectors.

### Therapeutic Applications and Pharmaceutical Compositions

Lentiviral vectors of the invention are useful for a wide variety of therapeutic applications. In particular, they are useful in any context for which gene therapy is contemplated. For example, lentiviral vectors comprising a heterologous nucleic acid segment operably linked to a promoter are useful for any disease or clinical condition associated with reduction or absence of the protein encoded by the heterologous nucleic acid segment, or any disease or clinical condition that can be effectively treated by augmenting the expression of the encoded protein within the subject. For example, lentiviral vectors comprising a nucleic acid segment encoding the cystic fibrosis transmembrane conductance regulator (CFTR) or encoding α1-antitrypsin may be used for the treatment of cystic fibrosis and α1-antitrypsin deficiency, respectively. Lentiviral vectors comprising a nucleic acid segment encoding Factor VIII or Factor IX may be used for treatment of hemophilia A or B, respectively. Lentiviral vectors comprising a nucleic acid segment encoding gamma c gene can be used for treatment of X-linked severe combined immunodeficiency (Hacein-Bey-Abina, 2002).

Inventive lentiviral vectors that comprise an expression cassette for synthesis of an RNAi agent (e.g., one or more siRNAs or shRNAs) are useful in treating any disease or clinical condition associated with overexpression of a transcript or its encoded protein in a subject, or any disease or clinical condition that may be treated by causing reduction of a transcript or its encoded protein in a subject. For example, many cancers are associated with overexpression of oncogene products. Delivering a lentiviral vector that provides a template for synthesis of one or more RNAs that self-hybridize or hybridize with each other to form an RNAi agent such as an shRNA or siRNA targeted to the transcript encoding the oncogene product may be used to treat such cancers. The high degree of specificity achieved by RNA interference allows selective targeting of transcripts comprising single base pair mutations while not interfering with expression of the normal cellular allele. Lentiviral vectors that comprise an expression cassette for synthesis of one or more RNAs that self-hybridize or hybridize with each other to form an RNAi agent targeted to a transcript encoding a cytokine may be used to regulate immune system responses (e.g., responses responsible for organ transplant rejection, allergy, autoimmune diseases, inflammation, etc.): Lentiviral vectors that provide a template for synthesis of one or more RNAs that self-hybridize or hybridize with each other to form an RNAi agent targeted to a transcript of an infectious agent or targeted to a cellular transcript whose encoded product is necessary for or contributes to any aspect of the infectious process may be used in the treatment of infectious diseases.

Gene therapy protocols may involve administering an effective amount of a lentiviral vector whose presence within a cell results in production of an RNAi agent to a subject either before, substantially contemporaneously, with, or after the onset of a condition to be treated. Another approach that may be used alternatively or in combination with the foregoing is to isolate a population of cells, e.g., stem cells or immune system cells from a subject, optionally expand the cells in tissue culture, and administer a lentiviral vector whose presence within a cell results in production of an RNAi agent to the cells in vitro. The cells may then be returned to the subject, where, for example, they may provide a population of cells that produce an RNAi agent, or that are resistant to infection by an infectious organism, etc. Optionally, cells expressing a therapeutic RNAi agent can be selected in vitro prior to introducing them into the subject. In some embodiments of the invention, a population of cells, which may be cells from a cell line or from an individual other than the subject, can be used. Methods of isolating stem cells, immune system cells, etc., from a subject and returning them to the subject are well known in the art. Such methods are used, e.g., for bone marrow transplant, peripheral blood stem cell transplant, etc., in patients undergoing chemotherapy.

Compositions comprising lentiviral vectors of the invention may encode an RNAi agent targeted to a single site in a single target transcript, or alternatively may encode multiple different RNAi agents targeted to one or more sites in one or more target transcripts. In some embodiments of the invention, it will be desirable to utilize compositions comprising one or more lentiviral vectors that collectively encode multiple different RNAi agents targeted to different genes, which may be cellular genes or, where an infection is being treated, genes of an infectious organism. Some embodiments of the invention provide templates for more than one siRNA or shRNA species targeted to a single transcript. To give but one example, it may be desirable to provide templates for synthesis of one or more RNAs that self-hybridize or hybridize with each other to form at least one RNAi agent targeted to coding regions of a target transcript and at least one RNAi agent targeted to the 3′ UTR. This strategy may provide extra assurance that products encoded by the relevant transcript will not be generated because at least one agent will target the transcript for degradation while at least one other inhibits the translation of any transcripts that avoid degradation. The invention encompasses “therapeutic cocktails,” including approaches in which a single lentiviral particle provides templates for synthesis of one or more RNAs that self-hybridize or hybridize to form RNAi agents that inhibit multiple target transcripts. The invention further encompasses compositions comprising a lentiviral vector of the invention and a second therapeutic agent, e.g., a composition approved by the U.S. Food and Drug Administration.

Inventive compositions may be formulated for delivery by any available route including, but not limited to parenteral (e.g., intravenous), intradermal, subcutaneous, oral (e.g., inhalation), transdermal (topical), transmucosal, rectal, and vaginal. Commonly used routes of delivery include parenteral, transmucosal, rectal, and vaginal. Inventive pharmaceutical compositions typically include a lentiviral vector in combination with a pharmaceutically acceptable carrier. As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions.

In some embodiments, active agents, i.e., a lentiviral vector of the invention and/or other agents to be administered together with a lentiviral vector of the invention, are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such compositions will be apparent to those skilled in the art. Suitable materials can also be obtained commercially from Alza Corporation and Nova Pharmaceuticals, Inc. Liposomes can also be used as pharmaceutically acceptable carriers. These can be prepared according to methods known to those skilled in the art, for example, as described in U.S. Pat. No. 4,522,811. In some embodiments the composition is targeted to particular cell types or to cells that are infected by a virus. For example, compositions can be targeted using monoclonal antibodies to cell surface markers, e.g., endogenous markers or viral antigens expressed on the surface of infected cells.

It is advantageous to formulate compositions in dosage unit form for ease of administration and uniformity of dosage. Dosage unit form as used herein refers to physically discrete units suited as unitary dosages for the subject to be treated; each unit comprising a predetermined quantity of a lentiviral vector calculated to produce the desired therapeutic effect in association with a pharmaceutical carrier.

Pharmaceutical compositions can be administered at various intervals and over different periods of time as required, e.g., one time per week for between about 1 to about 10 weeks; between about 2 to about 8 weeks; between about 3 to about 7 weeks; about 4 weeks; about 5 weeks; about 6 weeks, etc. For certain conditions such as HIV it may be necessary to administer the therapeutic composition on an indefinite basis to keep the disease under control. The skilled artisan will appreciate that certain factors can influence the dosage and timing required to effectively treat a subject, including but not limited to the severity of the disease or disorder, previous treatments, the general health and/or age of the subject, and other diseases present. Treatment of a subject with a lentiviral vector can include a single treatment or, in many cases, can include a series of treatments.

Exemplary doses for administration of gene therapy vectors and methods for determining suitable doses are known in the art. It is furthermore understood that appropriate doses of a lentiviral vector that encodes an RNAi agent, i.e., a vector that comprises a template for synthesis of one or more RNAs that self-hybridize or hybridize with each other to form an RNAi agent such as an shRNA or siRNA may depend upon the potency of the RNAi agent and may optionally be tailored to the particular recipient, for example, through administration of increasing doses until a preselected desired response is achieved. The appropriate dose level for any particular subject may depend upon a variety of factors including the activity of the specific RNAi agent employed, the age, body weight, general health, gender, and diet of the subject, the time of administration, the route of administration, the rate: of excretion, other administered therapeutic agents, and the degree to which it is desired to inhibit gene expression or activity.

Lentiviral gene therapy vectors can be delivered to a subject by, for example, intravenous injection, local administration, or by stereotactic injection (see, e.g., Chen et al. 1994, Proc. Natl. Acad. Sci., USA, 91:3054). In certain embodiments of the invention, vectors may be delivered orally or inhalationally and may be encapsulated or otherwise manipulated to protect them from degradation, enhance uptake into tissues or cells, etc. Pharmaceutical preparations can include a lentiviral vector in an acceptable diluent, or can comprise a slow release matrix in which a lentiviral vector is imbedded. Alternatively or additionally, where a vector can be produced intact from recombinant cells, as is the case for retroviral or lentiviral vectors as described herein, a pharmaceutical preparation can include one or more cells which produce vectors. Pharmaceutical compositions comprising a lentiviral vector of the invention can be included in a container, pack, or dispenser, optionally together with instructions for administration.

### EXEMPLIFICATION

### Example 1

### Selection of a Candidate Type 1 Diabetes-Associated Gene for Analysis by RNAi

### Materials and Methods

Congenic NOD Strains

The Idd5.1 and Idd5.1/Idd5.2 strains used have been reported previously as NOD.B10 Idd5R193 and NOD.B10 Idd5R444, respectively (Wicker, 2004). The Idd5.2 strain is a novel congenic strain developed from the Idd5.1/Idd5.2 by marker-assisted breeding as detailed previously (Hill, 2004).

The development of the NOD.B10 Idd5.1/Idd5.2 (R444) N13, NOD.B10 Idd5.1/Idd5.2 (R444s) N14 and Idd5.1/Idd5.2 (R193) N16 congenic strains and the extent of disease protection due to their protective alleles have been detailed (Wicker, 2004). R444s and R193 define the distal and proximal boundaries, respectively, of Idd5.2. The Idd5.1 interval was initially defined in the context of a protective allele at the Idd5.1 region (Wicker, 2004; and Hill, 2004). The NOD.B10 Idd5.1 (R52) N14 strain is a novel strain and its reduced frequency of diabetes as compared to the NOD strain indicates that a protective allele at Idd5.2 is evident in the absence of a protective allele Idd5.1. The recombination event defining the R52 congenic strain was identified by screening progeny following the intercross of (R444×NOD) F1 mice. Mice homozygous for the congenic region were identified following an intercross of heterozygous congenic mice derived from the selected recombinant mouse. The NOD.B10 Idd5.1/Idd5.2 (R444) N14 and Idd5.1/Idd5.2 (R193) are available from Taconic, Inc. via the Emerging Models Program as lines 1094 and 2574, respectively. Idd5.1 congenic strains, with protection from diabetes equal to that of R52, are also available (lines 3388 and 6146).

Measurement of Diabetes Frequency

Mice were considered diabetic when urinary glucose was >500 mg/dl; as measured with Diastix (Bayer Diagnostics). Diabetic mice also exhibited polydipsia, polyuria, and weight loss.

### Results

Type 1 diabetes (T1D) is an autoimmune disease influenced by many different genetic loci. More than 20 insulin-dependent diabetes (Idd) loci have been identified in the nonobese diabetic (NOD) mouse model by congenic strain positional cloning (Makino, 1980; and Todd, 2001); but because direct targeted gene-disruption is not yet possible in this strain, few gene variants have been shown to be causal (Ueda, 2003; and Vijayakrishnan, 2004). Nramp1 (also known as Slc11a1) encodes for a phagosomal ion-transporter that affects resistance to intracellular pathogens and influences antigen presentation (Vidal, 1993; Vidal, 1995; and Wojciechowski, 1999). This gene is the strongest candidate amongst the 42 genes in the protective Idd5.2 locus in which a naturally occurring mutation confers loss-of-function to the NRAMP1 protein (Wicker, 2004).

Genetic analysis of the NOD model of type 1 diabetes (T1D) by a congenic strain positional cloning strategy has helped uncover numerous genetic intervals linked to disease. However, the reduction of a congenic interval to include only one disease-associated gene is nearly always technically impossible, particularly in gene-dense regions. Breeding knock-out (KO) alleles from a different mouse strain into the NOD background, besides being a very lengthy process, introduces genes closely linked to the KO allele that may themselves affect disease incidence (Kanagawa, 2000).

RNAi has been demonstrated to be achievable in mice (Rubinson, 2003; and Tiscornia, 2003). We therefore decided to test the feasibility of using RNAi to study causal genes in the NOD model of T1D. We selected a target gene that fulfills three criteria. First, the gene of interest had to be a likely candidate for a known disease-linked locus. Second, the polymorphism of this gene between disease-susceptible and disease-resistant alleles had to give rise to either a gain or loss of function that can be compensated or mimicked, respectively, by RNAi. Lastly, strains congenic for the locus of interest had to be available to permit direct comparison of disease incidence between congenic strains and animals in which the gene is silenced by RNAi. We found Nramp1 to fulfill all three criteria.

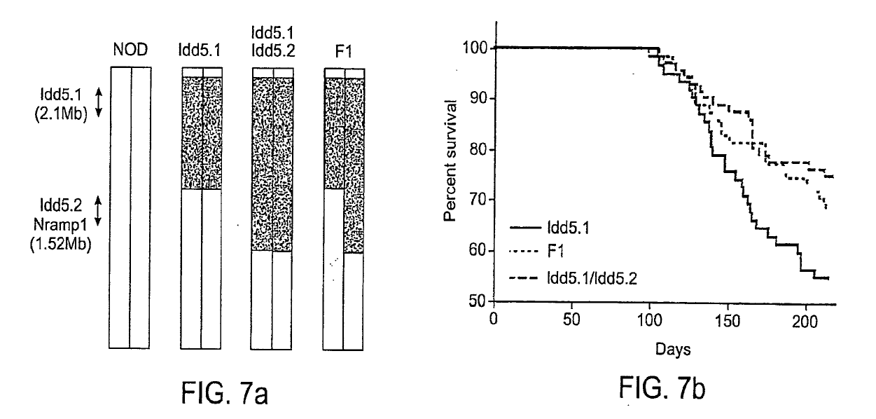

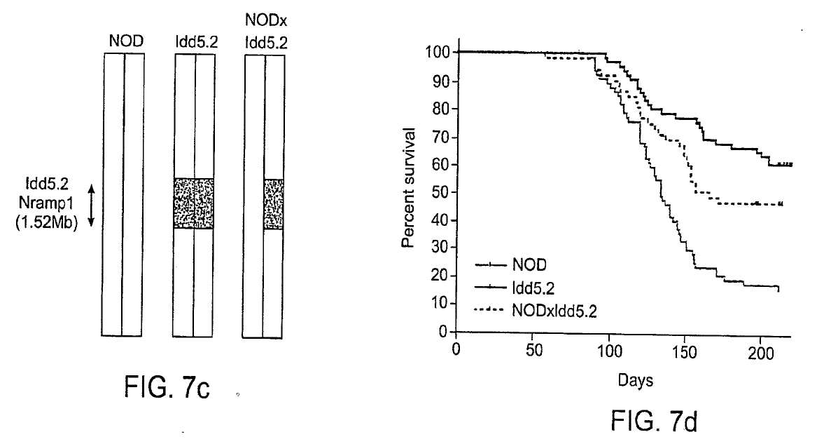

The Nramp1 gene has been determined to be the most likely candidate for the Idd5.2 locus (Wicker, 2004). FIG. 7a is a schematic representation of the Idd5.1 (2.1 Mb) and Idd5.2 (1.52 Mb) B10-derived regions (filled area) on chromosome 1 in NOD congenic mice. FIG. 7c is a schematic representation of the chromosome 1 region in Idd5.2 congenic mice. Filled regions are B10-derived. The Idd5.2 region contains 42 genes, including Nramp1. The protective allele of this locus comprises a mutation that confers a loss-of-function phenotype to the NRAMP1 protein (Vidal, 2003). Interestingly, this mutation also confers susceptibility to intracellular pathogen infection and has a clear role in other immune processes (Vidal, 1993; Vidal, 1995; Wojciechowski, 1999).

F1 mice are B10 homozygous at Idd5.1 and heterozygous at Idd5.2. We analyzed the development of diabetes over time in Idd5.1 (n=62), Idd5.1/Idd5.2 (n=55), and F1 (Idd5.1/Idd5.1, Idd5.21+; n=71) mice (FIG. 7b). In addition, we analyzed the development of diabetes over time in NOD (n=67), Idd5.2 (n=67), and Idd5.2/+ (n=53) female mice. FIG. 7d illustrates the dose effect of the Idd5.2 locus in isolation of the protective Idd5.1 locus. Note that in this animal model diabetic mice die within 2-3 weeks of diagnosis; therefore development of diabetes is essentially equivalent to death in these mice. As shown in FIGS. 7b and 7d, congenic mice having only one dose of the protective allele at Idd5.2 had a reduced frequency of T1D, demonstrating that the protective Idd5.2 allele is dominant, particularly within the context of protective Idd5.1 alleles (Ueda, 2003; and Wicker, 2004; FIG. 7b). If the protection mediated by Idd5.2 is indeed due to a nonfunctional NRAMP1 protein, the inventors anticipated that the dominant protection would enable even incomplete silencing of Nramp1 by RNAi to have a detectable effect on diabetes incidence.

### Example 2

### Design and Construction of a Lentiviral Vector Showing Reduced Variegation after Transgenesis

### Materials and Methods

Generation of the pLB Vector

pLL3.7 is a lentiviral transfer plasmid that comprises a U6 promoter located upstream of a multiple cloning site suitable for insertion of a template for transcription of an shRNA (Rubinson, et al., 2003). Anti-repressor #40 (ref. 17) was amplified from genomic DNA using the following primers: 5′ sense-ATATGGGCCCGGTGCTTTGCTCTGAGCCAGCCAC (SEQ ID NO: 123), 3′ antisense-ATATGGGCCCTGGCAGAAATGCAGGCTGAGTGAG (SEQ ID NO: 124) and cloned into the ApaI restriction site of pLL3.7. The human IFN-β SAR element (Klehr, 1991) was kindly provided by Dr. J. Bode and cloned into the blunted KpnI restriction site of pLL3.7.

Generation of Lentivirus and Embryo Transgenesis

Lentiviral production was done as described previously (Rubinson, et al., 2003; and U.S. Patent Publication 2005/0251872). Briefly, lentiviral pLL3.7 or pLB vector was co-transfected with packaging vectors into 293FT cells, and supernatants were collected at 48 hours and 72 hours. Combined supernatants were ultracentrifuged at 25,000 rpm for 1.5 hours in a Beckman SW32Ti rotor. Virus was resuspended in 50 p. 1 phosphate-buffered saline and titered as described (Rubinson, et al., 2003). Concentrated virus preparation (>5×10⁸ infectious units (IFU)/ml) was injected into the perivitelline space of single-cell embryos of the NOD or NOD Idd5.1 genotype that were then reimplanted into the oviduct of pseudo-pregnant recipient females.

Flow Cytometry

Peripheral blood, lymph node cells, splenocytes or thymocytes were stained with fluorochrome-conjugated anti-TCR, anti-B220, anti-CD4, anti-CD8, and anti-CD11b, as indicated (all from BD Pharmingen). Cells were washed and analyzed on a FACScalibur or FACScanto flow cytometer (Becton-Dickinson). Data analysis was performed using FlowJo software (TreeStar Inc.).

### Results

To first assess the potential use of RNAi in vivo in the NOD background, we initially targeted the T cell surface receptor CD8, a gene that is easily monitored and highly expressed. pLL3.7 is a lentiviral vector previously shown to mediate silencing in vivo in the C57BL/6 background (Rubinson, et al., 2003). Using this vector, a portion of which is shown in FIG. 6a, we generated lentivirus encoding a CD8-targeting short-hairpin RNA (shRNA), as shown in FIG. 6b. This virus was micro-injected into the perivitelline space of single-cell NOD embryos which, subsequent to re-implantation into pseudo-pregnant recipients, developed into transgenic adult NOD mice.

As shown previously, the expression of pLL3.7-CD8 shRNA decreased cell surface expression of this molecule on CD8⁺ T cells (Rubinson, et al., 2003). FIG. 8a shows a flow cytometry analysis of peripheral blood from a pLL3.7-CD8 shRNA lentiviral transgenic NOD mouse (right panels) and a non-transgenic littermate (left panels). The top panels show CD3 expression in the lymphocyte population. The middle panels show CD4 and GFP expression (gated on CD4⁺ cells). The bottom panels show CD8 and GFP expression (gated on CD8″ cells). CD4 and CD3 expression were unaffected by CD8 shRNA expression in T cells, suggesting a specific effect on the targeted gene. Expression of the GFP marker protein correlated well with silencing: few, if any, cells that expressed GFP retained wild-type levels of CD8. Conversely, no reduction of CD8 expression was detected in GFP-negative cells.

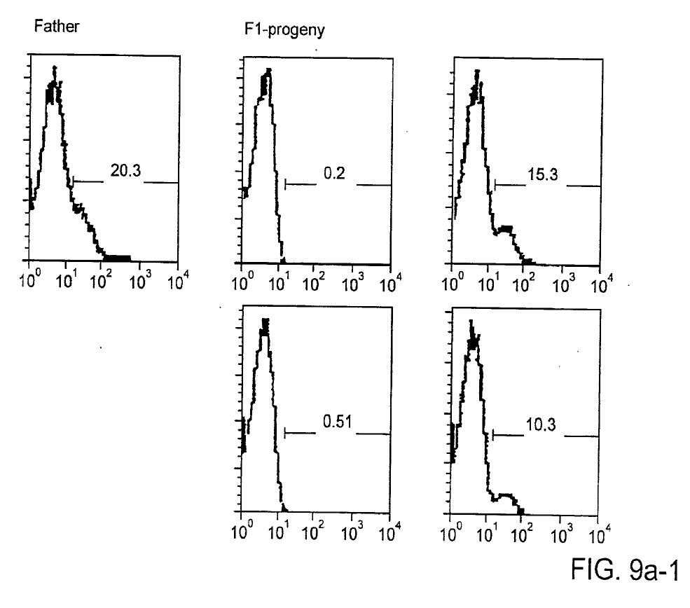

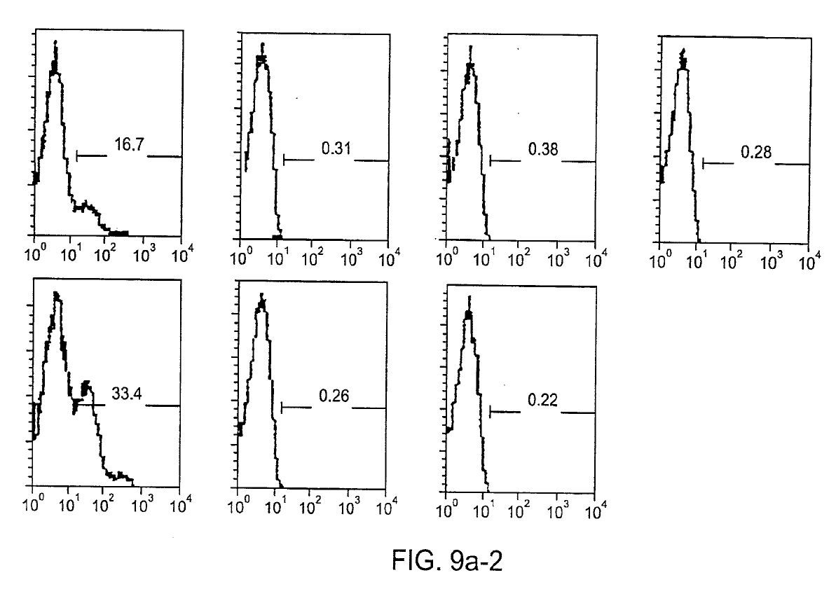

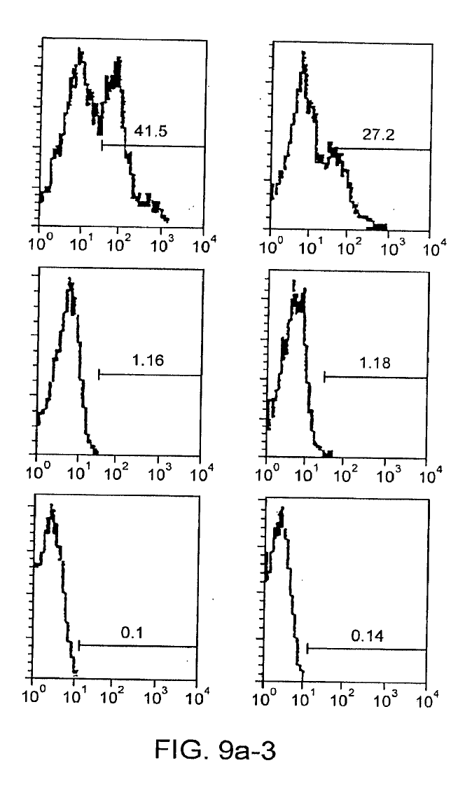

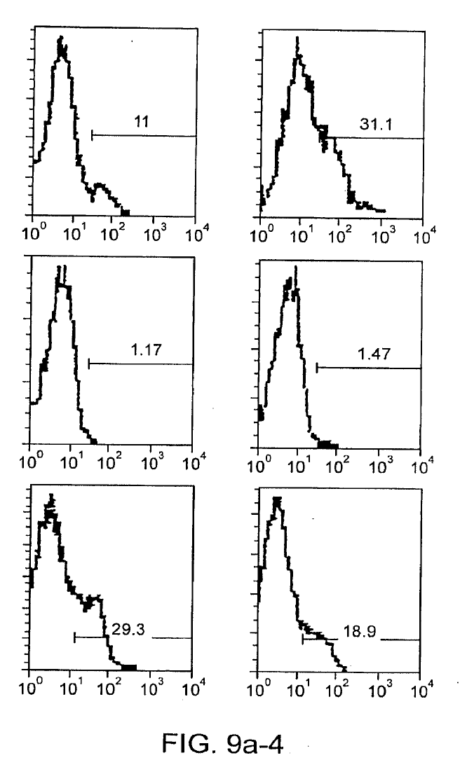

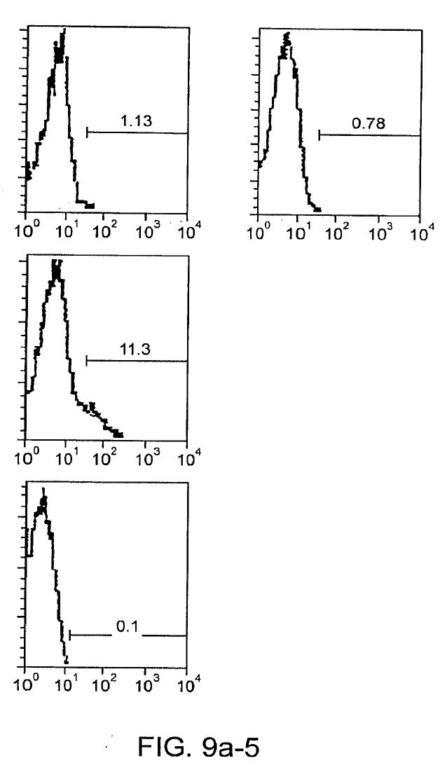

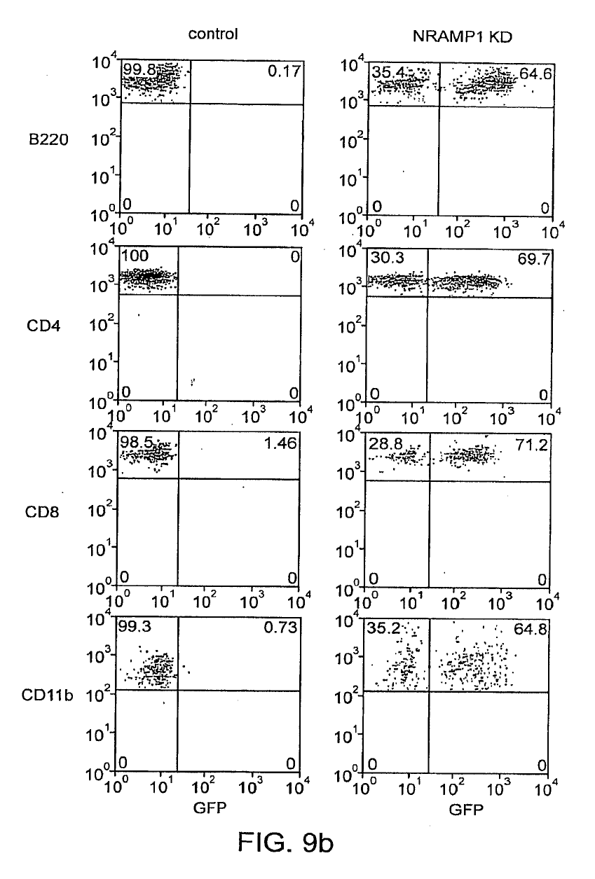

As shown in FIG. 8a, it became apparent that only a relatively low percentage of cells actually expressed the lentiviral construct. Expression was also variable between cell lineages. For example, GFP was detected in 34% of CD4 T cells, but in only 11% of B cells and 17.5% of granulocytes (FIG. 8a and data not shown). This variegated expression was consistently observed in several founder mice generated with different pLL3.7 constructs in both C57BL/6 and NOD animals. While not wishing to be bound by any theory, we believe that variegation was most likely due to epigenetic silencing, rather than mosaicism, since the progeny of lentiviral transgenic animals displayed similar variegation (FIG. 9a). To date, no reports have yet quantitatively demonstrated consistent and ubiquitous systemic expression of lentiviral constructs after transgenesis, regardless of integrant copy-numbers (Lois, 2002; Rubinson, 2003; and Lu, 2004).

In order to address this issue of variegated expression, we decided to modify the pLL3.7 vector by adding two genetic elements that we hypothesized would reduce the variegation. The upper portion of FIG. 8b shows a schematic diagram of a portion of pLL3.7 prior to modification. The U6 and CMV promoters drive shRNA and GFP expression, respectively. (Certain elements present in the vector and depicted in FIG. 6a are not shown here.) We modified pLL3.7 by inserting a fragment of one anti-repressor element (#40) (Kwaks, 2003) upstream of the U6 promoter and another element, termed scaffold-attached region (SAR) (Klehr, 1991) downstream of GFP to flank the expression cassette. The resulting vector was termed pLB. A portion of pLB, showing the positions of the added genetic elements, is presented in the lower portion of FIG. 8b.

We used the new pLB vector to generate transgenic NOD mice and analyzed GFP expression in hematopoietic cells isolated from these mice using flow cytometry, as shown in FIG. 8c. Peripheral blood from a pLB lentiviral transgenic NOD mouse (right panels) and a non-transgenic littermate (FIG. 8c, left panels) was stained for TCR (T cell marker), B220 (B cell marker), and CD11b (macrophage marker) for analysis by flow cytometry. The top, middle and bottom panels of FIG. 8c are gated on TCR⁺, B220⁺, and B220″ CD11b⁺ cells, respectively. Lineage marker and GFP expression are shown for each population. Transgenic mice generated with pLB vector displayed more consistent expression throughout hematopoietic lineages than mice generated with pLL3.7. Variegation was reduced, as some founders expressed the new lentiviral construct in 70% of peripheral blood cells in multiple lineages.

### Example 3

### Design and Testing of shRNA to Target Nramp1 mRNA

### Materials and Methods

Short Hairpin RNA Design

Nramp1 target sequences were selected according to criteria described previously (Schwarz, 2003; Khvorova, 2003; Reynolds, 2004): 545-GGACGGCTATCTCCTTCAA (SEQ ID NO: 125), 666-GCTTTCTTCGGTCTCCTCA (SEQ ID NO: 127), 870-GGTCAAGTCTAGAGAAGTA (SEQ ID NO: 126), 915-GCCAACATGTACTTCCTGA (SEQ ID NO: 128), 2196-GGCTCACAACCATCCATAA (SEQ ID NO: 129). These target sequences were used for the design of shRNA sequences as described previously (Rubinson, 2003). The complete sequences of the two oligos that were used for the 915 shRNA are as follows:

Forward:
(SEQ ID NO: 130)
5′TGCCAACATGTACTTCCTGATTCAAGAGATCAGGAAGTACATGTTGGC
TTTTTTC 3′
Reverse:
(SEQ ID NO: 131)
5′TCGAGAAAAAAGCCAACATGTACTTCCTGATCTCTTGAATCAGGAAGT
ACATGTTGGCA 3′

The resulting shRNA sequences were cloned into the pLB vector using the HpaI and XhoI restriction sites. FIG. 6d shows the Nramp1 stem loop sequence and the Nramp1 shRNA predicted to form following transcription.

Dual-Luciferase Reporter Assay

Nramp1 cDNA (gift from Dr. J. Blackwell) was cloned into the psiCHECK2 dual-luciferase reporter vector (Promega). 293FT cells (10⁵) were co-transfected with 50 ng psiCHECK2-Nramp1 or empty psiCHECK-2 vector, and 150 ng pLB vector (with or without NRAMP1 shRNA) using FuGene-6 transfection reagent (Roche Diagnostics). Cell lysates were analyzed using a Dual-Luciferase assay system (Promega) with a Veritas luminometer (Turner Biosystems). Ratios of Renilla/firefly luciferase activity were calculated and normalized to empty pLB transfection measurement (i.e. empty pLB=100% activity). Results are given in percent of relative luminescence units (RLU).

### Results

We designed several shRNA sequences to target Nramp1 mRNA using an algorithm that incorporates the most recently published criteria. These shRNA sequences were validated with a dual-luciferase reporter assay. The full-length Nramp1 cDNA was cloned into the 3′ UTR of the Renilla luciferase gene, and efficiency of silencing was assessed after co-transfection of the luciferase/Nramp1 reporter vector together with different shRNA sequences cloned into pLB. RNAi mediated by an effective shRNA targeted to Nramp1 should result in degradation of the luciferase/Nramp1 mRNA encoded by the reporter vector, thereby reducing Renilla luciferase expression (FIG. 8d). Several sequences potently silenced Renilla luciferase, with the best sequences tested inhibiting up to 85% of luciferase activity. Silencing was specific for the Nramp1 sequence, as shRNA expression did not affect luciferase activity in the absence of Nramp1 cDNA. The shRNA sequence 915 was consistently found to be most effective against Nramp1 cDNA and was used in the generation of lentiviral transgenic NOD mice as described in Example 4 below.

### Example 4

### Generation and Characterization of Lentiviral Transgenic NOD Mice Expressing shRNA Targeted to Nramp1

### Materials and Methods

Generation of Lentivirus and Embryo Transgenesis.

These were performed as described in Example 2.

Detection of NRAMP1 Protein

Mice were injected intra-peritoneally with 1 mg Concanavalin A (Sigma-Aldrich)5 days prior to peritoneal lavage. Peritoneal exudate cells (PEC) were stained for CD11b and sorted for CD11b and GFP expression by flow cytometry. Sorted macrophages were immediately lysed and analyzed by western blotting for NRAMP1 expression using a rabbit polyclonal antibody (clone H-100) followed by goat anti-rabbit HRP-conjugated antibody (both from Santa Cruz Biotech). HRP activity was detected with Western Lightning reagent (Perkins-Elmer). Protein loading was controlled by stripping the membrane and reprobing with γ-tubulin antibody (Sigma-Aldrich).

### Results

The shRNA sequence 915 was consistently found to be most effective against Nramp1 cDNA and was used in the generation of lentiviral transgenic NOD mice. Single-cell embryos from Idd5.1 congenic NOD mice (FIG. 7) were injected with pLB-915 virus and reimplanted into pseudo-pregnant recipients. Two out of the three pups born following injection expressed high levels of GFP. In one founder in particular, approximately 65% of all peripheral blood cells expressed the lentiviral construct. Separate cell lineages differed to some degree, with 70% of T cells and 65% of B cells and macrophages being GFP-positive (FIG. 9b). To assess the possibility of establishing large, homogenous cohorts of lentiviral transgenic NOD mice, we extensively bred this founder and its progeny with Idd5.1 mice over four generations.

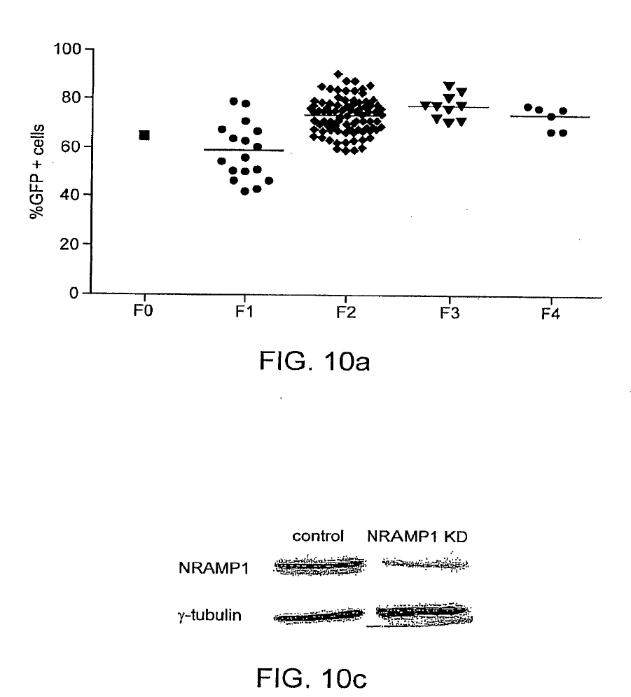

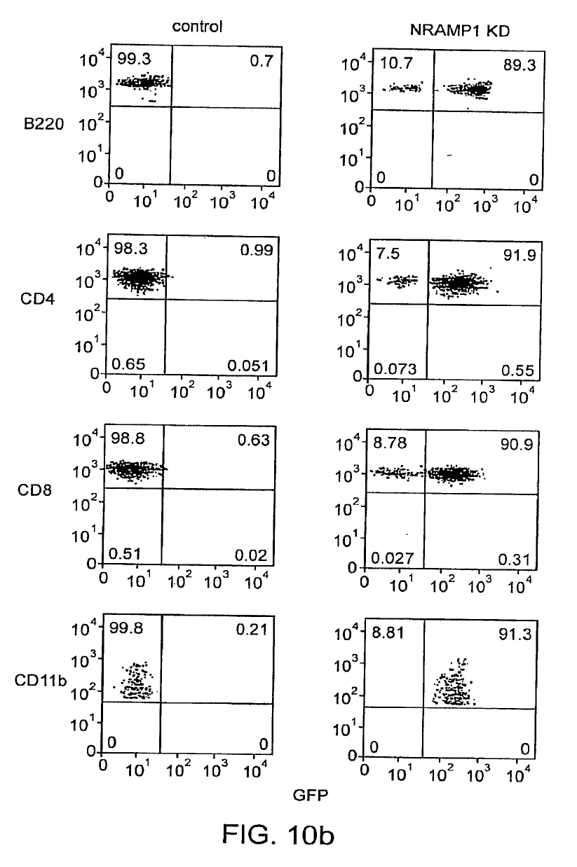

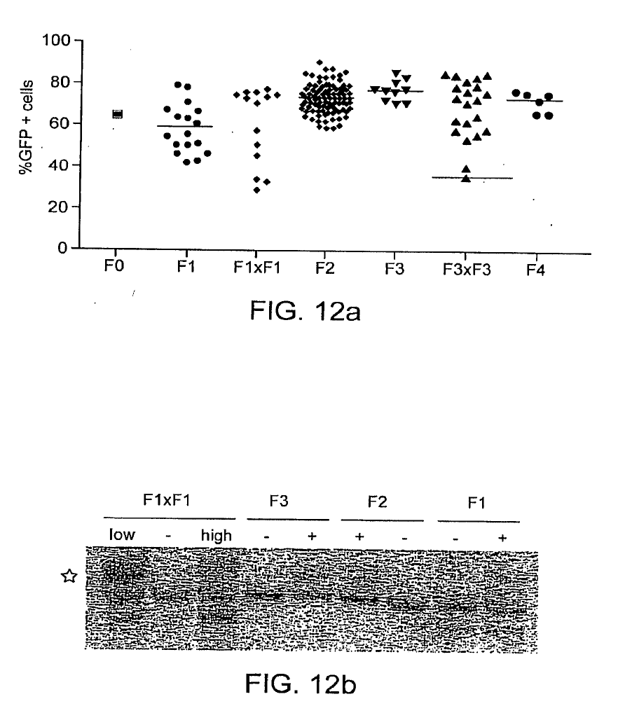

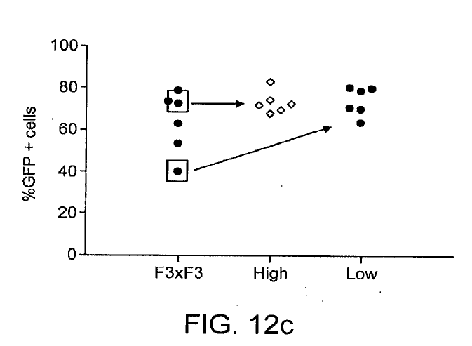

Approximately 50% of the progeny expressed the lentiviral construct (165/362). Southern-blot analysis confirmed that the GFP-positive phenotype correlated with the inheritance of a single locus (not shown). GFP expression was detected in 45%-75% of hematopoietic cells in F1 mice (FIG. 10a). F2 mice expressed significantly higher levels than the F1 generation (average 73%, unpaired t-test: P<0.0001), independently of parental expression (F2 mice were from five separate breeders), with the highest levels of expression reaching 90% in the peripheral blood. F3 and F4 mice displayed high expression levels (average 77% and 73%, respectively), similar to the F2 generation. Analysis of thymocytes, splenocytes, and lymph node cells confirmed that expression was consistently over 75%, and as high as 90% in some animals (FIG. 10b and data not shown). Without wishing to be bound by any theory, the variability in the F1 generation could be attributed to interference between lentiviral integrants (FIGS. 12a-12b), the exact mechanism of which remains elusive. However, expression remained stable and consistent throughout the F2, F3, and F4 generations.

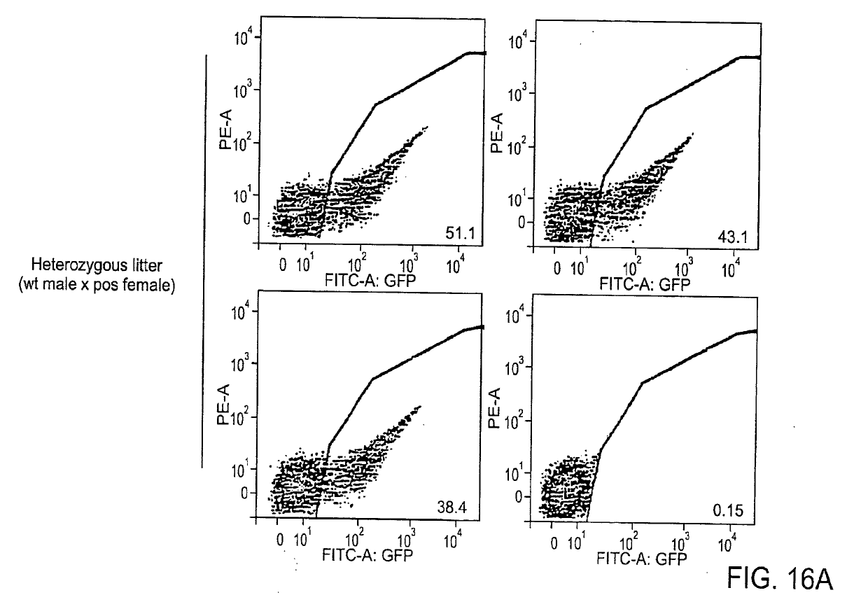

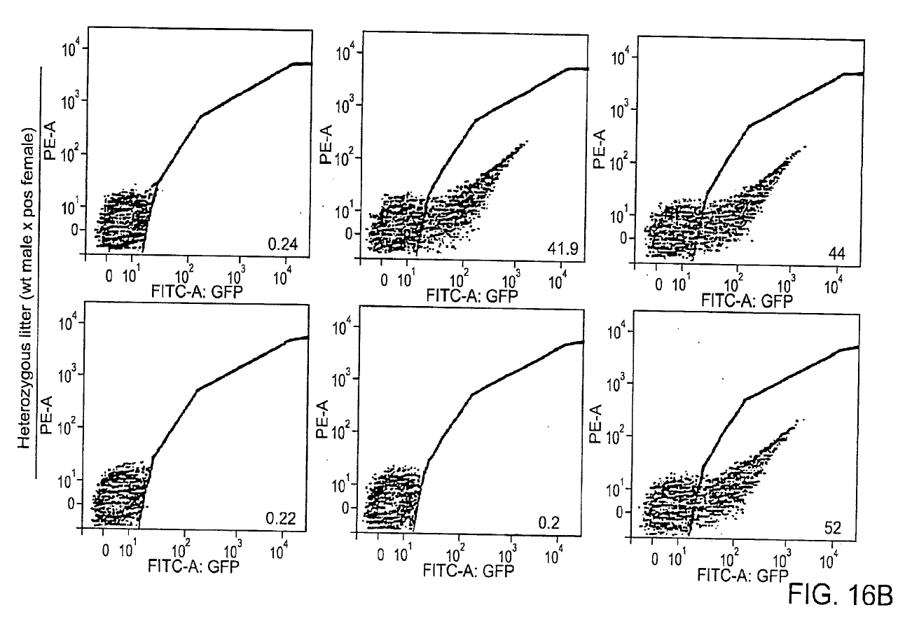

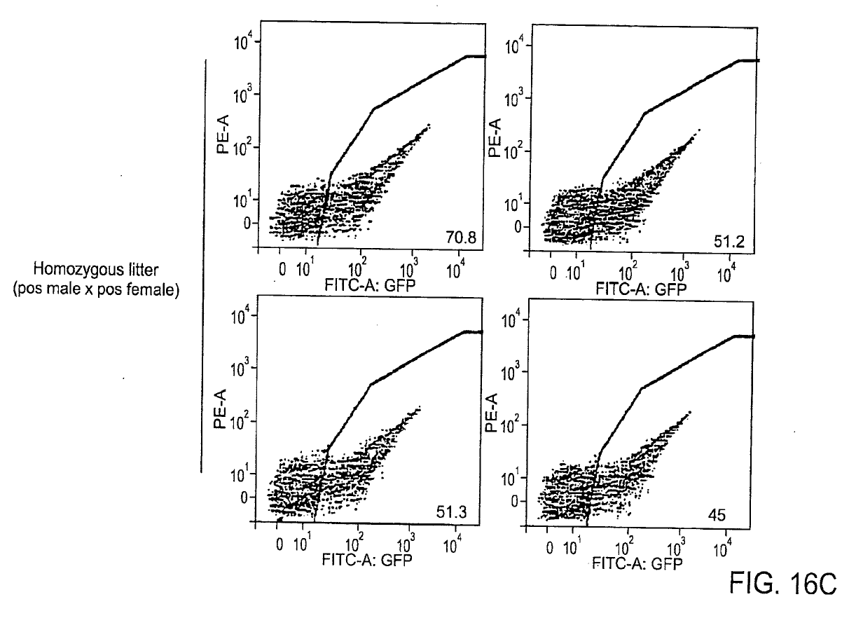

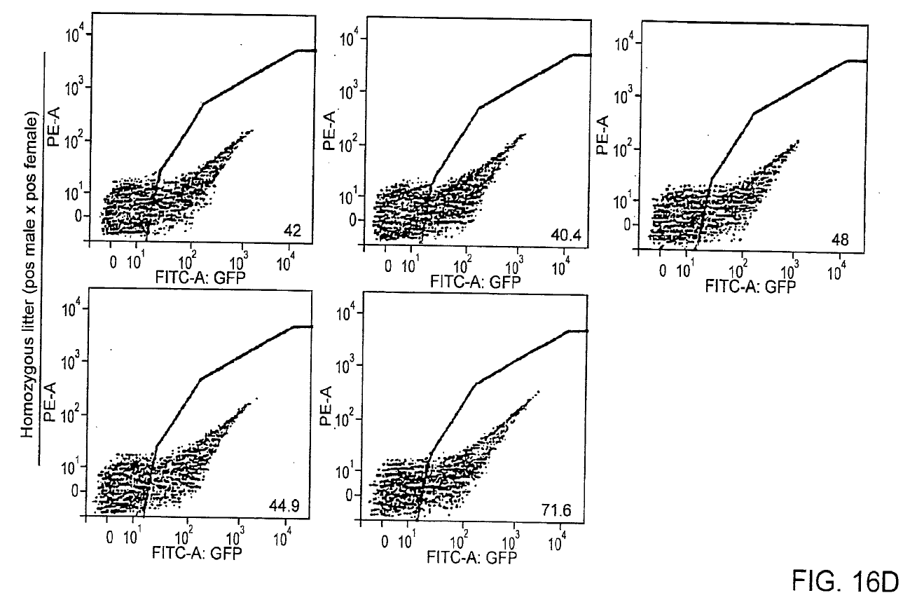

To determine whether the number of copies of the transgene affect levels of transgene expression, lentiviral construct expression was determined in pLB-915 transgenic heterozygotes and homozygotes. A non-transgenic male and a heterozygous pLB-915 transgenic founder Idd5.1 congenic mouse were crossed, yielding progeny which have either one or no copies of the lentiviral transgene. In addition, two heterozygous pLB-915 transgenic founder Idd5.1 congenic mice were crossed, yielding mice which have either two, one, or no copies of the lentiviral transgene. Flow cytometry of peripheral blood cells from the progeny of these crosses was performed, and GFP expression was determined for all of the littermates from both crosses (FIG. 16). Mice with blood cells displaying approximately 0% GFP expression are likely to have no copies of the transgene; mice with blood cells displaying approximately 50% GFP expression are likely to have one copy of the transgene; and mice with blood cells displaying approximately 70% GFP expression are likely to have two copies of the transgene (FIG. 16). These data show that the variegated expression level of the lentiviral transgene is independently regulated between the two copies and that the total expression in homozygous mice is therefore higher (albeit not in an additive manner) than in heterozygous offspring. Therefore, the present invention encompasses the recognition that, even in a heterozygote transgenic line which displays a lower transgene expression relative to other heterozygote lines, breeding a homozygous cohort can improve expression.

To measure silencing at the protein level in vivo, activated peritoneal macrophages were isolated and lysed immediately after cell-sorting for detection of NRAMP1 protein. As shown in FIG. 10c, NRAMP1 levels were much reduced (>70%) in cells expressing pLB-915, confirming that this construct effectively inhibited Nramp1 expression in transgenic mice.

### Example 5

### Nramp1 Silencing by Lentiviral Transgenesis Mimics the Protective Effect of the Idd5.2 Locus Against Diabetes and Partially Protects Against Infection

### Materials and Methods

Salmonella Infections

Male mice that were approximately 8 weeks of age were injected intravenously with approximately 1×10⁷ CFU of Salmonella enterica serovar Montevideo (SH5770) and checked daily for survival.

Measurement of Diabetes Frequency

This was performed as in Example 1.

Results

Although gene silencing seemed potent in the hematopoietic lineage cells analyzed as described in Example 4, it was uncertain whether the expression of the lentiviral construct observed in vivo would suffice to significantly affect systemic immune responses. Since NRAMP1 plays an essential function in protecting against intracellular pathogens (Vidal, 1995), we tested whether Nramp1 silencing in vivo conferred susceptibility to Salmonella enterica infection, as would be predicted if gene function was lost.

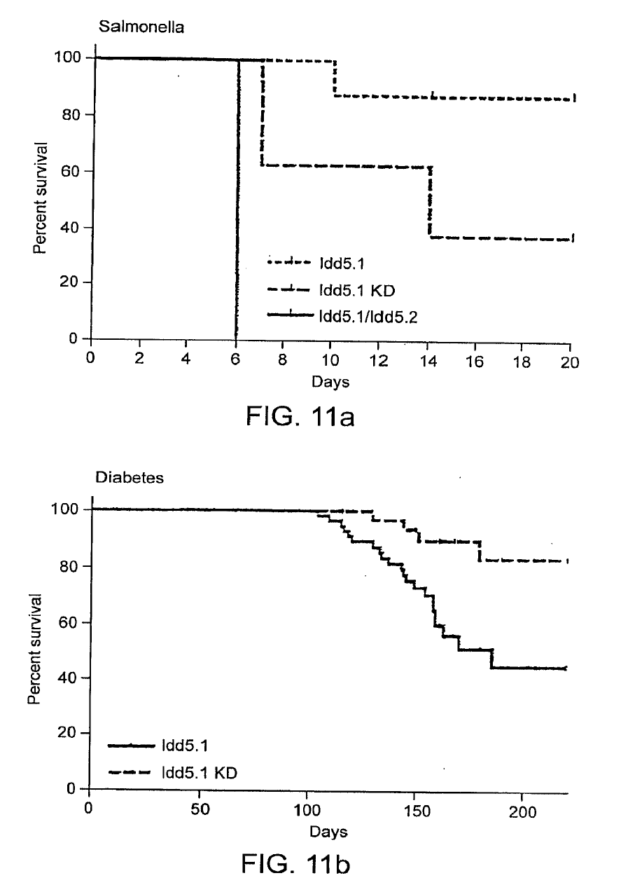

pLB-915 transgenic Idd5.1 mice, their non-transgenic littermates, and mice congenic for resistance alleles at both Idd5.1 and Idd5.2 were injected intravenously with Salmonella and monitored daily. Non-transgenic Idd5.1 mice had a fully functional allele of Nramp1, and all but one (out of eight) survived the bacterial challenge (FIG. 11a). Idd5.1/Idd5.2 mice possessed a mutated, non-functional allele and, as expected, succumbed to infection (7/7). Similarly, most Nramp1 knock-down Idd5.1 mice (5/8) failed to Survive the infection, demonstrating that gene silencing by lentiviral transgenesis was sufficient to partially mimic the gene-deficiency phenotype.

Finally, in order to assess the role of NRAMP1 in the development and onset of diabetes, we established large cohorts of Nramp1 knock-down female Idd5.1 mice and of their non-transgenic female littermates. Disease frequency was significantly reduced in pLB-915 transgenic mice (FIG. 11b). Nramp1 silencing mimicked the protective effect of the Idd5.2 locus (compare with FIG. 7b), demonstrating Nramp1 to be Idd5.2.

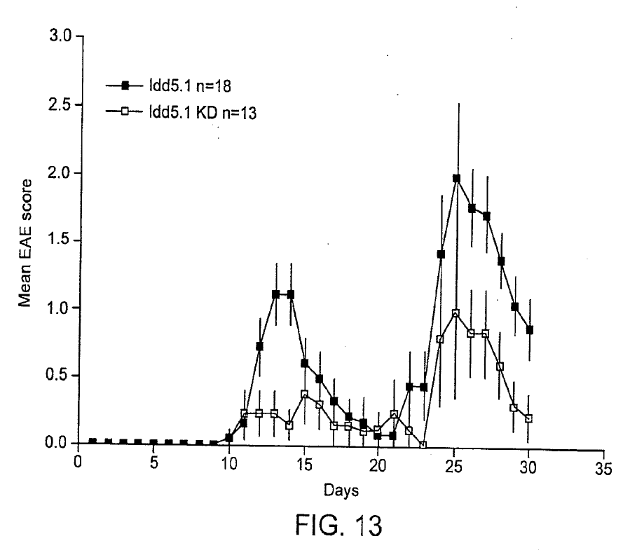

Several human studies have suggested an association of NRAMP1 with autoimmunity (Nishino, 2005; Takahashi, 2004; Sanjeevi, 2000; and Shaw, 1996). To investigate the effect of Nramp1 silencing on another autoimmune disease, we evaluated the susceptibility of Nramp1 knockdown Idd5.1 mice and of their nontransgenic littermates to experimental autoimmune encephalomyelitis (EAE), a widely used model for multiple sclerosis (Steinman, 2005). Nramp1 knockdown mice were (Idd5.1 KD, n=13) and nontransgenic Idd5.1 littermates (n=18) were immunized subcutaneously with MOG 35-55 peptide (100 μg) emulsified in CFA, and were administered pertussis toxin (200 ng) intraperitoneally the same day and 2 days later. Mice were scored daily for signs of disease: 1-limp tail, 2-partial hind-limb paralysis/impaired righting reflex, 3-complete hind-limb paralysis, 4-fore-limb and hind-limb paralysis, 5-moribund or dead. FIG. 13 shows combined results of two similar experiments shown as mean disease score=/−SEM. Nramp1 silencing again protected against disease (FIG. 13), further supporting a role for Nramp1 in autoimmunity (disease incidence: Idd5.1 18/18; Idd5.1 KD 8/13).

A concern sometimes raised with regards to RNAi experiments is the possibility of off-target effects (Qiu, 2005; Jackson, 2004). The risk of misinterpreting the effects of RNAi is likely to be more prevalent in experiments with unpredicted outcome, for instance in large-scale genetic screens. In the present system, RNAi replicates the previously demonstrated effect of NRAMP1 deficiency on Salmonella infection, as well as the kinetics and level of protection from diabetes provided by the mutant allele, in the absence of any unexpected phenotype. Together with the judicious design of Nramp1 shRNA, these results minimize the possibility that off-target effects caused the observed phenotype.

The present results demonstrate for the first time that RNAi can be effectively harnessed to study mammalian genetics within the context of a complex multigenic disease model. We generated a new lentiviral vector with dramatically improved in vivo expression, and showed that constitutive and inheritable RNAi can be used to phenocopy, at least in part, loss of gene-function. We employed this approach to determine the identity of the Idd5.2 locus in the NOD model. The protection from diabetes afforded by loss of NRAMP1 correlated with increased susceptibility to infection, as previously proposed in humans (Searle, 1999) where some reports have also suggested an association between NRAMP1 expression and several autoimmune diseases (Nishino, 2005; Takahashi, 2004; Sanjeevi, 2000; and Shaw, 1996) including diabetes. We anticipate that inventive systems and lentiviral vectors will lead the way in firmly establishing in vivo RNAi and lentiviral transgenesis as tools for the study of type 1 diabetes and other multigenic diseases in mammalian model organisms.

### REFERENCES

Makino, S et al. Breeding of a non-obese, diabetic strain of mice. Jikken Dobutsu 29, 1-13 (1980).
Todd, J. A. & Wicker, L. S. Genetic protection from the inflammatory disease type 1 diabetes in humans and animal models. Immunity 15, 387-395 (2001).
Ueda, H. et al. Association of the T-cell regulatory gene CTLA-4 with susceptibility to autoimmune disease. Nature 423, 506-511 (2003).
Vijayakrishnan, L. et al. An autoimmune disease-associated CTLA-4 splice variant lacking the B7 binding domain signals negatively in T cells. Immunity 20, 563-575 (2004).
Fire, A. et al. Potent and specific genetic interference by double-stranded RNA in Caenorhabditis elegans. Nature 391, 806-811 (1998).
Zamore, P. D., et al., RNAi: double-stranded RNA directs the ATP-dependent cleavage of mRNA at 21 to 23 nucleotide intervals (2000).
Elbashir, S. M., et al. RNA interference is mediated by 21- and 22-nucleotide RNAs. Genes Dev. 15: 188-200 (2001a).
Elbashir, S. M. et al. Duplexes of 21-nucleotides RNAs mediate RNA interference in cultured mammalian cells. Nature 411, 494-498 (2001b).
Fraser, A. G. et al. Functional genomic analysis of C. elegans chromosome I by systematic RNA interference. Nature 408: 325-330 (2000).
Vidal, S. M., Malo, D., Vogan, K., Skamene, E. & Gros, P. Natural resistance to infection with intracellular parasites: isolation of a candidate for Bcg. Cell 73, 469-485 (1993).
Vidal, S. M. et al. The Ity/Lsh/Bcg locus: natural resistance to infection with intracellular pathogens is abrogated by disruption of the Nramp1 gene. J. Exp. Med. 182, 655-666 (1995).
Wojciechowski, W., DeSanctis, J., Skamene, E. & Radzioch, D. Attenuation of MHC class II expression in macrophages infected with Mycobacterium bovis bacillus Calmette-Guerin involves class II transactivator and depends on the Nramp1 gene. J. Immunol. 163, 2688-2696 (1999).
Wicker, L. S. et al. Fine mapping, gene content, comparative sequencing, and expression analyses support Ctla-4 and Nramp-1 as candidates for Idd5.1 and Idd5.2 in the nonobese diabetic mouse. J. Immunol. 173, 164-173 (2004).
Lois, C., Hong, E. J., Pease, S., Brown, E. J. & Baltimore, D. Germline transmission and tissue-specific expression of transgenes delivered by lentiviral vectors. Science 295, 868-872 (2002).
Kanagawa, O., Xu, G., Tevaarwerk, A. & Vaupel, B. A. Protection of nonobese diabetic mice from diabetes by gene(s) closely linked to IFN-γ receptor loci. J. Immunol. 164, 3919-3923 (2000).
Rubinson, D. A. et al. A lentivirus-based system to functionally silence genes in primary mammalian cells, stem cells and transgenic mice by RNA interference. Nat. Genetics 33, 401-406 (2003).
Tiscornia, G., Singer, O., Ikawa, M. & Verma, I. M. A general method for gene knock-down in mice by using lentiviral vectors expressing small interfering RNA. Proc. Natl. Acad. Sci. USA 100, 1844-1848 (2003).
Lu, W., Yamamoto, V., Ortega, B. & Baltimore, D. Mammalian Ryk is a Wnt coreceptor required for stimulation of neurite outgrowth. Cell 119, 97-108 (2004).
Kwaks, T. H. et al. Identification of anti-repressor elements that confer high and stable protein production in mammalian cells. Nat. Biotech. 21, 553-558 (2003).
Klehr, D., Maass, K. & Bode, J. Scaffold-attached regions from the human interferon beta domain can be used to enhance the stable expression of genes under the control of various promoters. Biochemistry 30, 1264-1270 (1991).
Schwarz, D. S. et al. Asymmetry in the assembly of the RNAi enzyme complex. Cell 115, 199-208 (2003).
Khvorova, A., Reynolds, A. & Jayasena, S. D. Functional siRNAs and miRNAs exhibit strand bias. Cell 115, 209-216 (2003).
Reynolds, A. et al. Rational siRNA design for RNA interference. Nat. Biotech. 22, 326-330 (2004).
Qiu, S., Adema, C. M. & Lane, T. A computational study of off-target effects of RNA interference. Nucleic Acids Res. 33, 1834-1847 (2005).
Jackson, A. L. & Linsley, P. S. Noise amidst the silence: off-target effects of siRNAs? Trends Genet. 20, 521-524 (2004).
Searle, S. & Blackwell, J. M. Evidence for a functional repeat polymorphism in the promoter of the human NRAMP1 gene that correlates with autoimmune versus infectious disease susceptibility. J. Med. Genet. 36, 295-299 (1999).
Nishino, M. et al. Functional polymorphism in Z-DNA-forming motif of promoter of SLC11A1 gene and type 1 diabetes in Japanes subjects: Association study and meta-analysis. Metabolism 54, 628-633 (2005).
Takahashi, K. et al. Promoter polymorphism of SLC11 A1 (formerly NRAMP1) confers susceptibility to autoimmune type 1 diabetes mellitus in Japanese. Tissue Antigens 63, 231-236 (2004).
Sanjeevi, C. B. et al. Polymorphism at NRAMP1 and D2S1471 loci associated with juvenile rheumatoid arthritis. Arthritis Rheum. 43, 1397-1404 (2000).
Shaw, M. A. et al. Linkage of rheumatoid arthritis to the candidate gene NRAMP1 on 2q35. J. Med. Genet. 33: 672-677 (1996).
Hill, N. J. et al. NOD Idd5 locus controls insulitis and diabetes and overlaps the orthologous CTLA-4/IDDM12 and NRAMP1 loci in humans. Diabetes 49, 1744-1747 (2000).
McManus, M. T., Haines, B. B., Dillon, C. P., Whitehurst, C. E., van Parijs, L., Chen, J. & Sharp, P. A. siRNA-mediated gene silencing in T-cells. The Journal of Immunology, 2002, 169: 5754-5760.
Brummelkamp, T. R., Bernards, R. & Agami, R. A System for Stable Expression of Short Interfering RNAs in Mammalian Cells. Science 21, 21 (2002).
Paddison, P. J., Caudy, A. A., Bernstein, E., Hannon, G. J. & Conklin, D. S. Short hairpin RNAs (shRNAs) induce sequence-specific silencing in mammalian cells. Genes Dev 16, 948-58. (2002).
Sui, G. et al. A DNA vector-based RNAi technology to suppress gene expression in mammalian cells. Proc Natl Acad Sci USA 99, 5515-20. (2002).
Yu, J. Y., DeRuiter, S. L. & Turner, D. L. RNA interference by expression of short-interfering RNAs and hairpin RNAs in mammalian cells. Proc Natl Acad Sci USA 23, 23 (2002).
Paul, C. P., Good, P. D., Winer, I. & Engelke, D. R. Effective expression of small interfering RNA in human cells. Nat Biotechnol 20, 505-8. (2002).
Bernstein, E., Caudy, A. A., Hammond, S. M. & Hannon, G. J. Role for a bidentate ribonuclease in the initiation step of RNA interference. Nature 409, 363-6. (2001).
Martinez, J., Patkaniowska, A., Urlaub, H., Luhrmann, R. & Tuschl, T. Single-Stranded Antisense siRNAs Guide Target RNA Cleavage in RNAi. Cell 110, 563-574 (2002).
Brummelkamp, T. R., Bernards, R., and Agami, R. Stable suppression of tumorigenicity by virus-mediated RNA interference. Cancer Cell (2002).
Naldini, L. Lentiviruses as gene transfer agents for delivery to non-dividing cells. Curr Opin Biotechnol 9, 457-63 (1998).
Naldini, L. et al. In vivo gene delivery and stable transduction of nondividing cells by a lentiviral vector. Science 272, 263-7 (1996).
Jaenisch, R., Fan, H. & Croker, B. Infection of preimplantation mouse embryos and of newborn mice with leukemia virus: tissue distribution of viral DNA and RNA and leukemogenesis in the adult animal. Proc Natl Acad Sci USA 72, 4008-12 (1975).
Pfeifer, A., Ikawa, M., Dayn, Y. & Verma, I. M. Transgenesis by lentiviral vectors: lack of gene silencing in mammalian embryonic stem cells and preimplantation embryos. Proc Natl Acad Sci USA 99, 2140-5 (2002).
Hacein-Bey-Abina, S. et al. Sustained correction of X-linked severe combined immunodeficiency by ex vivo gene therapy. N Engl J Med 346, 1185-93 (2002).
Schmidt, E. V., Christoph, G., Zeller, R. & Leder, P. The cytomegalovirus enhancer: a pan-active control element in transgenic mice. Mol Cell Biol. 10, 4406-11 (1990).
McManus, M. T., Petersen, C. P., Haines, B. B., Chen, J. & Sharp, P. A. Gene silencing using micro-RNA designed hairpins. Rna 8, 842-50. (2002).
Miyoshi, H., Blomer, U., Takahashi, M., Gage, F. H. & Verma, I. M. Development of a self-inactivating lentivirus vector. J Virol 72, 8150-7 (1998).
Devroe, E. a. S., PA. Retrovirus-delivered siRNA. BMC Biotechnology 2 (2002).
Miyagishi M, et al., Optimization of an siRNA-expression system with an improved hairpin and its significant suppressive effects in mammalian cells, Gene Med. 2004 July; 6(7):715-23.
Dull, T., Zufferey, R., Kelly, M., Mandel, R. J., Nguyen, M., Trono, D., & Naldini, L., A Third-Generation Lentivirus Vector with a Conditional Packaging System. Journal of Virology, 72(11), 8463-8471 (1998).
Zufferey, R., D. Nagy, R. J. Mandel, L. Naldini, and D. Trono. Multiply attenuated lentiviral vector achieves efficient gene delivery in vivo. Nat. Biotechnol. 15:871-875 (1997).
Yuan, B, et al. siRNA Selection Server: an automated siRNA oligonucleotide prediction server. Nucl. Acids. Res. 32:W130-W134 (2004).
Santoyo J, Vaquerizas J M, Dopazo J. Highly specific and accurate selection of siRNAs for high-throughput functional assays. Bioinformatics. 21(8):1376-82, 2005.
Novina C D, Sharp P A. The RNAi revolution. Nature, 430(6996):161-4, 2004.
Dykxhoom D M, Novina C D, Sharp P A. Killing the messenger: short RNAs that silence gene expression. Nat Rev Mol Cell Biol. 4(6):457-67, 2003.
Hofmann A et al., Efficient transgenesis in farm animals by lentiviral vectors. EMBO Rep 4: 1054-1058, 2003.
Fässler, R., et al., Lentiviral transgene vectors: Green light for efficient production of transgenic farm animals, EMBO reports 5, 1, 28-29, 2004.
Pfeifer A. Lentiviral transgenesis. Transgenic Res. 13(6):513-22, 2004.
Houdebine, L-M., et al., Transgenic animal bioreactors, Transgenic Res., 9, 305-320, 2000.
Lillico, S. G., et al., Transgenic chickens as bioreactors for protein-based drugs, Drug Discovery Today, 191-196, 2005.
McManus, M. T., Haines, B. B., Dillon, C. P., Whitehurst, C. E., van Parijs, L., Chen, J. & Sharp, P. A. siRNA-mediated gene silencing in T-cells. The Journal of Immunology, 2002, 169: 5754-5760.
Steinman, L. and Zamvil, S. S. Virtues and pitfalls of EAE for the development of therapies for multiple sclerosis. Trends Immunol. 26, 565-571 (2005).

### EQUIVALENTS

Those skilled in the art will recognize, or be able to ascertain using no more than routine experimentation, many equivalents to the specific embodiments of the invention described herein. The Examples below are provided to illustrate the invention and are not limiting. Alternative procedures known to one of ordinary skill in the art might also be used. The scope of the present invention is not intended to be limited to the above Description, but rather is as set forth in the appended claims.

In the claims articles such as “a,” “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. In particular, any claim that is dependent on another claim can be modified to include one or more limitations found in any other claim that is dependent on the same base claim. Furthermore, where the claims recite a composition, it is to be understood that methods of administering the composition according to any of the methods disclosed herein, methods of using the composition for any of the purposes disclosed herein are included, and methods of making the composition according to any of the methods of making disclosed herein are included, unless otherwise indicated or unless it would be evident to one of ordinary skill in the art that a contradiction or inconsistency would arise. The invention includes embodiments that encompass every possible permutation of (i) an ARE, (ii) a SAR, (iii) lentivirus derived sequences for reverse transcription and packaging, (iv) regulatory sequences (e.g., promoters) for transcription of an operably linked nucleic acid, (v) heterologous nucleic acid (e.g., to be included in a lentiviral vector.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. Where ranges are given herein, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Wherever the claims or description recite a lentiviral vector having particular features or comprising particular sequence elements, the invention also includes (i) methods of producing the lentiviral vector using any of the techniques described herein and (ii) methods of using the lentiviral vector for any of the purposes described herein including, but not limited to, (a) expressing a heterologous nucleic acid in an isolated eukaryotic cell (e.g., a mammalian or avian cell) or transgenic nonhuman animal (e.g., a mammal or avian); (b) generating a transgenic nonhuman animal; (c) inhibiting expression of a gene in an isolated eukaryotic cell or transgenic nonhuman organism (wherein the lentiviral vector comprises an expression cassette that encodes an RNAi agent such as an shRNA); (d) treating a subject by administering the lentiviral vector to the subject; (iii) an isolated eukaryotic (e.g., mammalian or avian) cell comprising the lentiviral vector; (iv) a transgenic nonhuman animal (e.g., mammal or avian) generated using the lentiviral vector; (v) kits comprising the lentiviral vector as a component. Any of the embodiments of the invention that include administering a lentiviral vector to a subject can include a step of providing a subject, e.g., a subject at risk of or suffering from a disease, disorder, or condition. The methods may include a step of diagnosing a subject as suffering from or at risk of a disease, disorder, or condition.

In addition, it is to be understood that any one or more embodiments, variations, elements, sequences or sequence elements, diseases, conditions, genes, cell types, RNAi agents, etc., may be explicitly excluded from any one or more of the claims. For purposes of brevity, these various embodiments in which one or more elements, sequences or sequence elements, diseases, conditions, genes, cell types, RNAi agents, etc., is excluded from the claims are not set forth individually herein but are included in the invention.

1. A lentiviral vector comprising a nucleic acid comprising (i) a eukaryotic anti-repressor element (ARE); and (ii) sequences sufficient for reverse transcription and packaging, wherein said sequences are at least in part derived from a lentivirus.

2. The lentiviral vector of claim 1, wherein the ARE is derived from either human or mouse genome.

3. The lentiviral vector of claim 1, wherein the nucleic acid comprises a eukaryotic scaffold attachment region (SAR).

4. The lentiviral vector of claim 1, wherein the ARE is ARE 40 or a functional portion thereof.

5. The lentiviral vector of claim 1, wherein the ARE is selected from the group consisting of human and mouse ARE 40 or a functional portion thereof.

6. The lentiviral vector of claim 1, wherein the nucleic acid comprises the IFN-β SAR or a functional portion thereof.

7. The lentiviral vector of claim 1, wherein the lentiviral derived sequences are derived from HIV-1.

8. The lentiviral vector of claim 1, wherein the nucleic acid comprises a lentiviral FLAP element and an expression-enhancing posttranscriptional regulatory element.

9. The lentiviral vector of claim 1, wherein the nucleic acid comprises a self-inactivating (SIN) LTR.

10. The lentiviral vector of claim 1, wherein the vector is a lentiviral transfer plasmid or an infectious lentiviral particle.

11.-12. (canceled)

13. The lentiviral vector of claim 1, wherein the nucleic acid further comprises a regulatory sequence sufficient for transcription, wherein the regulatory sequence is flanked by lentivirus derived sequences.

14. (canceled)

15. The lentiviral vector of claim 13, wherein the nucleic acid comprises a SAR and the regulatory sequence is located between the ARE and the SAR.

16.-28. (canceled)

29. A kit comprising the lentiviral vector of claim 1.

30.-37. (canceled)

38. A cell comprising the lentiviral vector of claim 1 or at least some lentiviral sequences derived from the lentiviral vector.

39. (canceled)

40. A transgenic animal, at least some of whose cells contain the lentiviral vector of claim 1 or at least some lentiviral sequences derived therefrom.

41. (canceled)

42. A method of expressing a heterologous nucleic acid in a target cell comprising:
introducing a lentiviral vector of claim 1 into the target cell, wherein the lentiviral vector comprises a nucleic acid comprising regulatory sequences for transcription operably linked to a heterologous nucleic acid; and
expressing the heterologous nucleic acid in the cell.

43.-44. (canceled)

45. A method of silencing a gene in a target cell comprising:
introducing a lentiviral vector of claim 1 into the target cell, wherein the lentiviral vector comprises a nucleic acid comprising regulatory sequences for transcription operably linked to a nucleic acid that encodes an RNAi agent targeted to the gene; and
expressing the nucleic acid in the cell, thereby producing an RNAi agent that inhibits expression of the target gene.

46.-47. (canceled)

48. A method of creating an animal model of a disease comprising:
creating a transgenic nonhuman animal using the lentiviral vector of claim 1, wherein the lentiviral vector comprises a disease-associated gene.

49. A method of creating an animal model of a disease comprising:
creating a transgenic nonhuman animal using the lentiviral vector of claim 1, wherein the lentiviral vector encodes an RNAi agent targeted to a disease-associated gene.

50. A transgenic nonhuman animal that expresses a lentivirally transferred transgene, wherein at least 50% of the cells of 2, 3, 4, or more different cell types in the animal express the transgene.

51.-69. (canceled)


SEQUENCE LISTING (131 sequences)

SEQ ID NO: 1 - DNA, 749 bp, Artificial sequence
  sequence of STAR1
atgcggtgggggcgcgccagagactcgtgggatccttggcttggatgtttggatctttctgagttgcctgtgccgcgaaagacaggtacatttctgattaggcctgtgaagcctcctggaggaccatctcattaagacgatggtattggagggagagtcacagaaagaactgtggcccctccctcactgcaaaacggaagtgattttattttaatgggagttggaatatgtgagggctgcaggaaccagtctccctccttcttggttggaaaagctggggctggcctcagagacaggttttttggccccgctgggctgggcagtctagtcgaccctttgtagactgtgcacacccctagaagagcaactacccctatacaccaggctggctcaagtgaaaggggctctgggctccagtctggaaaatctggtgtcctggggacctctggtcttgcttctctcctcccctgcactggctctgggtgcttatctctgcagaagcttctcgctagcaaacccacattcagcgccctgtagctgaacacagcacaaaaagccctagagatcaaaagcattagtatgggcagttgagcgggaggtgaatatttaacgcttttgttcatcaataactcgttggctttgacctgtctgaacaagtcgagcaataaggtgaaatgcaggtcacagcgtctaacaaatatgaaaatgtgtatattcaccccggtctccagccggcgcgccaggctccc

SEQ ID NO: 2 - DNA, 883 bp, Artificial sequence
  sequence of STAR2
gggtgcttcctgaattcttccctgagaaggatggtggccggtaaggtccgtgtaggtggggtgcggctccccaggccccggcccgtggtggtggccgctgcccagcggcccggcacccccatagtccatggcgcccgaggcagcgtgggggaggtgagttagaccaaagagggctggcccggagttgctcatgggctccacatagctgccccccacgaagacggggcttccctgtatgtgtggggtcccatagctgccgttgccctgcaggccatgagcgtgcgggtcatagtcgggggtgccccctgcgcccgcccctgccgccgtgtagcgcttctgtgggggtggcgggggtgcgcagctgggcagggacgcagggtaggaggcggggggcagcccgtaggtaccctgggggggcttggagaagggcgggggcgactggggctcatacgggacgctgttgaccagcgaatgcatagagttcagatagccaccggctccggggggcacggggctgcgacttggagactggccccccgatgacgttagcatgcccttgcccttctgatcctttttgtacttcatgcggcgattctggaaccagatcttgatctggcgctcagtgaggttcagcagattggccatctccacccggcgcggccggcacaggtagcggttgaagtggaactctttctccagctccaccagctgcgcgctcgtgtaggccgtgcgcgcgcgcttggacgaagcctgccccggcgggctcttgtcgccagcgcagctttcgcctgcgaggacagagagaggaagagcggcgtcaggggctgccgcggccccgcccagcccctgacccagcccggcccctccttccaccaggccccaa

SEQ ID NO: 3 - DNA, 2126 bp, Artificial sequence
  sequence of STAR3
atctcgagtactgaaataggagtaaatctgaagagcaaataagatgagccagaaaaccatgaaaagaacagggactaccagttgattccacaaggacattcccaaggtgagaaggccatatacctccactacctgaaccaattctctgtatgcagatttagcaaggttataaggtagcaaaagattagacccaagaaaatagagaacttccaatccagtaaaaatcatagcaaatttattgatgataacaattgtctccaaaggaacaaggcagagtcgtgctagcagaggaagcacgtgagctgaaaacagccaaatctgctttgttttcatgacacaggagcataaagtacacaccaccaactgacctattaaggctgtggtaaaccgattcatagagagaggttctaaatacattggtccctcacaggcaaactgcagttcgctccgaacgtagtccctggaaatttgatgtccagtatagaaaagcagagcagtcaaaaaatatagataaagctgaaccagatgttgcctgggcaatgttagcagcaccacacttaagatataacctcaggctgtggactccctccctggggagcggtgctgccggcggcgggcgggctccgcaactccccggctctctcgcccgccctcccgttctcctcgggcggcggcgggggccgggactgcgccgctcacagcggcggctcttctgcgcccggcctcggaggcagtggcggtggcggccatggcctcctgcgttcgccgatgtcagcatttcgaactgagggtcatctccttgggactggttagacagtgggtgcagcccacggagggcgagttgaagcagggtggggtgtcacctcccccaggaagtccagtgggtcagggaactccctcccctagccaagggaggccgtgagggactgtgcccggtgagagactgtgccctgaggaaaggtgcactctggcccagatactacacttttcccacggtcttcaaaacccgcagaccaggagattccctcgggttcctacaccaccaggaccctgggtttcaaccacaaaaccgggccatttgggcagacacccagctagctgcaagagttgtttttttttttatactcctgtggcacctggaacgccagcgagagagcacctttcactcccctggaaagggggctgaaggcagggacctttagctgcgggctagggggtttggggttgagtgggggaggggagagggaaaaggcctcgtcattggcgtcgtctgcagccaataaggctacgctcctctgctgcgagtagacccaatcctttcctagaggtggagggggcgggtaggtggaagtagaggtggcgcggtatctaggagagagaaaaagggctggaccaataggtgcccggaagaggcggacccagcggtctgttgattggtattggcagtggaccctcccccggggtggtgccggagggggggatgatgggtcgaggggtgtgtttatgtggaagcgagatgaccggcaggaacctgccccaatgggctgcagagtggttagtgagtgggtgacagacagacccgtaggccaacgggtggccttaagtgtctttggtctcctccaatggagcagcggcggggcgggaccgcgactcgggtttaatgagactccattgggctgtaatcagtgtcatgtcggattcatgtcaacgacaacaacagggggacacaaaatggcggcggcttagtcctacccctggcggcggcggcagcggtggcggaggcgacggcactcctccaggcggcagccgcagtttctcaggcagcggcagcgcccccggcaggcgcggtggcggtggcgcgcagccaggtctgtcacccaccccgcgcgttcccagggggaggagactgggcgggaggggggaacagacggggggggattcaggggcttgcgacgcccctcccacaggcctctgcgcgagggtcaccgcggggccgctcggggtcaggctgcccctgagcgtgacggtagggggcgggggaaaggggaggagggacaggccccgcccctcggcagggcctctagggcaagggggcggggctcgaggagcggaggggggcggggcgg

SEQ ID NO: 4 - DNA, 1625 bp, Artificial sequence
  sequence of STAR4
gatctgagtcatgttttaaggggaggattcttttggctgctgagttgagattaggttgagggtagtgaaggtaaaggcagtgagaccacgtaggggtcattgcagtaatccaggctggagatgatggtggttcagttggaatagcagtgcatgtgctgtaacaacctcagctgggaagcagtatatgtggcgttatgacctcagctggaacagcaatgcatgtggtggtgtaatgaccccagctgggtagggtgcatgtggtgtaacgacctcagctgggtagcagtgtgtgtgatgtaacaacctcagctgggtagcagtgtacttgataaaatgttggcatactctagatttgttatgagggtagtgccattaaatttctccacaaattggttgtcacgtatgagtgaaaagaggaagtgatggaagacttcagtgcttttggcctgaataaatagaagacgtcatttccagttaatggagacagggaagactaaaggtagggtgggattcagtagagcaggtgttcagttttgaatatgatgaactctgagagaggaaaaactttttctacctcttagtttttgtgactggacttaagaattaaagtgacataagacagagtaacaagacaaaaatatgcgaggttatttaatatttttacttgcagaggggaatcttcaaaagaaaaatgaagacccaaagaagccattagggtcaaaagctcatatgcctttttaagtagaaaatgataaattttaacaatgtgagaagacaaaggtgtttgagctgagggcaataaattgtgggacagtgattaagaaatatatgggggaaatgaaatgataagttattttagtagatttattcttcatatctattttggcttcaacttccagtctctagtgataagaatgttcttctcttcctggtacagagagagcacctttctcatgggaaattttatgaccttgctgtaagtagaaaggggaagatcgatctcctgtttcccagcatcaggatgcaaacatttccctccattccagttctcaaccccatggctgggcctcatggcattccagcatcgctatgagtgcacctttcctgcaggctgcctcgggtagctggtgcactgctaggtcagtctatgtgaccaggagctgggcctctgggcaatgccagttggcagcccccatccctccactgctgggggcctcctatccagaagggcttggtgtgcagaacgatggtgcaccatcatcattccccacttgccatctttcaggggacagccagctgctttgggcgcggcaaaaaacacccaactcactcctcttcaggggcctctggtctgatgccaccacaggacatccttgagtgctgggcagtctgaggacagggaaggagtgatgaccacaaaacaggaatggcagcagcagtgacaggaggaagtcaaaggcttgtgtgtcctggccctgctgagggctggcgagggccctgggatggcgctcagtgcctggtcggctgcaagaggccagccctctgcccatgaggggagctggcagtgaccaagctgcactgccctggtggtgcatttcctgccccactctttccttctaagatcc

SEQ ID NO: 5 - DNA, 1571 bp, Artificial sequence
  sequence of STAR5
agcagagatcttatttcccgtattcccttgtggcacagcacctcccacgccaaagcaaaccaaagcaaaggagcccttgatgaggaggggccttcccccaacctggtctcccacaggtcctacatacgtacccaccccagacacacagagctgcttcctgctctcacaccagactgagctgtgcccagacatttcccctagcactaaccaactctttcaaaaatacatttttctctaaaaagaacaagtttaaacaaagttgactcattttaagaactgtttagaagataaccttgtgtttattaattatgtatttgcagaaattggaggcagaaggttaccaacattgcctggtgtccagccaggaggtagagcgtggtggcatccagaaccttcctccaactcctgcctggcgtggtttttattcatctttgtattcccaagaaacttctcagtgtctcaggagtgttaggcactcagtacgtgtttggtagttacatgaatgaatgcataatgactaagtgagttaatggatgaagctaattgtctctcccttttgcttttccagagctttccaaggtgaaagtgttggacactctttcttcatctcagatttaatcaactaagaatgctgcaaattgaacaccagtccacaaaactcaggaatacatgaaaagcattgtgccttatttttaactaactcaaattctatgtcagtctcccttttatgctggatgttggcgctaaatctcagtgggttcctcattctgccagacctgtgtccagtttgggggcttcacatagagccaccccatcacaggagagggaagggtcttgctcttggttgccatcactccaccctcttgtcttccgagctttgatgttcactttccttttcaccactcggaagcttcctgccatgatacattgagacctcaatgttaatgccaattggggtttggggttctcataaactcagaagtccaggaaaatcgcctgctgcctcccacaacactctgagggcattctggaatcctaccacttacctggagcctgctggcctcaactgttttgaagtctgtgtctgggccatgcaggtaaatgggaggatgttctgtggccataaaaatacccgaagtcccacctaaagttgatgcagggtcttctgcatttcattgcaaaattgttctatcatttctatagttttcagcctacagtcaggggccaggactttgcacccttggtaaacctcaatctcttctccttcctggcttctactcctttctccctcaatcccaaatcaaggcccttgattgtctggaggtaggaaagcctggttctggctcatgatatagtctacatcatagcctttgtcatctcatggattcactcaacaaccgtgtgtggatggggccacccaatatgtgccaggagttgaggacacgcagggttatgatgatgaaatagataaggggcccacactcacggaccctgcaggacagtggagctgtggacccagcatgcgagtaaagacccagtgagctcaccagacagatcatttaaatcaggtg

SEQ ID NO: 6 - DNA, 1173 bp, Artificial sequence
  sequence of STAR6
tgacccaccacagacatcccctctggcctcctgagtggtttcttcagcacagcttccagagccaaattaaacgttcactctatgtctatagacaaaaagggttttgactaaactctgtgttttagagagggagttaaatgctgttaactttttaggggtgggcgagagggatgacaaataacaacttgtctgaatgttttacatttctccccactgcctcaagaaggttcacaacgaggtcatccatgataaggagtaagacctcccagccggactgtccctcggcccccagaggacactccacagagatatgctaactggacttggagactggctcacactccagagaaaagcatggagcacgagcgcacagagcagggccaaggtcccagggacagaatgtctaggagggagattggggtgagggtaatctgatgcaattactgtggcagctcaacattcaagggagggggaagaaagaaacagtccctgtcaagtaagttgtgcagcagagatggtaagctccaaaatttgaaactttggctgctggaaagttttagggggcagagataagaagacataagagactttgagggtttactacacactagacgctctatgcatttatttatttattatctcttatttattactttgtataactcttataataatcttatgaaaacggaaaccctcatatacccattttacagatgagaaaagtgacaattttgagagcatagctaagaatagctagtaagtaaaggagctgggacctaaaccaaaccctatctcaccagagtacacactctttttttttttccagtgtaattttttttaatttttattttactttaagttctgggatacatgtgcagaaggtatggtttgttacataggtatatgtgtgccatagtggattgctgcacctatcaacccgtcatctaggtttaagccccacatgcattagctatttgtcctgatgctctccctcccctccccacaccagacaggccttggtgtgtgatgttcccctccctgtgtccatgtgttctcactgttcagctcccacttatgagtgagaacgtgtggtatttggttttctgttcctgtgttagtttgctgaggatgatggcttccagcttcatccatgtccctgcaaaggacacgatc

SEQ ID NO: 7 - DNA, 2101 bp, Artificial sequence
  sequence of STAR7
atcatgccagcttaggcgacagagtgagactggacataataacaataataataaaaataaataaataaaacaattatctgagaggaaaaatttgattcataataaagagaataaaggtttttggcgtgtttgttttgttttcacctaagaacagctgttcccctcattgggttagttttatttgcaagcagaaatcatctccgcatgatttccagggtgatggaaaactgaatatgaatccaccttctgccatctattcacttgtcacatttaataagacactcatgcctattttagcatgttttcttccctaccaaatgagttagtaacatcaagagattaaaataacacaaataagaacattgaaggtattcaaatgttacatacaaatattaaacacaatattattataattattcctggaaatgacattgcctctactctcaaggtaaaggtcatttttcttgatttaaacttttttctcaagtttgaaatctctaagtttcaacccgtaatctatttgcaagtttgtgcaaattttagggattgaatccatagtaattagtgatttattgtggtgtagggagacaagtcaaaagaatcaggactgctaggtagatgactaaggaaaggatggttcacgaggtgacataaagcactcagaagaaaaaggtcaggaaacggaggacagaaaaaaacctaagttctgctgggtgatgctgaatttgtcatcacaaaatctgcattgtggaagctttagctattgaggagattgctcaagtgtagaactgagaacaataggcagtgaacccgagagaacatcaagagactgagagaaaatgaaccagacttccaggtgctccatgttccaaccaacattttgtattgtcagaaggaattgagaggcaaaaggaaacccaataaaaaataaaacaggaaagggcatacatgattaccaccccttttctcaccagctgctcatggaccagctttctcctagtgctattttcttggtcactgcatcactctgctaacatagtttccccactagctctgaggctgtcccagaggggaagccagctgtcatctccttcttccacactctgttggaggaacctgtcattagcagctccctactaaacgcatttatgacaaacaggcaggagataattaactagaaagtgaacaaactcaaacttcagagcctctcatttgtatgaatgcccttgtaaggtcttgggcctattttaatatttataaatgtgttattttcttctaaagaaaaccaccaaattgtataagctacagaatctgcaaaactgaggtccatccatgcactcaggatacattcatagcatctctgagctggaaaatatcttaaaggtcatatatgtcctccaacactgcaagaatctctctggcagcattcttttaaaatcatcatctaaaagagggaaatccccagctgtgtttggattttgctctgtcacttgtccagtttccccatccataaaagggcaacaatatgaatttcctgataaggtagttgttaatataaatacaaagtgcgtagccacttccctaagaaaaatatggggtttctgcttcacagtctagggagaggaaaaaaaaggggggtcagaagtgattattattatcattctatattggaatgttttcagacataaaaagctcaccacgtcttaggccagacagatgcattatgaaagttaagctaagtcttcctcatcatgagctgcacctatatccccattacttcttctagaactgcataatttatttattctttcttcaaaagtttgagagagccattcttgtcctctaagatttttttttttttttttggagacagagtctccgtctgttgcccaggctggagtgcaatggcactatctcagctcactgcaacctctgcctcccagattcaagtgattctcctgcctcagcctcccgagtagctgggattacaagcacgcaccaccacaaccagctaatttttcgtattttttagtagagacgaggttttaccatgttggccaggctggtcttgaactcctgacctcgggtgatccacccacct

SEQ ID NO: 8 - DNA, 1821 bp, Artificial sequence
  sequence of STAR8
gagatcacctcgaagagagtctaacgtccgtaggaacgctctcgggttcacaaggattgaccgaaccccaggatacgtcgctctccatctgaggcttgctccaaatggccctccactattccaggcacgtgggtgtctcccctaactctccctgctctcctgagcccatgctgcctatcacccatcggtgcaggtcctttctgaagagctcgggtggattctctccatcccacttcctttcccaagaaagaagccaccgttccaagacacccaatgggacattccccttccacctccttctccaaagttgcccaggtgttcatcacaggttagggagagaagcccccaggtttcagttacaaggcataggacgctggcatgaacacacacacacacacacacacacacacacacacacacacacgactcgaagaggtagccacaagggtcattaaacacttgacgactgttttccaaaaacgtggatgcagttcatccacgccaaagccaagggtgcaaagcaaacacggaatggtggagagattccagaggctcaccaaaccctctcaggaatattttcctgaccctgggggcagaggttggaaacattgaggacatttcttgggacacacggagaagctgaccgaccaggcattttcctttccactgcaaatgacctatggcgggggcatttcactttcccctgcaaatcacctatggcgaggtacctccccaagcccccacccccacttccgcgaatcggcatggctcggcctctatccgggtgtcactccaggtaggcttctcaacgctctcggctcaaagaaggacaatcacaggtccaagcccaaagcccacacctcttccttttgttatacccacagaagttagagaaaacgccacactttgagacaaattaagagtcctttatttaagccggcggccaaagagatggctaacgctcaaaattctctgggccccgaggaaggggcttgactaacttctataccttggtttaggaaggggaggggaactcaaatgcggtaattctacagaagtaaaaacatgcaggaatcaaaagaagcaaatggttatagagagataaacagttttaaaaggcaaatggttacaaaaggcaacggtaccaggtgcggggctctaaatccttcatgacacttagatataggtgctatgctggacacgaactcaaggctttatgttgttatctcttcgagaaaaatcctgggaacttcatgcactgtttgtgccagtatcttatcagttgattgggctcccttgaaatgctgagtatctgcttacacaggtcaactccttgcggaagggggttgggtaaggagcccttcgtgtctcgtaaattaaggggtcgattggagtttgtccagcattcccagctacagagagccttatttacatgagaagcaaggctaggtgattaaagagaccaacagggaagattcaaagtagcgacttagagtaaaaacaaggttaggcatttcactttcccagagaacgcgcaaacattcaatgggagagaggtcccgagtcgtcaaagtcccagatgtggcgagcccccgggaggaaaaaccgtgtcttccttaggatgcccggaacaagagctaggcttccggagctaggcagccatctatgtccgtgagccggcgggagggagaccgccgggaggcgaagtggggcggggccatccttctttctgctctgctgctgccggggagctcctggctggcgtccaagcggcaggaggccgccgtcctgcagggcgccgtagagtttgcggtgcagagt

SEQ ID NO: 9 - DNA, 1929 bp, Artificial sequence
  sequence of STAR9
atgagcccccaaaaatgatcctctggcttatgacaacctgatgcagcccaggaaatgcctgcaacatgcccactagcagctgggaacccctctgtgaggaagagaacgttttacattaagaaaccctttgttttgcagcagagactattcaggtcacacatgtgtggcctctcagttctttgagccatttgaagttctctatccttgctgggaggctgagctctccatggaaacctggtccgatagtgagaggagcagaccctctggaaacacctttttacacctgaccaaagcagccagtcatgggccagtgatgcaacaaggtcaaccggtgcattctggcccctcagaaaagcagcccccgggaaggtcaggaggaggctgctgactccctcttcccctgcagccgccccaagcacacccaggagccctgcaggtttgggttcaccaggtgccagcaggtcccacgatgctgcatttcttacgagctcctggaggatgcagatggtcctggtcagaggctgcattctgagtatcaggagccatggggcaacgtttctgcgattgaggaaggggcatttctggggtgggcagaacaaaggtctttggctgagctggagcatccgcctccatcagtgttttccggcaactgtactatccatcgtcttcccttcccacagctgaccatggctttggaaaatgctctgaaactttcttttcagaagagttgactcccaactccacacttaggggaagtcaagcctacttctcagaattcagagaaggcataaaaaagaattcatttctaaaggccctttagaagtaacttcaggtctgacagcggccagctaatttctggtcgccttccaggaatcttctgactgcaaaaaaaaagcatttaccacctgaacacaaacccagttacagatagaaaaacatagtcatttaaatagaatataagcatctggcctctgcccatcataatggagtaacacaaaaatctattttcaaaaggaaactaaatattattgaccaaaacatgaatggggagacctcagggtgatacagctcttgcctggatggaatttgtaatcaagaggatgagacaggattgtaacttgtgccaatgtgaaagggtttgctcaggtatcattcattttgcttaaatgcatgggtaatttccaaagttctttggagctgaatttcacaatttagtgcaggtcctggtgagcccaccttgacttatctcacagtacaatgcagtggcgtggctacaatgctgggcaagagaagccaatgtcaacagcccaggagtggctgggtccttaccaggctcccaggcatgcttcatggtgggccctgggctgggaggaacagcacctttgcctggtccatgagtatctgggtcaaactctcctgtggacacagaaggccatggcgacaggcattcccaggaaaagaaaagggcagcagctgaaatcgtcaggtggagaaggcagtcatccttgctcagtcaactctaatccggctgcctcctcctcagcttcagggtgaacctctcctaagctgtgtctttggtatctgatgggcattaggtgctggtgaaaaagctggagggtcctttgggatattacagaagcccaatctagccttgtattcaatatctaggcactctcacccctgaagttctacgtttccagatttctgaaaacatgggaaagcatgtgtgtgatgtctgaggtccccctcagcctctggtgtagggttaggagggctctaaagggtggcagctccagtgtcccagtggggcctgaagttggtcccttcccttcccagctcccatccatggtttagcccaatcccttccgtacctaagagtactgcacatggatgctccacgcagagcctctgctccactcccaggaagtg

SEQ ID NO: 10 - DNA, 1167 bp, Artificial sequence
  sequence of STAR10
aggtcaggagttcaagaccagcctggccaacatggtgaaaccctgtccctacaaaaaatacaaaaattagccgggcgtggtggggggcgcctataatcccagctactcaggatgctgagacaggagaattgtttgaacccgggaggtggaggttgcagtgaactgagatcgcgccactgcactccagcctggtgacagagagagactccgtctcaacaacagacaaacaaacaaacaaacaacaacaaaaatgtttactgacagctttattgagataaaattcacatgccataaaggtcaccttctacagtatacaattcagtggatttagtatgttcacaaagttgtacgttgttcaccatctactccagaacatttacatcacccctaaaagaagctctttagcagtcacttctcattctccccagcccctgccaaccacgaatctactntctgtctctattctgaatatttcatataaaggagtcctatcatatgggccttttacgtctaccttctttcacttagcatcatgtttttaagattcatccacagtgtagcacgtgtcagttaattcatttcatcttatggctggataatgctctattgtatgcatatccctcactttgcttatccattcatcaactgattgacatttgggttatttctactttttgactattatgagtaatgctgctatgaacattcctgtaccaatcgttacgtggacatatgctttcaattctcctgagtatgtaactagggttggagttgctgggtcatatgttaactcagtgtttcatttttttgaagaactaccaaatggttttccaaagtggatgcaacactttacattcccaccagcaagatatgaaggttccaatgtctctacatttttgccaacacttgtgattttcttttatttatttatttatttatttatttttgagatggagtctcactctgtcacccaggctggagtgcagtggcacaatttcagctcactgcaatctccacctctcgggctcaagcgatactcctgcctcaacctcccgagtaactgggattacaggcgcccaccaccacaccaagctaattttttgtatttttagtagagacggggtttcatcatgtcggccaggntgtactcgaactctgacctcaagt

SEQ ID NO: 11 - DNA, 1377 bp, Artificial sequence
  sequence of STAR11
gattctgggtgggtttgatgatctgagagtcccttgaataaaaagaattctagaaaagctgtgaaacttcacctttcccctattcttaaccttacttgcctttgggaggctgaggcaggaggatgacttaaggccaggagtttgagaatgtagtgagctatgaccacaccggttacactcaagcctgggcgagaccacaacaaaaaccttacctgccaactgctccatgctggaaatttatttcgtttcttggattgtggaaagaactggcttactgaaaaccacacttctctaaaacccttcttccagttaggtgttaagattttaacagcctttcctatctgaataaaaactgcacacaaagtaaacttaagagatgtcaacaactcatctgtttgttacaagatgagtctccatgcttcatcgcctgtggggaatcctcatcagcgtctagtggcaaagactcctgtgtgctcaccgaaacgctccccttcctccagggcacacagtcacatggatttcccatgcaccctggcagctcagcaggagtccatgacttaagaaggccaatggactgtgggtgaagtctgtggacggggaagccacatgcgtcacttccaggcctgggcgtgtgcatcctccactctcttcccctgtgggtgcagaaggcggggcagagggccctgaaaccttggaggtcggtggagcccaaaatgaaggagcgtgggcctctgggtcttcatgtaaatttaggtaacactgaactgtcaggtgaacaagaaataaacgtcaaatgtattcagtcgattagatttggtgatggttgttacagcggttaccctccctcaacataataaattttcaaacaactcataatggctcactcatgtataaaatattccatatgaaatcccgggataacatgcttattctagctcaagcttaatcagagtagtccatctgagggaggagatagtagagggcagcaaggggttgtcactgaagataactagccttgctaaaagaatggttgaagaagtgagctacagatagggtaaatccacatctcagacattctgtgatggtcctgatattatcctaaagtaaaatgtagagttgaaccattttaattagattctagaattctattaatttataagatgggcatttccacaaaggactaaacaaagtacaagaggattaaataatcatccacatgggaggcaccgccttgcactttaaaatgatggagcttatcaagactggctgtggatatctgtccctgggagggttttttcccccatttttttcctttttgagacatgttctcgctatgttgcccaggctggtcttgaactcctgggctcaagtgatcct

SEQ ID NO: 12 - DNA, 1051 bp, Artificial sequence
  sequence of STAR12
atcctgcttctgggaagagagtggcctcccttgtgcaggtgactttggcaggaccagcagaaacccaggtttcctgtcaggaggaagtgctcagcttatctctgtgaagggtcgtgataaggcacgaggaggcaggggcttgccaggatgttgcctttctgtgccatatgggacatctcagcttacgttgttaagaaatatttggcaagaagatgcacacagaatttctgtaacgaataggatggagttttaagggttactacgaaaaaaagaaaactactggagaagagggaagccaaacaccaccaagtttgaaatcgattttattggacgaatgtctcactttaaatttaaatggagtccaacttccttttctcacccagacgtcgagaaggtggcattcaaaatgtttacacttgtttcatctgcctttttgctaagtcctggtcccctacctcctttccctcacttcacatttgtcgtttcatcgcacacatatgctcatctttatatttacatatatataatttttatatatggcttgtgaaatatgccagacgagggatgaaatagtcctgaaaacagctggaaaattatgcaacagtggggagattgggcacatgtacattctgtactgcaaagttgcacaacagaccaagtttgttataagtgaggctgggtggtttttattttttctctaggacaacagcttgcctggtggagtaggcctcctgcagaaggcattttcttaggagcctcaacttccccaagaagaggagagggcgagactggagttgtgctggcagcacagagacaagggggcacggcaggactgcagcctgcagaggggctggagaagcggaggctggcacccagtggccagcgaggcccaggtccaagtccagcgaggtcgaggtctagagtacagcaaggccaaggtccaaggtcagtgagtctaaggtccatggtcagtgaggctgagacccagggtccaatgaggccaaggtccagagtccagtaaggccgagatccagggtccagggaggtcaag

SEQ ID NO: 13 - DNA, 1291 bp, Artificial sequence
  sequence of STAR13
ctgccctgatcccttaatgcttttggcccagagcaccccgctaagtccaaccccagaggggcctcatccgcaaagcctcgggaagaggacagtgacggaggcggctgccctgtgagctgcacggggcagaatgtccttttggcgtcatgttggatgtccacacatccatatggggtcagttctattaggattccttcgggaagaggtagagggtaggaggggttaagccacgagacgaggcatgcagaggggtggcctggatgggtctgcactgctgtccatgcacacggggagcgttgcaaattgtgcttcccagcccatagtgcccccacagaggagcccgggagtccctggtgggcgtctgtgttcctgcaaggagccagtggagatggccccgtgaactctcatcccccttgccttggtggggtctctggcaggtttatggagccgtacatctttgggagccgcctggaccacgacatcatcgacctggaacagacagccacgcacctccagctggccttgaacttcaccgcccacatggcctaccgcaagggcatcatcttgtttataagccgcaaccggcagttctcgtacctgattgagaacatggcccgtgactgtggcgagtacgcccacactcgctacttcaggggcggcatgctgaccaacgcgcgcctcctctttggccccacggtccgcctgccggacctcatcatcttcctgcacacgctcaacaacatctttgagccacacgtggccgtgagagacgcagccaagatgaacatccccacagtgggcatcgtggacaccaactgcaacccctgcctcatcacctaccctgtacccggcaatgacgactctccgctggctgtgcacctctactgcaggctcttccagacggccatcacccgggccaaggagaagcggcagcaggttgaggctctctatcgcctgcagggccagaaggagcccggggaccaggggccagcccaccctcctggggctgacatgagccattccctgtgatgttcactctcctcccaaagcaaaccacagccaagcctgtctgagctgggagtccccttccccagccctgggtcagcggcatcctcagtcgttgttacttactcagctgatgtcacagtgcagacatccaccgttccaccacagaaccagtggctgagcggaccaacgttgccatgtgcgtttgctctgtggggaacagagcacagagggtgagcgacatgtgcagaacggccccttggctgcagttaggacctcagtggct

SEQ ID NO: 14 - DNA, 711 bp, Artificial sequence
  sequence of STAR14
agcaaggaccagggctctgcctccccagtcagcatgagcagagcagactcctttgagcagagcatcagggcagaaatagaacagtttctgaatgagaaaagacagcatgagacccaaaaatgtgatgggtcagtggagaagaaaccagacacacatgaaaattcggcgaagtcactctcgaaatcccaccaagagccggctacaaaggtggtgcaccggcagggcctgatgggcgtccagaaggagttcgccttctgcagacctcccccggttagcaaagacaaacgtgcagcccagaagcctcaggtccaaggtcacgaccacgaccacgcaggagaaggagggcagcacaaagccagcaacccccaccgcccttcagaagcagtacagaataaaagtgggattaaaaggaacgccagcaccgcaaggaggggaaagcgagtcacgagcgccgtacaggcgcccgaggcgtccgactccagcagcgacgacggcattgaggaggccatccagctgtaccaggtgcagaaaacacacaaggaggccgacggggacccgccccagagggtccagctccaagaggaaagagcacctgcccctcccgcacacagcacaagcagcgccacaaaaagtgccttgccagagacccacaggaaaacacccagcaagaagaagccagtgcccaccaagaccacggaccctggtccaggg

SEQ ID NO: 15 - DNA, 1876 bp, Artificial sequence
  sequence of STAR15
cagtacatgcagaactgagtccaaacgagacggacagcaaacccggcagtgggctcccagacattcctgggggaaagggatcctaaccacaggcagttaaagtcatctcctccaaccctctatgacacaggctgtgcgctgtcatttaaaagctgagtgaaatttaacccttttcccatttagaaaaacaaagcgcagctggctgccagcactcatttaattttacataaacgtgctctttgaggctgaagcaaatctgactgattttcaatgtgaaaataaaatgtaaaaactgttcttggaattatttctaaacagaacatcagaatcgtctgaatcatcagaatcggctattttggaaaaatcggattcatcaaacgaatcttcggccaacaactgttagagaacgatgttaacaccacgcataggaatgttacattttctagaatttgacattttcattgacggaaaattactgtatcttgtatatggaaataccactactaaaaacataatgctataaatagaatgatgtcttttgtttccaaagtcaatatactcgagcaatgcaaaaataataataaaagtgagatacttcatggcaaagctgccgcaggataaacattgcagccacaagtgcccccagtattctcggggcaaactggaaaagggctaacaggcaacattttcatgttattctactgagtgcagtaattatttttaaaaatatacatgaataatgaaaaaactgtggtatggttttaaagaaatttccataacctggtgaaactcttcacacagggtaataggttcataaagccttggtcctctgcaaaacaagcatcaacttgacaatgactaaaagaagcaacagcaaaactgtcacgcatttggagccatggcctgggttgggccggtgtaaagctctccgccctctggagcaagtctgggccccagcggctggcatgtgggcactgcagggcctgggttgggcaggtgtgcagctctccgtcatctgagcctagtctgaggcctggtggctggcacgtgggccctgcagggcctctacttctcaccccagctccacttccctccctgccctcactgggtctcacagagccaatgaacactggggtcagattcagggcccagcatccactgcagtgggcactgcccttccacaaggcctggctccaggaagcaacccccacctcagccacacagtagggcaacaggaaatcccattcccccatgccagtgactacaccagggaaggggctcacgtgaggctggccccaggcctgctgtgagaccgcgttgtctatgagcttggatttaaggaacttgggagcaagaagctttctttcattacgggccaccagcagggaaaaaagttagcccaacgcagttgacagtcacacccccaccaggaccccagggcacagaaggagggaagaggacaacagaggatgaggtggggccagcagagggacagagaagagctgcctgccctggaacaggcagaaagcatcccacgtgcaagaaaaagtaggccagctagacttaaaatcagaactaccgctcatcaaaagatagtgtaacatttggggtgctataattttaacatgtcccccaaaaggcatgtgttggaaatttaatccccaacaaaccagggctgggaggtggagcctcatgagaggtggtgaggccatgagggtggagtgaatggatgaatgccattgtctcgggaatgggcctcttctacaaggacgagttcagcccccctttctcttgctcaccctctctttgccctttcgctagggagtgacgtaacaagaaggccctcacaagatgctggcaccttgatcttggactc

SEQ ID NO: 16 - DNA, 1282 bp, Artificial sequence
  sequence of STAR16
cgcccacctcggctttccaaagtgctgggattacaggcatgagtcactgcgcccatcctgattccaagtctttagataataacttaactttttcgaccaattgccaatcaggcaatctttgaatctgcctatgacctaggacatccctctccctacaagttgccccgcgtttccagaccaaaccaatgtacatcttacatgtattgattgaagttttacatctccctaaaacatataaaaccaagctatagtctgaccacctcaggcacgtgttctcaggacctccctggggctatggcatgggtcctggtcctcagatttggctcagaataaatctcttcaaatattttccagaattttactcttttcatcaccattacctatcacccataagtcagagttttccacaaccccttcctcagattcagtaatttgctagaatggccaccaaactcaggaaagtattttacttacaattaccaatttattatgaagaactcaaatcaggaatagccaaatggaagaggcatagggaaaggtatggaggaaggggcacaaagcttccatgccctgtgtgcacaccaccctctcagcatcttcatgtgttcaccaactcagaagctcttcaaactttgtcatttaggggtttttatggcagttccactatgtaggcatggttgataaatcactggtcatcggtgatagaactctgtctccagctcctctctctctcctccccagaagtcctgaggtggggctgaaagtttcacaaggttagttgctctgacaaccagcccctatcctgaagctattgaggggtcccccaaaagttaccttagtatggttggaagaggcttattatgaataacaaaagatgctcctatttttaccactagggagcatatccaagtcttgcgggaacaaagcatgttactggtagcaaattcatacaggtagatagcaatctcaattcttgccttctcagaagaaagaatttgaccaagggggcataaggcagagtgagggaccaagataagttttagagcaggagtgaaagtttattaaaaagttttaggcaggaatgaaagaaagtaaagtacatttggaagagggccaagtgggcgacatgagagagtcaaacaccatgccctgtttgatgtttggcttggggtcttatatgatgacatgcttctgagggttgcatccttctcccctgattcttcccttggggtgggctgtccgcatgcacaatggcctgccagcagtagggaggggccgcatg

SEQ ID NO: 17 - DNA, 793 bp, Artificial sequence
  sequence of STAR17
atccgaggggaggaggagaagaggaaggcgagcagggcgccggagcccgaggtgtctgcgagaactgttttaaatggttggcttgaaaatgtcactagtgctaagtggcttttcggattgtcttatttattactttgtcaggtttccttaaggagagggtgtgttgggggtgggggaggaggtggactggggaaacctctgcgtttctcctcctcggctgcacagggtgagtaggaaacgcctcgctgccacttaacaatccctctattagtaaatctacgcggagactctatgggaagccgagaaccagtgtcttcttccagggcagaagtcacctgttgggaacggcccccgggtccccctgctgggctttccggctcttctaggcggcctgatttctcctcagccctccacccagcgtccctcagggacttttcacacctccccacccccatttccactacagtctcccagggcacagcacttcattgacagccacacgagccttctcgttctcttctcctctgttccttctctttctcttctcctctgttccttctctttctctgtcataatttccttggtgctttcgccaccttaaacaaaaaagagaaaaaaataaaataaaaaaaacccattctgagccaaagtattttaagatgaatccaagaaagcgacccacatagccctccccacccacggagtgcgccaagacgcacccaggctccatcacagggccgagagcagcgccactctggtcgtacttttgggtcaagagatcttgcaaaagagg

SEQ ID NO: 18 - DNA, 492 bp, Artificial sequence
  sequence of STAR18
atctttttgctctctaaatgtattgatgggttgtgttttttttcccacctgctaataaatattacattgcaacattcttccctcaacttcaaaactgctgaactgaaacaatatgcataaaagaaaatcctttgcagaagaaaaaaagctattttctcccactgattttgaatggcacttgcggatgcagttcgcaaatcctattgcctattccctcatgaacattgtgaaatgaaacctttggacagtctgccgcattgcgcatgagactgcctgcgcaaggcaagggtatggttcccaaagcacccagtggtaaatcctaacttattattcccttaaaattccaatgtaacaacgtgggccataaaagagtttctgaacaaaacatgtcatctttgtggaaaggtgtttttcgtaattaatgatggaatcatgctcatttcaaaatggaggtccacgatttgtggccagctgatgcctgcaaattatcct

SEQ ID NO: 19 - DNA, 1840 bp, Artificial sequence
  sequence of STAR19
tcacttcctgatattttacattcaaggctagctttatgcatatgcaacctgtgcagttgcacagggctttgtgttcagaaagactagctcttggtttaatactctgttgttgccatcttgagattcattataatataatttttgaatttgtgttttgaacgtgatgtccaatgggacaatggaacattcacataacagaggagacaggtcaggtggcagcctcaattccttgccacccttttcacatacagcattggcaatgccccatgagcacaaaatttgggggaaccatgatgctaagactcaaagcacatataaacatgttacctctgtgactaaaagaagtggaggtgctgacagcccccagaggccacagtttatgttcaaaccaaaacttgcttagggtgcagaaagaaggcaatggcagggtctaagaaacagcccatcatatccttgtttattcatgttacgtccctgcatgaactaatcacttacactgaaaatattgacagaggaggaaatggaaagatagggcaacccatagttctttttccttttagtctttccttatcagtaaaccaaagatagtattggtaaaatgtgtgtgagttaattaatgagttagttttaggcagtgtttccactgttggggtaagaacaaaatatataggcttgtattgagctattaaatgtaaattgtggaatgtcagtgattccaagtatgaattaaatatccttgtatttgcatttaaaattggcactgaacaacaaagattaacagtaaaattaataatgtaaaagtttaatttttacttagaatgacattaaatagcaaataaaagcaccatgataaatcaagagagagactgtggaaagaaggaaaacgtttttattttagtatatttaatgggactttcttcctgatgttttgttttgttttgagagagagggatgtgggggcagggaggtctcattttgttgcccaggctggacttgaactcctgggctccagctatcctgccttagcttcttgagtagctgggactacaggcacacaccacagtgtctgacattttctggattttttttttttttttattttttttgtgagacaggttctggctctgttactcaggttgcagtgcagtggcatgatagcggctcactgcagcctcaacctcctcagcttaagctactctcccacttcagcctcctgagtagccaggactacagttgtgtgccaccacacctgtggctaatttttgtagagatggggtctctccacgttgccgaggctggtctccaactcctggtctcaagcgaacctcctgacttggcctcccgaagtgctgggattacaggcttgagccactgcatccagcctgtcctctgtgttaaacctactccaatttgtctttcatctctacataaacggctcttttcaaagttcccatagacctcactgttgctaatctaataataaattatctgccttttcttacatggttcatcagtagcagcattagattgggctgctcaattcttcttggtatattttcttcatttggcttctggggcatcacactctctttgagttactcattcctcattgatagcttcttcctagtcttctttactggttcttcctcttctccctgactccttaatattgtttttctccccaggctttagttcttagtcctcttctgttatctatttacacccaattctttcagagtctcatccagagtcatgaacttaaacctgtttctgtgcagataattcacattattatatctccagcccagactctcccgcaaactgcagactgatcctactg

SEQ ID NO: 20 - DNA, 780 bp, Artificial sequence
  sequence of STAR20
gatctcaagtttcaatatcatgttttggcaaaacattcgatgctcccacatccttacctaaagctaccagaaaggctttgggaactgtcaacagagctacagaaaagtcagtaaagaccaatggacccctcaaacaaaaacagccaagcttttctgccaaaaagatgactgagaagactgttaaagcaaaaaactctgttcctgcctcagatgatggctatccagaaatagaaaaattatttcccttcaatcctctaggcttcgagagttttgacctgcctgaagagcaccagattgcacatctccccttgagtgaagtgcctctcatgatacttgatgaggagagagagcttgaaaagctgtttcagctgggccccccttcacctttgaagatgccctctccaccatggaaatccaatctgttgcagtctcctttaagcattctgttgaccctggatgttgaattgccacctgtttgctctgacatagatatttaaatttcttagtgctttagagtttgtgtatatttctattaataaagcattatttgtttaacagaaaaaaagatatatacttaaatcctaaaataaaataaccattaaaaggaaaaacaggagttataactaataagggaacaaaggacataaaatgggataataatgcttaatccaaaataaagcagaaaatgaagaaaaatgaaatgaagaacagataaatagaaaacaaatagcaatatgaaagacaaacttgaccgggtgtggtggctgatgcctgtaatcc

SEQ ID NO: 21 - DNA, 607 bp, Artificial sequence
  sequence of STAR21
gatcaataatttgtaatagtcagtgaatacaaaggggtatatactaaatgctacagaaattccattcctgggtataaatcctagacatatttatgcatatgtacaccaagatatatctgcaagaatgttcacagcaaatctctttgtagtagcaaaaggccaaaaggtctatcaacaagaaaattaatacattgtggcacataatggcatccttatgccaataaaaatggatgaaattatagttaggttcaaaaggcaagcctccagataatttatatcatataattccatgtacaacattcaacaacaagcaaaactaaacatatacaaatgtcagggaaaatgatgaacaaggttagaaaatgattaatataaaaatactgcacagtgataacatttaatgagaaaaaaagaaggaagggcttagggagggacctacagggaactccaaagttcatggtaagtactaaatacataatcaaagcactcaaaatagaaaatattttagtaatgttttagctagttaatatcttacttaaaacaaggtctaggccaggcacggtggctcacacctgtaatcccagcactttgggaggctgaggcgggt

SEQ ID NO: 22 - DNA, 1380 bp, Artificial sequence
  sequence of STAR22
cccttgtgatccacccgccttggcctcccaaagtgctgggattacaggcgtgagtcactacgcccggccaccctccctgtatattatttctaagtatactattatgttaaaaaaagtttaaaaatattgatttaatgaattcccagaaactaggattttacatgtcacgttttcttattataaaaataaaaatcaacaataaatatatggtaaaagtaaaaagaaaaacaaaaacaaaaagtgaaaaaaataaacaacactcctgtcaaaaaacaacagttgtgataaaacttaagtgcctgaaaatttagaaacatccttctaaagaagttctgaataaaataaggaataaaataatcacatagttttggtcattggttctgtttatgtgatggattatgtttattgatttgtgtatgttgaacttatctcaatagatgcagacaaggccttgataaaagtttttaacaccttttcatgttgaaaactctcaatagactaggtattgatgaaacatatctcaaaataatagaagctatttatgataaacccatagccaatatcatactgagtgggcaaaagctggaagcattccctttgaaaactggcacaagacaaggatgccctctctcaccactcctattaaatgtagtattggaagttctggccagagcaatcaggcaggagaaagaaaaggtattaaaataggaagagaggaagtcaaattgtctctgtttgcagtaaacatgattgtatatttagaaaaccccattgtctcatcctaaaaactccttaagctgataaacaacttcagcaaagtctcaggatacaaaatcaatgtgcaaaaatcacaagcattcctatacaccgataatagacagcagagagccaaatcatgagtgaagtcccattcacaattgcttcaaagaaaataaaatacttaggaatacaactttcacgggacatgaaggacattttcaaggacaactaaaaaccactgctcaaggaaatgagagaggacacaaagaaatggaaaaacattccatgctcatggaagaatcaatatcatgaaaatggccatactgcccaaagtaatttatagattcaatgctaaccccatcaagccaccattgactttcttcacagaactagaaaaaaactattttaaaactcatatgtagtcaaaaagagtcggtatagccaagacaatcctaagcataaagaacaaagctggatgcatcacgctgacttcaaaccatactacaaggctacagtaaccaaaacagcatggtactggtaccaaaacagatagatagaccgatagaacagaacagaggcctcggaaataacaccacacatctacaaccctttgatcttcaa

SEQ ID NO: 23 - DNA, 1246 bp, Artificial sequence
  sequence of STAR23
atcccctcatccttcagggcagctgagcagggcctcgagcagctgggggagcctcacttaatgctcctgggagggcagccagggagcatggggtctgcaggcatggtccagggtcctgcaggcggcacgcaccatgtgcagccgcccccacctgttgctctgcctccgccacctggccatgggcttcagcagccagccacaaagtctgcagctgctgtacatggacaagaagcccacaagcagctagaggaccttgtgttccacgtgcccagggagcatggcccacagcccaaagaccagtcaggagcaggcaggggcttctggcaggcccagctctacctctgtcttcacacagatgggagatttctgttgtgattttgagtgatgtgcccctttggtgacatccaagatagttgctgaagcaccgctctaacaatgtgtgtgtattctgaaaacgagaacttctttattctgaaataattgatgcaaaataaattagtttggatttgaaattctattcatgtaggcatgcacacaaaagtccaacattgcatatgacacaaagaaaagaaaaagcttgcattccttaaatacaaatatctgttaactatatttgcaaatatatttgaatacacttctattatgttacatataatattatatgtatatgtatatataatatacatatatatgttacatataatatacttctattatgttacatataatatttatctataagtaaatacataaatataaagatttgagtagctgtagaacattgtcttatgtgttatcagctactactacaaaaatatctcttccacttatgccagtttgccatataaatatgatcttctcattgatggcccagggcaagagtgcagtgggtacttattctctgtgaggagggaggagaaaagggaacaaggagaaagtcacaaagggaaaactctggtgttgccaaaatgtcaagtttcacatattccgagacggaaaatgacatgtcccacagaaggaccctgcccagctaatgtgtcacagatatctcaggaagcttaaatgatttttttaaaagaaaagagatggcattgtcacttgtttcttgtagctgaggctgtgggatgatgcagatttctggaaggcaaagagctcctgctttttccacaccgagggactttcaggaatgaggccagggtgctgagcactacaccaggaaatccctggagagtgtttttcttactta

SEQ ID NO: 24 - DNA, 939 bp, Artificial sequence
  sequence of STAR24
acgaggtcacgagttcgagaccagcctggccaagatggtgaagccctgtctctactaaaaatacaacaagtagccgggcgcggtgacgggcgcctgtaatcccagctactcaggaggctgaagcaggagaatctctagaacccaggaggcggaggtgcagtgagctgagactgccccgctgcactctagcctgggcaacacagcaagactctgtctcaaataaataaataaataaataaataaataaataaataaataaatagaaagggagagttggaagtagatgaaagagaagaaaagaaatcctagatttcctatctgaaggcaccatgaagatgaaggccacctcttctgggccaggtcctcccgttgcaggtgaaccgagttctggcctccattggagaccaaaggagatgactttggcctggctcctagtgaggaagccatgcctagtcctgttctgtttgggcttgatcctgtatcacttgattgtctctcctggactttccatggattccagggatgcaactgagaagtttatttttaatgcacttacttgaagtaagagttattttaaaacattttagcaaaggaaatgaattctgacaggttttgcactgaagacattcacatgtgaggaaaacaggaaaaccactatgctagaaaaagcaaatgctgttgagattgtctcacaaacacaaattgcgtgccagcaggtaggtttgagcctcaggttgggcacattttaccttaagcgcactgttggtggaacttaaggtgactgtaggacttatatatacatacatacatataatatatatacatatttatgtgtatatacacacacacacacacacacacacacagggtcttgctatcttgcccagggtggtctccaactctgggtctcaagcgatcctctgcctccccttcccaaag

SEQ ID NO: 25 - DNA, 1067 bp, Artificial sequence
  sequence of STAR25
ataaaaaaataaaaaaccctgctctaatttgcaaaggctctatctttcctcccaaccacctgaaattttagtgaaaacggggcttcctgtaggaaggagtagctagctatcccggtccgctacaggttatcagtgcgtgaataccctgactcctaaggctcaggatttgactgggtcgcctcgtccgactgccccgcccccaacgcggacccacgtcaccgcgcgccagcctgcggccgtcctgacctcgcgggatttgagcttcggtgccaacaaacactcccaccgcggctgcgtccactttacctgccggcggcgaccagcttctgaagaaaagtgtccaccatggtgtcgaggagcttcaccctcgaaatggtagtgccgggtggcacagattccgaagacgacccctcatgccttttttcctcacagccgctgcctagattggcgctacttgcttcggccatgttgaagttgaacctccaaatctaactggcccggcctccccgcctgccggagctcccgattggccgctcccgcgaagggtgcctccgattggaagcagtagaacgtctgtcaccgagcagggcgggggcggggaagtcatcggaggctgagggcagcggggaggcgaggctctgcgcggtgggatgtccgcgaccggaaaaatacgcgcaagccaaagctcgggggctcaataaaaacttttaattacatttcagagacttcgtacagtgcaacagtgaatattcactgttaattttcacaagagtccatttcatcaaacgttcagagagtctgccttttcattcccttgttcctcagtgctccaatcaggtttccagtctcccagaggtttcttttagttttgattaccgaccaaaactccagtttagggagaatggaagtccaccgtcccatccccaccaaaacatatttcagtcaaacccaatcccagtccctaaagaattaggaaagtatgggccaagggtccttttaattatacacacatcacccttaaaactgcgtgtgtgtacgagaaataaagaaaaacacaagaggggctg

SEQ ID NO: 26 - DNA, 540 bp, Artificial sequence
  sequence of STAR26
ccccctgacaagccccagtgtgtgatgttccccactctgtgtccatgcattctcattgttcaactcccatctgtgagtgagaacatgcagtgtttggttttctgtccttgagatagtttgctgagaatgatggtttccagcttcatccatgtccttgcaaaggaagtgaacttatccttttttatggcttcatagtattccatggcacatatgtgccacatttttttaatccagtctatcattgatggacatttgggttggttccaagtctttgctattgtgaatagcaccacaattaacatatgtgtgcatgtatacatctttatagtagcatgatttataatccttcgggtatataccctgtaatgggatcgctgggtcaaatggtatttctagttctagatccttgaggaatcaccacactgctttccacaatggttgaactaatttacgctcccaccagcagtgtaaaagcattcctatttctccacgtcctctccagtatctgttgtttcctgactttttaatgatcatcattct

SEQ ID NO: 27 - DNA, 1520 bp, Artificial sequence
  sequence of STAR27
cttggccctcacaaagcctgtggccagggaacaattagcgagctgcttattttgctttgtatccccaatgctgggcataatgcctgccattatgagtaatgccggtagaagtatgtgttcaaggaccaaagttgataaataccaaagaatccagagaagggagagaacattgagtagaggatagtgacagaagagatgggaacttctgacaagagttgtgaagatgtactaggcagggggaacagcttaaggagagtcacacaggaccgagctcttgtcaagccggctgccatggaggctgggtggggccatggtagctttcccttccttctcaggttcagagtgtcagccttgaacttctaattcccagaggcatttattcaatgttttcttctaggggcatacctgccctgctgtggaagactttcttccctgtgggtcgccccagtccccagatgagacggtttgggtcagggccaggtgcaccgttgggtgtgtgcttatgtctgatgacagttagttactcagtcattagtcattgagggaggtgtggtaaagatggagatgctgggtcacatccctagagaggtgttccagtatgggcacatgggagggctggaaggataggttactgctagacgtagagaagccacatcctttaacaccctggcttttcccactgccaagatccagaaagtccttgtggtttcgctgctttctcctttttttttttttttttttttctgagatggagtctggctctgtcgcccaggctggagtgcagtggcacgatttcggctcactgcaagttccgcctcctaggttcataccattctcccacctcagcctcccgagtagctgggactacaggcgccaccacacccagctaattttttgtatttttagtagagacggcgtttcaccatgttagccaggatggtcttgatccgcctgcctcagcctcccaaagtgctgggattacaggcgtgagccaccgcgcccggcctgctttcttctttcatgaagcattcagctggtgaaaaagctcagccaggctggtctggaactcttgacctcaagtgatctgcctgcctcagcctcccaaagtgctgagattacaggcatgagccagtccgaatgtggctttttttgttttgttttgaaacaaggtctcactgttgcccaggctgcagtgcagtggcatacctcagctccactgcagcctcgacctcctgggctcaagcaatcctcccaactgagcctccccagtagctggggctacaagcgcatgccaccacgcctggctatttttttttttttttttttttttgagaaggagtttcattcttgttgcccaggctggagtgcaatggcacagtctcagctcactgcagcctccgcctcctgggttcaagcgattctcctgcctcagcctcccgagtagctgggattataggcacctgccaccatgcctggctaatttttttgtatttttagtagggatggggtttcaccatgtt

SEQ ID NO: 28 - DNA, 961 bp, Artificial sequence
  sequence of STAR28
aggaggttattcctgagcaaatggccagcctagtgaactggataaatgcccatgtaagatctgtttaccctgagaagggcatttcctaactctccctataaaatgccaagtggagcaccccagatgaaatagctgatatgctttctatacaagccatctaggactggctttatcatgaccaggatattcacccactgaatatggctattacccaagttatggtaaatgctgtagttaagggggtcccttccacatggacaccccaggttataaccagaaagggttcccaatctagactccaagagagggttcttagacctcatgcaagaaagaacttggggcaagtacataaagtgaaagcaagtttattaagaaagtaaagaaacaaaaaaatggctactccataagcaaagttatttctcacttatatgattaataagagatggattattcatgagttttctgggaaaggggtgggcaattcctggaactgagggttcctcccacttttagaccatatagggtatcttcctgatattgccatggcatttgtaaactgtcatggcactgatgggagtgtcttttagcattctaatgcattataattagcatataatgagcagtgaggatgaccagaggtcacttctgttgccatattggtttcagtggggtttggttggcttttttttttttttaaccacaacctgttttttatttatttatttatttatttatttatttatattttttatttttttttagatggagtcttgctctgtcacccaggttagagtgcagtggcaccatctcggctcactgcaagctctgcctccttggttcacgccattctgctgcctcagcctcccgagtagctgggactacaggtgcctgccaccatacccggctaattttttctatttttcagtagagacggggtttcaccgtgttagccaggatggtc

SEQ ID NO: 29 - DNA, 2233 bp, Artificial sequence
  sequence of STAR29
agcttggacacttgctgatgccactttggatgttgaagggccgccctctcccacaccgctggccacttttaaatatgtcccctctgcccagaagggccccagaggaggggctggtgagggtgacaggagttgactgctctcacagcagggggttccggagggaccttttctccccattgggcagcatagaaggacctagaagggccccctccaagcccagctgggcgtgcagggccagcgattcgatgccttcccctgactcaggtggcgctgtcctaaaggtgtgtgtgttttctgttcgccagggggtggcggatacagtggagcatcgtgcccgaagtgtctgagcccgtggtaagtccctggagggtgcacggtctcctccgactgtctccatcacgtcaggcctcacagcctgtaggcaccgctcggggaagcctctggatgaggccatgtggtcatccccctggagtcctggcctggcctgaagaggaggggaggaggaggccagcccctccctagccccaaggcctgcgaggctgcaagcccggccccacattctagtccaggcttggctgtgcaagaagcagattgcctggccctggccaggcttcccagctaggatgtggtatggcaggggtgggggacattgaggggctgctgtagcccccacaacctccccaggtagggtggtgaacagtaggctggacaagtggacctgttcccatctgagattcaagagcccacctctcggaggttgcagtgagccgagatccctccactgcactccagcctgggcaacagagcaagactctgtctcaaaaaaacagaacaacgacaacaaaaaacccacctctggcccactgcctaactttgtaaataaagttttattggcacatagacacacccattcatttacatactgctgcggctgcttttgcattacccttgagtagacgacagaccacgtggccatggaagccaaaaatatttactgtctggccctttacagaagtctgctctagagggagaccccggcccatggggcaggaccactgggcgtgggcagaagggaggcctcggtgcctccacgggcctagttgggtatctcagtgcctgtttcttgcatggagcaccaggggtcagggcaagtacctggaggaggcaggctgttgcccgcccagcactgggacccaggagaccttgagaggctcttaacgaatgggagacaagcaggaccagggctcccattggctgggcctcagtttccctgcctgtaagtgagggagggcagctgtgaaggtgaactgtgaggcagagcctctgctcagccattgcaggggcggctctgccccactcctgttgtgcacccagagtgaggggcacggggtgagatgtcaccatcagcccataggggtgtcctcctggtgccaggtccccaagggatgtcccatcccccctggctgtgtggggacagcagagtccctggggctgggagggctccacactgttttgtcagtggtttttctgaactgttaaatttcagtggaaaattctctttccccttttactgaaggaacctccaaaggaagacctgactgtgtctgagaagttccagctggtgctggacgtcgcccagaaagcccaggtactgccacgggcgccggccaggggtgtgtctgcgccagccatgggcaccagccaggggtgtgtctacgccggccaggggtaggtctccgccggcctccgctgctgcctggggagggccgtgcctgacactgcaggcccggtttgtccgcggtcagctgacttgtagtcaccctgcccttggatggtcgttacagcaactctggtggttggggaaggggcctcctgattcagcctctgcggacggtgcgcgagggtggagctcccctccctccccaccgcccctggccagggttgaacgcccctgggaaggactcaggcccgggtctgctgttgctgtgagcgtggccacctctgccctagaccagagctgggccttccccggcctaggagcagccgggcaggaccacagggctccgagtgacctcagggctgcccgacctggaggccctcctggcgtcgcggtgtgactgacagcccaggagcgggggctgttgtaattgctgtttctccttcacacagaaccttttcgggaagatggctgacatcctggagaagatcaagaagtaagtcccgccccccaccc

SEQ ID NO: 30 - DNA, 1851 bp, Artificial sequence
  sequence of STAR30
cctcccctggagccttcagaaggagcatggcataggagtcttgatttcagacgtctggtccccagaatgatgggagaatgaatttctgttatttaagccacccaacctgtggtgctttgttatagcagcctcaggaaactaacacactgcacgtgcccactattcccttttccagtatctttcaggacttgctggcttcctttgttctggcgtacacccatgcatggccccattccccacttcctaaaacaacaaccctgacttagtctgtttgggctgctagaacaaaatactatagactgggtgacttataaacaacagaaattcatttctcacattctggaggctgggaagtccaatatcgaggcaccatcacatttggtctctgctgaggcccccttcctagctcctcactgtgtccttacatggcagaaggggcaaggcagctctctggggtcccttttcaaggccacaaatcccattcattagggctgatgacttcatgacttaatcacctcctaatggccccacctcctaatcgcattgggcgttaggattcaacataaattttggggggacacacatattcagaccatagcaaaccccaacaataaaaaaccttcactttaaggttccaaatggactggcagttaaatcatgttcatatttacataaaagaaggagtaagtcaacaaattgataaacgcgtggagatttgttcggatggatgttcaccaaaatgctggccttaaagagtgagatgggaaatgggaactattacattcttcttcatactttttggtactgcctgcattgttaaaaaaaaaaaaaaagagcacagagcatttttacaatcaggaaaaaaacaatgaggttatcttcattctggaaaaaaatggaaaatgaaacagtggagtcacatcatggaaaatgcttatggtacaatttcatgtgacataaaacaatagaatagaggacctgttttatgactaaagcactgtaaaaatgacaggcctggaaggagagatgaaaaccactcatttgttaaggtagtcaggtggcaggtgatttctcttcttttgaaaatttccattttcattatatcgcagtttgtgcatttactaaaactttcggttggtacacatgcataaatagatagataaataagtagatagatgatagataaatagacggtaggtagatagatagatagatatgagaaataagtcccctgtacttggccttgcagccataactagtcattccccttcctctgtccattgctatgcctgatggacaaggcagtctgtgccctctggccccaattccaatgtgccctctgctcctggctgttagtccctttccaccccaatacaattgctccgaggtcacttctaagtgtgaagcccccagatcagatggcttcttctgtgtccttaccttacccaatttctaattataactaaaacacaatgaggctctagtaaaataccatgagacttcaggccctctgtataacttcactcatttaaacctaacaaggaaaacctaccatgaatccgaggcacagagcagctaaggaactcaccaaggtcacgcagctattggtgatggaaccatgagtcaagcttcacagcctgttggctctagaatagggtttcccaacctcagcactgtggacattttcaggctggataattctctgttgtggggggctgttctgtgccttgtaggatattaggagcatctctggcctctacccactagacgcagcagcactcccatgcccagttgtgacaacaagcaatgtctcccaccattgccaagtgtcccctgggtggaaatgcaccc

SEQ ID NO: 31 - DNA, 1701 bp, Artificial sequence
  sequence of STAR31
cacccgccttggccccccagagtgctgggattacaagtgtaaaccaccattcctggctagatttaattttttaaaaaataaagagaagtaggaatagttcattttagggagagccccttaactgggacaggggcaggacaggggtgaggcttcccttanttcaagctcacctcaaacccacccaggactgtgtgtcacattctccaataaaggaaaggttgctgcccccgcctgtgagtgctgcagtggagggtagagggccgtgggcagagtgcttcatggactgctcatcaagaaaggcttcatgacaatcggcccagctgctgtcatcccacattctacttccagctaggagaaggcggcttgcccacagtcacccagccggcaagtgtcacccctgggttggacccagagctatgatcctgcccaggggtccagctgagaatcaggcccacgttctaggcagaggggctcacctactgggactccagtagctgtagtgcatggaggcatcatggctgcagcagcctggacctggtctcacactggctgtccctgtgggcaggccatcctcaatgccaggtcaggcccaagcatgtatcccagacaatgacaatggggtggaatcctctcttgtcccagaagccactcctcactgttctacctgaggaaggcaggggcatggtggaatcctgaagcctgctgtgagggtctccagcgaacttgcacatggtcagccctgccttctcctccctgaactagattgagcgagagcaagaaggacattgaaccagcacccaaagaattttggggaacggcctctcatccaggtcaggctcacctcctttttaaaatttaattaattaattaattaatttttttttagagacagagtcttactgtgtggcccaggctgtagtgcagtggcacaatcatagttcactgcagcctcaaactccccacctcagcctctggattagctgagactacaggtgcaccaccaccacacccagctaatatttttatttttgtagagagagggtttcaccatcttgcccaggctggtctcaaactcctgggctcaagtgatcccgcccaggtctgaaagcccccaggctggcctcagactgtggggttttccatgcagccacccgagggcgcccccaagccagttcatctcggagtccaggcctggccctgggagacagagtgaaaccagtggtttttatgaacttaacttagagtttaaaagatttctactcgatcacttgtcaagatgcgccctctctggggagaagggaacgtgactggattccctcactgttgtatcttgaataaacgctgctgcttcatcctgtgggggccgtggccctgtccctgtgtgggtggggcctcttccatttccctgacttagaaaccacagtccacctagaacagggtttgagaggcttagtcagcactgggtagcgttttgactccattctcggctttcttctttttctttccaggatttttgtgcagaaatggttcttttgttgccgtgttagtcctccttggaaggcagctcagaaggcccgtgaaatgtcgggggacaggacccccagggagggaaccccaggctacgcactttagggttcgttctccagggagggcgacctgacccccgnatccgtcggngcgcgnngnnacnaannnnttccc

SEQ ID NO: 32 - DNA, 771 bp, Artificial sequence
  sequence of STAR32
gatcacacagcttgtatgtgggagctaggattggaaccccagaagtctggccccaggttcatgctctcacccactgcatacaatggcctctcataaatcaatccagtataaaacattagaatctgctttaaaaccatagaattagtagcgtaagtaataaatgcagagaccatgcagtgaatggcattcctggaaaaagcccccagaaggaattttaaatcagctttcgtctaatcttgagcagctagttagcaaatatgagaatacagttgttcccagataatgctttatgtctgaccatcttaaactggcgctgtttttcaaaaacttaaaaacaaaatccatgactcttttaattataaaagtgatacatgtctacttgggaggctgaggtggtgggaggatggcttgagtttgaggctgcagtatgctactatcatgcctataaatagccgctgcattccagcttgggcaacatacccaggccctatctcaaaaaaataaaaagtaatacatctacattgaagaaaattaattttattgggtttttttgcatttttattatacacagcacacacagcacatatgaaaaaatgggtatgaactcaggcattcaactggaagaacagtactaaatcaatgtccatgtagtcagcgtgactgaggttggtttgttttttcttttttcttctcttctcttctcttttctttttttttgagacggagctttgctctttttgcccaggcttgattgcaatggcgtgatctcag

SEQ ID NO: 33 - DNA, 1368 bp, Artificial sequence
  sequence of STAR33
gcttttatcctccattcacagctagcctggcccccagagtacccaattctccctaaaaaacggtcatgctgtatagatgtgtgtggcttggtagtgctaaagtggccacatacagagctctgacaccaaacctcaggaccatgttcatgccttctcactgagttctggcttgttcgtgacacattatgacattatgattatgatgacttgtgagagcctcagtcttctatagcacttttagaatgctttataaaaaccatggggatgtcattatattctaacctgttagcacttctgttcgtattacccatcacatcccaacatcaattctcatatatgcaggtacctcttgtcacgcgcgtccatgtaaggagaccacaaaacaggctttgtttgagcaacaaggtttttatttcacctgggtgcaggtgggctgagtctgaaaagagagtcagtgaagggagacaggggtgggtccactttataagatttgggtaggtagtggaaaattacaatcaaagggggttgttctctggctggccagggtgggggtcacaaggtgctcagtgggagagcctttgagccaggatgagccagaaggaatttcacaaggtaatgtcatcagttaaggcagggactggccattttcacttcttttgtggtggaatgtcatcagttaaggcaggaaccggccattttcacttcttttgtgattcttcacttgcttcaggccatctggacgtataggtgcaggtcacagtcacaggggataagatggcaatggcatagcttgggctcagaggcctgacacctctgagaaactaaagattataaaaatgatggtcgcttctattgcaaatctgtgtttattgtcaagaggcacttatttgtcaattaagaacccagtggtagaatcgaatgtccgaatgtaaaacaaaatacaaaacctctgtgtgtgtgtgtgtgtgagtgtgtgtgtatgtgtgtgtgtgtgtattagagaggaaaagcctgtatttggaggtgtgattcttagattctaggttctttcctgcccaccccatatgcacccaccccacaaaagaacaaacaacaaatcccaggacatcttagcgcaacatttcagtttgcatattttacatatttacttttcttacatattaaaaaactgaaaattttatgaacacgctaagttagattttaaattaagtttgtttttacactgaaaataatttaatatttgtgaagaatactaatacattggtatatttcattttcttaaaattctgaacccctcttcccttatttccttttgacccgattggtgtattggtcatgtgactcatggatttgccttaaggcaggagg

SEQ ID NO: 34 - DNA, 755 bp, Artificial sequence
  sequence of STAR34
actgggcaccctcctaggcaggggaatgtgagaactgccgctgctctggggctgggcgccatgtcacagcaggagggaggacggtgttacaccacgtgggaaggactcagggtggtcagccacaaagctgctggtgatgaccaggggcttgtgtcttcactctgcagccctaacacccaggctgggttcgctaggctccatcctgggggtgcagaccctgagagtgatgccagtgggagcctcccgcccctccccttcctcgaaggcccaggggtcaaacagtgtagactcagaggcctgagggcacatgtttatttagcagacaaggtggggctccatcagcggggtggcctggggagcagctgcatgggtggcactgtggggagggtctcccagctccctcaatggtgttcgggctggtgcggcagctggcggcaccctggacagaggtggatatgagggtgatgggtggggaaatgggaggcacccgagatggggacagcagaataaagacagcagcagtgctggggggcagggggatgagcaaaggcaggcccaagacccccagcccactgcaccctggcctcccacaagccccctcgcagccgcccagccacactcactgtgcactcagccgtcgatacactggtctgttagggagaaagtccgtcagaacaggcagctgtgtgtgtgtgtgcgtgtatgagtgtgtgtgtgtgatccctgactgccaggtcctctgcactgcccctggg

SEQ ID NO: 35 - DNA, 1193 bp, Artificial sequence
  sequence of STAR35
cgacttggtgatgcgggctcttttttggttccatatgaactttaaagtagtcttttccaattctgtgaagaaagtcattggtaggttgatggggatggcattgaatctgtaaattaccttgggcagtatggccattttcacaatgttgattcttcctatccatgatgatggaatgttcttccattagtttgtatcctcttttatttccttgagcagtggtttgtagttctccttgaagaggtccttcacatcccttgtaagttggattcctaggtattttattctctttgaagcaaattgtgaatgggagtncactcacgatttggctctctgtttgtctgctgggtgtataaanaatgtngtgatnttngtacattgatttngtatccntgagacttngctgaatttgcttnatcngcttnngggaaccttttgggctgaaacnatgggattttctaaatatacaatcatgtcgtctgcaaacagggaacaatttgacttcctcttttcctaattgaatacactttatctccttctcctgcctaattgccctgggcaaaacttccaacactatgntngnaataggagntggtgagagagggcatccctgttcttgttgccagnttttcaaagggaatgcttccagttttggcccattcagtatgatatgggctgtgggtngtgtcataaatagctcttatnattttgaaatgtgtcccatcaatacctaatttattgaaagtttttagcatgaangcatngttgaatttggtcaaaggctttttctgcatctatggaaataatcatgtggtttttgtctttggctcntgtttatatgctggatnacatttattgatttgtgtatatngaacccagcctnncatcccagggatgaagcccacttgatccaagcttggcgcgcngnctagctcgaggcaggcaaaagtatgcaaagcatgcatctcaattagtcagcacccatagtccgcccctacctccgcccatccgcccctaactcngnccgttcgcccattctcgcccatggctgactaatntttttannatccaagcggngccgccctgcttgancattcagagtnnagagnnttggaggccnagccttgcaaaactccggacngnttctnnggattgaccccnnttaaatatttggttttttgtnttttcannggnga

SEQ ID NO: 36 - DNA, 1712 bp, Artificial sequence
  sequence of STAR36
gatcccatccttagcctcatcgatacctcctgctcacctgtcagtgcctctggagtgtgtgtctagcccaggcccatcccctggaactcaggggactcaggactagtgggcatgtacacttggcctcaggggactcaggattagtgagccccacatgtacacttggcctcagtggactcaggactagtgagccccacatgtacacttggcctcaggggactcaggattagtgagcccccacatgtacacttggcctcaggggactcaggattagtgagccccacatgtacacttggcctcaggggactcaggactagtgagccccacatgtacacttggcctcaggggactcagaactagtgagccccacatgtacacttggcttcaggggactcaggattagtgagccccacatgtacacttggacacgtgaaccacatcgatgtgctgcagagctcagccctctgcagatgaaatgtggtcatggcattccttcacagtggcacccctcgttccctccccacctcatctcccattcttgtctgtcttcagcacctgccatgtccagccggcagattccaccgcagcatcttctgcagcacccccgaccacacacctccccagcgcctgcttggccctccagcccagctcccgcctttcttccttggggaagctccctggacagacaccccctcctcccagccatggctttttcctgctctgccccacgcgggaccctgccctggatgtgctacaatagacacatcagatacagtccttcctcagcagccggcagacccagggtggactgctcggggcctgcctgtgaggtcacacaggtgtcgttaacttgccatctcagcaactagtgaatatgggcagatgctaccttccttccggttccctggtgagaggtactggtggatgtcctgtgttgccggccaccttttgtccctggatgccatttatttttttccacaaatatttcccaggtctcttctgtgtgcaaggtattagggctgcagcgggggccaggccacagatctctgtcctgagaagacttggattctagtgcaggagactgaagtgtatcacaccaatcagtgtaaattgttaactgccacaaggagaaaggccaggaaggagtggggcatggtggtgttctagtgttacaagaagaagccagggagggcttcctggatgaagtggcatctgacctgggatctggaggaggagaaaaatgtcccaaaagagcagagagcccaccctaggctctgcaccaggaggcaacttgctgggcttatggaattcagagggcaagtgataagcagaaagtccttgggggccacaattaggatttctgtcttctaaagggcctctgccctctgctgtgtgaccttgggcaagttacttcacctctagtgctttggttgcctcatctgtaaagtggtgaggataatgctatcacactggttgagaattgaagtaattattgctgcaaagggcttataagggtgtctaatactagtactagtaggtacttcatgtgtcttgacaattttaatcattattattttgtcatcaccgtcactcttccaggggactaatgtccctgctgttctgtccaaattaaacattgtttatccctgtgggcatctggcgaggtggctaggaaagcctggagctgtttcctgttgacgtgccagactagt

SEQ ID NO: 37 - DNA, 1321 bp, Artificial sequence
  sequence of STAR37
atctctctctgccaaagcaacagcggtccctgccccaaccagactaccccactcagtggggttacggatgctgctccagcatcctaacactgcccagctggtgcctgcctgtgctcacccacaacccccaggccggccttccctgcagcctgggcttggccaccttggcctgattgagcactgaggcctcctgggcacccagccccatcactgcacctgctgcttccagccccaccccaccggctcaggggttcttcccagcggcgctgatcatgaagtcaacatgcacgcaagtcgtctcaggaaactttttaatgaaagtgtcggccacggtggtgtgtaggtggctgagctcagattgcagctgctaagacaccagccacttaccaagagaaagccaggctgcttcaaacccagggccggaggcaaaaaagcatcacttccagccggggagtctggaagccacgccttgtgggaggtcacactggcatctaggccttcgcctgcactgcagaaggagagccgggtccccctcctggagaacgctgcgttccccagccccacaccggctttgccaccacacaggctgttgaggcaggaggcgggtaagacgtagctgtagacccaaagcaaccaccagccctgggaccctgcgggagaggagcacttttagaacatggaaaaatgtggtcatcccatcattagacagcacacatcctacataaataaaaagtcgtatggggaaggaggttggggagggaataaaaaattggcacagacattgatagactggtttccagtttcaaggtaacagatgcacatcatgagaccagaggaggcagagacaagggctgaatttggcttttctaagcaacatgtgttcctgcgcagggctgaatggtcgctgagacagagatggaagccaggacaagggagcccaccgggcccagataggtacagagagcagaggctcctgttctgtcctcgccacccatgagggtgacactgcttgtaaatggtggctgtgctctcccagcaagaaaaaagcacaactaaatccacactgcacacagacgcagacagaaagccttcaagtggctctgttttctgctccctgccttgccaggtccacaagcagagaggagtgtcaggcacatggccccgctgtcaggctccccagtgagctgtaggctcagcaggagctgcccactgacacacaggggacacccactcctgccaccttgggagcggttgccagacagagccgcactgggtgctggtgtcatccagggaccccacacacttccttaaatgtgatcct

SEQ ID NO: 38 - DNA, 1445 bp, Artificial sequence
  sequence of STAR38
gatctatgggagtagcttccttagtgagctttcccttcaaatactttgcaaccaggtagagaattttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtatnagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttgtttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaaatcctaaagacaatacatttttattaactatagccctcatgatgtacattagatcgtgtggttgtttcttccgtccccgccacgccttcctcctgggatggggattcattccctagcaggtgtcggagaactggcgcccttgcagggtaggtgccccggagcctgaggcgggnactttaanatcagacgcttgggggccggctgggaaaaactggcggaaaatattataactgnactctcaatgccagctgttgtagaagctcctgggacaagccgtggaagtcccctcaggaggcttccgcgatgtcctaggtggctgctccgcccgccacggtcatttccattgactcacacgcgccgcctggaggaggaggctgcgctggacacgccggtggcgcctttgcctgggggagcgcagcctggagctctggcggcagcgctgggagcggggcctcggaggctgggcctggggacccaaggttgggcggggcgcaggaggtgggctcagggttctccagagaatccccatgagctgacccgcagggcggccgggccagtaggcaccgggcccccgcggtgacctgcggacccgaagctggagcagccactgcaaatgctgcgctgaccccaaatgctgtgtcctttaaatgttttaattaagaataattaataggtccgggtgtggaggctcaagccttaatccccagcacctggcgaggccgaggaggga

SEQ ID NO: 39 - DNA, 2331 bp, Artificial sequence
  sequence of STAR39
tcactgcaacctccacctcccaggttcaagtgattctcctgcctcggcctcccgagtagctgggactacaggtgcatgacaccgcacctggctagtttttgtatttttagtagagacagggtttcactatgttggccaggttggtctcgaactcctgaccttgtgatccgcccacctcggcctcccaaagtgctgggattacagagtgagccactgcgcctggcctgcaccccttactattatatgctttgcattttcttttagatttgaagaacctcattataaactctagcactaatcttatgtcagttaaatgcatagcaaatatctcctgacgtgggagaatatatatttgcaagtcttcttgtgaacatatgttttcagttctagggagccagacgcctatgagtgaaaagcctagtcatcgtggagaagtgcattcaactttgtaagaaactgccaaacctttattcataatggttgtataaattttacattaccaccaataatgtatgagagttccagttgcttcacatcctcaccagcattttgttttgtctgtcttttttcctttggttattctagtgggcataagatataatagtatcccttgtggtttaatgtaaattccactgaagactaataacatttgcatatttctaattaataagcctttttaagtgacttttcaagtctttgctcatttttattagatatttgccttcttattattgatttgaaagaattatatttatatgcttatattctggttataagccctttgtcattattttccaaaacaatatttggttgtttctgtactactttccttgctcctttgaattgacttggtgccttggccaaaaatcaattgaccacatacatgtgggtgcatctccagactaccacattccgtttatctatttgtctctccttgtgtcaataacactctgtcttgataatggtaagttttgagatcaggttgtgtaagtcctcctaatttttcctgggttttcaatattgctttgctttttaaaaattttgtattttcatttacattttaaaataaacttgttagtgggattttgattggcattgcactgaactcgtggatcaatttggggagattggacattcttatatatggatcccgtggtcatcaactttaagaactctttctcatccattagtaactcaatctaggttcagatgctactcgttttctgctcagtctgtgtctgagccccttatgctcttcattttgtcatccaattaacctcagctttgcatcaatactatttcttgctttggtgcctgttacctctcctctaatcaccaatccacaacttacctccaaattcagggcttgtctcattcttcccaggaggagtgctgctcagtctatctacttagtattataatttctctggcttggtatcaaggcactcccatttccggcttccatgagatgtctcagagggcatgctgcccggtgtagctgcatggtcaagcttcttcatatctcttgcctcatcacttaaactcactattttgtactcctgcttcagctatagggagctactgttagtttcttgaagacatatgctctctctctctctcacatctggacctgagcacatcctgttactgctgcttgaaacaatgtgatccccaggcacacaccattagcttagaagcctcccctgattcttcaaggctggttgagtcccttctctgtgctctcatgacaacagttggcaattcctcgttgcagcacctagcccatgatgctctttggaggcagagactgagtctttctcactattgaatttccagcattcatcacagagcctggcatatataaagccctccatcatatgtattaagtgaatggataaatgaaaaaaagttatatatatgtacatatatgtgtatatatgtatatgtatatatgtgtatatatgtgtgtatatgtgtgtgtatatatgtacatatatatgtatctatgtacatatatgtatatatgtatatatatgtgtgtgtatatgtgtgtgtgtatgtatatatattacaatgaaatactattcagccttaaaaaggcagggaatcctgtcatttaacacaatatggataaacctagaggactctaaaggcaaataccacatgttctcactcacaaaatctaaacaagttgaactcctacaagtagagagtaggatgatggttaccaagggctgggggacgggagaggatggggaaagcatagctgtccatcaaagggtagaaagtttcatttagacaagaggaatcagctttagtgatctatttcac

SEQ ID NO: 40 - DNA, 1071 bp, Artificial sequence
  sequence of STAR40
gctgtgattcaaactgtcagcgagataaggcagcagatcaagaaagcactccgggctccagaaggagccttccaggccagctttgagcataagctgctgatgagcagtgagtgtcttgagtagtgttcagggcagcatgttaccattcatgcttgacttctagccagtgtgacgagaggctggagtcaggtctctagagagttgagcagctccagccttagatctcccagtcttatgcggtgtgcccattcgctttgtgtctgcagtcccctggccacacccagtaacagttctgggatctatgggagtagcttccttagtgagctttcccttcaaatactttgcaaccaggtagagaattttggagtgaaggttttgttcttcgtttcttcacaatatggatatgcatcttcttttgaaaatgttaaagtaaattacctctcttttcagatactgtcttcatgcgaacttggtatcctgtttccatcccagccttctataacccagtaacatcttttttgaaaccagtgggtgagaaagacacctggtcaggaacgcggaccacaggacaactcaggctcacccacggcatcagactaaaggcaaacaaggactctgtataaagtaccggtggcatgtgtattagtggagatgcagcctgtgctctgcagacagggagtcacacagacacttttctataatttcttaagtgctttgaatgttcaagtagaaagtctaacattaaatttgattgaacaattgtatattcatggaatattttggaacggaataccaaaaaatggcaatagtggttctttctggatggaagacaaacttttcttgtttaaaataaattttattttatatatttgaggttgaccacatgaccttaaggatacatatagacagtaaactggttactacagtgaagcaaattaacatatctaccatcgtacatagttacatttttttgtgtgacaggaacagctaaaatctacgtatttaacaaaaatcctaaagacaatacatttttattaactatagccctcatgatgtacattagatctctaa

SEQ ID NO: 41 - DNA, 1135 bp, Artificial sequence
  sequence of STAR41
tgctcttgttgcccaggctgcagtgcaatggcgctgtctcggctcatcgcaacctccgcctcccagattcaagtgattctcctgcctcaccctcccaagtagctgggattaccagtatgcagcaacacgcccggctaattttgtatttgtaatagagacggggtttcttcatgttggtcaggctggtctcaaattcctgccctcaggtgatctgcccaccttggcctcccaaagtgctgggattacaggcatgagccactgtgcccggcctgggctggggcttttaaggggactggagggtgaggggctggaaaattgggagagttgattggtggggcaagggggatgtaatcatcagggtgtacaaactgcactcttggtttagtcagctcctcgtggggtccttcggagcagctcagtcagtagctccatcagtatacaggacccaaaggaatatctcaaagggaaaacagcatttcctaaggttcaagttgtgatctacggagcagttaggggaactacaatcttgtgacagggtctacatgcttctgaggcaatgagacaccaagcagctacgaggaagcagtcagagagcacgccgacctagtgactgatgctgatgtgctgcgagctgggttcattttcatttctcccctccccctgccctcattaattttgtaaagtttatagggaacatttcacccactctgctgtggatccctgtcacttacggagtctgtcatcttggctgtatgggctgtggcctctgcggtgcccattctcaggaggtgtgagacccatgaggaccggaggtggacaaggctagagaccacacccccccgctccatccaatcatgttttcctgggtgcttggtttctatgcaggctgcatgtccttagtccctgcatgggaacagctcctgtggtgagcaggcccctgaggaaggccttgagcgggaatggagcctaggcttaggctgcctggtaagagctggagggaaccagccgaggcttgtgctacttttttttccagaatgaaatacgtgactgatgttggtgtcctgcagcgccacgtttcccgccacaaccaccggaacgaggatgaggagaacacactctccgtggactgcacacg

SEQ ID NO: 42 - DNA, 735 bp, Artificial sequence
  sequence of STAR42
aagggtgagatcactagggagggaggaaggagctataaaagaaagaggtcactcatcacatcttacacactttttaaaaccttggttttttaatgtccgtgttcctcattagcagtaagccctgtggaagcaggagtctttctcattgaccaccatgacaagaccctatttatgaaacataatagacacacaaatgtttatcggatatttattgaaatataggaatttttcccctcacacctcatgaccacattctggtacattgtatgaatgaatataccataattttacctatggctgtatatttaggtcttttcgtgcaggctataaaaatatgtatgggccggtcacagtgacttacgcccgtagtcccagaactttgggaggccgaggcgggtggatcacctgaggtcgggagttcaaaaccagcctgaccaacatggagaaaccccgtctctgctaaaaatacaaaaattaactggacacggtggcgtatgcctgtaatcccagctactcgggaagctgaggcaggagaactgcttgaacccaggaggcggaggttgtggtgagtcgagattgcgccattgcactccagcctgggcaacaagagcgaaattccatctcaaaaaaaagaaaaaagtatgactgtatttagagtagtatgtggatttgaaaaattaataagtgttgccaacttaccttagggtttataccatttatgagggtgtcggtttc

SEQ ID NO: 43 - DNA, 1227 bp, Artificial sequence
  sequence of STAR43
caaatagatctacacaaaacaagataatgtctgcccatttttccaaagataatgtggtgaagtgggtagagagaaatgcatccattctccccacccaacctctgctaaattgtccatgtcacagtactgagaccagggggcttattcccagcgggcagaatgtgcaccaagcacctcttgtctcaatttgcagtctaggccctgctatttgatggtgtgaaggcttgcacctggcatggaaggtccgttttgtacttcttgctttagcagttcaaagagcagggagagctgcgagggcctctgcagcttcagatggatgtggtcagcttgttggaggcgccttctgtggtccattatctccagcccccctgcggtgttgctgtttgcttggcttgtctggctctccatgccttgttggctccaaaatgtcatcatgctgcaccccaggaagaatgtgcaggcccatctcttttatgtgctttgggctattttgattccccgttgggtatattccctaggtaagacccagaagacacaggaggtagttgctttgggagagtttggacctatgggtatgaggtaatagacacagtatcttctctttcatttggtgagactgttagctctggccgcggactgaattccacacagctcacttgggaaaactttattccaaaacatagtcacattgaacattgtggagaatgagggacagagaagaggccctagatttgtacatctgggtgttatgtctataaatagaatgctttggtggtcaactagacttgttcatgttgacatttagtcttgccttttcggtggtgatttaaaaattatgtatatcttgtttggaatatagtggagctatggtgtggcattttcatctggctttttgtttagctcagcccgtcctgttatgggcagccttgaagctcagtagctaatgaagaggtatcctcactccctccagagagcggtcccctcacggctcattgagagtttgtcagcaccttgaaatgagtttaaacttgtttatttttaaaacattcttggttatgaatgtgcctatattgaattactgaacaaccttatggttgtgaagaattgatttggtgctaaggtgtataaatttcaggaccagtgtctctgaagagttcatttagcatgaagtcagcctgtggcaggttgggtggagccagggaacaatggagaagctttcatgggtgg

SEQ ID NO: 44 - DNA, 1586 bp, Artificial sequence
  sequence of STAR44
tgagttggggtcctaagccagaagttaactatgctttcatatattcttgcaagtagaagtacagtgttggtgtaaattccccttagatggatagctaagcccagaggaaataatggtaattggaaccatatgaccgtatgcaattcatgtgcatatttatatcaagaaaagaacattataggtcgggtgagaccctattttgttctgacaatgtcatctgtatttacatgtctgtttcgggagtttggatgtcaagggattctgtgctggattgtaaagcatgtgcttctgcttgatgtagctactcaattttgtattcttgactaataaagtcataaacataattcaacctctgtgtgcgtgctctccttccattaatttatactttagcaaaaagtattgaatgtgtgtgttatgtaacaatttcctataaattatattaaatgatttattagctttattcaataaagttttaagtgttttcttctatgactacattatttgttaacaagaaatttctttaactgaaaacttcaaggaagactatctgggtaactctttcaaaaagaattgtccctgtattttgggattgaatatattaatttcttgtactgttttaacagcacataattttacaagacaagccactttttcaaagcctgcttctcctcccattttccctatctctgtgattgacacctccaacccctgtagcctgcctctgctctctcttaaccagtcctactgatactacttcctaagtatttttcagccctgtccttcctctccatcatgatggattcacttccagttgaaatccttatggtaccctccctggattatggcagtaatcagagagctggtctccttaactcaggattcacttcttctcatctgttgttcacagtgacatcagaaagatattttaaaatgatgaactagaattaattatataaaacacacatacacacataaataatacttaaatttttcaatgatgttccaattatgtaaaatataatataggaggcactttatgttctggcctcaatctttcaattcaaacttatctcctgccactatctcctttgaacattgtattccagctactttagaataataataatacataatattcatagagcccttcctgggttcctatcaccgtacaaaatacttcacatataacatttaatctttgacaactttattaggcatgcacaattattatctatctatatatctatatctatatatataaaatctatattttatagataagaaaatagagggtaaaaacttgccaaaattacaaagcttagaagtgtagcagttgggatttgaatctaggcatcctgcctctatagtctacagtggctttcttgtgccaaaagccttgcagttccctagacttaacatttctcaaaatctgtgtctttcacatgctcttccaattgtctggaaaatctttcccaacctcagtctaactgtggtactcatgttcaccccacaagaattgactccatctgtcccctctccatgaaaatttctttgaatctcagcactttgggaggctgaggcaggtg

SEQ ID NO: 45 - DNA, 1981 bp, Artificial sequence
  sequence of STAR45
cacgccccagcgtgccctggactactgctccgcaggactcctgttctgctgcaccctggactacggcaccagaggacccagctcccgccggcctgagctatggcaccagaggacccagctcccggcagcctggactatggcaccagaggacccagccccccgcttcctgggctaaggcacagtaggaccctgcctcatcgtgtactcctgctcaggaggaccctcgcagggcggcgcactggactaagctactgaaggagccccacccctgcctaaccctggactaaggcactggagaactcttgctccgcagagccacggactcttgcacaagagaacctcagcccagccgtgccctggactgtggcacagtagggcccacaccacgccatggactcctgtattggaggaagagtagtgataaatgtccaggtttacaacttgaaaagtagcaatcaatgtgccacaatagatggatgtgatgtaaaattataaatgatgaaaacattatgtgtaattgcctagccagaacagttacacaagacaaagacgtaaaagaaatccacatagggaaggaagaggtaagattgtttctgttttttgaaaatataatcttaagatagagaaaatcttaaagattccaccaaaataaatggttatagctgatgaagaaattcaataaagttaatagttacaaaatcaacatacaaatatcattattgtttctattaactaatgacaaactattacctgaaaaataaaggcaattcaatttataatagaatcaaaacagatatataaatatataaaagacaggagtaaatttaatcaaaaccataaaagatttacatactgaaaactatagcacattgatgaaaaaaattaaaatggcataaataaatggagaaacatccttcattgatggattcaaaaattagtattgtaaaagtgtcaatgctacccaaagcaatctacagattaaatgcaaccactatcaaattccaatgtcattcttcacagaaatagaaaaattactgctaaaatttgtatggaaccacaaaagacctggaccaaccaaagcaatcttgaacaaaaagaacaaagctggaggcatcagactacctgactccaaactctattacaaagctataggaattaaaacagcatagcaatggcataaaaacagacatgtaaaacagtacaaagggatatagaacctgtaaataaatccgtgtgtctgtggtcaattgattttttgataaaataactaaaaatacacagtgaagaaagaaaattattttcaataaatggtgtagacaaaactgactatccacatacagaagaataaaatttgacttttattttgctctttatacaagcatcaaatcaaaattaaagtttaaatgtaaaactactacaaggaaatatagaaggagactgtatgacattggcctgagctatgattttctgtagattattccaaaaggcaacaaaagcaaaacacacaaatgagactgcataaaacttaaaacttttccacaggaaaagaagcaatgatagaattaagagaacccacaaatgggataatatttttaaaccatacatcaggtaaggggctcatataataatatataagcaactcaacctactcaaaaataagaaaaaaactatgcttattaaaaaataagcaaagaatcagaatagacatttcctacatcatacaaaaggccaaccaggtacatgaaaaaatcataaacattcctaattatcagagaagtgcaaatcaatgccacaatgagatatcacctcacacattttactagggctattataaaaaaagatggaagataagtgttggtgaggatgtggagaaaaagaaaccctgtacactgttggtaggaatggaaattagtacagccatcttggaaaacagtacgaagctttctcaagaaattataaatttatttaccctatgatccat

SEQ ID NO: 46 - DNA, 1859 bp, Artificial sequence
  sequence of STAR46
attgtttttctcgcccttctgcattttctgcaaattctgttgaatcattgcagttacttaggtttgcttcgtctcccccattacaaactacttactgggtttttcaaccctagttccctcatttttatgatttatgctcatttctttgtacacttcgtcttgctccatctcccaactcatggcccctggctttggattattgttttggtcttttattttttgtcttcttctacctcaacacttatcttcctctcccagtctccggtaccctatcaccaaggttgtcattaacctttcatattattcctcattatccatgtattcatttgcaaataagcgtatattaacaaaatcacaggtttatggagatataattcacataccttaaaattcaggcttttaaagtgtacctttcatgtggtttttggtatattcacaaagttatgcattgatcaccaccatctgattccataacatgttcaatacctcaaaaagaagtctgtactcattagtagtcatttcacattcaccactccctctggctctgggcagtcactgatctttgtgtctctatggatttgcctagtctaggtatttttatgtaaatggcatcatacaacatgtgaccttttgtttggcttttttcatttagcaaaatgttatcaaggtctgtccctgttgtagcatgtattagcacttcatttcttatatgctgaatgatatactttatttgtccatcagttgttcatgctttatttgtccatcagttgatgaacatttgcgtttttgccactttgggctattaagaataatgctactgtgaacaagtgtgtacaagttcctctacaaatttttgtgtggacatatcctttcagttctctcaggtgtatatctgggaattgaattgctgggtcgtgtagtagctatgttaaacactttgagaaactgctataatgttctccagagctgtaccattttaaattctgtgtatgaggattccacgttctccacttcctcaccagtgtatggatttgggggtatactttttaaaaagtgggattaggctgggcacagtggctcacacctgtaatcccaacacttcaggaagctgaggtgggaggatcacttgagcctagtagtttgagaccagcctgggcaacatagggagaccctgtctctacaaaaaataatttaaaataaattagctgggcgttgtggcacacacctgtagtcccagctacatgggaggctgaggtggaaggattccctgagcccagaagtttgaggttgcagtgagccatgatggcagcactatactgtagcctgggtgtcagagcaagactccgtttcagggaagaaaaaaaaaagtgggatgatatttttgacacttttcttcttgttttcttaatttcatacttctggaaattccattaaattagctggtaccactctaactcattgtgtttcatggctgcatagtaatattgcataatataaatataccattcattcatcaaagttagcagatattgactgttaggtgccaggcactgctctaagcgttaaagaaaaacacacaaaaacttttgcattcttagagtttattttccaatggagggggtggagggaggtaagaatttaggaaataaattaattacatatatagcatagggtttcaccagtgagtgcagcttgaatcgttggcagctttcttagtagtataaatacagtactaaagatgaaattactctaaatggtgttacttaaattactggaataggtattactattagtcactttgcaggtgaaagtggaaacaccatcgtaaaatgtaaaataggaaacagctggttaatgtt

SEQ ID NO: 47 - DNA, 1082 bp, Artificial sequence
  sequence of STAR47
atcattagtcattagggaaatgcaaatgaaaaacacaagcagccaccaatatacacctactaggatgatttaaaggaaaataagtgtgaagaaggacgtaaagaaattgtaaccctgatacattgatggtagaaatggataaagttgcagccactgtgaaaaacagtctgcagtggctcagaaggttaaatatagaacccctgttggacccaggaactctactcttaggcaccccaaagaatagagaacagaaatcaaacagatgtttgtatactaatgtttgtagcatcacttttcacaggagccaaaaggtggaaataatccaaccatcagtgaacaaatgaatgtaataaaagcaaggtggtctgcatgcaatgctacatcatccatctgtaaaaaacgaacatcattttgatagatgatacaacatgggtggacattgagaacattatgcttagtgaaataagccagacacaaaaggaatatattgtataattgtaattacatgaagtgcctagaatagtcaaattcatacaagagaaagtgggataggaatcaccatgggctggaaatagggggaaggtgctatactgcttattgtggacaaggtttcgtaagaaatcatcaaaattgtgggtgtagatagtggtgttggttatgcaaccctgtgaatatattgaatgccatggagtgcacactttggttaaaaggttcaaatgataaatattgtgttatatatatttccccacgatagaaaacacgcacagccaagcccacatgccagtcttgttagctgccttcctttaccttcaagagtgggctgaagcttgtccaatctttcaaggttgctgaagactgtatgatggaagtcatctgcattgggaaagaaattaatggagagaggagaaaacttgagaatccacactactcaccctgcagggccaagaactctgtctcccatgctttgctgtcctgtctcagtatttcctgtgaccacctcctttttcaactgaagactttgtacctgaaggggttcccaggtttttcacctcggcccttgtcaggactgatcctctcaacta

SEQ ID NO: 48 - DNA, 1242 bp, Artificial sequence
  sequence of STAR48
atcatgtatttgttttctgaattaattcttagatacattaatgttttatgttaccatgaatgtgatattataatataatatttttaattggttgctactgtttataagaatttcattttctgtttactttgccttcatatctgaaaaccttgctgatttgattagtgcatccacaaattttcttggattttctatgggtaattacaaatctccacacaatgaggttgcagtgagccaagatcacaccactgtactccagcctgggcgacagagtgagacaccatctcacaaaaacacataaacaaacaaacagaaactccacacaatgacaacgtatgtgctttctttttttcttcctctttctataatatttctttgtcctatcttaactgaactggccagaaaccccaggacaatgataaatacgagcagtgtcaacagacatctcattccctttcctagcttttataaaaataacgattatgcttcaacattacatatggtggtgtcgatggttttgttatagataagcttatcaggttaagaaatttgtctgcgtttcctagtttggtataaagattttaatataaatgaatgttgtattttatcatcttatttttttcctacatctgctaaggtaatcctgtgttttccccttttcaatctcctaatgtggtgaatgacattaaaataccttctattgttaaaatattcttgcaacgctgtatagaaccaatgcctttattctgtattgctgatggatttttgaaaaatatgtaggtggacttagttttctaaggggaatagaatttctaatatatttaaaatattttgcatgtatgttctgaaggacattggtgtgtcatttctataccatctggctactagaggagccgactgaaagtcacactgccggaggaggggagaggtgctcttccgtttctggtgtctgtagccatctccagtggtagctgcagtgataataatgctgcagtgccgacagttctggaaggagcaacaacagtgatttcagcagcagcagtattgcgggatccccacgatggagcaagggaaataattctggaagcaatgacaatatcagctgtggctatagcagctgagatgtgagttctcacggtggcagcttcaaggacagtagtgatggtccaatggcgcccagacctagaaatgcacatttcctcagcaccggctccagatgctgagcttggacagctgacgcct

SEQ ID NO: 49 - DNA, 1015 bp, Artificial sequence
  sequence of STAR49
aaaccagaaacccaaaacaatgggagtgacatgctaaaaccagaaacccaaaacaatgggagggtcctgctaaaccagaaacccaaaacaatgggagtgaagtgctaaaaccagaaacccaaaacaatgggagtgtcctgctacaccagaaacccaaaacgatgggagtgacgtgataaaaccagacacccaaaacaatgggagtgacgtgctaaaccagaaacccaaaacaatgggagtgacgtgctaaaacctggaaacctaaaacaatgcgagtgaggtgctaacaccagaatccataacaatgtgagtgacgtgctaaaccagaacccaaaacaatgggagtgacgtgctaaaacaggaacccaaaacaatgagagtgacgtgctaaaccagaaacccaaaacaatgggaatgacgtgctaaaaccggaacccaaaacaatgggagtgatgtgctaaaccagaaacccaaaacaatgggaatgacatgctaaaactggaacccaaaacaatggtaactaagagtgatgctaaggccctacattttggtcacactctcaactaagtgagaacttgactgaaaaggaggatttttttttctaagacagagttttggtctgtcccccagagtggagtgcagtggcatgatctcggctcactgcaagctctgcctcccgggttcaggccattctcctgcctcagcctcctgagtagctgggaatacaggcacccgccaccacacttggctaattttttgtatttttagtagagatggggtttcaccatattagcaaggatggtctcaatctcctgacctcgtgatctgcccacctcaggctcccaaagtgctgggattacaggtgtgagccaccacacccagcaaaaaggaggaatttttaaagcaaaattatgggaggccattgttttgaactaagctcatgcaataggtcccaacagaccaaaccaaaccaaaccaaaatggagtcactcatgctaaatgtagcataatcaaa

SEQ ID NO: 50 - DNA, 2355 bp, Artificial sequence
  sequence of STAR50
caaccatcgttccgcaagagcggcttgtttattaaacatgaaatgagggaaaagcctagtagctccattggattgggaagaatggcaaagagagacaggcgtcattttctagaaagcaatcttcacacctgttggtcctcacccattgaatgtcctcacccaatctccaacacagaaatgagtgactgtgtgtgcacatgcgtgtgcatgtgtgaaagtatgagtgtgaatgtgtctatatgggaacatatatgtgattgtatgtgtgtaactatgtgtgactggcagcgtggggagtgctggttggagtgtggtgtgatgtgagtatgcatgagtggctgtgtgtatgactgtggcgggaggcggaaggggagaagcagcaggctcaggtgtcgccagagaggctgggaggaaactataaacctgggcaatttcctcctcatcagcgagcctttcttgggcaataggggcagagctcaaagttcacagagatagtgcctgggaggcatgaggcaaggcggaagtactgcgaggaggggcagagggtctgacacttgaggggttctaatgggaaaggaaagacccacactgaattccacttagccccagaccctgggcccagcggtgccggcttccaaccataccaaccatttccaagtgttgccggcagaagttaacctctcttagcctcagtttccccacctgtaaaatggcagaagtaaccaagcttaccttcccggcagtgtgtgaggatgaaaagagctatgtacgtgatgcacttagaagaaggtctagggtgtgagtggtactcgtctggtgggtgtggagaagacattctaggcaatgaggactggggagagcctggcccatggcttccactcagcaaggtcagtctcttgtcctctgcactcccagccttccagagaggaccttcccaaccagcactccccacgctgccagtcacacatagttacacacatacaatcacatatatgttcccatatagacacattcacactcataccttcacacatgcacacgcatgtgcacacacagtcactcatttctgtgttggagattgggtgaggacattcaatgggtgaggaccaacaggtgtgaagattgctttctagaaaatgactcctgtctctctttgccattcttcccaatccgatggagctactaggcttttccctcatttcatgtttaataaaccttcccaatggcgaaatgggctttctcaagaagtggtgagtgtcccatccctgcggtggggacaggggtggcagcggacaagcctgcctggagggaactgtcaggctgattcccagtccaactccagcttccaacacctcatcctccaggcagtcttcattcttggctctaatttcgctcttgttttcttttttatttttatcgagaactgggtggagagcttttggtgtcattggggattgctttgaaacccttctctgcctcacactgggagctggcttgagtcaactggtctccatggaatttctttttttagtgtgtaaacagctaagttttaggcagctgttgtgccgtccagggtggaaagcagcctgttgatgtggaactgcttggctcagatttcttgggcaaacagatgccgtgtctctcaactcaccaattaagaagcccagaaaatgtggcttggagaccacatgtctggttatgtctagtaattcagatggcttcacctgggaagccctttctgaatgtcaaagccatgagataaaggacatatatatagtagctagggtggtccacttcttaggggccatctccggaggtggtgagcactaagtgccaggaagagaggaaactctgttttggagccaaagcataaaaaaaccttagccacaaaccactgaacatttgttttgtgcaggttctgagtccagggagggcttctgaggagaggggcagctggagctggtaggagttatgtgagatggagcaagggccctttaagaggtgggagcagcatgagcaaaggcagagaggtggtaatgtataaggtatgtcatgggaaagagtttggctggaacagagtttacagaatagaaaaattcaacactattaattgagcctctactacgtgctcgacattgttctagtcactgagataggtttggtatacaaaacaaaatccatcctctatggacattttagtgactaacaacaatataaataataaaagtgaacaaaagctcaaaacatgccaggcactattatttatttatttatttatttatttatttattttttgaaacagagtctcgctctgttgcccaggctggagtgtagtggtgcgatctcggctcactg

SEQ ID NO: 51 - DNA, 2289 bp, Artificial sequence
  sequence of STAR51
tcacaggtgacaccaatcccctgaccacgctttgagaagcactgtactagattgactttctaatgtcagtcttcattttctagctctgttacagccatggtctccatattatctagtacaacacacatacaaatatgtgtgatacagtatgaatataatataaaaatatgtgttataatataaatataatattaaaatatgtctttatactagataataatacttaataacgttgagtgtttaactgctctaagcactttacctgcaggaaacagttttttttttattttggtgaaatacaactaacataaatttatttacaattttaagcatttttaagtgtatagtttagtggagttaatatattcaaaatgttgtgcagccgtcaccatcatcagtcttcataactcttttcatattgtaaaattaaaagtttatgctcatttaaaaatgactcccaatttcccccctcctcaacctctggaaactaccattctattttctgcctccgtagttttgcccactctaagtacctcacataagtggaatttgtcttatttgcctgtttgtgaccggctgatttcatttagtataatgtcctcaagttttattcacgttatatagcatatgtcataattttcttcacttttaagcttgagtaatatttcatcgtatgtatctcacattttgcttatccattcatctctcagtggacacttgagttgcttctacattttagctgttgtgaatactgctgctatgaacatgggtgtataaatatctcaagacctttttatcagttttttaaaatatatactcagtagtagtttagctggattatatggtaattttatttttaatttttgaggaactgtcctacccttttattcaatagtagctataccaattgacaattggcattcctaccaacagggcataagggttctcaattctccacatattccctgatacttgttattttcaggtgttttttttttttttttttttttatgggagccatgttaatgggtgtaaggtgatatttcattatagttttgatttgcatttccctaatgattagtgatgttaagcatctcttcatgtgcctattggccatttgtatatcttctttaaaaatatatatatactcattcctttgcccatttttgaattatgtttattttttgttattgagtttcaatacttttctatataacctaggtattaatcctttatcagacttaagatttgcaaatattctctttcattccacaggttgctaattctctctgttggtaatatcttttgatgctgttgtgtccagaattgattcattcctgtgggttcttggtctcactgacttcaagaataaagctgcggaccctagtggtgagtgttacacttcttatagatggtgtttccggagtttgttccttcagatgtgtccagagtttcttccttccaatgggttcatggtcttgctgacttcaggaatgaagccgcagaccttcgcagtgaggtttacagctcttaaaggtggcgtgtccagagttgtttgttccccctggtgggttcgtggtcttgctgacttcaggaatgaagccgcagaccctcgcagtgagtgttacagctcataaaggtagtgcggacacagagtgagctgcagcaagatttactgtgaagagcaaaagaacaaagcttccacagcatagaaggacaccccagcgggttcctgctgctggctcaggtggccagttattattcccttatttgccctgcccacatcctgctgattggtccattttacagagtactgattggtccattttacagagtgctgattggtgcatttacaatcctttagctagacacagagtgctgattgctgcattcttacagagtgctgattggtgcatttacagtcctttagctagatacagaacgctgattgctgcgttttttacagagtgctgattggtgcatttacaatcctttagctagacacagtgctgattggtgggtttttacagagtgctgattggtgcgtctttacagagtgctgattggtgcatttacaatcctttagctagacacagagtgctgattggtgcgtttataatcctctagctagacagaaaagttttccaagtccccacctgaccgagaagccccactggcttcacctctcactgttatactttggacatttgtccccccaaaatctcatgttgaaatgtaacccctaatgttggaactgaggccagactggatgtggctgggccatgggga

SEQ ID NO: 52 - DNA, 1184 bp, Artificial sequence
  sequence of STAR52
cttatgccatctggcggtgccatgtggaacttcgctgaagaagctaaatttactgaccatctgtgcctagagcgggtttctccaaggaaaggctctgtaaatctcgtccttttgaaatctaggggaaaacagcctccttcactgaggattaatttaaagaaagggggaaataggaaaattccatgcgttggaagtccatttagatttctacatgaaccatcatatatgtgcactacataattcttatttttttatttttaaaaaagggataatttatattccagtgacaagtttgggaaaggccaaggcaagcaattgagttgaacattatgtagcgtttatatagaccttgcagacgtctgtgcaatatccaccactgaacacgtgaggtcgtactcaagtctctctggcccctggtaatgtgactcccttcctttatttgcatgaatcgcctggattgggtgtcaggtttttaaaacgtcaaggtttacgcctattgttgtcaaccaatcagcatcctactttgacgtgattggcttctactgtaggtgtcaatcatccaaaatttgcatactactcctcaggccgccgggagcctgtcagtcggctgtggcagctggaagagaaggaatcggacggagaagaatgaaaaatcactttgctttcgcaaagcgaaagaaaagtattcttttcctcattatttttaaataaatttgattgtatatttacctaataaaataaacattcaattaaacaaaaataagcaactatcaaagatttgtttactaattttcgtaatgtttactgtttcaataagtagccaaaggaatattaaaacacaaaaatatgaatgctgataattttatgtcataaagaccattttaaaactaaaagtgaacatggggtttctaaataaaattaccgtggtagcgtaaaaacactgctttcaatacttgggcatgctgaaagtgctgcatcctaagataaaaaatacaccaagggggggatttcaaagaacattattttgcttttaataatcctgtatttctgtcactttgccctttttatttatttaccgtgaactcacagacagaatattacttggagtttctgaaatacttgtgtttgtacatttctcatcttacacgtacccacacaccccaaaataaaaaaacaaagaagag

SEQ ID NO: 53 - DNA, 1431 bp, Artificial sequence
  sequence of STAR53
ccctgaggaagatgacgagtaactccgtaagagaaccttccactcatcccccacatccctgcagacgtgctattctgttatgatactggtatcccatctgtcacttgctccccaaatcattcccttcttacaattttctactgtacagcattgaggctgaacgatgagagatttcccatgctctttctactccctgccctgtatatatccggggatcctccctacccaggatgctgtggggtcccaaaccccaagtaagccctgatatgcgggccacacctttctctagcctaggaattgataacccaggcgaggaagtcactgtggcatgaacagatggttcacttcgaggaaccgtggaaggcgtgtgcaggtcctgagatagggcagaatcggagtgtgcagggtctgcaggtcaggaggagttgagattgcgttgccacgtggtgggaactcactgccacttatttccttctctcttcttgcctcagcctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttccccagttgtttgctcgtgccattattgtgcacacagctctgtgcacgtgtgtgcatatttctttaggaaagattcttagaagtggaattgctgtgtcaaaggagtcatttattcaacaaaacactaatgagtgcgtcctcgtgctgagcgctgttctaggtgctggagcgacgtcagggaacaaggcagacaggagttcctgacccccgttctagaggaggatgtttccagttgttgggttttgtttgtttgtttcttctagagatggtggtcttgctctgtccaggctagagtgcagtggcatgatcatagc

SEQ ID NO: 54 - DNA, 975 bp, Artificial sequence
  sequence of STAR54
ccataaaagtgtttctaaactgcagaaaaatccccctacagtcttacagttcaagaattttcagcatgaaatgcctggtagattacctgactttttttgccaaaaataaggcacagcagctctctcctgactctgactttctatagtccttactgaattatagtccttactgaattcattcttcagtgttgcagtctgaaggacacccacattttctctttgtctttgtcaattctttgtgttgtaagggcaggatgtttaaaagttgaagtcattgacttgcaaaatgagaaatttcagagggcattttgttctctagaccatgtagcttagagcagtgttcacactgaggttgctgctaatgtttctgcagttcttaccaatagtatcatttacccagcaacaggatatgatagaggacttcgaaaaccccagaaaatgttttgccatatatccaaagccctttgggaaatggaaaggaattgcgggctcccatttttatatatggatagatagagaccaagaaagaccaaggcaactccatgtgctttacattaataaagtacaaaatgttaacatgtaggaagtctaggcgaagtttatgtgagaattctttacactaattttgcaacattttaatgcaagtctgaaattatgtcaaaataagtaaaaatttttacaagttaagcagagaataacaatgattagtcagagaaataagtagcaaaatcttcttctcagtattgacttggttgcttttcaatctctgaggacacagcagtcttcgcttccaaatccacaagtcacatcagtgaggagactcagctgagactttggctaatgttggggggtccctcctgtgtctccccaggcgcagtgagcctgcaggccgacctcactcgtggcacacaactaaatctggggagaagcaacccgatgccagcatgatgcagatatctcagggtatgatcggcc

SEQ ID NO: 55 - DNA, 501 bp, Artificial sequence
  sequence of STAR55
cctgaactcatgatccgcccacctcagcctcctgaagtgctgggattacaggtgtgagccaccacacccagccgcaacacactcttgagcaaccaatgtgtcataaaagaaataaaatggaaatcagaaagtatcttgagacagacaaaaatggaaacacaacataccaaaatttatgggacacagcaaaagcagttttaggagggaagtttatagtgatgaatacctacctcaaaatcattagcctgattggatgacactacagtgtataaatgaattgaaaaccacattgtgccccatacatatatacaatttttatttgttaattaaaaataaaataaaactttaaaaaagaagaaagagctcaaataaacaacctaactttatacctcaaggaaatagaagagccagctaagcccaaagttgacagaaggaaaaaaatattggcagaaagaaatgaaacagagactagaaagacaattgaagagatcagcaaaacta

SEQ ID NO: 56 - DNA, 741 bp, Artificial sequence
  sequence of STAR56
acacaggaaaagatcgcaattgttcagcagagctttgaaccggggatgacggtctccctcgttgcccggcaacatggtgtagcagccagccagttatttctctggcgtaagcaataccaggaaggaagtcttactgctgtcgccgccggagaacaggttgttcctgcctctgaacttgctgccgccatgaagcagattaaagaactccagcgcctgctcggcaagaaaacgatggaaaatgaactcctcaaagaagccgttgaatatggacgggcaaaaaagtggatagcgcacgcgcccttattgcccggggatggggagtaagcttagtcagccgttgtctccgggtgtcgcgtgcgcagttgcacgtcattctcagacgaaccgatgactggatggatggccgccgcagtcgtcacactgatgatacggatgtgcttctccgtatacaccatgttatcggagagctgccaacgtatggttatcgtcgggtatgggcgctgcttcgcagacaggcagaacttgatggtatgcctgcgatcaatgccaaacgtgtttaccggatcatgcgccagaatgcgctgttgcttgagcgaaaacctgctgtaccgccatcgaaacgggcacatacaggcagagtggccgtgaaagaaagcaatcagcgatggtgctctgacgggttcgagttctgctgtgataacggagagagactgcgtgtcacgttcgcgctggactgctgtg

SEQ ID NO: 57 - DNA, 1365 bp, Artificial sequence
  sequence of STAR57
tccttctgtaaataggcaaaatgtattttagtttccaccacacatgttcttttctgtagggcttgtatgttggaaattttatccaattattcaattaacactataccaacaatctgctaattctggagatgtggcagtgaataaaaaagttatagtttctgattttgtggagcttggactttaatgatggacaaaacaacacattcttaaatatatatttcatcaaaattatagtgggtgaattatttatatgtgcatttacatgtgtatgtatacataaatgggcggttactggctgcactgagaatgtacacgtggcgcgaacgaggctgggcggtcagagaaggcctcccaaggaggtggctttgaagctgagtggtgcttccacgtgaaaaggctggaaagggcattccaagaaaaggctgaggccagcgggaaagaggttccagtgcgctctgggaacggaaagcgcacctgcctgaaacgaaaatgagtgtgctgaaataggacgctagaaagggaggcagaggctggcaaaagcgaccgaggaggagctcaaaggagcgagcggggaaggccgctgtggagcctggaggaagcacttcggaagcgcttctgagcgggtaaggccgctgggagcatgaactgctgagcaggtgtgtccagaattcgtgggttcttggtctcactgacttcaagaatgaagagggaccgcggaccctcgcggtgagtgttacagctcttaaggtggcgcgtctggagtttgttccttctgatgttcggatgtgttcagagtttcttccttctggtgggttcgtggtctcgctggctcaggagtgaagctgcagaccttcgcggtgagtgttacagctcataaaagcagggtggactcaaagagtgagcagcagcaagatttattgcaaagaatgaaagaacaaagcttccacactgtggaaggggaccccagcgggttgccactgctggctccgcagcctgcttttattctcttatctggccccacccacatcctgctgattggtagagccgaatggtctgttttgacggcgctgattggtgcgtttacaatccctgcgctagatacaaaggttctccacgtccccaccagattagctagatagagtctccacacaaaggttctccaaggccccaccagagtagctagatacagagtgttgattggtgcattcacaaaccctgagctagacacagggtgatgactggtgtgtttacaaaccttgcggtagatacagagtatcaattggcgtatttacaatcactgagctaggcataaaggttctccaggtccccaccagactcaggagcccagctggcttcacccagtgg

SEQ ID NO: 58 - DNA, 1401 bp, Artificial sequence
  sequence of STAR58
aagtttaccttagccctaaattatttcattgtgattggcattttaggaaatatgtattaaggaatgtctcttaggagataaggataacatatgtctaagaaaattatattgaaatattattacatgaactaaaatgttagaactgaaaaaaaattattgtaactccttccagcgtaggcaggagtatctagataccaactttaacaactcaactttaacaacttcgaaccaaccagatggctaggagattcacctatttagcatgatatcttttattgataaaaaaatataaaacttccattaaatttttaagctactacaatcctattaaattttaacttaccagtgttctcaatgctacataatttaaaatcattgaaatcttctgattttaactcctcagtcttgaaatctacttatttttagttacatatatatccaatctactgccgctagtagaagaagcttggaatttgagaaaaaaatcagacgttttgtatattctcatattcactaatttattttttaaatgagtttctgcaatgcatcaagcagtggcaaaacaggagaaaaattaaaattggttgaaaagatatgtgtgccaaacaatcccttgaaatttgatgaagtgactaatcctgagttattgtttcaaatgtgtacctgtttatacaagggtatcacctttgaaatctcaacattaaatgaaattttataagcaatttgttgtaacatgattattataaaattctgatataacattttttattacctgtttagagtttaaagagagaaaaggagttaagaataattacattttcattagcattgtccgggtgcaaaaacttctaacactatcttcaaatctttttctccattgccttctgaacatacccacttgggtatctcattagcactgcaaattcaacattttcgattgctaatttttctccctaaatatttatttgttttctcagctttagccaatgtttcactattgaccatttgctcaagtatagtgacgcttcaatgaccttcagagagctgtttcagtccttcctggactacttgcatgcttccaacaaaatgaagcactcttgatgtcagtcactcaaataaatggaaatgggcccatttactaggaatgttaacagaataaaaagatagacgtgacaccagttgcttcagtccatctccatttacttgcttaaggcctggccatatttctcacagttgatatggcgcagggcacatgtttaaatggctgttcttgtaggatggtttgactgttggattcctcatcttccctctccttaggaaggaaggttacagtagtactgttggctcctggaatatagattcataaagaactaatggagtatcatctcccactgctcttgt

SEQ ID NO: 59 - DNA, 866 bp, Artificial sequence
  sequence of STAR59
gagatcacgccactgcactccagcctgggggacagagcaagactccatctcagaaacaaacaaacacacaaagccagtcaaggtgtttaattcgacggtgtcaggctcaggtctcttgacaggatacatccagcacccgggggaaacgtcgatgggtggggtggaatctattttgtggcctcaagggagggtttgagaggtagtcccgcaagcggtgatggcctaaggaagcccctccgcccaagaagcgatattcatttctagcctgtagccacccaagagggagaatcgggctcgccacagaccccacaacccccaacccaccccacccccacccctcccacctcgtgaaatgggctctcgctccgtcaggctctagtcacaccgtgtggttttggaacctccagcgtgtgtgcgtgggttgcgtggtggggtggggccggctgtggacagaggaggggataaagcggcggtgtcccgcgggtgcccgggacgtggggcgtggggcgtgggtggggtggccagagccttgggaactcgtcgcctgtcgggacgtctcccctcctggtcccctctctgacctacgctccacatcttcgccgttcagtggggaccttgtgggtggaagtcaccatccctttggactttagccgacgaaggccgggctcccaagagtctccccggaggcggggccttgggcaggctcacaaggatgctgacggtgacggttggtgacggtgatgtacttcggaggcctcgggccaatgcagaggtatccatttgacctcggtgggacaggtcagctttgcggagtcccgtgcgtccttccagagactcatccagcgctagcaagcatggtcccgagg

SEQ ID NO: 60 - DNA, 2067 bp, Artificial sequence
  sequence of STAR60
agcagtgcagaactggggaagaagaagagtccctacaccacttaatactcaaaagtactcgcaaaaaataacacccctcaccaggtggcatnattactctccttcattgagaaaattaggaaactggacttcgtagaagctaattgctttatccagagccacctgcatacaaacctgcagcgccacctgcatacaaacctgtcagccgaccccaaagccctcagtcgcaccaagcctctgctgcacaccctcgtgccttcacactggccgttccccaagcctggggcatactncccagctctgagaaatgtattcatccttcaaagccctgctcatgtgtcctnntcaacaggaaaatctcccatgagatgctctgctatccccatctctcctgccccatagcttaggcanacttctgtggtggtgagtcctgggctgtgctgtgatgtgttcgcctgcnatgtntgttcttccccacaatgatgggcccctgaattctctatctctagcacctgtgctcagtaaaggcttgggaaaccaggctcaaagcctggcccagatgccaccttttccagggtgcttccgggggccaccaaccagagtgcagccttctcctccaccaggaactcttgcagccccacccctgagcacctgcaccccattacccatctttgtttctccgtgtgatcgtattattacagaattatatactgtattcttaatacagtatataattgtataattattcttaatacagtatataattatacaaatacaaaatatgtgttaatggaccgtttatgttactggtaaagctttaagtcaacagtgggacattagttaggtttttggcgaagtcaaaagttatatgtgcattttcaacttcttgaggggtcggtacntctnacccccatgttgttcaanggtcaactgtctacacatatcatagctaattcactacagaaatgttagcttgtgtcactagtatctccccttctcataagcttaatacacataccttgagagagctcttggccatctctactaatgactgaagtttttatttattatagatgtcataataggcataaaactacattacatcattcgagtgccaattttgccaccttgaccctcttttgcaaaacaccaacgtcagtacacatatgaagaggaaactgcccgagaactgaagttcctgagaccaggagctgcaggcgttagatagaatatggtgacgagagttacgaggatgacgagagtaaatacttcatactcagtacgtgccaagcactgctataagcgctctgtatgtgtgaagtcatttaatcctcacagcatcccacggtgtaattattttcattatccccatgagggaacagaaactcagaacggttcaacacatatgcgagaagtcgcagccggtcagtgagagagcaggttcccgtccaagcagtcagaccccgagtgcacactctcgacccctgtccagcagactcactcgtcataaggcggggagtgntctgtttcagccagatgctttatgcatctcagagtacccaaaccatgaaagaatgaggcagtattcangagcagatggngctgggcagtaaggctgggcttcagaatagctggaaagctcaagtnatgggacctgcaagaaaaatccattgtttngataaatagccaaagtccctaggctgtaaggggaaggtgtgccaggtgcaagtggagctctaatgtaaaatcgcacctgagtctcctggtcttatgagtnctgggtgtaccccagtgaaaggtcctgctgccaccaagtgggccatggttcagctgtgtaagtgctgagcggcagccggaccgcttcctctaacttcacctccaaaggcacagtgcacctggttcctccagcactcagctgcgaggcccctagccagggtcccggcccccggcccccggcagctgctccagcttccttccccacagcattcaggatggtctgcgttcatgtagacctttgttttcagtctgtgctccgaggtcactggcagcactagccccggctcctgt

SEQ ID NO: 61 - DNA, 1470 bp, Artificial sequence
  sequence of STAR61
cagcccccacatgcccagccctgtgctcagctctgcagcggggcatggtgggcagagacacagaggccaaggccctgcttcggggacggtgggcctgggatgagcatggccttggccttcgccgagagtnctcttgtgaaggaggggtcaggaggggctgctgcagctggggaggagggcgatggcactgtggcangaagtgaantagtgtgggtgcctngcaccccaggcacggccagcctggggtatggacccggggccntctgttctagagcaggaaggtatggtgaggacctcaaaaggacagccactggagagctccaggcagaggnacttgagaggccctggggccatcctgtctcttttctgggtctgtgtgctctgggcctgggcccttcctctgctcccccgggcttggagagggctggccttgcctcgtgcaaaggaccactctagactggtaccaagtctggcccatggcctcctgtgggtgcaggcctgtgcgggtgacctgagagccagggctggcaggtcagagtcaggagagggatggcagtggatgccctgtgcaggatctgcctaatcatggtgaggctggaggaatccaaagtgggcatgcactctgcactcatttctttattcatgtgtgcccatcccaacaagcagggagcctggccaggagggcccctgggagaaggcactgatgggctgtgttccatttaggaaggatggacggttgtgagacgggtaagtcagaacgggctgcccacctcggccgagagggccccgtggtgggttggcaccatctgggcctggagagctgctcaggaggctctctagggctgggtgaccaggnctggggtacagtagccatgggagcaggtgcttacctggggctgtccctgagcaggggctgcattgggtgctctgtgagcacacacttctctattcacctgagtcccnctgagtgatgagnacacccttgttttgcagatgaatctgagcatggagatgttaagtggcttgcctgagccacacagcagatggatggtgtagctgggacctgagggcaggcagtcccagcccgaggacttcccaaggttgtggcaaactctgacagcatgaccccagggaacacccatctcagctctggtcagacactgcggagttgtgttgtaacccacacagctggagacagccaccctagccccacccttatcctctcccaaaggaacctgccctttcccttcattttcctcttactgcattgagggaccacacagtgtggcagaaggaacatgggttcaggacccagatggacttgcttcacagtgcagccctcctgtcctcttgcagagtgcgtcttccactgtgaagttgggacagtcacaccaactcaatactgctgggcccgtcacacggtgggcaggcaacggatggcagtcactggctgtgggtctgcagaggtgg

SEQ ID NO: 62 - DNA, 1011 bp, Artificial sequence
  sequence of STAR62
agtgtcaaatagatctacacaaaacaagataatgtctgcccatttttccaaagataatgtggtgaagtgggtagagagaaatgcatccattctccccacccaacctctgctaaattgtccatgtcacagtactgagaccagggggcttattcccagcgggcagaatgtgcaccaagcacctcttgtctcaatttgcagtctaggccctgctatttgatggtgtgaaggcttgcacctggcatggaaggtccgttttgtacttcttgctttagcagttcaaagagcagggagagctgcgagggcctctgcagcttcagatggatgtggtcagcttgttggaggcgccttctgtggtccattatctccagcccccctgcggtgttgctgtttgcttggcttgtctggctctccatgccttgttggctccaaaatgtcatcatgctgcaccccaggaagaatgtgcaggcccatctcttttatgtgctttgggctattttgattccccgttgggtatattccctaggtaagacccagaagacacaggaggtagttgctttgggagagtttggacctatgggtatgaggtaatagacacagtatcttctctttcatttggtgagactgttagctctggccgcggactgaattccacacagctcacttgggaaaactttattccaaaacatagtcacattgaacattgtggagaatgagggacagagaagaggccctagatttgtacatctgggtgttatgtctataaatagaatgctttggtggtcaactagacttgttcatgttgacatttagtcttgccttttcggtggtgatttaaaaattatgtatatcttgtttggaatatagtggagctatggtgtggcattttcatctggctttttgtttagctcagcccgtcctgttatgggcagccttgaagctcagtagctaatgaagaggtatcctcactccctccagagagcggtcccctcacggctcattgagagtttgtca

SEQ ID NO: 63 - DNA, 1410 bp, Artificial sequence
  sequence of STAR63
gcgtctgagccgctgggaacccatgagccccgtccatggagttgaggaagggggttcgccccacggggtgggcgccctctacacagcgcgcttcctcttctctcgttagcgccgcgggaccagcctctggttctgcacctcgcgctctgggagcagcgcccggctttggcgagcgcttccccggggctgcccagcctctgctccgctcgccccgccaggcccggctccgcgaagcccccagggtccagtccaaggccccgattccccaaggccagggccccggggcagcattggaacagggcgcggacgccagtcctccgagcatggagtaactgcagcttttgagaaaagaaagcggaccccaccccatcgagaacgcggcgccttgtttagggacgttcctgggccgtcacggagtgtcgccggctcctcggcccctccctcctccaagcccccacccccgacagcggcctccctggggacctcccctcgggctgcgctttcagcccaaacacagggaggtcttccaggagcctgcccagtccccacagcagcccagagacccccactcccacctgtacctgccaagccttcagagagggcggcctggacatgccccgcacgggaggagccccgcctcagcacccctgcaagtggcagcaacccagaacacccgtgagaggcctctgagcagcccaggaagtggctggaagacgcataggcagctcactcctctgtaagagcaaggaccggagaacacatgctgacccctgcttttgcagaggggcgatgcttcaggacaggcgcgctcagcaggtgtccatcttatttcacacctttgtgtttatatcatcttattttgcattttatgtctaattaacaatatgcagctggccaggcgcagtggctcaagcctctaatcccagcactttgggaggccgaggcaggtgtatcacttgagggcaggagttcgggaccggcctgggcaacatagcaaaaccccattgctaataaaaatacaaaaattagccagccatggtggcgggcacctgcagtcccagctactccggaagctgaagcaggagaatcacttgaacccaggaggcggaggtggcagtgagctatcaagccattacactccagcctgggcaacagagaaagactgtctcaaaaaaaaattaatacgcagcagaatattatgtggtcagcccaagcagtcccccccactcagccctctgtccctacagctccaggcactcccccagcccctcccctggacaagaggtaatgcccagagggtgaaaatccaccaaggttaagccagaaacaaaaagctcaaagcttcggcatctccctccgctcagacccttagagcagattcctctcatcgacagcacgatcaggctgtgg

SEQ ID NO: 64 - DNA, 1414 bp, Artificial sequence
  sequence of STAR64
agagatcttttaagggctcaaaagaccctgcggctcccctgccaatagctctgccatcgtccccagagctttcgaggaccctccaccatcggcgccaaccccagctgagctgggtgctcgtctgcaggcctctgctccatctcagcctgagcatgaggctctgctgtgctgcttccagcagcagggacagggctgatgagcctggcccttgcaagcatcttcctgtgccgaatacaattccacagacagaggatttaaaatccaagtggaggtgacaggaaagaaaggaaaacctccaggtatcagaagaaaggagggggtgtgaagacagtatgggaggaaggtcaggctggggctcagctctgggaagtgccagcctgaacaggagtcacgcccgggtccacatgcaagggaatgaggaccgaggccctgcatgtggcagggccttccgcaggctgccccgtctgtgaacaggacaccagaagaagtctgccttccagcctggcaaagtggcaaggaacctctgggtgggaaaacaaatcaacaaacaaattgtcagtaaaaaacagaaacctcacactttcctttctcttgacctcttgaaaaaagcaaatccactgcagctcaccaaaggcaaagagaaaaccttaagaatacccagagagaaaagacacgttacttgcaaaagaacatctaatgcagggagataatgaaaatacagactcttcaaagggctgaaggaaaaaaaccgtccacctagaattctatccccaaactgtcatctgagagcaagggcaaaacaaacgctttctcagacaggctggacgaggtcgctcacgcctgtaatcctagcactttgggaggccaaggtgggaggaccgctttaagccagaagtttgagaccagtgtgggtaacataatgagaccccatctctaagaaaaagaaattaaataagacaagactttttcagacaacaagtgctctgagagctggcctatcttggctgtcttgtaaagaattgctgcgagacacctcattaggaaagagactgaatctagaaggaaagagcagagcatgaggtacaatgaggagcaaataaacaggtcaccatataagcaaacccaaatacacattcactatacgaaacaataaaaatgactcatttggggggttaaaacactgttgaactaaaatcctggataacagcagcatgaaaggtggggtggtggtcccaggaaagcattcaaaggtccatgtctcatttgggaggagggtagggagactcatgaacttgaggctcccttcaggcaagcacagtgcaaaaaaattataataatgggaaacagatacagtagactgtgatgtacaactctcagagcagtagaagggagggtataaaacaaatctgatcca

SEQ ID NO: 65 - DNA, 1310 bp, Artificial sequence
  sequence of STAR65
tcgagaccagcctggccaacatggtgaaacattgtctctattacaaatacaaaaattagccaggtgtagtggtgcatgcctgcagtcccagccatttgggaggctgaagttggagaatcgcttaaacctgggaggtggaggttgcattgagccgagaagcactccagcctggatgacggagcaagactgtctcaaaaagaaaaaaaaaagaagcagcagcaaatatccctgtcctgatggaggctatataacaaccaaacaagtgaatgcataagacaatttcaaggttatggtagataccataagtgggagatgaacaatgagaacacatggacacagggaggagaacatcacacactggggcctctcggggggtggggaaatagggggtgatagcattaggagaaatacctaatgtcgataacaggttagtgggtgcagcaaaccaccatggcacgtgtatatctatgtaacacacctgcacgttctgcacatgtatcccagaacttaaagtataataaaaaaagacattaaaaaattatgatataaaatcccaattcaagttgttttaaaaagagaaaacaattatctttatataatagcggaaaatatagatggcggaattaaagcctcgtcatattttctaacagaactttctgataaacttgattaaataaaaattttaaatatcactaaacacatagaagaaataaatttaaaccttcacaaaaaataaagtacaatgaatgaagacaaggtgtacttgaaaaaagaactgaataaatattctacatataaaaaaaatctgatgatattgtggtgattctttactttgctactagtttctctttttttcttctgaaaaatttcttgggatgtatttggtttcattagtaaaattctaagtttctttgcaatctgaacattggagcttcatccatagccagtatgccctaacattatctttggacaactgtaaaattagaacactgccagacatatttaatgtatgatgtatatcaacactgggacacattttatactatctttattccaaaatcaaatgattcactgtggtttataaatgtacatggatatatctctacctaagcagatagttaggagagttagtaaaaatgaggtggaaaataggagtcactgtcccttcacagggagagaattctgcttttctcctaatataccctttgcttgaacagactccaacccctcatcttttgtcctttaaatgaccacatttattttaactttgataaacaacacagaaagatatttgatccatcaacattcac

SEQ ID NO: 66 - DNA, 2500 bp, Artificial sequence
  sequence of T2F (STAR66F)
gcaggttggatggtgctgacccctcctcgggttggcttcctgtctccaggtggacgtcctgtactccagggtctgcaagcctaaaaggagggacccaggacccaccacagacccgctggaccccaagggccagggagcgattctggccctggcgggtgacctggcctaccagaccctcccgctcagggccctggatgtggacagcggccccctggaaaacgtgtatgagagcatccgggagctgggggaccctgctggcaggagcagcacgtgcggggctgggacgccccctgcttccagctgccccagcctagggaggggctggagacccctccctgcctccctgccctgaacactcaaggacctgtgctccttcctccagagtgaggcccgtcccccgccccgccccgcctcacagctgacagcgccagtcccaggtccccgggctgccagcccgtgaggtccgtgaggtcctggccgctctgacagccgcggcctccccgggctccagagaaggcccgcgtctaaataaagcgccagcgcaggatgaaagcggccagcctcgcagcctgctcttcttgaaagctgggcgggttggggcggggggcttctctggaaggcttggagctgtcccctctggccttgggggactggctgcccccggggcgcccgggcctagccgaggcggtgctcctgccggccagactctcggtcagtgcgggcacggggtcccagccactcctagggggcagcgcagccggcagggtggccgcccccgggtgggacttggaccctggactccacgggagggctccgccacccagcctggtgttacataaggggtggtggaggtgggcagtcgagcgttaaagagtaacctgctgccgggaagcccgccaagcaatcgcggccccttccccggctctggcagctctgcgagcgcgcccgtggggaacgggccctccccggcggggcgcgcgggcgcgcgaggtgggcggaggcctcggagctgtgccgggccgggcctccctccctaggccagcgcgggagcgacccggagggggcgggcccggggcggggcctcgaagcgctggccggcgggagcgcggccggccgggcccgcccgcctgcggtgtggacgccgcgcggccaatgcgcgcgccgggacgggacgggacggggcggggcggggcgggacgagacggggcggggcggggcgggccgggcagcctccgggcggcgcggcgcgggcggcggccggatccagggcgggggtcggcggcccggccagcccggcccggcccggggccgcgtcctgagagtcagccctcgccgctgcagcctcggcgcccggccggccggccatggagcgccccccgccccgcgccgccggccgggaccccagtgcgctgcgggccgaggcgccgtggctgcgcgcggagggtccggggccgcgcgccgcgcccgtgacggtgcccacgccgccgcaggtaccgggcgccggtgggcgggggcgccgaccaagtttctctcgctgcaaagatggcgtcagtgctgcccaaacttcgggcccccgggggcggggcagcggggagggcggccgcgtcggtccgcgcgtgtccgtgggtcccgccggggctgcgccgggcggccggggagcccttcccgccgcgccgggctgggggcggggccgggggcggggccgcgccgtccacaccggccgcagccggttttcgaggcgggcgccgagcggatccgcggcggaggttgagggacccccctcccccggccaccgcctccgctgagtctgccccctccccatccgcagggctcttccgtgggcggcggcttcgcgggcttggagttcgcgcggccgcaggagtcggagccgcgggcctcggacctgggggccccccggacgtggacgggggcggcggcggggccccggactccgtcggcgcacatccccgtcccagcgcagaggtgagcgggaggcccggtgcctcgggactcggtgtgcgcaggggcggtgggtggggtgcggagacaccggccccgacggaggccaggtcagggccccaggtttgtaattaccagccacccccaagctcttcagccctggaggagctgagcagaaatgatcgatgactgggagtccctacacctccctccaccgcagttcctcggggctagagctcagaacccggagcgggtggctgtgcgtctctgtgcagaagaggctgcgcggtcggcatggggcgactgtccaggaatccctggggctcctgaccgccacctcccaacccctgccaggccggacacctcggtctggctgccagggcaggggcgggccctggcctggctcgctggggcctggggagctgcccgtgcttccagcccagtctccccctggctgctgccggctgctggccactcccacctcccaggcctggcgtgaggcccacagctgctgttgcacaaccctggttaatgtgtgatg

SEQ ID NO: 67 - DNA, 2500 bp, Artificial sequence
  sequence of T2R (STAR66R)
gtttggggtagagagaacatactgattatgggactttgctttgcagcttagtgctgtcctgtcagtgggaagcaacagggggcagaactcagcttgtgcccatagagggaatgtttatactaggcctgtccagaggcaaatcatccatcctagcaattggaacctgacttttggcaagtcctgccaccatgggctaaagtgttctggggttctaaataaacatgaaaggcaacctagaccacaaggactgcaattcctgcacaagtcctggtgctgtgttgggcttggagccagggaacttggagtgcatggaacctagtgagataccagctgagacaaccaaggaagtgcttgtgtcacccctccaccaaccccaggcagtacagattgtacctccaagaccccttccatctgcttgaggaaggtggaggggaagaggactttgttttgcaacttggattccagcccatccacagtagaataaggcaacgggcagactcctaaggcccccatcccagaccctagctcctggatgacatttctaaacacaccatgggccagaagggaacccattgccttgaagggaagggcccagtcctggcagaatttatcatgtgctgaataaacagcccttgggccctgaataattagtattggtagccaggcagtatttaccacaggccttgggtgagacccagagccatgttggcttcaggtgtgacccagcacattcccagctgtggtaactttggggagagaccacttctgcttgagaaaaggagacagaagagtaaaggggtctttatcttgcagcctggtaccagcttggccgcagtggggtagagcaccaagagagcacctgggataaacaaaatcaaaaaacctttagctagactaagagtaaagagagaagacccaagtaaatataatcaaagacaaaaaaggagagacattacaaccaatacctcagaaattcaaagtatcattagcagctactttgaacaactatatgccagtaaattggaaaacctagaagaattatataaattcctaacatatacaacctaccaagattgaaccatgaagaaatttaaagcctgaataggccaataacaagcaatgagattggagccctaatacaaagtttacaatgagaaacattgctcaaacaaatcatagatgacacaaacaaatggaaaacatccaatgctcatggacaggaaaaaatatttaaatttctatactgcccaaagcagtttatacattcaatgctattcctgtcaaaataccaatcttattcttcacaaaaaaaaaattaaaaattacacagaaccaaaaaagagcccaaatacccaaggcaattttaagcaaaaagaacaaagctggaggcatcacgttacctgtgatccacactatagggctacagtaaatgaaacagcaaggtgctggtatacaaacagacacataaaccaatggaatagaataaagagcttagaaataatgctccacacctccagccatccgatgtttgagaaagtagacataaacaagcaatgaggagaggactccctattcattaaatcaactcaagacggaccaaaaacctaaatgtaaaacaaacaaacaaaaaaaataactgctaaaaccctgggagatgacctaggaaataccattctggacagtacctggtgaaaatttcatgctgaagacaccaaaaacaattgcagcaaaagaaaaaattgacatatgggatcaaattaaactttagagcttttgcacagcaaaataaactatcaacagagtaaataggcaccctacaggaagggagaaaatattttcaatctgtgctctgacaaagtcctaatatccagagcctataaggaacttaaacaaatttacaaacaaaaaacaaacaacactattacgagttggaaaaggacatgaatcgacacttttcaaaagaagacatacatgtggctaacaagcatatgaaaaaaatgctcaacattactaatcattagagaaatgcaaatcaaaaccacaatgagataccatctcaaccagtctgaatggctgttattaaaaaaatcagaaaaaaacagatgctggcaaggttgtggagaaaaggaaacacttatacattgttggggggagtgtaaattaattcagccattgtggaaagtattgtggtgattttctaaagaactaaaaaggaattactattttacctggaaatttcattattgggtatatacccaaagaaatatgaattattttactataaagacagatgcatgcatgtgttcattgtagcactattcacagtagcaaagacatgttatcaacctaaatgcccattaacagtaaactggataaggaaaatatggtacatatacactgtggaatactatgcagtcataaaaagaatgagataatgttcattgcagcaacatggatggaactggagaccattatccttgggaaactaacaaagcaacag

SEQ ID NO: 68 - DNA, 2501 bp, Artificial sequence
  sequence of T3F
agatttgccctcaagattacaactgctggggctaaagtggtacagagcctgagttcagtaggcttccatagtctcactcaagaatgcaagtttacctctcaatctttcaatcatcacaattataacaactttaaaaagagccaacatgatatttgcttatcacttttctactcacattccagtattaactcaaaagtgtcaacacaaccttcgtgataaatactattaacgtcatcattcctactgtacagatgatgatagtgacacataggttaagttgcccaaggtcttattattaagggtcatagccaggatttgatctcttcagtaaagttctagtcaatgctcttaaccattaagccatgcaacacacccagagccaactgggttgtgttgatgattataatatttgttttaacaaacaataatttttcctaaatataatatagattttccataaataccataaattcttgattatttatttcactttattccaaaaggaagttgaattctgagatttaaatgaatagcaaacaacagttgcttaatttcactacttttgtcacttgtagccagtacttaaaaagagatacataatttatttttgttgatttgcatttcacatataattgtaagatcctggagaataaagactatatgtgttataccattttactctctcacacagtgtgtaggcctaggctttgtgcatagcaagtgttaaaaagtaatgtgactcgtgatagttattagatttattgaaattcagaaatttagggaaatgcacaataaaatgtacattttgtgattccggtcaaattacttaaaaattatatttttcctatgaataatttttatttcacttaaattatgtataacaaaataacatgcataattaaacatttaccacaaagaaaatatttgtactattgttatcacaataaagaacttgctacataaattcaattacacttttgtggaaagtatcttcattatataaaaacaatctacatttagaataggaaaattgtacaaaacatgaaaatataaacaaattaagcgagaattatctaaaaagcaactcttcagaatttagaagaattgtctagaataaaaagaatttagaagaattatctaagaaacaaccataaatattctgatgtatttaagactcatattctagaatcctgactattattttttatacttctatggctaatctcaagtttagctttatttttctaaagcaatgaggcctgtagaatattttttcagaattctctgaggttttttcttttttgtctttcctgtcatagtatgccaattattcatgggtttatagaatatgtatgcactgctaagagcagcaaaacaaaagatatatgtgctatttattaattcatgttgctttatttaaattacttgaaaatgataaagaaaaaactattgtatttacaacagcaaccaaatatagactacctgtaactacatctaacagaataaataaaatataacatacaatatgtagtaaatatatttataatatatatgttcactaaatagttaacctgtaacttacttacagtaaatatatataatatctactgagatagtaccacattttattaaggattaaacttttaataattcagaagaataaatataataaatttcatttgttctcaaactaatttgtttttatttgtttgttttttgtattttaatttgacagtagttccaagatattttggggtatataatgaggtgataattgcaaagaaaattctgaaaaggaaaagactaagcgtgaattgaaagtaaaattcgttaaaaggtataataaactgtgatactgtaacaataattgaaaatagataaagaaaaaggtaacatcaataaatagtctattatatatgtgaattatgttaataaaagtgacattttattttcaatccacaatttctgaaatatatatggcaatatttttctgttttattttttcaacctctgattactttattacatttttttctttttctagaatttacttgtattttctctgtgtctaatatatgattatttctgaactagcatcattggtcctggaaccagactatattattcccaaggtagagcatcaaaatataacaattaaataaatacttttagttactttaacaaccttttgtctttcattataattttggaattatagtttagtacaatacagatagttttaatatctgttagagtgaagatatatatatatgtgtgtgtgtttttgagatggagtctcactctgttgcccaggctggagtacagtggtgccatctcggctcacggcaacctctgcgtcccaggttcaagcaattctcctgcctcagcctcccgggtagctgggactacaggcgagtgtcaccacgcctggctaattttttgtatttttagtagagacagggtttcaccatattagccaggatggtctcggtctcctgact

SEQ ID NO: 69 - DNA, 2511 bp, Artificial sequence
  sequence of T3R
cttttggtgccctgtcccttataatttcctcgtgtgtcctttcccatttgcttatccgatgacttgcttctctcacccattggattgtgagcctcttgtggtcaggggcagtgctctgtaagctgctgtgtccccagaatctggcccagtgtaggcactcagcagctatagactgatgttaagagaaaatgcacatttcatctcagcctcagagcagttctgggaaacagataggaaaccaaagctctgcaagaacgtgggactctctcagggccatcacaacactgttgttggtctcatgtttggtgactgggtctcctattcctggtctctttcctaggcataatgcttttatataaagtcccttccattgtttttttgtttgttttcttttttcagcctaaataacttagtttctctaaacttttctcccagggactcttttttaaccctttgaattattgctgattattatcttaataacttttattttttttccattttgcatgtcatattttagcaaagcattaaaaggaacacggcacaaagcacacccatatttttggatgctgtggatttcatcatgctgcttattccattatatctagtcagtacctccaaggcattaatgctgccttacctccttcattcgaagacttccctgtgcaaggtggaatatacgtaaggaggcaaacagactgggttatatgcctgctctgctttacagaggcctcttccaggagtgtaatacgggggttgctcatactctgaagaagatagtggcaggctattactgtcatgagagccagaacgtggctggcttcttacagacatggcttcataggggcatgccacgtgattcctgagtaagccttctggtgtgaattccctgctcactggggtgattcttcacttcccacagttcaacctgctgtattatcctcttacctatgcttttctgtgatccatagaggtaatttaattttcagtccatgtacctaccctgcctacttagtttcttctcagtgccacacttaattccttcacatttactgattaattaaatgagaagactatgccaggtgaaggttcagcatcttcagaactctacatgatgcattccctgaggctgcctttcaataactgaggtgatattctttgagcagtgtgacctgttagaggtgcccagtcaggtccgatgaaaagccctctgatttgttgaaatagtgcattagtaaagtattatagtttattttcacaaagctagattagttgttacatgttggtttttgttttgcctagccctaacaagtatggaggtgaccttgatgtgtctatagaatatcaggaatatctggctgggtgggtggctcacacctgtaatcccaacaatttgggaggccgaggtgggcggatcacctgaggtcaggagtttgagagaggcctggccaacatggtgaacccccgtctctactaaaaatacaaaaattagccaggtgtggtggcaggtgcctgcaatctcagctactccggaggctgatgcaggggaatcacttgaacccgggaggtagaggttgcagtgagccaagattgtgccactgcactccagcctgggcaacagagcgagattctgcctcaaaaaaaaaaaaaaaaaaaaaaaaaaaaaaagaatatcaggaatatccattttatgtctcaactcacatacctcacagttttctggtccaatttttaggcactttatcaggccctcatatgttttcaaaaataattgctaatgactttgatgaagctaggcaagatattttttggttttagggcagtttgggctatagtttgcagccttcctactttaatagaagaatttttaaactagattctcccccttctcagggtggctttctgcctttccattctagtgcttcacacagaaatgacaagctcacaggggacttatctagaaaaggccgagataaaaataagtacaatgttaaaaaaatctatcttatagtatcatttatttagagcttcctctccttttctaatgaaaggctgctgtagtttccttttgtgctttttttgctgaaggcttttcagtaatattcccgtgtgtcccctgtgatgctaaaagcatgagcttgggggcaggttgactggcattcaggtctttgctcagcctccagccgcaagacaaggcgaataatattgatctcatggagctgaaatgaaaattaacttttctaatctgtgaaaatgctttgttataatccttaaatacatgaatacataggttgaaatagcaagtaccaagtgctgacattatgtccacaattgccacatgccatgtccttatgatttttgccagatgtttaataagattataaatgaataggttattaaatgggcatctcctactctctaggtgtttctgtttctgcttctctgttttctgtttgtatctccatttattttaatgcctaccattatgtgaagtctgccaccttcctatac

SEQ ID NO: 70 - DNA, 1500 bp, Artificial sequence
  sequence of T5F
gaactcaataggggtcttgtacggagcaggggcttggtccctcgtacctctggccatacctatggagcccaggggatgcttggcagcacctgggaggtgccaaccccgggtggcaagggagggccggtcccacgctcacattgtcttctgttctctctctctctttatctgtgtcgatgtctctctctcttcccccgtgcccgtgccatcctctccacccctggattcctgtctctgcttggctttcacccacttctcctccccacccacggctgctcctcctcctgtccccacctcctccccgggtgcaggacgggcctcttcacacctgacctcgcttttgaagccacagtgaaaaagcaggtgcagaagctcaaagagcccagtatcaagtgtgtggatatggtagtcagtgagctcacagccaccatcagaaagtgtagcgaaaaggtatgacggccgcctgggcggggctgggcctggccgtccattccttgtggccacagcctcccgtgggcagaaggatctgctgagccggcctcacggctacccgcagggacccagccctagtgtttcctgccagtttctaaccctgggtacttgcactcatgacccctccaggcccccatcccagaagacttgactccaacccaagcctccttggtggcacctatgctagtgatgaagatgatgttaaggagatggcagctgtttactgagcacctactatgtgccaagcacacgctaagtgcttgcccttactatctgactcagtcctctcaaccaccctaagacgtgggtagtgttgttattcccattttgcagatggcaaaacagagtctcagaaaagagaagcagagtgtgattcagttttaggaaggacagaggaaggggtctgaggtcagggcctcctgggcagggggagctgtcctagttcctcaaaaccaatttgcctgaaagcatattggattactcactttacagtaatccgtgcgtgagagacaggggcggtctcttttgagttgtctgtgactttttagatgcctttttcctatttgtctgcttttgggcattttgaggatttttagccaggttgtctaaagcagttcttcccaggggagtgcgagagaatcagttgcctgcaggagcttctccagcaggctaaatcagaggtgccaggggtgagcccagcctcacctatatctgaaggacttccctatgctggtgggtggaggcacatccaccttagcattgagtttcaaataagcatcaatcatctccattcctttttttttttttttttttttttgagatggaatcttgctctgtcgcccaggctggagtgcagtggcaccatcttggctcactgcaacctctgcctcctgggttcaagtattctcctgcctcagcctcccgggtggctgggattactagcatgtaccaccacacctggctaatttttgtatttttagtagagatggggttttgccacgttggcca

SEQ ID NO: 71 - DNA, 1500 bp, Artificial sequence
  sequence of T5R
gattacaggcgtgagccaccacacctggcccagtggggtccttctaaaatgcaaagctgatcatgtctcttcttccaggcttaaagccctcccatggcttcctgcagccctggtgcacgccttacgccaagcctgaaaacactctgcacacccaccccttccctgcacaaacgggcctctgcacactacctgccccggccatgcccccgcaaccagccctctctgcttatctaccttggccttctctctggtcaagccccaggcccgtccctgcccctaggccttcacttagagcctcagaagcacttcttgcaggaagccctccagactccagaatgggtccagaacctacttccttttcgtggcatttctgtattctttttttttttcttccatagagccagggtctcactgtgtttcctaggctagtctcgaactcctgggctcaactgatcctcctgccttggccttccatagtgctgggattacaggcatgagtcattgcacccggcctccacagtcttaattaattggttggagcattatttgcattaatatctctcaccaccctccccattcctgtccaagacctcagggagggccaggccagatgtatcatctgcaccagggagtcccctgcaggggcttccagatgtctgctaaatgaacacacagctctctctggccagtccaaggcaccccaggaggccaccagaagcctgcagcctccctccctccctcctgctaagcccaaggaatgagcactgagcagggaatggtaatctggacacatccatactctgcccttcagaaactacctagctgtcaccctgcacgaaacaggcaccagcctgagagtcaggaggcctgggctctgggtccacctagacagctgtggggcgcaggaccaaccgcaccccaatctctaagcctgggtttttccatacgtaaaaaaatgagggcagggcgggttagacactagaccagatctgtgatgacaggcccgttggaaggctggaggcggggcccctcgctgaaggaaaatgccttacctccagaagtggcccgccctggagtggccagcaaagggggcattgcccctgcgctggaatacacccagaagcagggtgtgagcaggagctgcggagaccttcagggacaggacagtctagggagggggtgagccctttgcagatctcctgcttatgccaggagaaaggtaaacacctctcaaacacacaaggagccagggggctgtgggctggaacctatagccggcaacagcgtatagcttaggattttatagcattgttctaccctagttatgtttcctatacttttgtttgtttgtttgtttgtttgtttgtttgtttgaaatggagtctcactctgtcgcccaggctggagtgcaatggcacgatcttggctcactgcagcctctgtcttccaggttcaagtgattctcctacttcagccttcctgagtagctggaattacagg

SEQ ID NO: 72 - DNA, 1199 bp, Artificial sequence
  sequence of T7
ccatcttataaatatatcataatttactgaaaaatatttcagtaatgttgaaaggcctctgtgccatttccagcttgaggctattcctaaaaatccttgcacatgtctttcagtgcacacatgtatacatttcggttgggtatgcctaggagtggaatgactggttatagggtacacttacgttgagctttggtagatactaccaactgccagttttccaaagttgtaccaatttacattcctaccaccagtacatgagggttccagatgctgaacgtcctcactaatgcttggtaatgtctgcctttttcattttagtcattctggaggtagtgtgataatatctcatcgtggttatttgctttagcctgatgattaacgatcctgaccattttttggaacatttggagatcatcttttgtgaagtaactactcaaatattttgcccattttgctactgggttgttcaaaagattcattaaaagaacttcttttatatatgggtttgtagttgttatttagatattctagagactagccagatccctatactacaaatactttctcctactttgtagtttgcctttttactttcttttatatacatataatttttccccctccaaaagacagggtcttgctctgttgcccaggctggagtgtagtggtgcaatcatagctcactgcagccttgaactcctaagctcaagcaatcctccttcctcagactctggagtagttggaacaataggcacatggcattatgcgcagtcaactttaaaaaaaaaaaaaattgtagagatgaggtcttactatgttgttgcccaggctgatctttaactcctggtctaaagcaatcctcctgcctcagcctccctcccaagtagctaagaatacaggtgtgcaccaccacatctagctttactttcttaatggcgtcttttaatgaacagataattcctaagtttgatgtagtcaaatcatcattttttcctttatagtcagcatttatatccagttcaagtaaagaatatcatgaaaacattcttctttgttttcttttagaaactttcataaagtagcatttaaaatgtgaattttcctataatcctagcacttcaggaggctgtgccaccgcactctagcctgggcaacagagcgagaccttgtctcaaaataaaaaattaaaaaaaaaaaat

SEQ ID NO: 73 - DNA, 1602 bp, Artificial sequence
  sequence of T9F
tgagcatctctgaactattgcgccatgtatttccaattttcatattgtgtatttgtatattttatatgtaatagtataggtgtaatatgtaaatatattttatatgtatttaaaatctttatattttgaagggttttgtttcaactattacttgttaatttcacagtccctttctttgatgttagcaaatagtaccttcatgaacctcagaggacttggatctgaatgtgcaatgccctctagtatttcaaataatagttcagttggtatagtatttttttaatctgcaaaaaacaatacttgctaatatagctatgttagagtaaacaataaatcgagaataaatttatagcctttgaaacaaaacaaaccaaaaattttactcctttttggctttcatccctgcactggtatcttaacttctgtttgtataaaagaataccattttttcacagaagacaaagaacaatcagccaatctaataattattttatggccatgctctgaaatacaattaaaattatgattgtggacaatatgccttttcgggacctggctgatggtatttctggtgtgaccccaactttccagtcagttcagggcaataaacattggatacaggacagctttggggatgaaatagaattaaatttagtgtagtttttgccacttttagctggatgcctggcgaggggttttgtgccctctgagagcctccgtcttctcaactgaggggtggttgtgagttttgggtcaaatgcttggtgtttagtagatgcttggagcttccatgaaacatgcaaccacggcgttgctgctatttgttcagatgcgagaggaacatgacttttggctgcctgagtgttctcatagcatctgggccttccttgtgagatcgtcagaaagtgtttcctgcacaaagcctgtactgcggccctggcgtggggctgattgtcccgctactctgctgtgatggctgaattcaaagagtggccgataggagcacgtatggtgggtgccttgttaacagctcatagcagaaacgtgacaagcgggagagggctttgggttgtcctgaacttcaaacacctgtaactgctgcgggaagagcggcacgtggatgaaacggacacagagggggaataggcaggaaaggacgcgggctcttttcgaagcagcaggtctcaaggcggccagccactggcgcagctgcagctgaagccacggcagagtctccatccttcccactatctgctgaatcagagaaagtggcaggcaacatttttagtgccttaaatttagaacgcttgctcaaaatcagaccctacttaaaataaggagcgataccctcatttcttaaatagtaaaaatgccctcagcagaattaacgggagtatcttccaacttcatatcctgaatggaaaagtctgtccaccatcccgaggacgtgtttgaagcgcagtgtgaaaatccagcacgtcgtggaccggccagacccctgtgccgtgagaggcggggcggcggggccgtggggcgctcgcactcccgagctcatcgtggcatgcgctgagccgaaaaccacgaggtagagggaatgagatc

SEQ ID NO: 74 - DNA, 1602 bp, Artificial sequence
  sequence of T9R
gagcttgattgtctggccgcgaaaacagggcaggcccgtgtccaacatgatagtgaccagggagacgaccacatccatgtagggcctggggagagacaggagggagcggtgggctgaggccagcctaggtggtggccctgcctgtagtcctgtggactggctgatgccaacagcctcaggtgtgggctcctgccacccacctcgcctgccacatcttgcacatccccgaggcaactttcgatctgctgcactcggtcacccgtactgcccaggcaagggctgcccatacgcactctggacaggctgagtgtcctgccctgtcccccacataaggctgccggccatggcttctgcacctgggtgggatgcagacacgctgacctgcctttctctgcggggcagtggggatgaacccaggttggactgtggccttggccaagtgacctgtatatgaaactgggacaaagcccatctttggcacgtagcctgtggggtggcaggtgctcaggctttggtgacaaggtggatgggatgcccagaaagggagagcccatggctgaaggcgtgggcaggattgtggggaaggtggttggaattagatgcccagagcaagaatttattggcacaggtgggcagacagaggtgaccaaaggacaggtgtaggtcagcaggtggctgctagcacctacctcactctctggaacccgattcccttcatcctaaaggggatctcagaacgttccacacaccccctccgcctccaccctggccctcacccaggctcaccgcacagccaggtagcctggacacacatctccatgaaccacttgaagggtgtggcctccatcttgccccccatgatcatcaccatctcatccgtcagcttgatgtcgggttcccagccgagattgccgcccggcgagctttcaaacatgaagccaaagtctgcaaaaccccaaagagctgcctgtgactgggtaggagccagggcgggcaaggacgagtggtctgttttgaggagtggaaaaggactcttcaacaggagcaccccctccacccccaaaaggcaggttgtgttttcttggagacagtgatggggtgggtggtggggcagcaggcagagaaagagaagggaggaagtggaggaaggagccaagctggggcactgaacctggaccagccccactccgcccagctccagcttctgactcagagcaatggcggctctcgccccagctccctggggccggggccaggcaccctctacagcagaacagcttggtggccgacagttcggacctcagagctggaccctgacactcctggcagggtggtcctgggcattctcctctctgtggggtggggatccctatccacccctgggtgccggggtgaagggagaggagggtggcgctgtggctggctgaccgatgtggatgatatggcccttcttgtccagcataatgttgccgttgtgtctgtccttgatctgcagcaggaacagcaggaggctgtaggcggccatgcttcggatgaagttgtagcgggcctgtgcagagagcgccctgggctcaaaaaggccctggggcctgtgggcattct

SEQ ID NO: 75 - DNA, 1301 bp, Artificial sequence
  sequence of T10F
aatcaaactggacccttatcttccaccatatacaaaaattaatgcaaggtggattaaagatttaattgtaaggcctcaaactataaaatcttaaaaggaaacctaggaaataccatctggacatcagccttgggacataatttataactaagtcctcaaaagcaattgcaacaaaaaacaaaaactgacaagtgagacctaattaaactaaagaacttttgcacagcaaaagaaactatcaacagaataaacagacaacctacagaatgggagaaaatacttgcaaactatgcatccaacaaaggtttaatatccagaatccataaggcacttaaacaactcaacaaacaaaaaacaaataacttcatttaaaaaaagacatgaacagacacttctcaaaagaagacatacaagtagacaaaaaacataggaaaaaaatacttaccatcactaatcatcagaaaaatgcaaatctaaaccataatgagatatcatctcacaccagtccaaatggccattaataaaaagacaaaaaacaacagaagctggcaaggctgtggagaaaaaggaacacttatacacttttggtgggaaagtaaattagttcagccactgtggaaagcagtttggagatttctcaaagaactaaaaatagaactaccatatgacccaacaattccattactggttagatacccagaggaaaataaattgttctacaaaaaagacatgtgcacttgtatgttcattgcagcactattcacaatagcaaagacatgaaatcaacctaggtgcctgtcagcagtgaattggataaagaaaatgtggtacatatacaccatggaatactacacagccataatagaagaatgaaatcatgttctttgcagcaacatggatccagctggaggccatcatcctaagcgaattaacagaggaacaaaaaaccaaataccacatgtcctcacttgcaaatgagaggtatatatagacataaacatgggaacaatggacactggggactcctggaggagggaaagaagtggcaggcaaagggttgaaaaactacttattgggtactatactcactacctgggtaatccgctagtagggatcatttgttccccaaacctcagtatcacataatatacccatgtaacaaacctgcacatgtacccccgaatctaaaataaaagttgcaattattaaaataaaataaaaataaagctagcaatgagccctatacatgaaaatcaataaaacataatcatggctgtatagaggggcttgtcatttatagc

SEQ ID NO: 76 - DNA, 1300 bp, Artificial sequence
  sequence of T10R
aattttacacacacacacacacacacacacacacacacacacaatatcgctcagccttaaaaacatgctactaatcggctttaagaaaagaagaaaattctgtcatttctgacaccatggaagaacttcaacattacgttaggtgaactaattcaggtacagaagaatactacagtatctcacttatatatggaatgtaaaaatgttgaactcaaaagtagagaatggaatggtggttaccaggccttgagagagaggggtaaaggttggtcaaaagatgcaaaatttcagttaagaggaaggagtacaagagatttattgtacatcatggtgactataattgataacaatgtgcttttttcttgacaattgctaagagtagaatttgtttatgggcaccaagcttgattccaagtctttgctattgtgaatagtgctgccatgaacatgcaaatgcgtgtgtctttttggtagaatgatttgttttcttttggatatatacccactaatgggattgctgggtcaaatggtagttctaagttctttgagaaatctacaaactgctttctgtggtggccaaactaatttacactcccattaactgtgtctaagtgttcccttttctccatgtcctcaccagcatctgttgtttttttgactttttaataatagccattctgactggtgtaaggaggtatgccattgtggtttgatttgcatttctctgattagtaaaatgaagcattttttgtatgtttgtcagccatgtatatgtcttcttttgagaaatatctgttcatttattttgcccacttttaaatgaggttatttggttttgcttgttcaattgtttaaattctttatcgatgctgtatattagacctttgttgaatgtgtagttttgagaatattttctctccttctgtaggttgtctgtttactcttttgatagtttattttgctgtgcagaaactctttagtttaattgggcctcatttgtcaatttttgctttcgttgtacttgcttttggtgacattgtcacaaattctttcctaaggtcaatgttcaaaatggtgtttcctaggtcttcttctaaaagtcttatagtttgagggtttacatttaaatctttaatctatcttaagttaatatttgtatatggtgagagaaaggggtccagtttaattcttttgcatatgactagccagctatcccagcactatttattaaatagggagtactttcctcattgcttatttttgtcgactttgttcaagatcagatggctgtaggtgtgtg

SEQ ID NO: 77 - DNA, 2001 bp, Artificial sequence
  sequence of T11F
tctttggggtatgattatatgtctaggtaaaactcttttaagaagatgaagcagagaggattgaattgacaaagacagctctttaaaaattaaggttatttcaagactaagaacataactgcttaattgcaggtaataacagaaaaaacttggaaataaacatcccattatttgacctccaaggcagaagactggcaccaaggaaatggcagcttcgtccctttcctgtcttgggcattggtaaaaggagttgtctagacatgtttgatttctgtttcagcccttattagtagttatgccatggcaaattattcaatttctctgactcagtttccttattcagaaaatggaagcataattcttgcctcatagggccatgaagattaaatgaggggtgtcttgaagtgtctgggacataaatcttcaataaaagctaattccttttttttacagttatctcaaaccttttagtgaattggtgcttatcagtgagctttttaggtgatgcaaagaccctgctttgctcattttaaggaacagttatttttctttctccattttgaagtttcttgtttgctgcctggttgatatggtttggctgtgtccccacccatatctcatcttgaattgtagttcccataatccccacatgtcatgggagggacctggtgggaggtaattgaaccatgggggtggttaccctcatgctgttcttgtgatagtgagtgagttctcacaagagctgatggttttataaggggcttcccccttcgcttggcactcattctctctcctgttaccctgtgaagaggtgtctcctgccgtgattgtaagtttcccgaggcctcccggccatgtgaaactgtgagtcaattaaacctcttttctttataaattaccaagtcttgggtattccttcaaagcagcatgagaacagactaatacattggtttaaattagaatgccaaaatttaaataatttttatcttgaatagtagatggaattaactttctcttgaaagatatattttaaaaaattgaacttacacagacagttttgaaatggtcttattttagttttatttatttatttattttgagacagagtctcacagtgtcgcccaggctggagtgcaatggcacaatctcggctcactgcaacctccacctccagggtcaagcgattctcttgcctcagcttcctgagtagctgggattataggcgcccaccaccatgcccagctaatttttgtgtttttagtagagacggggtttcaccatgttggccaggctggtctcgaactcctgacatcgtgattctcccacctcggcctcccaaagtctcaggattacaggcatgaaccaccgcgcctggctgaaattgtttttattatagatgttgcttgtgcagttttgttagaagttcgtgacttttaacagtgatgaaaatacttcgtcattcaacaggttatttttctgctggttgtaggttatttgtaaggaactgttagtctcctatctgggtggacatgtaatagtatcagttactgaaccagaactttaaacacctttctgatactcacactgggaggtcaccaagtatctcagaataaaatgtcccaaactgaacctaccatgttcccagaaacccagcccttctcaaattcccagacttggtgaatgggagcctgtccttgcagtcttgtagcccaaaacctagggcttaagaacaccttcttccttactcccatatgcaacccatcaagttccatgcatttcatctcctaatctcaaatcccttcacccatctccacagccaccccgctagtccgggctgccattgtctctcacttaaaatgttgttattgtctaactgaccttcctgaaccctttcttgcctctttccagtttattttccacactacagccagaaaaagcttttcaaaatacgcatctggtcacctgcatacctgtctccagaccacatacaataagccttca

SEQ ID NO: 78 - DNA, 2001 bp, Artificial sequence
  sequence of T11R
tctgccagcggctcccgcgccaggtcctcgaagcgcaccaggcggtagcggccgcgcaggaagggtggcggcttgagtgtggcggcctcggcgatgcgcacgtggctgcggcacacctcgcgaatcaggcgcaggtgagggtcggcctccacccacttgccgttggtgcccagcacgatgccgttgtcgcgtgccagtatcgggcccgccgcctcccgggagcgcagcacggcccgcgggtcgcgcaccaggtgcacgatgcgcaggttgagcgcggggtcgctgagcagcgggtagagcacctgcaggttgaagaagcgcacctccttgagcaccacgtggctgtaggagcggcaggcctcccgggccaggctgaatggctgccgcgtgcacagtgtcttgcatacgtcctgcttgctgatggtgcctcggggaaaggcgctgcaggcgggcggcgagcacagcgcgcggctcgttgcccagttgaaaaaggcggacaggtttcggctctgtggcatgtaggcatcaaacacgtccatgtcgcacaaaaagatagagcgcatcaggtcgcgcacggccatgtgcagcgttgccgcgctgccctgcgacagggtggtccacacatgccacgcgggctccatcaggtagaagacgtcggggtgctggctgaagagctggcccaagaaggatgagcccgagcgccacgaggacagcaccagcacgtgcacacgatcctcgccgccggctggggatgagggccctggccgggagatgatgaagagcaggaggcaggtggtctgtgccaggaggagcactgtcactgtcttgctggagaaccgtggcagccacatgcgggcggctgggggccttcgggtggagtgggcaactttagggacccgggccctcatgcccatcccatgccccaattactgcccagtgccctcagggatcagccctcagattcggctaccctacccattggacttcccaagactcccaaggtctcagtcgagcactttcccaggaatacggagtcaagacataggccagaatatagtctgtgctcacagcagaagtccagttgcagaataatgtgggatatcatcaaactgtctacctacccacccacccacctacttacatacctacaggctatctatctgtagagagaaatactatgtttcaaagagaactcctgtcttttgcttcaggatacctcttagagagacccttttaggttgtggagctaaaagggcttgatgggggcttcggtggatgtcagagcaccaccaggctcgccgaggttgaatcctggctctgccacttcctagcctatgatcttgcttatgaagatcacttaaatctctctgtgacggatcactttacccgtgtgtgaaagagggataattccggtacctggctcacaggatctggggggattggggggttattataatgaagatgggggaagggaacacgcagtcatgcccataactgaggattgcaccttttacaaggtgtgcttctgtattatataatttttttaacaggcaggtataaaacttttgtcagccaggcgcggtggctcacgcctgtaatcccagcattatgggaggccgaggcgggcggatcacgaggtcaggagatcgagaccatcctggctaacacagtgagaccccatctctactaaaaatacaaaaaattagccaggcgtgatggtgggcgcctgtagtcccagctactcgggaggctgaggcaggagaatggcgtgaacctgggaggcagaggtggcagtgagctgagattgcgccactgcactgcagcctgagtgaagagtgagactccgtttcaaaaaaaaaaaaaaaaacaacaaaaaaaaaacttttgtcattaaagataaacaagtaaataaagtggacaaagaacagcaactgttgtcatcactggtggggagtgaagtgctgtaggcagcatgggctccagaaggagggtgtcctggag

SEQ ID NO: 79 - DNA, 2100 bp, Artificial sequence
  sequence of T12
tggcatccagcatggagcccacagcttccctttgtagaattgcccagttgttgcagagtgctttggtctcaatgggtctaaagctcttgatgatataagagcttcaacttccttttccctctcctccccgcaggctgcacaatgtcctggtgaatcacctgggacttcagagctctgccaccctgggtgtgaagctcaggtctgctcttggtagcttggtcagtgtgaagtacaccgtgattttgggcaagctgcttaacctccctggccctccgtttcctcatctgtagaatggggatattcacagaacctacttgtagggccatggtgaggattaaatgatgaacagtgctggcaaacaggaaatgctatataagtgtccctagcaatatacacaccgcacatcctcagtcaccacgtgtgttcactgaggtatgggccatgtgtgggtggaattgtgttccctaaaaagatatgttgatgtgctaacttgaggtccctgtgaatgcaggaaaccaaaatatttcttctcaaaatagtgaggattgttaagttaaagacactgaaaatgcaggggaacactgccttggcctctacttgcctgatgacaggcacgaatccttccttacttaagacacatcacttgcttatcagcccagagaaagcacctgcaggcaccaggaaaatctaggaacagattttactctcttcccacattttcccactttttcaaacactgaaactgctctctcctttgtcttgtcactagataggatttatggctctttgttaaaatattgtttaagcaaggcttctacgccactagcttgagagagaaatacttttgaactgaggcctcttccgcatgataggcagagcatgcattaatacatttctgcttgtttctcttttgttaatctgacttttgttttccagagtgtctcaaataagaacataaaagggaggggagaaattatagtttctcccctacatgaacttattcggatatagggtctttgcagatgtaatcaagttaagatgaagtcatatttgattaggataggccctaattaaatatggttgctgtctttataaaatgagaagaagagaccaggtgtggtggctcacacctataatcccagaactttgggatgccaaggcaggaggattgcttgaggccaggagtttgagactagcctgggcaacacagcaagactccatctccaaaaaaattaaaaattagctgggcatggtggcatgcacctgtagccccagctacttggtgggctgaggcaggaggatcaattgatcccaagagttcaaagctgcagtgagctatgatggcaccacggcaacctgggtgacagagcgagaccctgtctcttaaagaagaaaaaaagaggagaaaaaaacagagacacagaaaaaagtccttgggatgataaatgcagaaattggagccatatatccacaagacaaggaaccaccaggattcttgggaactccagaagctaagaagagggcatggaacaggttctaccctagggccttcagagggagcgcagccctgcagacaccctgagttcagacttctggcctccagaactgcgaaagaataactttctgttgttacagcagccctaaggcactagtacaggtgacatgtattgctcttctgaagagcagggtgtctacagcggcagaggtctgggtcctggcacgtgccctttaggattccaatatccttaggggcctgctggtgctgacagttccagaaccataagacagaattcctgcgggccagtttggaagcagagacaggaaactggaagagcccttagcctgtgcttgggcttaaagccctttagcttgtggctttaactctgaaacttctagagggcatcttgcaggtcagtgtgaggtacagaagttgtcacaagcttcctggctcaaagaaagtgagacttcacgaacttttctggacatcacaccagcacttatgaagttatcttgttaagcacagatgaaatcagaaatacaggcattcaccatcacttaaacaaagctcagattgtagagtgcgaggaagaatcggtggga

SEQ ID NO: 80 - DNA, 1700 bp, Artificial sequence
  sequence of T13F
cagatctctaaagtattgggtgtggactagagctctggacggcctaaaggaaaggaatgtgccggttcacagggacccgcggctaagctcaagggtaaaatacagctttacaaagcatctttaggctgttccttcccaaacgtgcttagaagggaacagggaaaggcgggtgtgttttctcactgaggttcttctagtggctggaatctgatagagtaccaagttgtagggatatggatatattttccctttggcactccataaagctaaatgttgggctgaaaaaaggatgcagcctataaacaagtatttttcctgaaaccaactgcatgaggaaacgctgcgctccccctcagggagcagtttctgaagccagctgagcacagctggcactggccagagggagccctccaccctcccaccacgtatgcccacctgcaaacctgggttctgagtccccatgcaggggacagacctgaaaattccagtttgtgtcctttcaggtcatcgacaggaatgacagcctggcaagctgcagtgactgcacacagctaccctgtgagctccacttgtgtgggtgcaggtgggcgacaggagtgtgtgacacagacaggcactccaccaggaggaaacccacagcagacgtcaaccatcgctttattaaggctgcgagtcggggggctgagtcatgcactccacagacacccccactgctcccaaggtccacttttggatgaccctgaaggcagagactcctgagatctgggccacaatctagggtgagccacccacagtgccctgctggacaggggggtatgcggactgcacgggggggccctcagcaggggtcttcctgcctagggtggggctggctccagtgggtcctgggctcaggcagggggggtggcagggaggcagggacatccccccgccctctggcctatggctttgttgccctattgccaccagcgcagaagcaatgtgctataccgtgaggtgatgaagaagagccccgggagggagcaggcagctctgtgcctggggcctggccagacctcaggggtgctgtggccctgctcctgttccccctcagctcctcccagcaatgggtctcctccagtggaggtcagtcactcagaagtggacccgcagcacgtcttggctagcaaccggccgctggcaggctgtgcacgtcatgggcagggagcgttgcttctcacccaggcagggtcggcacaggaggtggccgcagggcagctggtacaccggctcctttttgaagtagggagaaaatactcttttgcaggaggcacattcggggcccaggatgctcccaggctgctctggtaaatcaggaaggaaaacaggccagggttaggaaagctgctccatggtccaggctgctctgaggggcagagccttcccaccgtgctgctgcagcatctggcttcatccctcccgagtccatcccagtctgatcaggtaggggagtggaagcgggagagggagcctgggaacccgggaggcctcttctctatcatctttgaccaaatctcagtgcctctacgaatgcttgagaagagctggcttctgagggcagcaggcaggactgggcccttcctcctggtctcccagcaaggtttactttcccctgcgataggtggccaaggctggagcaaggcacagctcactctgacaag

SEQ ID NO: 81 - DNA, 1701 bp, Artificial sequence
  sequence of T13R
gaatctgaccactcagtcccacatcccaggattcagagaaaaagaattccagtgagggctctggaccccacacagctaaggcttccagggtttaggcaagccctgagggacacccatcataattacccagacgggggcccagcatcccgccccagcattctgccttgcaaggagctccctcaccagggctcagggaagggacagcctgcagttccagcaagggaggcctgcagagtcagccacaggtggccactatcggttgcttggtgccaacttagtgtgagggggcagggcccagactcgagggtgccattaccgtcccccatcgtgtacttcttttcctcgtagcttgagtctgtgtattccaggagcaggcggatggaatgggccagctgggagagatggcccacagctcgggtcagagatggagggtccctgactttgtgacgactctgcacaaggggagccccatctcctcctctcgttcctgcctcacccgcccccaccccgcacgcccagccacacgcacagacagcggcaagcacagaccccgctgtcagggacagccctgaagaggaaccgtccctagagcccgtcctgcagctgctccacacttccccgcccccacgcacccccgtcccaccgcccagcggaccctggctcaccccgcggatgttccagtaccccagtgtcatgggcatggtgctggttgctgtggattctgcagacaggcctcagcggggcggggctcagcgtttgtgagaggcccagagagggtagaggggaagccttgctgcgaccccgccccacggcccgccctgcccccgaaacgggccaatctggaggcctggagcgcgctcatggggctaggagtaggatctcctcccacctcccagccccgtgggtttcaggagagagatcaggacgcccagaagcccagggcgggggagaactggttgagtccaggggttcaagactgaactgagctatgatcgcgccgctgcactctaggttaggcaagaaagaaaggctctctctaaaacagagagattctgaataaagtaataatagcctaataaagaaaaataacacaaaagaacatttggtgctcagggattcactggataagttttcaaaacttttcaatgtatgatagagattgttataaactgcggacatacgtggcatgacagacctaacgtgggaaggacaacacaggcaaggatgattataactcactgtcacttatcagcctaaatccaaacgtcaggaataccgcctcagagaaaagaaaatgatgtttttgtcataagtggtgctgtgctcctagggagcttgctgggtgggaagagagacagaaaggtggggagcaggggctggtggacttggggagggaggagaaagcccatgtggaaacgttagaatctggggtaatcagaggtctttgtattcattcgttttgtaaatttctcaaactctcatgttaaatcaaaataaaaagttaaaaaaaaaaaactaccaggacagacatacacaaatattattaactgaaataaatgttccatcaaaaaggacttaccttaactacatgagttatattatgatttctattattattattattattattttaatattagtatccatccagcacaccactggtcttcaagtggaggtaactttgcccctcaggggacatgt

SEQ ID NO: 82 - DNA, 1482 bp, Artificial sequence
  sequence of T14
atcagcccccacatgcccagccctgtgctcagctctgcagcggggcatggtgggcagagacacagaggccaaggccctgcttcggggacggtgggcctgggatgagcatggccttggccttcgccgagagtnctcttgtgaaggaggggtcaggaggggctgctgcagctggggaggagggcgatggcactgtggcangaagtgaantagtgtgggtgcctngcaccccaggcacggccagcctggggtatggacccggggccntctgttctagagcaggaaggtatggtgaggacctcaaaaggacagccactggagagctccaggcagaggnacttgagaggccctggggccatcctgtctcttttctgggtctgtgtgctctgggcctgggcccttcctctgctcccccgggcttggagagggctggccttgcctcgtgcaaaggaccactctagactggtaccaagtctggcccatggcctcctgtgggtgcaggcctgtgcgggtgacctgagagccagggctggcaggtcagagtcaggagagggatggcagtggatgccctgtgcaggatctgcctaatcatggtgaggctggaggaatccaaagtgggcatgcactctgcactcatttctttattcatgtgtgcccatcccaacaagcagggagcctggccaggagggcccctgggagaaggcactgatgggctgtgttccatttaggaaggatggacggttgtgagacgggtaagtcagaacgggctgcccacctcggccgagagggccccgtggtgggttggcaccatctgggcctggagagctgctcaggaggctctctagggctgggtgaccaggnctggggtacagtagccatgggagcaggtgcttacctggggctgtccctgagcaggggctgcattgggtgctctgtgagcacacacttctctattcacctgagtcccnctgagtgatgagnacacccttgttttgcagatgaatctgagcatggagatgttaagtggcttgcctgagccacacagcagatggatggtgtagctgggacctgagggcaggcagtcccagcccgaggacttcccaaggttgtggcaaactctgacagcatgaccccagggaacacccatctcagctctggtcagacactgcggagttgtgttgtaacccacacagctggagacagccaccctagccccacccttatcctctcccaaaggaacctgccctttcccttcattttcctcttactgcattgagggaccacacagtgtggcagaaggaacatgggttcaggacccagatggacttgcttcacagtgcagccctcctgtcctcttgcagagtgcgtcttccactgtgaagttgggacagtcacaccaactcaatactgctgggcccgtcacacggtgggcaggcaacggatggcagtcactggctgtgggtctgcagaggtgggatccaagct

SEQ ID NO: 83 - DNA, 1680 bp, Artificial sequence
  sequence of T17
ggcgccactacgggattaagcctgaaacccgagcggccccggcccccgccacggccgcctccaccacctcctcctcctccacttccttatcctcctcctccaaacggactgagtgctccgtggcccgggagtcccaggggagcagcggccccgagttctcgtgcaactcgttcctgcaggagaaggcggcagcggcgacggggggaaccgggcctggggcagggatcggggccgcgactgggacgggcggctcgtcggagccctcagcttgcagcgaccacccgatcccaggctgttcgctgaaggaggaggagaagcagcattcgcagccgcagcagcagcaacttgacccaagtaagtgcaaaagaaattgccccctgatttattgctgaaacctgtaaggctcgaatgtgcaaaactgatagttttactaacctataaaaacgtctagacgcctacccaagcctaggcgaacaacatgcatccataaaaagagcttcccataaccacctaccctgggcgctcagttagtacggtaaacagagcgcgagcattaaggctttttatgataattccccacaagttgtgaaaagcgaccatccttggtgaaattaatttaacgacctctcttccccaccctgtggtctctccctgcctcccctcctctcctctctccccgtctccaaacctccctctttgtagacaaccccgccgcgaactggatccacgctcgctccacccggaaaaagcgctgtccctacaccaaataccagacgcttgagctggagaaagaattcctcttcaacatgtacctcacccgggaccggcgctacgaggtggccaggattctcaacctaacagagagacaggtcaaaatctggtttcagaaccgtaggatgaaaatgaaaaagatgagcaaggagaaatgccccaaaggagactgacccggcgcggtgctggcgggagcgctcaagggcagcggatttgttgttgttgctgttttcctttgtgggtgtttggtgcttgatttccagaaactctccagcgacttggacttcttcttctttttttttttctttttagatagaagtgactgtgtggttggtctctgaggtatttgggggactctgtatttgctcgtttacgtgttggaaaaaccaagtggctttggggtttcgccctatcccactccctctctttcctgctccattggttccttaagaaatgctatattttgtgagtgcaagctggcttggggagccctctcttgtgtaaatgtcccccatgtttctgaaaagtgctgtagtttagtcccctcacccccagcactgcccaaacaggggccaagtgcgccccaattccaagaatgaaggcagagcgacaacagtgcggacaccccggctgctagcccacggtgaagcccggcggggttgcccaccagttgcgaaagccccctttcctcagggagcacgcgggacctcggtggagatctccagtgaggcttagaggagcccagggcctcgggcgggttggggtttgtcctcagtgcattggacgcgctgctctctcccctgaaggctgggctcgcgtgggcggccgcgggtggtggccctcccggttcctgcccgaggaccagttgtaaatgttactgcttcctactaataaatgctgacctgatca

SEQ ID NO: 84 - DNA, 919 bp, Artificial sequence
  sequence of T18
gatcatctactaggttgaaaggagagaatatgacttccagaacagcactgatgcttaaaaaggatgcctctggaagaaaaggaggaagaggagcaagtgatgggagaatacagtgggactttgggcaccatagggtcatcctgagtttttcaccaaaatcaggaacagcggcaaaactggtttcactgaagaagacacacgtttggagacatgtgtagtctccaaggattctcacttaacaaagcctatttctgttgttaaaaacccctgcataatgcacccacacacaaacacaaggcttggtctgtgttcctggccacctaaagaaactgattcccagtaagtttaaacctgaatgaaatgtttctgcaaattcagcctcaaaattcctcctctacctggcatccctggcttgtaaactatgtgtctcattagttcataaacaaagcagccctgactttgccttgtactcaaccacagccctaggagccagtagaatttgtccagaggtgctgggctttggagcccaagtggacaaagtcagaccccctttcctcagggcaaagccctcccacagggctgggaccccaaaggctatgctggaagcaggttcagcagcaggatatcaaggggcaaagctcctaattcaaaatcttcctggcttctgaacaaccattaggatggacagagaaaacttttgccctgctctgagagggtcccacagggcttttggaagcagagccaccattgagaaatccctttcaacctgagtagtaattcagatttttctcccactcctgcacaacttaatttgctgaatggaaaattcagccagaagtgatgggctgcttgaaatcaacaaaacttgacacattcttcccattttcattttactttattgttaaacacataattgatc

SEQ ID NO: 85 - DNA, 1174 bp, Artificial sequence
  sequence of STAR A1
gatcaatagaagaatggagtttgtgtttgctagccatagttttgacgtgtgggagagttggagtctagaaggttctctggacgaatgtcggcttgttaactgcaggaattcctctgtaagtctctgtccttacagaaaatggcccgaaattgaaaaaccctacttcttggaaaacagaaataatttgtgtaatgaatgttgcaggcggtgttggacgttcgtgtggagatattggcaatggtaggagacgatggtatcacacgttggatcgattaaaaagaaaaacagagtctctccatttgtgagtttctctcttttaattacttttgttactttaacatccttaggattcacagacgaaaaacagagacacccaatttttgtgtttcgagactgtgtcgtgtgttgtgtagttggtatcaaccaacttatatctgtaatcattgtttctttttatttattctcggtttgcagaaacatccgatgagcttgtcttagagggacgtttgttgttgttttctgggtctggtcgtgatgaactcgaaagcattgtgtgtttggttagtagtttgaaataggtgtgtgtattgtatttgtatatgctgcgtttgtgttttagagatcatcgtacataaaacacatcatcgtacataactaaaatttgagctaaactacaaaagaaagtaaccttcatttttagtcgaaccaggccccagctaggcagctatctcgtaaataagattgctggcttacgatcgtattccacgtggcaatttatgtgccgtggatttaaatttgtacgtggcatgagtgttaggagaatgtccacatggcttgtagttgttagtcccacgctctgaaccagagcaaccggctccttacacgtgttcggcttaaatccatttttcgaatgagattacacttctaaccttgtctccctctcccgcttataccaccaccactctcacacaagtctctcaagtcacaaactctgtttcaaaccaaaagggaactttgtgtgtgttgtcgagttttatggtgactgtaaaccctagccaagctcattgtttgcctatgaaaatgagtctaccgggtttcaatactcttccccacacggcaacaacgataccggtttccatacggagcaataggacgatgtcgttttttgaggatc

SEQ ID NO: 86 - DNA, 910 bp, Artificial sequence
  sequence of STAR A2
gatcaaaattttggtttcttcgctttgattttcttcttcttcttcttcttcttccctcaagttccttagaatatctttctcatccattttttttggttcttgttttgttaagtgaacattttagttgattttaaagtgctaaacttaaatgcagcattttactaatataaaattacgctccattattgaccttatatacatagaacaaaataatgttataatcttcgacttttttctaacaaatattaaccaatcatgtcactaagaaattaaaaaatactagtatataggaatctagtccattgtatatatcgtaaacatggacacttcaccaacgaacatgcatggggtctttttataaggttctttataccgaaaccattgttttggtttttatgataattgagttagttttgtggcttttccgttcaactaaaagtctcattatgtcaactgctattaaaccggcgcacatggcatgttttatgaaattaaggtcaattggactccaacttttcaattattaaaaaaaaagaaaaatgattgttgtatgccttggcgaagaagaaaagccgctagctttattcattatcaaacgaaacaaaaacaacaacacatcactaagaatcttaaactcttaaccttacatcaaagtaacttttattacattgcatacaagaaaagaacaaaccagcattattaggtttgagattaaacctgttcccacacatatacatagagatatgaactctacaatttcaaaccagagccttgaagtttctcctcaacaatcatgtcgattttgttttccatttcaggagtcatataactcttccaatcaccaacttcccctttacggaaaaaactcttgaaacttactccttccgacaagcttcctgttttgttgatc

SEQ ID NO: 87 - DNA, 906 bp, Artificial sequence
  sequence of STAR A3
gatcattaatcgcagatttttacaagacagcagcttggagagcaacttacaagtgtgttataaactctgaactcaacttggaagatgttgacgttccaaatgaaattggaagacaaactatcttcccaccaaggacaagaaggccgtctgggaggccaaaaaggctacgtatcaaatccattggcgaatatccggttcgtatttgtaggagtcccattttttcgactttatctttattccgtatttaattttcaattttatgtggtttaacagaaatcaaagagcgtgaaggtgaagattaacaggtgtggcagatgcaaaaagactggacacaacaggacaagctgtagtaatccaatctgaagatgttttaaaatcggctatattgatagaacgatgaccattttattattgtttttgtgtttggaaatggttatttttggataaaatatgttgcattctattttataattttagtttcgacttattacatataaatctagtaaggtaatatattagcaaattacagataatgatgaaaaacatggacaggtataggtggataagatataaataaggtaggactgaattgttacccgttaataatgaaagaatatacgaaatactaaacattaaataaggaagttactaattattggacaacaaaaagtttaattcctttaaaaagaaattggaatacagacagtttcattgacctaattaagtacttctttgaaaaaaatcaaactaggagaatagaagttgtaaataattgaagggaaacgtcgattcggtgaaaaggttttttaattagtatttaaagggaaatatcttctcttatacagaatatcttgccccagaacaaatcgcctcaaatactaaaagtgtgtacatcttctcttgatc

SEQ ID NO: 88 - DNA, 782 bp, Artificial sequence
  sequence of STAR A4
gatcaaattcatatgcttatttgtgattatactttgctttgattcaggaaatcaaagaagatagctccaccttacagggtgatactacacaatgacaacttcaacaagagggaatatgtggttcaggtgttgatgaaggtaatacccggcatgactgtagacaacgcggttaacattatgcaagaagctcatatcaacggtttggcagttgtgattgtttgtgctcaggctgatgcagagcaacactgtatgcagctgcgcggtaacggccttctcagttctgttgaacctgatggtggaggctgctgaaactaattaaactcagtatagattttcccaccttccaggactctctatttagtcaaaaacatttgttgttttaatgtatataatatcagaaatttggtacaagactgttactatatgcaatgaaccttgcccctacatagatctgttgtgagttttaagtgttttcatttggaacttcagaatgcaaataaacaaaactttattgaagtcaaatggtgttacagatgaatctttctgattctgtaatcactaatgtaaatgtatctaagcaattgtaagggagtgacgtgtttcggtttcatctcgcccaaaaaagcattcaaacccaagaaacctgcagtttcaagacattgatgggataccatatagatgtatcaagcatcaaccggagtaagaagcgactgaatgccgaagataatgaaaagcattccaccggaaagagccacctgcaacaacataagagctatttgatc

SEQ ID NO: 89 - DNA, 1356 bp, Artificial sequence
  sequence of STAR A5
gatcctgtaaaacataaagttagagataattgtccgatttgtttgcccttttaatttggagagatatgaaccaaaaacatatttcggaatgggtccctttttcatcgtgtgtaacagttttaccaaacagtaatactttgtgaaagttttgattaattaatgcaaaaagattagaaaaaagcgaaactaatttttggattacactagaaaaaggttaaaatcaataaccaaaaaaagaaaaaggttaaagttacaaaacacaccggtttatagagtgaaatgattattgttctgttgaattgacgtgccagcttagcatcaccttactattatcagtcacctatatatcacaattcacaggcttcttgctttctctcattggctcgtcttcttccctttcttctccaatcaccttagcttgctgatcaggtaaactagattggtgtttcgtgttgttttcttctcaacttaggtgtttgatttgagaagtttttctatgtatgttggcatgttgcgttcgtagcattgcatatcaacggataggtttgaataggtagaattaatttgattgatatatgaaagaatgtttgtatatatactctaggtctaggttattgaatattgagaaatttattttgttaggtttagatgaattattcttcgatgagtggttcaaagttcaattggcaagtcttttcaatgattgtagtattttggtgatgataagtaagttgttaatgactctcaagtctgaattcatgttttggttttgtttccttgtaaaaatgtgaacgtttttcttacagaagctttcacaaacaaagtatggttaattgagtgactaatccactaattctcttttgttgttttatatcgtttattaggtaatgttttttttttttgggtgtgtaaaatatgatactgactcaagattttatcatatttctgaatccataagctaaagtacatttgagagaagcaagagagatagaatggggcgtggagttagtgcaggtggaggacaaagttctttgggatatctttttgggagcggagaggctccaaagctagcagccgttaacaaaactccagctgaaactgagtcttctgctcatgctccacctactcaagctgctgctgcaaacgctgttgatagcatcaaacaagttcctgctggtctcaatagcaactctgcaaacaattacatgcgtgcagaaggacaaaacacaggcaatttcatcacggtatgtctttaattctttcgctgaatcgagtcctgtgtgctggttatcggatagcaaaaacatctgtatctttacttttcttagattagttgtctgaaaatgaaagaagatc

SEQ ID NO: 90 - DNA, 1452 bp, Artificial sequence
  sequence of STAR A6
gatcgactggtacaatgctagaagccctagaggttgtaggtgatagccacgatacatccttaggtgatgtaagtcaactgaatataaatggccatttacgtagacttcatgtcctagatgatccctcctattataacgtgaatctcggtttcttggtgtggaaaacgaaatgattgatatgtttttgtcagggatttgaggtggtgaacagtcgttatatgactagttatgatgatgaagatacaccgccaggaagtggattcaggacaaaactaagagagttccataagaggtaaatgacgcattaactcatgcctctcaacattttgtcggcattcaaacagatgcattcaagtctcttttaataaacacaagaatcccatttgtttattgttttgtttgtatgcagtgcggcatcattcacagaactagataggaattacctaacaccgttcttcacaagtaacaacggagattatgatgatgagggtaacatggagcaacaccatggtaacaacataattctctgatctcttgtttcactattatttttgttgttattccgcacccaaaaccatgaaatttacaattggggttattgcagaagaacgaatcccatttactagaagaggaaatctaaataaccgcggctaagtttccgagatgagaaatctaatagtgttttttcagcggcatatatatgtacataaaacaaactggatgtatgggaggaggtagtgacaaaggatttgttctaagctaggtttctctataatatggtactgtgttgttggtgtaaacctgaatggatattgttaggttgaaactaattacattcacacaaagaaagaaaaaaacttgaagaaggccatggctggtttatactgaaccacgaattttgttagttttaaactcttagggaaaatgctataatgccttttttgtcttgtagtcgtgtttggtttgaattaaaaaaaaaatagagaacgtcacggcacgccaaaagtgtggaccttgtttattcgccggaagtaagtaaccaaaaacgcttctaatctttcgtttacaacaaatatctctctctctctcgctctctctcgctctctctttcttcttcttcatcttctttcatggctgttactggctgggcaatcacaatctgaattctttcttcctccttgtctctctgattttcgccgagttttgggggctcttgttgttacacgatgagtctggtggttggtcagtctctgggtttaactctagtcggtgatggtctttcgttacgcaattccaaaataaatgtcggaaaatcaaagtttttctcggtaaatcggaggagattggcgcgtgcggccctggtacaagctaggcctaaggaagacggagcggcggcaagtccttccccatcgtcgagaccggcgtcagttgtgcagtaccgacgagctgatc

SEQ ID NO: 91 - DNA, 1085 bp, Artificial sequence
  sequence of STAR A7
gatctatcttatattgttagttcatgtttgtttttaaagactgtttttatgtttcaatggtatattactgactggggcagtaatattgttgaagtctgtagattatggtcgcatggctgaaatactggtgcagagggctgcttctcctgatgaattcactcgattaacagccatcacgtgggtaagcagaataaaccatgcttctgcttggcgtcttccagttatatagattggtactattttgacttctcgggagattcatatactaagaatatctgctttttattaaatgttgtagataaacgagttcgtaaaacttgggggagaccagctcgtgcgttattatgctgacattcttggggctatcttgccttgcatatctgacaaagaagagaaaatcagggtggtaagtttgcttctcctcctcagtgatggaaactgtaggttttgtatgcatctttttactttctttgttttttgatttttatttgcataaggttgctcgtgaaaccaatgaagaacttcgttcaatccatgttgaaccctcagatggttttgatgttggcgcaattctctctgttgcaaggaggttagtttttctctattgttgtttttatatccgtttgaatattattaaatcgcgcctgtttatttgtgagtttttgcattgagcaggcagctatcaagtgagtttgaggctactcggattgaagcattgaattggatatcaacacttttaaacaagcatcgtactgaggtgaagaaactggtttttgcttgggcatcattcttttctagttagcctttttgtttatcgcgttatagctaaattggtaatgctgcaacaggtcttgtgcttcctgaatgacatatttgacacccttctaaaagcactatctgattcttctgatgacgtaagttctatctccctgactgttcgtttgattggttggtgaactttataatataaaggtttggttttgtctagtaataaacttatttgatatttgaactatctggacttggaaatatactttaggtggtgctcttggttctggaggttcatgctggtgtagcaaaagatc

SEQ ID NO: 92 - DNA, 696 bp, Artificial sequence
  sequence of STAR A8
gatcatctttttctaggtagggaattgcttatctcggtaagctaagaatgttagaaacaaagaactaggacagaacgggaaatggagaaggaggttagaatcaaagaacagtaaatggagaaggaggttaatgtgtatttcattctatctacattttaactaattgagtgtatccagtcttatccattaatgtaattacaagaagaatagtaccaagcatgtaggttatagttttcactttactgggtgaaggtttctgtagttcaagtgggtcaaaagtggtttgcggaaacatatctctaataatttgattgagaggctcctcgcactcacatggacttaaacttttgtgtattatacaaacatgattcacatacacatctcgtgtatattgcaatacatttggtaaattatctgaaaataataatgaaggtttcttcaaaagaggtccaggagctatttccattaacactgttatactgaacagtatacaaaagaagactgcagtgcgagaatttatggaggatgataatgcatttgagatattcttctgaacactttcatatcttttatgtaaaacatttttgatgagaaaatcaccagtagtatccaaacactttaatccagatgatgggaaaatgctttgtttaaacctactacgaagtatgcttaatacttcattattaccagttgatc

SEQ ID NO: 93 - DNA, 925 bp, Artificial sequence
  sequence of STAR A9
gatctggtttcggtaattgttgtttccgggaattgagtatagaaacacaaatacatatttaaccctgatgaaagagggtgtaaacttgtgcagatagatgcgaaaacaacgcacgacaaacttgtgaagttggtgctcgatgataaagttagacgaaatgttgtatctcttattgttttgcgacaaatttacatgtcacggctgagttatatgcttaagggaagatgaaaagttcagtcaatttacatgtcaccactgagttatacgttccaggaaagacgaaaggttcgatagaattacattacggttgagttatatgcttaagggagaacgaaacgttcagtcaatttacatgtcacggctgagttatatgttccagggaagacgaaaggttcggtaaaattacattacggatgagttatatgtttaagggaagacatctataaatttacatgtcacggctgagttatatgttcaagggcaaacgaaagatgagtgtaaattatatgttacggctgagttatatgcttcaaggaagacgaaaggttcggtaaattacatgtcacggctgagttatcattcagggaagacgaaaggttgtgtaaattatatgttacggctgaggtacatcacgttaaggctgagttataatacagatcggaaaacaacatttttctggggaagacaatatgaaatttattggccaaagaacaacaatcaaattaagaaacgtaagaatatgtttgagggatacataggaggaagacgaaactatatgaatcaaaacattgatagaagtagaaatatctctaaatagatcgattgagaggaaaactaaacgagagacatataaaatcaaagtaaaagagtagttattcttgattcaactcaaacctgtaacaaatcatataaaattctatagatc

SEQ ID NO: 94 - DNA, 1753 bp, Artificial sequence
  sequence of STAR A10
gatctgaatgagatgtgttggcgaacgcatatagtttttgtttcttgctgttcataactttgcttatggaattttatttatgtctttctctatacctctttggaccagtgttccatttgcaatagagagtcactcgtgaaaaaaacaaataatgtgtgtgtatcaattattccctctcggccttatattttgtcttctttttgctaattatatactattgatttagatatttacttatattcatgacgtcttcttcttatattcttatttaatttgaagttagaaaattaacgttacaacttacaactattaaattattgttaattggttttataataagtatcgctcttgtctccattcacttgtcttttattgtccccagtaccaaactaccaaatacaattcatattcactaattaattagtttgatgcaaaggatgatgcaatgttaagaaaattgaaactctaccacattctaaaatgaagcaactctaccatatttaatttctttagacttggaatagtcacaatatgaatgcttaggtagttacggttagttaggagtatcacacagaattgaaaataccaaaccacaattttaatcaggtgattcggtactaatttttattaatgaataaaaacataaccgaaccaactcaaagcagatattaacctgaaaatgaactcaccaaaacaataatagaaagactcaaatcgagccggaaaccagattgagcaacgaactcatgggaatatcatatctatttatgtccagactattaatatacatacctatgacaaaatactatgcatgcaatgcaagactgaagtaaccatatttttttgggtaaaccattgataagctaaacttgaatatccatagtacttcatcgtactatgtatcaatagtatagtaagtttgacacaattacattcagtttgatttttatcatataaacctcccaacaatatttaaaaccgtatctatatataaatttatttgattaaatcagcctagaagtttatagttcagtgcagataaattcaaattttgatatatatcttaattgaattaaccgtcttttggttaaattattgttacaagcttacaaaatccactatacaccaagttggacttagatatcatatatgagattaacagccgattacacttgtacattgacctgacctatacaaacgactacaactttatgtatatatatttctctatttttggaaactcgtttgatttgttttcacatgtcgtgaaatttacagctttgtttcctactctcaaaaatagagcatagagctggctgatcacacttcaaattaaaaccaacaacgtatataaactataacccatgtgaacacaaaaatttagaccttttttcaaaaccattccaatttctaacaaaaacaaaattagaaatcctaaaatctgcaaggtgtatggaaggcaaaaaaggctaacaggattaaaaacagtttacattagttattctctttaaaatagaaagaagattttcgataaaaacgtcgtcgtatcttcgtcgacgtctccgtctttaatgggggagcaaagggcaagcggtgcttcctcctccaccgactcatattcaactccttcgccgtctgcgtcaccgtctccatctccggctccacgtcaacatgtcacgttactcgaaccatctcatcaacacaagaagaaaagcaaaaaagtcttccgagtttttcgttcggttttccgatc

SEQ ID NO: 95 - DNA, 1908 bp, Artificial sequence
  sequence of STAR A11
gatctcactcaagctcatgctcacgttcaaggactttccaaccgcaaggttatcttcaacttgtactcattaaggcctctcaatattcatgtgttatgttcatgtagatgtccggtccagttcaacaactgtttcattgctttagttgtcacgagaaatatttgtatatattattatggtgtgcaaaacatagtaaaatgttgttcaattggcagatgatgatgatgaaaatggaaagtgaatgggttggagcaaatggagaagcagagaaggcaaagacgaagggtttaggactacatgaagagttaaggactgttccttcgggacctgacccgttgcaccatcatgtgaacccaccaagacagccaagaaacaactttcagctcccttgacctaatctcttgttgctttaaattatttcatattgtaaattactttctgctttatcggttttaccatttcgggagtcttttttgtgtgcaatctgtttcgtttggtaagcttgtagtttcatgaaagtgaatgtaagatatgcattacgtttgttgctgaagtgaatgtaagatacgcactattatatctcatgattttctaagaaaaccctcttaaaacgaagatgtctatagcattacgtttctatttccatataatacgttaaaatttatggtttttacgtataaaatgcaaaataaagacacaagtatatctccaaagcaatgtaccgttgggaaaatttattagtacgttttcaattgtcaatgcaaataattaatggatgtgatagtcacaattaaacatacaataataaaaatgatgatgatgattcgatgatgtggtgggaaggataaattaaaccgactttggggcagtgacaggcagtgtcagtgtcaaagacaaccatttgtagtcactatttctatcgaaggttgcaaattgaatggtggaggagtatcaaaacgacacacatacttgaaaagatattttaataatataaaaaaattggtgatggcgtaataacaaacctagagctaattattatccttaatgataccaaatctatatgatacgatatttgttttaaaaagagtaaagactgacacttgagatgtgacactggcgatttcgctcacgtcaccacttttcccacctcaaataacgcttacggctttatccattaattctaagtataattttaagtgtattttttcttgccaaattcaaatatatcttactaaatggatgaacattataaaattgttatcaaaaccattaaatgttcttataatttctttcgttcctccaatgtcatcccaagactttttgacctaatatatgatatatctaacttgctttggaatcgtatgacatatatcttcaaatacatatttcgtatttttttttcacgaaaactaatttagaaagtagaaaaccagctattttaaagaaaataaagtgtgtttatatatattctaaaacaatgctataagaacataagaccaagatatatacaatgttattttatatttattattaagcattaacattgaaattaaaaatattaaacatgtataccaaagtaatcaacattgtagttattactactctctctgttcatttttgtttgattgtttagaaaaaacacacatattaagaaaacatattaaatattgattataaatgtattatttttaatgttttacagttttctataactttaaaccaatgataattaactatttttttaaaaaattaccattcacctatactaaccaataaagattacatagaaaactaaaaaaattaatcttttaaaaacaaattttttttctaaacaatcaaacaaaaaggaacagagggggaatattattttaatttaatttagattaccattgtagttagtaattgatc

SEQ ID NO: 96 - DNA, 1403 bp, Artificial sequence
  sequence of STAR A12
gatctattgctgtttatggcaggctgtcatttcagaaaagaatggtggtttgggatgtaatgttggtgaagatggtggtcttgctccagatatctcgaggtacatatatttttcctctctgatgctaatctgcttgcatctgtagattgtcgaaactgagaaaaccatgttatggtttgatggcttagtgcctaatatgtgtaattgcaactgtatgcagcctcaaggaaggtttggagcttgtaaaagaagctatcaaccgaacagggtacaatgataagataaagatagccattgatattgccgccactaatttttgtttaggtaattttctgcttcctggctaactgattttttgcggcttcttgtagtcatggatagtcttggtttggttctcggcattgtcattcacaattggctagtgagacgaataagatgttaaatcatcaaatgtgtagcctatcaatatcttgctcttgcaagtttcaactatgttatacgtttttgtgtattatttcttaccttgtggaactgttctttcctgaacaggtaccaagtatgatttagatatcaagtctccaaataaatctgggcaaaatttcaagtcagcggaagatatgatagatatgtacaaagaaatttgtaatggtatgtctggctcgtctgaacaatattttttgtgtctatcttagtactcttgcagtattgtaacgaccagattctctgtttggtctccttgtgggtttagattatccaattgtgtctatagaagacccttttgacaaggaggactgggaacacaccaagtatttttcgagtcttggaatatgtcaggtccaactcggttcccctactattaacggttcacatagattttgtgttctttcagatcacactgtcttctgattcttttctcagagtcaaatatctaaagagagagacccttaaatcttcttgtacaatcattttccttgtctaaattctcagtgttaaactcttgtaggtggtaggtgacgatttgttgatgtcaaattcaaaacgagttgagcgtgccatacaggagtcttcttgtaatgctcttcttctcaaggtatttcgtccgtcctattttgtttattactatgtattacctgtgcacatattgtatgtttactgcctaagaacgacaaagacataatgtgcatacggtgatacaggtgaatcagattggtacagtaacagaagccattgaagtagtgaaaatggcaagggatgcccagtggggtgtggtgacatctcatagatgtggagaaacagaggactctttcatctctgacttatctgtgggtctcgcaacaggtgtgattaaagctggtgctccttgcagaggagaacgtactatgaagtataaccaggtctggatc

SEQ ID NO: 97 - DNA, 1140 bp, Artificial sequence
  sequence of STAR A13
gatccatttcatatacatattaccaattttggcttttataggtttgtatccagaaggccttttcgtggctacgattaaggaaaatacgaaaacaaaagtgaattttactacttttgtagcatggtttattctactttatatacctaagaaatatgagcaacaattacttctgtaatgactttttactacttcgtagttggtacaaactacaaaagattgtgttgtttttacatgatactttataatatctatattaatatatttagtcgtgtttaatcaaaaaagcaccagtggtctagtggtagaatagtaccctgccacggtacagacccgggttcgattcccggctggtgcattgagctatgatgatataggcttcagcattggttgggtccattgcattcttctgaactatcagttgatgtatgccacacctctgagctcttctttttttttcctcgtcaattaattttttaaagttttgtctgcctaaaaactttcttctttttgattaatcatattaagcatctcggctataaaaaccacggtctactaacttaacatgcattggactagttttagtggagagtgttcgagttaaaatgagaagctcacgattgcataacggaacatttgattcgctaggcatctccatttgtaaaagtagccactccaatacaaaatggtcgatgatggtgagtgggtgagacaaacccaccaccacctcaagaagatatatttctctggttaagaatttgaatggttgacaaagaaacggtcactctatatacttagaaaatatagtcatacatagacaccatcggtctagttataataataaccactggattaatgcccagtgaaaataattgagtagccaaaacatgaatataacaatatcccaatttacatacaacaacacaaaggaggttttacacgattctatagtacaaactcataacaacaaaaaatcacacttttgtttaacagttgcctttatggctttactacagtatcttgtccagggttttcacacataacaatcacagtaaatcgtttccttttctttgcatcttccattccttttgtacacgtaacatctccggcttcccgaccatcagctaagaaccagatgcgatc

SEQ ID NO: 98 - DNA, 2125 bp, Artificial sequence
  sequence of STAR A14
gatccagcaactaagtcttatgctcaagtgtttgctccccaccatggatgggctatacggaaagctgtttctcttgggatgtatgctcttcccacaagggctcacctacttaatatgctcaaagaggatggtgagttcatcaactagttaatatgctcaaagtggatggtgtgtttgataaactagtagtttaagtagtcagattagtttcaaggtcttcacaggattaggtagatatcacggcaatatttggcctgtataagtcctggtatcataagagagaactctttgagattcacattggttttaagttcatttggcagtaggatattagattttgaattttccaatactatctctgtttgagatttcataaatcgagtttcttcttcattatgttcgctgacgatattgtttttttcatttatttatgaatgttgttacagaggcggcggctaagatacatatgcaaagctatgtcaattcatcggcaccattaatcacgtatcttgataatctattcctctccaagcaactcggtattgattggtgaagagcctgaaaaaaaggcataactattgttactctttagacaaaataacctatgttctcacatcaagctatgtaatgtcataacaacagcgacgaaatacattggaataaattgagtatgtccttaatctgtcgttttatctcttcttttaataaacacagtttatctcatagtaagcagaagaagctttacacgggttgtaggaacgtattaaacggtttgtttcaatttcactctctttggttttgaaattctagtataaaccaaagtagttggtgcttcaagttgtgttacttattcaacaaaaaaatatattatttttaatttttaattttcgtaggtaagattacatagtaacaaaatgttaaatttaacaatgtaagattactatgtaaatgcatgggcaccagtaatcacgtatcttgatgatatatatccctaatccaagcgagtcggcatttattggtgaagaatctcaagactcatagtcatcgctagttaacaatctttttcggacaaaagcgtcttcgttaaaattcggcattattaacctttttgcccttttaaaatcagaaaatttctgttttactggtatttttctttgacgattcaattttttagttgtattatatatatgaaagaagcttaactctctctcacagcttgatatgtcagtatctaaaacaagcaatacataatttaattaatttatcataaaatatttatgattaaaaagtaaagaagataaatattaaaaagctaaatgtctcttataatttaaaaataaaaattaaaaaggattgaaaagtaaagaagataaatataaagaaactattagtatcttataaataaataaataaactaaaaattgaaatataattattttagttttgaattaagaaaatattaaatataaaaaaaattaaacataaagaaactatatatatcttgtaattaaaaaattaaaaaaaaatgaaaaatgagaaaaaaaatataaactcttcatcatataattaatgaaatttaaaaacttattgcttttaattttttgtacaataattaaggaaatttagaaattaattattaattttagaagaaaaatgttaaaatagtttaatagttttgattcactaaatacatgtgtacatatatgatggtatgaggatcaagaaagtgccgtaaaatgtaaaacttccaatgttccttagtgaaaaatgttaacttttctgttgacaagacgtgtatataaacatcacctataccggagaagaagaagacacaaaacaaagttaaaaagaagaaatttttggtgcagtgaattcgaagagcaatatgaagaatattggttacattattatagccaccttgcttgttggtctcctcctcatcatggctctagtggcgagtttctattgggccaaacgacatgtcaaatgttgtggcggagagggactgtcgtcaaaggatgtgttcaatttacttatacaattggttgcttttattctgctttgtggtttatttgcttatttggtatttttggtttagattagtaacctaaagccatagcagatc

SEQ ID NO: 99 - DNA, 1196 bp, Artificial sequence
  sequence of STAR A15
gatcagcaattacagttggatggaaaaagagagacgagaatgtatctgctgctggtgactttaaggtaggctgagtaccaaattgcattctgactgttcttacctcgaccacctttcttactttccctagctctaatcttgctattactagattgaatctggtggactcggagcatcagctcgttatactcgtaaactttcatccaaatctcatggtcgcattgtgggtagaatcggaaggtatgttttattgacaatcccgagcaacctaatgtatgatgtgcgagaggatagaaatcattttttaagttgtctttacatgtgtggcgcaatcattgttctcattttactttggaattttttttttaacttattcagcaatgctcttgagattgagctcggtggtggaaggcaaatttctgagttcagtacagtaagaatgatgtatacagtaggactcaaggtaaactactctttaaaactttcggagccatcttagccattatgcaatctgcttatttccggtactcttatactttgtttgtagggtattttctggaaagtagagctacaccgtggtagccaaaagctgattgttcccgtgagtgttactttcttcctttcttttcttgtggtgtcatgtctgctgtcttcggataagaaccgaacagattgtgtcttaatctgtggagtagaatatattaaaaaagcataaaccaatagaaccaaagaccaatcctaaaagcctagggatggattctagagcattatccttgactctctgaaacctttacccaactcaattatggacaaagacaaacatccgtattactctggggaagtctttcacttttgacaccttcatgatgattatctttgaaacgtgcagattctactctccgcacatttagctccagtatttgcaactggagcattcattgttccaacatctctttactttttgttaaaggtgagtgattggaccctctaaatataatctacttttggtctattgttataagctgtttaccttattaaacattttcactgttccacgcagaaatttgtggtgaagccatatttgcttaaaagagaaaaacaaaaggccttggagaatatggagaaaacttggggccaggtgattgttacttccgagtttggtagccaagcgagattccttgtaattgtagatgatc

SEQ ID NO: 100 - DNA, 692 bp, Artificial sequence
  sequence of STAR A16
gatcgctttcagtctatcatgttttgagccttattttgggagcgatgtattaatattttgcctgttctttattttttgtgttgcagacatacaatgaagtgcagcggtgttttctgactgttggcttggtttaccctgaggatttgtttacatttcttcttaacgtaaggacatcttttgttttatgattatggctctagttattctttgtatatgtaacgcaaaacggtggcaatacctagcactcatattagactcaagaactattccttgccacacatctgtgtgatatttatatgggctttttatcttacatatttgaaatccctgtcttccttgtatactttcaccagaaatgcaagttgaaagaagaccctttgacgtttggtgctctttgcatcttgaaacatctgcttccgaggtgtattcttttatccttcatcagtataacttatcattcagagttaatttaccatcctaacttaatgatgttgcattgtgttcgaaggttgtttgaagcatggcactcaaaacggcctcttttggtggatactgcaagttctttgttagatgagcaaagtttagctgttcgaaaagccctttcagaggtactgagctggcgtagattttcttatttactactaaaatatgcatgctttagcatagtgcttctactttaatgacagttgatc

SEQ ID NO: 101 - DNA, 1826 bp, Artificial sequence
  sequence of STAR A17
gatcacgataattttccttaattatctaattctaagatagtctaaccatgaatattcttataatatcttaactgtataggagattctattttcatccctaaattatattcgtaattttattcggatatacttgcttttattttcgtcaacagatatatatatatatatatatatatattatttatttttaattttcattaaaattagtgatttaattctctattatttgtgtactatataaaacaaacaaatgaatcttataatgtttgctttttcgtccataaatatttccgggaaaaatcgttagatataaatcgaacctagtggtgagtgactcacacacatgtgacaattcccaaaataagtcccccacgtacgctatgtctgttttagtgtgcatgtagtaactattatttactgatttagaatataactagcatttggcccctatttagggataacattgttttagattatatctgttacaacttttaactaaaaattttaaaataaagcagacagtattaatatacaacaaatttattatcattgatcgaagaatatacaaagattaagaaaaagatataaagaaggtacaacttttctacccaatgaatcaattgcgataggcaataactaacaaatcaagagtttagaaatataagagagtataagtacgaaaattatgctgggtatatacatgtccgcttatttcatcattagctccaaccaattgtaatgtgttcttcttctcatcatcagtaattcagtttacaaacattcgttgacacccaaagcttggaagtctaaaaaaaaatgtaaaatgtgcacaaataagtaactacatgacgcagacgctgcctttgaaacaatatcaaagatattgcagatataaagaagtaaaataagagatgactttaaaattgaagtatttgtattaatacaaaaatcttgcgtgaaaatacaattgcagtttaatacaaaaaagaaattgcagatataaagaagtaaaataagagatgaaagaagaatagtaaaaagtatgagaattaatttaccatcaaaaaaacacttgagcttcgattaagatattaaactcacccttgttttaaggcaactgttcagatgagaagccaaaatttgtcgttgttccttgagtgtttgtgagacgggagaatcataggcattgattgtattaaagaataatcctatggaaaaatggagatgtatgagagaaatcgaattcagtcaaataaagcagaaacaaagcaaaaaaaaaaaaaaaccatagaaatctagaagaaggatatatgattttcggatctatggaaaatttctatatatataaaacaaaattacaaacagaaatagaagatggtaaattggttcattgagatgaacaaagtacctgatttctgagtaatcgattaatgatgttgagaaacccatttttgagattttacacagtagtcatggagtttttggaagagagaaagtggagatgtggagatcgtggggatgaaagagaaaatcatttgagaaagaaacaaagttaaataaaaacgacacatactatgcgtaaaaatgaaaaaataaaaaatagtactaagctgatgtgtcaatcactgaatgcattagttattggaaaagtgactgctgatttagtatatttagattagagaaaataaatacttgtaatcatttttcttattagcaatgttgaagtgaaaaaaaaaagaagaaaaaagtgtatatttatcatactcatagtgggaaattgataattcaaaattgctgataaacgttatgaaagaaggtggaggatc

SEQ ID NO: 102 - DNA, 1590 bp, Artificial sequence
  sequence of STAR A18
gatctgttgattggttaaatcgacgatctcaacggcggaggaagtgacgatgaaggcgcggcagagaggacaattagagtgagatttcaaccaagtatcaatacaaggaacgtgaaacgcgtggttgcatttaggtaacaatctcaagctctcgttctcttgaaactcgcttaaacaaacagagcaatctgaagattcaacaaatccatccatctttctgtatttgtaaacagttatcgatttaatcagagattcatcgagtccatcgccaccaccaccaccaatcgtttgattcggattcgtagctccgttgttgttgttgttgttggttccttgccaggtgtaatctgatgagattctgtttatagctgcggcggaggtagaggaggagttgtggcgacggcggtggcagtatttggagatgagagtgtagtagctgacgaggatgaaggcgctagcgaggattccgatgagagcgatgaggagaggagagaaatcagaggaggaagagtcgtcttcgtcgtcgagatagaaggaaggaggaggagggaagatgacgtaacaccattgagggcaatagacactgcatactccttgagaacagtctctgtatgaatcgtatgttgtaccccatggattagggtttcctgttgaacccattatttgattgttggagaaagatagagagagagagcaaggaagaagatggaggtgtcaagtgtctctctcctttttctttgggctctgcttttgtctggtaagtgtctatttttttatttcgagttaattggtattattagaggagataatgaataaatatatatgttcatgaaagcttttgcatgatggtgttaatactaattgaatgatgtttatagtgaatgttctactttatcaaatttttatttctagtatgaataaaggtgtagaatttgctttattcatttttattctttagctttctctttatgcttccattttttttaaagataaattaatacattagtaaaataaatggagttcattttttttttttttgattttattttgagaaatgagaacgtaacataagaagtgttttagtgttgacgaaataaaaagagagagagggtttagtctatttcaaggcataaaaaaatggttggtgaagtgttgacgaaggtggaatactataacatgggccacgtggatgacaaatttactcctcgacgtatctattaaagttgtggtcagaaatacagtacaatttaccgactacctacatggaagaagaatattttcatttcatttcaactacagtagtataacattcacgttatacgatttttcatttttgttttgtaatcaaagtaatgattttccaaaaaaatcattgctatgattcgaatacatacagttttatattagtttacatatttatgacaactataatacaaaattttaatagttgttcaagggacgattgatgtgaactcgccaaccatatgccctacgtacaaaataacatatttacatgtagaagttgaaaataataataataaagtgtgattaaaaacaattatacaaatgctaacaataggctacgagatc

SEQ ID NO: 103 - DNA, 706 bp, Artificial sequence
  sequence of STAR A19
gatcttgatgtgtgttttgtgtttttgttattgcaggatgtatgtttcatagtgagacagggcttaagagctttgaccatccgactaatatgatgaaggcaatgccgaggattgatagtgaaggtgttctttgtggagctagtttcaaagttgatgcttgttctaagatcaatagtatccctagaagaggaagtgaagctaactgggcgctggctaattctcgttgattttgcttctagtttcgttaactcttgcttctttgttgcgttttctttttatgtactcttgtttatgtaaatatagccttatgaagacgataaagaaataaaattgatttgcttcttcgtgacatagcagtctttacttagacaactgtgtgataaattcgcaatctcactctttgatagataagagggagggaagaaagcagtggtaaagacaaaactgtgttgattttgtgaatttagaagtttacaatagcaaaaaagaaactttggtcgacttttatcattcatcgttccacatgtctgtaaattcatcaggctccaatgggtttgagagttcatgcatctttcttcttgtttttgcctttattttcttagcaaatttcccagctttatttcttttctccaaagctcgaatctaaaaggcaggaaattggaatatatgagaactctgacagataatcatatatagcaatgtgatc

SEQ ID NO: 104 - DNA, 2064 bp, Artificial sequence
  sequence of STAR A20
atcgtttcaaagcatggtctaatgatgatcctgatctccgactgatccaataacggttaagcaacgctgtttttgatcctccattgttgtttgccatcgatcaacactcagaaataaggtaattaacgcatctcgagactcattgttttaacaatctttgttttgtttcttccaaattattctcgtgaatatccgtaatctctccgtcttttaatgaacaacacatatcatatgcttttgtttgttttgttttgttttttcaacatttcaataattttgtctttttttcttcgatttaatttgtttatttcctgctataataaacgaaaactataattccatgtaatgttcgttgttgttcatagtgatttatcataacgagcaacaacataaaaatcaagagaataagaaattagagttatgctgcttatttgaattagacaaaacctacttttacttgttaaggaaatgaaaagatgttaataaagatgagcacatcgtacgtggcgcacgtggaagcacttctgtacgacggacccagtccaactcgaaccccacacacatagcaaaggttgttaagttggctcgtaggtgaatttaatacctgttatttcctttatagctggctaattacctaaattcgatccataataacacattcctactatgccaacatttaaccctagtcaaactaattaaaacgtttcttactttttggcctattaaaacgtttcattatgttccgcaaatagtatgaaatatataaagattttctaacaaaaaattactaagaacagttagactgattgagattgtttttatttccttttatttaattttcttttattatactctgtttatttgtgtttaataattaggattctatttgtcttgtcttgtttgctatagttggagttttgttcataaagaatggcgtttaatacggctatggcgtctacatctccagcggcggcaaatgacgttttaagagaacatattggcctccgtagatcgttgtccggtcaagatctcgtcttaaaaggcggtggtatacggagatcgagttccgacaatcacttgtgttgtcgctccggtaataataataatcgcattcttgctgtgtctgttcgtccggggatgaaaacgagtcgatctgtgggagtgttctcgtttcagatatcgagttctataatcccaagtccgataaaaacgttgctatttgaaacggacacgtctcaagacgagcaagagagcgatgagattgagattgagacagagccaaatctagatggagccaagaaggcaaattgggtcgagaggctgcttgagataaggagacagtggaagagagagcaaaaaacagagagtggaaacagtgacgttgcagaggaaagtgttgacgttacgtgtggttgtgaagaagaagaaggttgcattgcgaattacggatctgtaaatggtgattggggacgagaatcgttctctagattgcttgtgaaggtttcttggtctgaggctaaaaagctttctcagttagcttatttgtgtaacttggcttacacgatacctgagatcaagggtgaggatttgagaagaaactatgggttaaagtttgtgacatcttcattggaaaagaaagctaaagcagcgatacttagagagaaactagagcaagatccaacacatgtccctgttattacatccccggatttagaatccgagaagcagtctcaacgatcagcttcatcttctgcttctgcttacaagattgctgcttcagctgcgtcttacattcactcttgcaaagagtatgatctttcagaaccaatttataaatcagctgctgctgctcaggctgcagcgtctaccatgaccgcggtggttgctgcgggtgaggaggagaagctagaagcggcaagggagttacagtcgctacaatcatctccttgtgagtggtttgtttgtgatgatccaaacacatacactaggtgctttgtgattcaggtaatatgtgttcaaagttactactttcaagcaaatcctctgtttcctcacatcatgatc

SEQ ID NO: 105 - DNA, 1834 bp, Artificial sequence
  sequence of STAR A21
gatcttcttctatatataccggtataagtcaactggcggctgaacaaaggtcgtgaggtaacaaaatatgagacaaatctacaggtcagattgggttctgaattctgataaggtcttaaaaaggagctcaccaacccacaaaaccatggattgaacaagtacaggtcattgccttcattttattctttacttttctaaggctcaagcttcctttattgcctttaataacaatatactaatgagtattttgcactcagtaacaaaattcaggagagtaattttttgccctaacatgttacttttatgtgttaagagtttagaattttggatctatgattttagtttttgttagggaatcatattcatataaataaaatattgccattgacttaattgttgttattcacctaatttctctccaaatttggtcatttacctcagttgattctatattatacttgctaagtgttctttgtctaattctctatcattgtttgatttaataataaccaaaccttaagacttggaagcaaagaagagagaaaatcccaattaatttttaataattcaaagagagatattgagtgacttccactaatacaaagaaagcttggtttgtgcaatattttgcggttaagctattaattgctgaggcaacaccttttcacactttgctttccttcttccaagttttcaacttttctttcttactctttctattaatcaaactgcaacacaaaaatcatttggataatacatgtttagaagatgattaagctttagttttatttcaagattatcataattgttatctgttgttacctacattcatataatcttatcaaaaacgataaagacaaaaaggggatacaatataggtttttattataaagaaacaggaaagaaagaaaagggttttcaccaaacgaaattagttcaatcatttaaattatctttatccttatgattagtgtctttatatctgtcatatgctgcttctccttccaacttcctttggattatattctcttctctttattttaatttccatttgtggtagctgttttattttttgtattttcacgccgtgtccctttaaaataatattaactacaccactaatgttggaacatgaaaaacatgaatgaggtaattatgatgatgaaccaaatgttaaggacaagctcggtgtaactaagaagataattagtgaaacagaacaagtcaataacttgtaagcatttcagaattgaaaataaagataagggaggatgaatatgaatttagtaaatgggtaatgaaagtgaaagaagaagagggaagggttggttactgtctcaagggtttgaaatggagacggttgcttgagaatgaggaaaaagagttagtaagtttttaactctctctttctctctccctctctctttttcaacgtcaattcctttaaggaatggcctctctctctctctgaaagtgtgtgtgtatatattaaacgactccatttctcctctgcttagaccaaaactcatcttctatactgcaacaaagaaggaggagccgttgagactacaaaatgactgcagcagaaaacccttttgtatctgacacctcttctctgcaaagccagcttaaaggttcttatttttctttctgtttattgttcatcaacccttatgagtaatttgcttgatgttgaggttgttctgctttcttttaattccactctgcagaaaaagagaaagagcttttggctgctaaagctgaagttgaggctttgagaacaaatgaagagctcaaagacagagtctttaaggaggtaacatgcatgatgatc

SEQ ID NO: 106 - DNA, 751 bp, Artificial sequence
  sequence of STAR A22
gatccattaagaagcagccgcaaaatcggattgagaacaggaaaagaggcggttaaggcttatgatgaagtcgttgatgggatggttgaaaaccattgtgcccttagctattgttcaactaaggagcactcggagactcgtggtttgcgtgggagtgaagaaacttggttcgatttaagaaagagacgaaggagtaatgaagattctatgtgtcaagaagttgaaatgcagaagacggttactggagaagagacagtatgtgatgtgtttggtttgtttgagtttgaggatttgggaagtgattatttggagacgttattatcttctttttgacagaaatacattgaaaactaccgttgctaatttgataggtatacatatatagacatgtatatattgtataattatatgtcaagattatttatttattttacatttttcacaaaaaaaaacgttaatctatttttctgtcacaagtgtgtttttattcatactacatactacaacgccaatttaacatgccaaatataaaacatacatgggcaaaggcccaacagccagtttaaagaactttgtctgaagagaaagttgttgtatatatcacaagggatatgtggtaattgggaaacatgttgggttgacacgtgggaaattgaaggagatggagtttccgtcactggtagaatcttctaacactagagagcttcaattcaggttgaaatcgtcagaaaactaatgcagacggtagatc

SEQ ID NO: 107 - DNA, 653 bp, Artificial sequence
  sequence of STAR A23
gatcaaaacttagtcaaatcgttccttccattttctttcagtttgattccactttaatggcgtcataatcatctcttaaatcaaacaatgactccactatctcgtttccgatctcttgttacataaagttttctgtagcattgagattgtccttttcggaattgcttttatttgcgcagcttgatggaaacaacaaacagtgtagtagtttagtagaaagactgagagataaaacgaagagtcaagttcctaagtccattacttgcattaaccgcttagagatatcgcgtatagcaccattacacgcaacgatgaatagcccgaaaggatttggacctcctcctaagaaaaccaagaagtcgaaaaagccaaaacccggaaaccaaagtgatgaagacgacgacgatgaagacgaagatgatgatgatgaagaagatgaacgtgagagaggtgtaattccagagatagtgaccaacagaatgataagcagaatgggatttacagtggggttaccactcttcattggtcttttgttcttcccattcttttactatctcaaagtgggattgaaagttgatgtgcctacatgggttccgtttattgtttcgttcgtcttctttggtacggctttagctggtgtgagctatgggatc

SEQ ID NO: 108 - DNA, 548 bp, Artificial sequence
  sequence of STAR A24
gatcagactgaactcgtgtactctgagccttgcttcttgtagctcttttagctttcacattttcatcagtattcacatcattcctgataattgtgccagaagtcccacgactatcttgttgctcactaatggttgctgctgcagatgattccatgttgtcctcttgtgaaaccccaatgcttcgtctagcaactgtatttcttgcacttcctgctttgcggtttttacatttggatgatgcaactttaactttaggtagcttcttttgagtaagatcaatctcatctctacctaggacctgcaaatcgatgaaatttgagttcatttcaacacacttgatgacactatcatagaaaacaaaaagaccttgctgtaccagagtgaagaacagcctttaccttggccttcacaggactaggtagaatctccggagaacaaggcctctgagtccattcaaacatttcgctatcaaacatgtcacctggattgggcttttgttgctcgtcttcctgaaacattcatcggaaaaaaagtaagatcaaaggatc

SEQ ID NO: 109 - DNA, 1000 bp, Artificial sequence
  sequence of STAR A25
gatccaaactctgcaatgtatattacgaagtcgtttgatataacacctctcttgataaaagatgattagaacctaaagtaattttaaaatatggtgaaaaattagactcttggagtatataaatggctcaatctgtattgcccgcaccgcccaaactcccatggcaaatccattgacgaaaccaaggtaaaaatcacatgctttgagcgtttttttaaaacagaagtgtaagcttaaattttttagtttaatagtagtaacaaattcaaccttgtgaagagatttattaataatattaaaatcattcccctaattatttgccttgagtttcgagccttctactgtaccactcacacattaaaaatcatcagactattcaaactttcttacatggttgattagttcatctcatatatgctcagtatcatactcttgcagattaatttttcattttaattatcaacgaattttttatttaattattcatgaccaaaatacatttattttttttaaataaaacaaataataaatttggaagtcaaaaatacaatcaatagaaaaaaaagtatgacagtgatagataatatttgcagaatattatgtgaaagctattttctctgtaacaataaatgagaaaatctttattattttacatgaaagaaaaagaaaacaaaacagagatatttttccagctgaaaagaacaaacatctctcattgatgttcagtgaacttgcaccaaacttcacttcttctatacttcttcatagccacaaactcagttctttgcaagaaacacaaacttaagtattcaaaatatcgtcatcatgttctcaagattccatgctctgtttcttctccttgttctttcagtaagaacatataaatgtgtatcttcatcttcttcttcttcttcttctttctcattctcttcattttcttcttcgtcttcttctcaaactcttgtcttgcctctaaagacccgaataaccccaacggatc

SEQ ID NO: 110 - DNA, 1926 bp, Artificial sequence
  sequence of STAR A26
gatcctcgattcttatctggatacagaagaaaacacctttttgtcttttaagtactcggagaaatctgagggtatctttttcttgagcagatggaggtgaagtcctgagttggggaggagggggctctggaagacttggccacggtcaccagtccagtctttttggcatcttaagaagtaacaggtttgttttacttaatttcaatatcgttttgtctctttctcatgcattttttgctcacaagaattttcccatttcctcctttactttatcatgattccttcataattttcttgtattgcactgtaaagtatccccctgattgcagtgagtttactccaaggcttatcaaggaacttgaggggatcaaggtaatctagtggtgaagaatatccaccttggatgaagagtttctagttacctagtggtggttttaatctttagactttcatgcttatgtttttccattctttctgtcgagcactaggtcacaaatgttgctgctggtctgctgcattcagcatgcactgatggtattgatttactttcttaaaagtatgaatgttgtgccatttaccgaactttatgaggtttgtttgcaaatgcagagaatggctctgctttcatgttcggagagaaatctataaacaagatggtaagaaaatgtctttttctttgatttctgtggtcatatatgtgaagctatctgatgggaaaatacagggctttggaggagtaagaaatgccacaacaccatcgattatcagtgaagtaccatatgcagaagaagttgcatgtggtggctaccacacatgtgtagttacaagtaatactctcttattatatcgttctttctttgatattgagtttgcttgtatactgcaaatgcctgtcctgctcaaatttctttttgttattctttatagaggcccaaaactgctctttagtttctgctaaatttatgaacatattgtgtttgtaagatggtcgataacaactcatcgtttgatgtttccttcgtttttggaaggaggtggggagctttacacctggggctcaaacgaaaatgggtgccttggaacagagtaagttacataccccgaaaaaatagaatgtttccccataagatgaaaacaaggttcttgaactgtacctatactcttatttcaaaaaattcagttcaacgtatgtctcacactcccctgtgagagttgaaggtcctttcttggagtctactgtatctcaggtatcttgtgggtggaagcacactgcagctatttcaggtagcatctcttttgagtaaaacatatttgtttcctctctcattgtataagttaattcaactcaatttctgaaacttgtttgcagataacaatgtcttcacctggggctggggaggatctcacggcacattctctgttgatggacattcctctggtggacaattggtttgtttcatcatcttatcttattgatcaaatctctgaaacaacattttcaagtgtcgaagagaataaatatggtatgcttaatatgtagggccatggtagtgatgtagactatgcaagaccagcaatggtggacttgggaaagaatgtaagagcagtgcatatatcttgtggcttcaatcatacagcagcagttcttgaacatttttgaagactcggtctcaagttaatatcatatacagatgtttagtttattcttgcttaaacatctatagactaaaaaaataataagaaatttacactattgaatagcgatcaattacaccattggttctaacttgaacaatttagtaaataggtggaatattcttgtcgtgtaaattattgattttatttatttatttttgaaaactacaacaaacgatagaagagttgaggaaatctctttgtaatcataattatgagaaaattaagatc

SEQ ID NO: 111 - DNA, 1109 bp, Artificial sequence
  sequence of STAR A27
gatcggaatcattttgggagtttgaaggaactaaacataatatgcatgtcgaagtcaacttattgcaaataattttgaaatgattctgaattggaaattcatgaagcttaattattttatctaaataagtttaatataggtttgagtgagatatcgagattaaatgataagagtctttcttcgaggagacattagaattctacacaaaaatcgaaattaatctagtccttgacaatcagttttcaattaatcaaaaacctataaaattcaactcaaaaccaatcgtatgaaacttcattataccatataatctggttacttagcttaaatctctacccggcgatgtttcatgcttgagagactaggtacataggacactaggagtactgcatatatggttacctcatgagttctcatcgtaaaatcatccaataaaaaatggtttcctgcttaggtatacggtataccatcttgtatcgttaaaatttatagctcagttcgttgctaacagtcaaatacgtctttccagggtaaaaaatgtggaaatttgttccactgtaaaaacctaataatttttgacattaataattaaaagggattataatgtaatatatacaaagataggggagacagagacgaaggcccacacatctttaacaaaagaacaacaagcccgtgaccccaaaataaaactagctttcagatttattatttttcatctgacataattgcaaccgttagatttcatttctcaggtcccattctgactcagatccaaccgtccatattcctctagtgtcttcaatagttgggccccttttctttttcctctcgccgtacactctccttccagcgccaacgccaccgcccgagccacttcttccgccggcgccaccgcgatttcctcgccggaatcccctccttcgccgcctttcccgtagaccacggaaaggatgcttatggcgtattctctccctctaccagccaatctcgccatcaccgctaccatcgccggcaccgtcatcgcgtgagcgcgaacctccgccgctccttctgccgttgtacacattagctcaagagcagctaaggctcgctccaccgctgagatc

SEQ ID NO: 112 - DNA, 1659 bp, Artificial sequence
  sequence of STAR A28
gatcgaactttggtaacatgcttgcttactgctttctattgtctgcaaaacctctgttctgggtgaccttctggcccctctctctcgaagcttcagaactatggaggagagattggataaaggagacaaaaggtgtggtgtggcgaaatgttagggtaccggcaattgtgtatgtatgagttgattttgttcttttctcataaagaggatttaacaaaggatgagaaaacaaatccaacttgagtactacgaggagataaaagcttttattgggtattgagtattgacacgttgttgaaagtctgatacattttagacttttactgcatatgtccaaatatttagatttttttttcgtttctcaaaaaagtaacttgtttaacaaaaaaaaatcgttattgggcttttcgtttcttttatattgggccttgagcctttttagcttttgtatttttagtccttttcgggtttatttatttattaataagataccaaaaacataacaaaaatgtagttttgtatttttaacctagtcttttaaatatttaaacttaattagaaaaattctatttaaaatattataaaaaaaacatgattttgtgattttcccatattttgtgtaactatttttgacaagcttttgaaacaacaaagacaaaatccatgtgataaggtcggtcaaaaatcttgcgtagtagaggagttaaagatttttggatggttacaatggtatactcttatttgatatcccatcaatggtatatagctttgaatggtaggacaagtgagagtaaaattttctcatcattgctaagttttattttaggttctacattgtttcacccttcttaagtatcctactctcaactagaaaaaaaaattgtgagggcggttttatcggctggaatgcagctcatgtagctcccacgacggagttttctggctaagaaactcggacacaacgttggcctccaatatcttcaaggcttcttcattcgtcaccgacctcggtgtcttatactgactcacagaagagcctctagacagaaagaagttcatgagcttgtcgaaagcgccaggcttaacaaccttaatctcaagtggtccaatgttcttatcattctttcgtccttttctgtaaaccgcgtccagagactcctcaatggtgaagcagcattcctccaaaacattctggtcaagctcaagcttggcgtccttgacttttctcccgagttcccagtagagcacgtagtgacctggatacgaggaggaatccacacggctagtgaaatccatgagcattaggtcatgtggctcaagcaggagactcgcgttagtcactgccttgaggaggtcttcgtcgtaggtcttgtccatgtcgatgctcagaacaactttctgtcttcccacgaaatgaaactgtggcgcattgttgtagaaaccagtcactcttaaaacgtcccctaaacggtacctatacaaacctgtataaagaattttgatacacattaagaaaattattaacatgtcatttagttttgaaattgagagagtaaacaagaaaaaacacttaccagcaaacgttgtgacaacaggttcataatcatgaccgattttaacatcgacaagatcgactacaacaggattctctgcgggatc

SEQ ID NO: 113 - DNA, 874 bp, Artificial sequence
  sequence of STAR A29
gatcagagtcacaaccataggagtcggagacggccatgcatgtgtcttgatagaagaattaaccggttctaaatctgaaaacgaatccggtcgtctcgaaccgaaatcaataaccggtccggtcaaagaaacggttgcacgagtgaaggaaacggttacgaaaacggagccgttaatatgcgatgacggagtgacaaaggggaagctgacgatgtgctacgaggtagacgttgacgttgacggtgggaggtgtgttaacggagatttaacggcagttagctacggaggaggtttgggtaattgtggcggggattggtgggagaaatgggatggagtggtgaggatgagaaatggtgatgacagttggtaccgttacgtggatttaacggtgattaatggaaatgtggtaaggttatgggatgacaacaaaacactagtaacggcggcatgtgtctaaattagagaagtttcatatttcggaaagtttttaaatcttgagaagctttcttggtttgaagtgttttttttttgttggttgattaagttgtaatttgtaaataattttcacacaagagaccaagaaggaacgcttaaatcaatatcaattggtgttgattcccagctttttctagtcgaacttaggtaacacgtccattgcgatgatgaattcgtgacaaggggtcaactatttgaacacaacaaacaagtgcgttttcttgttaaggcccatctaaaattgactacacacatttacttttaggcccattttaaacttgactgtagcctgtaggcatgtatttgttcgtgttactcccagcctcaaacccgcaaaatccacgaattcttcttacttagtctagactctggtctgatc

SEQ ID NO: 114 - DNA, 2138 bp, Artificial sequence
  sequence of STAR A30
gatctggctaatccgtttagcacacaaccagatgtaacattggttgcaaagattattgaagagtctcgatctaatgtaacacacctctgcgcattcaggagtgcttacgtcaacacattccgggaacgaaaaactgttagcgtatgtgtattttaaagtattaccatatttctttatatcttctagcacctcctcacaaatgtcacgtgcgtcctccgattccaaagcataatggttgcttccgaagagccgaaggtagacaccacccatgtgagcattaccagcacatatgtaaagaattgcgcatgcaagggttgcagcggcgttccttggggcgattcgctctaagtgtaggatagctccctggatgtcagactcatgtgtaacgagacgcagtccttcatggtaaatagccgtggggttaccagcttgtaagcaccgtttgaagaagggtctatagcgaccttcggagttgatgtcatttggatcgtggcctgccgcgtagaagtcatcgggatcgtcgcacatgctgaaaatgtttgcatttttgaggacatccggacagtagacaatgtctcttccacgaggaccggatttcaacataggtccgaggtaccaccaacatttgtcagccattttcttggctatcttcgcaagcaaatcgtcaggaatatttgggtttgtcatatttaggagtaaggtgtttcgagaaaatgaaatttgaacacttaaataagcatcattgaagatatggttgggtaagttatggttgtatttattgcaaaggtattaagtgatgatgtgtattcatattgtcaaatcaaagtaatagtattccatatataatttgttatcgttgttatgagcaacctctttttattaacagcttaaaactagacgtgtacgttttactgacggtcttagtgtacgtccacatttacatttctacatttactcaacaaacagtgtacgttgtagtgtatgttttagtgaacgtccacatttacatttctacatttgcccaacaaacagtgtacgttgtagtgtacgtccacatttacatttctacatttgcccaacaaacagtgtacgttgtagtgtacgtttaagtgtacgtccacatttacatttctacatttgcccaacaaacagtgtacgttgtagtgtacgttttagtgtacgtccatatttacatttctacatttactcaacagacagtgtacgctgtagtgtactattagtgtacgtccattcataaatatcaccatttatgagacaaaccaaagacctcatacgtttgcatgtgttattttttagtgtacgttagagttgatatctcatgctagtgaacgtccatatctagttttccgagacaaagaaaaaacctctaagtattatttggtagatgcacgtgtacggagttgtggacgcttagattttaatatccaaatttacatttactgcagtgtctaaatatcatatgtgaatttggctgaaaaatattcaacttgagaaacataacacaccttgcaaatttcttaagcaataatataatttcaacataaacataaacaacatagtagaaggcttatcataatttgaaacatgacatagcggataacataaacaaacatataaagtagaatggaataactatagcatttgactaacacgcctggcacacgaccagaggtaacagcggttgcaaacgttttggaaagctcctgataccatgtaacaatataaggcgcaaggaggcatactaattccatggctggtaggataagagaacgtaggaccatatgtattgctgtatggagggtcaaacttctttatttcctcgatgaactcatcacccaaaactcgagtggcaaccgagtccaatggataatggttgcgggtgaagagctgtagaaacaagccgcccatataatcatacccagcacatatgaatacaatggcgcatgcaagtgttgcatttgctcgtactggagcatgacgctgtaagagcctgatggctccattgatgtttcgttcatgcgttagaacacgaataccttcgtaatacacggccgtgggattattagctgcaaaacaccttaagaaaaatgttcgatgtcggccttcatcagcggatc

SEQ ID NO: 115 - DNA, 2092 bp, Artificial sequence
  sequence of STAR A31
gatcaaaagaatcgtacttgaaatatttagtggaacgcatatgtcagagttacagatatggtttaactctttttatctcctttttttaatggtgtttctctttttatctcctataatcttttgggaattttttattattaaatattaattaaaaagataaattcttagagaaaatcccaactgacttgttaactagtgagacatatcttatttattctctgcttatctaaaaagaaaatgaaaaagaaaaaaaaagtatatattagaagattaatataagtttagggggaaaatgattattattactatttataaaattagtatatttcaaaattgtacaattaattactaagccttaaaataaaaatgtaaaagaagattatcatcaagaatagtataccatctttgtttcaaaagaaaagtttactaaaagaaaaaacttttgtttaatttctactaaagctgaaaggaaaatgattgtcaatttgttattattattatttatatgatagatttcttaagaaacgtatagagttagttacaaattctaaattaaaaattgtatgataagattatcttaagaaagttatacaatatattcctaattctaaaagaaaatggttatttttttggaatagatatacacaacaaaacaaatttagtataagaagatatgttagattaactaaataaacatctcaggcatgaaactggattaggttaaccagaggtccagagacctatatatctctaggcattagggtttaactacggagcaaagcctcataatcaagtttatatcttgcgcatctttagcaaccaatcaattatctaagaagccatgactaatactaatgttgctgctacaaagcctctttctactatggtcgatgaatctcctagccttctccgtgattggtggtgagactctagatcaatgatttttcttacttttttcccattactatgttatgttacgtaacataagatggattaaactgaatctgatcctcttaaattatattggttgcagtatgaacaagaacctacaatacaactttgcgatgaacttcgtcatgataatcatcaacattgaagcaatcttgtctatcagaaaccacgaaaatcacgtaaggaaagattattcaacgattttgataatttccggtatgttcttgcctttcgcctattaagttgcgtttgttgggttggcgcaatcagggatgtgacattatgtgaactcgcctacatcttcggacgcatcagtcacaacataggctttattttcttcctagaactcctctattgtatttctccctacttggctctactcgttggtctacatgtaggccaatggtatctaacttccatgattggactgtctctatgggaaggaatgcaagcattacgaactgatatttaacctcgtttaatagtaaaatctaaacttatttagctgcatattttggtttaaggcaatcgagaatgtcttagcatctaaagcttacttcgtgggacgcatctgtcacacgttcggcttttgtattttcgtccacctcctctattcggtttctcctcacttggctctatacttcggtctcccttgtttgctaggtttcgtagccgtcatgattgcaccaagttgtccgtatcaatggaaaggcctatgcaacaaagtgcaagagttacgagactggtggaagcatgtgaatcgaccacaatcctcggttgttattgttcaaggatctccatttctaagatgtgaattttaggactcttttatcccttttgccttttaaattggaataccaacgtttattatgtgggttagttatgtgtgtatatgatatacaaatcaaacaacatatataaggagaagagatattgaatgttgattcttaatttacaggaacatgaagctcgggtctttccggcaatgccatcaatatccgaggcggtgcagtttcttcgtcagacgagaaaccagagagtctagtatcctaattttgaacaaatagagcataaaggaacaagttatatagcttcacataacccgaaacatgttttaagtttcaatatcaaagacaagatc

SEQ ID NO: 116 - DNA, 1290 bp, Artificial sequence
  sequence of STAR A32
gatctagacatatgtgtgagacgtttcattgtaggtatctgaatgtaaagctcaaagctttaacctttgaaccgataaacctctaaagctctctcttttccttggatgagtctcacaagttaagaacttcagtgaaataatctgactttattgaacccaaacttgggtatcactgtttatcttagcattacagagttttgtttttgttatgtacattggatttgaagtctacaatgtttttccaggtttataaaccggaagaatatagccgggttctagctatctgtggtcctgggaacaatggtggtgatggtttggtggcggcgaggcatttgcaccactttggatataaaccgtttatttgttatcccaaacgtacagccaagccactttatactggactggtcactcaggtttgtgtaaccagtgcttaatttatgggggatctttgttagctttctccgtttctttactgcctgctgaatttgcctgtttttgtagttggattcactctcagtcccttttgtttccgttgaggatctgccggatgacttgtcaaaggactttgatgttattgtagatgcaatgtttgggttttcattccatggtaactatttttgtgcatgaatcgttagaattcttcaaagcatgaaacaattataagaagtaaattcatcaaacttttgaacagcaagttttggaatcaaagtctcagagatgcaccttattcatttgcatcatgtttcagttggcctttgaaaatccattttttgcacatgtaggagctcccaggcctccttttgatgacctcatccggcgattagtatcgttacagaactatgagcagactcttcaaaaacacccagtcattgtctctgtggatattccctctggttggcacgttgaagaaggagaccatgaagatggaggaattaagcctgatatgttggtaagtcttagccgaaatgcttgtgtttctctttttctcttgtactcatttgttactatctgatataatgaaaactactttataaattgaacatatttactctttttaggtatctttgactgccccaaaattatgtgcaaagagattccgtggccctcatcactttttaggtgggagatttgtaccaccttctgttgcagaaaagtataagctggagctccctagttacccagggacatctatgtgtgttagaattggtaaacctcccaaagttgacatatctgctatgagagtgaactatgtctctccagaattgcttgaggagcaggttgaaactgatc

SEQ ID NO: 117 - DNA, 869 bp, Artificial sequence
  sequence of STAR A33
gatcccgttcatgtatttttgccagttcgagttggggttggttctgtttactttttctagtccatgtattttgcagacctattaaaaccattctgttttttttttggaccaacaaaacccatccgtttttagatacgaaaataaaattttattaaaaccattatttttcttggaccatcaaaacccatccgtttaaagatacgaaatgaaattcgattgataaatacaaaataaagttcaccaaacttaaataaaaaggcatagatgggaccaatgagaaagaaatttcttttctcctcaatttccccaaaaatatataaaccttaagtttacttttttgttgcaaggaaaaacattaatctttttcaactttctaaaaacaatcatttcaaacgttaaaggaacctcctcctttctttacgcgtttgcaatataacccaagaagaccgcttgtttgtacaactttccaaaaaccaaacagtagtgtaataaacctctgacttcttttttcttctctatttttgtgggtgataatcaattcactcggtttgaaatttcgtccacttttcaaagatgagtgaatgaaaaagccacgaaactttccatttcttcctctgtgtataactctcactgagtacgacttgccattttctcatccaaaaaaaatgtttatccaaatacatatttgtgaactttgcttttaaaccactcaagattcttccccatggcttcttcgtcttcttcttctcggtctcgcacctggagataccgcgtcttcacgaacttccatggacctgacgtccgtaaaacattcctcagccatttacgtaaacagtttagctacaacgggatttcgatgtttaatgatc

SEQ ID NO: 118 - DNA, 921 bp, Artificial sequence
  sequence of STAR A34
gatccatgcttttgagtttaagtgatttatttaagatcctctaaacttttttttcttcacttagtggtggttccagtcaatttagcaagtaagatgttgtatgtgtcaatgctataactgtgaattttcagctattgtagtttgatttttgtctttgttagcttcaggtgtcttgaatctgaatctgtggctatatttggtgctcggtggtgagcaggaagggagggggatattgtcagggttttaatgtacgtcagatgaatagagcaactaatgttactggcagtagaaggagggggtttattctcagcgtccgcgtctgggtatagtaagggattgacccttcttttctctggtgataaagacgtagataggcccatgagagttgtcccgtggaatcactaccaggtggttgaccaagagcctgaggctgaccctgttcttcagctggattctattaagaaccgagtttcccgcggttgcgctgcttccttcagttgttttggtggcgcttccgcgggacttgagaccccttctcctcttaaagttgaacctgtgcagcagcagcatcgtgaaatatcatcaccagagtctgttgttgttgtttctgaaaagggtaaagaccaaataagtgaagctgataatggcagcagcaaagaagctttcaaactctcgttgaggagtagcttgaagaggccctctgttgcggaatcacgctctctagaagatataaaagaatacgagacgttgagtgtggatggtagcgatctcactggtgacatggcaaggcggaaagttcagtggcctgatgcttgtggtagtgaactcactcaagttagagaatttgagccgaggtacgtgtgatatgttttcctcttattgagttgcttaaatcccaatacgagttaatttaagtagatc

SEQ ID NO: 119 - DNA, 1140 bp, Artificial sequence; identical to SEQ ID NO: 97
  sequence of STAR A35

SEQ ID NO: 120 - DNA, 381 bp, Artificial sequence
  mouse ARE sequence
gcccggtgctttgctctgagccagcccaccagtttggaatgactcctttttatgacttgaattttcaagtataaagtctagtgctaaatttaatttgaacaactgtatagtttttgctggttgggggaaggaaaaaaaatggtggcagtgtttttttcagaattagaagtgaaatgaaaacttgttgtgtgtgaggatttctaatgacatgtggtggttgcatactgagtgaagccggtgagcattctgccatgtcaccccctcgtgctcagtaatgtactttacagaaatcctaaactcaaaagattgatataaaccatgcttcttgtgtatatccggtctcttctctgggtagtctcactcagcctgcatttctgccag

SEQ ID NO: 121 - DNA, 13 bp, Artificial sequence
  FRT sequence
gaagttcctatac

SEQ ID NO: 122 - DNA, 6 bp, Artificial sequence
  oligonucleotide
aaaaaa

SEQ ID NO: 123 - DNA, 34 bp, Artificial sequence
  Anti-repressor #40 sense primer
atatgggcccggtgctttgctctgagccagccac

SEQ ID NO: 124 - DNA, 34 bp, Artificial sequence
  Anti-repressor #40 antisense primer
gagtgagtcggacgtaaagacggtcccgggtata

SEQ ID NO: 125 - DNA, 19 bp, Artificial sequence
  Nramp1 target sequence 545
ggacggctatctccttcaa

SEQ ID NO: 126 - DNA, 19 bp, Artificial sequence
  Nramp1 target sequence 870
ggtcaagtctagagaagta

SEQ ID NO: 127 - DNA, 19 bp, Artificial sequence
  Nramp1 target sequence 666
gctttcttcggtctcctca

SEQ ID NO: 128 - DNA, 19 bp, Artificial sequence
  Nramp1 target sequence 915
gccaacatgtacttcctga

SEQ ID NO: 129 - DNA, 19 bp, Artificial Sequence
  Nramp1 target sequence 2196.
ggctcacaaccatccataa

SEQ ID NO: 130 - DNA, 55 bp, Artificial sequence
  915 shRNA forward
tgccaacatgtacttcctgattcaagagatcaggaagtacatgttggcttttttc

SEQ ID NO: 131 - DNA, 59 bp, Artificial sequence
  915 shRNA reverse
tcgagaaaaaagccaacatgtacttcctgatctcttgaatcaggaagtacatgttggca